SEQUENCE LISTING (7 sequences)

SEQ ID NO: 1 - protein, 4 aa, Artificial Sequence
  Synthetic
GFLG

SEQ ID NO: 2 - protein, 4 aa, Artificial Sequence
  Synthetic
KPLR

SEQ ID NO: 3 - protein, 5 aa, Artificial Sequence
  Synthetic
VPGVG

SEQ ID NO: 4 - protein, 12 aa, Artificial Sequence
  Synthetic
HRHTKQRHTALH

SEQ ID NO: 5 - protein, 4 aa, Artificial Sequence
  Synthetic
YLLL

SEQ ID NO: 6 - protein, 9 aa, Artificial Sequence
  Synthetic
  MOD_RES (1)..(1): AMIDATION
LSPRTLNAW

SEQ ID NO: 7 - protein, 18 aa, Artificial Sequence
  Synthetic
  MOD_RES (1)..(1): 5′ Azid-PEG12
TESNKKFLPFQQFGRDIA

(19) United States
(12) Patent Application Publication
(10) Pub. No.: US 2023/0381112 A1
(43) Pub. Date: Nov. 30, 2023

(54) Compositions and Methods of Manufacturing Amphiphilic Block Copolymers that Form Nanoparticles in Situ

(71) Applicants: Vaccuitech North America, Inc., Baltimore, MD (US); Institute of Macromolecular Chemistry, Prague 6 (CZ)

(72) Inventors: Geoffrey Martin Lynn, Baltimore, MD (US); David Robert Wilson, Baltimore, MD (US); Ellen Wang, Plano, TX (US); Kyle James Fergie, Baltimore, MD (US); Lucie Woldrichová, Strakonice (CZ); Ladislav Androvic, Chribská (CZ); Richard Laga, Prague (CZ)

(73) Assignees: Vaccitech North America, Inc., Baltimore, MD (US); Institute of Macromolecular Chemistry, Prague 6 (CZ)

(21) Appl. No.: 18/027,346

(22) PCT Filed: Sep. 21, 2021
(86) PCT No.: PCT/US2021/051298

§ 371 Date: Mar. 20, 2023

Related U.S. Application Data
(60) Provisional application No. 63/081,729, filed on Sep. 22, 2020.

(51) Int. Cl. A61K 9/51; A61K 47/58

(52) U.S. Cl. CPC A61K 9/5138; A61K 47/58

(57) ABSTRACT

An amphiphilic block copolymer having any one of the formulas D-S—H, S(D)-H, S—H(D), D-S—H—S, D-S—H—S-D, S(D)-H—S, S(D)-H—S(D) or S—H(D)-S is disclosed. S is a hydrophilic block; H is a hydrophobic block; D is a drug molecule; ( ) denotes that the group is bonded directly or indirectly as a side chain or as part of a side chain group to the adjacent group; and the hyphen, “-” (or sometimes “-”), denotes that each of the adjacent S, H or D are linked either directly to one another or indirectly to one another via a linker, additionally wherein the hydrophilic block comprises a first hydrophilic monomer and the hydrophobic block comprises a first hydrophobic monomer and a second hydrophobic monomer.

### CROSS-REFERENCE TO RELATED APPLICATIONS

This application is a national stage entry under 35 U.S.C. § 371 of International Patent Application No. PCT/US2021/051298, filed Sep. 21, 2021, which claims priority to U.S. Provisional Application No. 63/081,729, filed on Sep. 22, 2020, the disclosure of which is hereby incorporated by reference in its entirety.

### SEQUENCE LISTING

The instant application contains a Sequence Listing which has been submitted electronically in ASCII format via EFS-Web and is hereby incorporated by reference in its entirety. Said ASCII copy, created on Jan. 3, 2022, is named “01304-0012-00PCT_ST25” and is 1.67 kilobytes in size.

### INTRODUCTION

The present disclosure relates to compositions of amphiphilic block copolymers comprising drug molecules that exist as unimers and have low viscosity at high concentrations during injection but self-assemble into nanoparticles in situ following administration to a subject.

### BACKGROUND

Small molecule drugs, as well as peptide and protein-based therapies often have suboptimal pharmacokinetics and/or biodistribution. This is particularly relevant for cytotoxic and immunostimulatory drugs with broad biodistribution that results in off-target activity as well as drugs that require targeting to specific tissues (e.g., CNS) or cells for efficacy. Further, many small molecule drugs, as well as peptide and protein-based therapies often have low bioavailability in tissues following oral or intravenous administration. To address these challenges, myriad drug delivery systems have been developed for modulating biodistribution and cell uptake, improving drug solubility and/or creating local drug depots for controlled release.

While drug delivery technologies continue to see increased adoption across therapeutic areas, individual drug molecules and therapeutic targets have unique challenges and often require a uniquely tailored approach. For instance, while various drug delivery systems have been approved by the FDA for intravenous delivery of cytoxic agents to tumors, there is still an unmet need for drug delivery systems that satisfy the requirements of sustained release of drug molecules in certain body cavities, such as ocular, bursal, articular, thecal, ventricular, pericardial, etc., which require small volumes of highly concentrated drug to allow sustained activity with infrequent dosing.

Ocular drug delivery is particularly challenging due to physical and physiological barriers including tear turnover, reflex blinking, and low biodistribution from traditional delivery methods such as topical eye drops (Patel, S. P. et al. Protein and peptide letters 2014, 21 (11), 1185-1200). Patient compliance with eye drop administration is often suboptimal and eye drops are generally unsuitable for administration of macromolecules including advanced biologic drugs such as anti-VEGF protein therapeutics (Campochiaro, P. A. et al. Ophthalmology 2019, 126 (8), 1141-1154). Routes of delivery including intravitreal injections, suprachoroidal injections and subconjunctival injections enable direct administration into the eye, enabling the vitreal or suprachoroidal space to serve as a depot-like reservoir for purposes of drug delivery with defined fluid turnover. Direct delivery to the choroidal space, where many ocular drugs have their site of action is hindered by both static and dynamic barriers. Static barriers include the sclera, choroid, and retinal pigment epithelium while dynamic barriers include lymphatic flow in the conjunctiva and blood flow in the conjunctiva and choroid (Patel, S. P. et al. Protein and peptide letters 2014, 21 (11), 1185-1200). Intravitreal and suprachoroidal administration are further limited by the available injection volume and needle diameter, which are effectively limited to 50-100 μL per administration in human patients. The narrow gauge needle (typically ≥28 gauge) required further limits the viscosity of administered therapeutics to <100 cP (Henein, C. et al. Pharmaceutics 2019, 11 (8), 371).

A major challenge for biologics based ocular drugs, such as anti-VEGF therapy, is that they often require frequent (monthly) intravitreal dosing to maintain effective therapeutic concentrations at the site of action in the choroidal space. The repeated dosing often causes major discomfort and high risk of infection with side effects such as endophthalmitis, hemorrhage, and retinal detachment that lead to low patient compliance (Campochiaro, P. A. et al. Ophthalmology 2019, 126 (8), 1141-1154). This high frequency of drug dosing is a direct result of the vitreal fluid turn-over rate that results in vitreal clearance rates that are proportional to drug size. For small molecules, clearance time following intravitreal injection is rapid with a clearance time of only a few hours (Gaudreault, J. et al. Retina 2007, 27 (9), 1260-1266); Fab molecules used for intravitreal injections such as Ranibizumab (Lucentis) have a molecular weight of approximately 46 kDa and hydrodynamic diameter of 4.5 nm, leading to a vitreal half-life of 3.2 days (Shatz, W. et al. Molecular pharmaceutics 2016, 13 (9), 2996-3003); and large macromolecules such as 500 kDa hyaluronic acid have a vitreal clearance half-life of approximately 30 days (Laurent, U. B. et al. Experimental Eye Research 1983, 36 (4), 493-503). While increasing the size of the therapeutic can improve half-life in the vitreous, particles much larger than 70 nm can appreciably scatter visible light and particles much larger than 200 nm may have limited mobility as the vitreal space has limited porosity with pore size between hyaluronan and other macromolecules measured to be effectively 550 nm for purposes of diffusion and convection (Xu, Q. et al. Journal of controlled release 2013, 167 (1), 76-84), with the latter being the primary mode of transport for drugs or particles during normal motion of the vitreous (Xu, Q. et al. Journal of controlled release 2013, 167 (1), 76-84).

Therefore, to overcome the challenges of ocular drug delivery, particularly for intravitreal injection, there is a need for drug delivery platforms that have sufficiently (i) large size (for example, >30 nm) to increase therapeutic (i.e. drug molecule) half-life but not too large (for example, <70 nm) so as to appreciably scatter visible light; and/or (ii) low viscosity (for example, <100 cP) at high concentrations (for example, >100 mg/mL) to enable small volume injections through a narrow-gauge needle.

Based on these requirements, synthetic and naturally occurring macromolecules as well as nano- and micro-particle assemblies based on hydrogels, emulsions, liposomes and polymer particles have been proposed for use as ocular drug delivery systems. However, while such technologies have demonstrated utility for modulating biodistribution and prolonging drug activity through sustained release for various applications, most fail to have either or both the optimal size or viscosity.

For instance, liposome (Bochot, A. et al. Journal of Controlled Release 2012, 161 (2), 628-634), polymersome and polyester based emulsions, such as PLGA copolymers, can be manufactured with sizes below the vitreous pore size of 550 nm and have been used to improve drug solubility, local residence time, and tissue retention (Liang, H. et al. Investigative Ophthalmology & Visual Science 2008, 49 (13), 2357-2357); however, such particles are often large enough to appreciably scatter visible light and have low and variable drug loading (<10% mass), thereby limiting the dose injectable with a single administration. Macromolecular polymer-drug conjugates offer an attractive alternative to particle-based emulsions. Multivalent macromolecules, such as linear polymers, can enable programmable, high drug loading (e.g., >20% m/m) and the molecular weight of the polymers can be tuned to modulate size and therefore vitreal half-life. However, as the viscosity of macromolecules increases non-linearly with molecular weight, it is often not practicable to use macromolecules above 200 kDa due to high viscosities that limit the concentrations that can be injected.

While conventional particle and macromolecular drug delivery technologies fail to satisfy key requirements for ocular drug delivery, macro-scale devices and self-assembling nanoparticle systems have emerged as promising alternatives.

Among macro-scale devices, port delivery systems have been explored for single-occasion implantation followed by yearly refilling with concentrated protein therapeutics, enabling slow release (Campochiaro, P. A. et al. Ophthalmology 2019, 126 (8), 1141-1154). Port delivery systems have the benefit of reducing dosing frequency to once per year, but have significant drawbacks, including surgical complication risks, while not solving the problem of influencing drug half-life in the intravitreal space (Campochiaro, P. A. et al. Ophthalmology 2019, 126 (8), 1141-1154).

In contrast, stimuli-responsive polymers that exist as unimers at high concentrations but self-assemble into nanoparticles in situ within the vitreous have the potential to systematically overcome the challenges of conventional particle drug delivery systems as well as port delivery systems. Indeed, polymers that exhibit temperature- or other stimuli-responsive properties enable injection of low viscosity solutions of the unimer form of the polymer, which can then form a multimeric assembly following injection. Temperature-responsive polymers often utilize poly(N-isopropylacrylamide) or p(NIPAM) (Keerl, M. et al. Journal of the American Chemical Society 2009, 131 (8), 3093-3097) as a primary constituent because it has a lower critical solution temperature (LCST) slightly above room temperature (˜20° C.) but below body temperature (e.g., 32° C.) allowing it to exist as unimers in solution at room temperature but undergo gelling once injected into a subject. To supplement its temperature-responsive properties, poly(NIPAM) is often combined with poly(ethylene glycol) (PEG) (Alexander, A. et al. European Journal of Pharmaceutics and Biopharmaceutics 2014, 88 (3), 575-585; Awwad, S. et al. Macromolecularbioscience 2018, 18 (2), 1700255), chitosan (Sapino, S. et al. Nanomaterials 2019, 9 (6), 884), or hyaluronic acid (Zhu, M. et al. Artificial cells, nanomedicine, and biotechnology 2018, 46 (6), 1282-1287) to further modulate its properties. Poloxamers and pluronics including PEG/PLGA and PEG/PLA diblock, triblock or pentablock (Patel, S. P. et al. Protein and peptide letters 2014, 21 (11), 1185-1200) copolymers (Bonacucina, G. et al. Polymers 2011, 3 (2), 779-811) as well as various formulations with poly(acrylic acid) (Ma, W.-d. et al. Drug development and industrial pharmacy 2008, 34 (3), 258-266) have also been explored their temperature-responsive gelling properties.

While existing temperature-responsive polymers, such as p(NIPAM), can be used as drug carriers and injected at high concentrations with low viscosity, the major limitations of current systems are (i) lack of control over the size of particles formed in situ; (ii) low and variable drug loading; (iii) and rapid elimination of the particles formed.

Thus, there is a major unmet need for polymer-drug conjugates that (i) can be injected at high concentrations with low viscosity into body cavities and (ii) assemble into stable particles of between about, for example, 30-70 nm, diameter, that are sufficiently large to prolong drug activity but not too large so as to occlude fluid escape from the cavity or in the case of ocular spaces, not too large so as to appreciably scatter visible light. Alternatively, or in addition, there is a need for polymer-drug conjugates that overcome one or more of the limitations of known polymer-drug conjugates.

### SUMMARY

The present disclosure is based in part on the findings that certain amphiphilic block copolymers can exist as a stable composition of unimers below a transition temperature and/or above a certain concentration of the unimers, and self assemble and exist in particle form above a transition temperature and/or below a certain concentration of unimers. Accordingly, one aspect described herein provides an amphiphilic block copolymer having any one of the formulas D-S—H, S(D)-H, S—H(D), D-S—H—S, D-S—H—S-D, S(D)-H—S, S(D)-H—S(D) or S—H(D)-S, wherein S is a hydrophilic block; H is a hydrophobic block; D is a drug molecule; ( ) denotes that D is bonded directly or indirectly as a side chain or as part of a side chain group to the adjacent S or H; and the hyphen, “-” (or sometimes “-”), denotes that each of the adjacent S, H or D are linked either directly to one another or indirectly to one another via a linker. The following additional embodiments are provided.

Embodiment 1 is an amphiphilic block copolymer having any one of the formulas D-S—H, S(D)-H, S—H(D), D-S—H—S, D-S—H—S-D, S(D)-H—S, S(D)-H—S(D) or S—H(D)-S, wherein S is a hydrophilic block; H is a hydrophobic block; D is a drug molecule; ( ) denotes that D is bonded directly or indirectly as a side chain or as part of a side chain group to the adjacent S or H; and the hyphen, “-” (or sometimes “-”), denotes that each of the adjacent S, H or D are linked either directly to one another or indirectly to one another via a linker, additionally wherein the hydrophilic block comprises a first hydrophilic monomer and the hydrophobic block comprises a first hydrophobic monomer and a second hydrophobic monomer.

Embodiment 2 is the amphiphilic block copolymer of embodiment 1, wherein the first hydrophobic monomer is selected from temperature-responsive monomers and the second hydrophobic monomer is selected from hydrophobic monomers comprising an aromatic group.

Embodiment 3 is the amphiphilic block copolymer of embodiment 1 or embodiment 2, wherein the first hydrophobic monomer comprises (meth)acrylates or (meth)acrylamides of the formula CH₂=CR₁₂—C(O)—R₁₁ (“Formula IV”), or combinations thereof, wherein each R₁₁ is independently —OR₁₃, —NHR₁₃ or —N(CH₃)R₁₃, each R₁₂ is independently H or CH₃, each R₁₃ is a hydrophobic substituent.

Embodiment 4 is the amphiphilic block copolymer of embodiment 3, wherein R₁₃ is an aliphatic group having three or more carbon atoms, which may be linear or branched or saturated or unsaturated, including linear chains such as —(CH₂)ₗCH₃, wherein l is an integer from 3 to 19; branched chains such as CH(CH₃)₂, (CH₂)ₗ_{^{*}}CH(CH₃)₂, wherein l* is an integer from 1 to 11; and cyclic rings, such as (CH₂)₁₊(C₅H₉), (CH₂)₁₊(C₆H₁₁), (CH₂)₁₊(C₇H₁₃) or (CH₂)ₗ₊(C₈H₁₈), wherein l⁺ is an integer from 0 to 6.

Embodiment 5 is the amphiphilic block copolymer of any one of embodiments 1 to 4, wherein the hydrophobic block comprises NIPMAM, NANPP, NVIBA, BEEP or TEGMA, or combinations thereof.

Embodiment 6 is the amphiphilic block copolymer of any one of embodiments 1 to 5, wherein the first hydrophobic monomer is NIPMAM, NANPP, NVIBA, BEEP or TEGMA.

Embodiment 7 is the amphiphilic block copolymer of any one of embodiments 1 to 6, wherein the second hydrophobic monomer comprises (meth)acrylates, or (meth)acrylamides of the formula CH₂=CR₁₂—C(O)—R₁₁ (“Formula IV”), or combinations thereof, wherein
each R₁₁ is independently —OR₁₃, —NHR₁₃ or —N(CH₃)R₁₃,
each R₁₂ is independently H or CH₃,
each R₁₃ is a hydrophobic substituent.

Embodiment 8 is the amphiphilic block copolymer of embodiment 7, wherein R₁₃ is an aliphatic group having three or more carbon atoms, which may be linear or branched or saturated or unsaturated, including linear chains such as —(CH₂)ₗCH₃, wherein l is an integer from 3 to 19; branched chains such as CH(CH₃)₂, (CH₂)ₗ_{^{*}}CH(CH₃)₂, wherein l* is an integer from 1 to 11; and cyclic rings, such as (CH₂)₁₊(C₆H₉), (CH₂)₁₊(C₆H₁₁), (CH₂)₁₊(C₇H₁₃) or (CH₂)₁₊(C₈H₁₈), wherein l⁺ is an integer from 0 to 6.

Embodiment 9 is the amphiphilic block copolymer of any one of embodiments 1 to 8, wherein the second hydrophobic monomer comprises aromatic groups.

Embodiment 10 is the amphiphilic block copolymer of any one of embodiments 1 to 9, wherein the second hydrophobic monomer comprises phenyl, fused phenyl or heterocyclic aromatic groups, or combinations thereof.

Embodiment 11 is the amphiphilic block copolymer of any one of embodiments 1 to 10, wherein the hydrophobic block comprises BnMAM.

Embodiment 12 is the amphiphilic block copolymer of any one of embodiments 1 to 11, wherein the first hydrophobic monomer is NIPMAM and the second hydrophobic monomer is BnMAM.

Embodiment 13 is the amphiphilic block copolymer of any one of embodiments 1 to 12, wherein the hydrophobic block is comprised of 50 to 95 mol % of the first hydrophobic monomer and of 5 to 50 mol % of the second hydrophobic monomer.

Embodiment 14 is the amphiphilic block copolymer of any one of embodiments 1 to 13, wherein the hydrophobic block is comprised of 70 to 85 mol % of the first hydrophobic monomer and of 15 to 30 mol % of the second hydrophobic monomer.

Embodiment 15 is the amphiphilic block copolymer of any one of embodiments 1 to 14, wherein the first hydrophilic monomer comprises (meth)acrylates or (meth)acrylamides, of the formula CH₂=CR₂—C(O)—R₁ (“Formula I”), or combinations thereof, wherein each R₁ is independently —OR₃, —NHR₃ or —N(CH₃)R₃, each R₂ is independently H or CH₃, each R₃ is independently H (except for ORs), CH₃, CH₂CH₃, CH₂CH₂OH, CH₂(CH₂)₂OH, CH₂CH(OH)CH₃, CHCH₃CH₂OH, or (CH₂CH₂O)ᵢH, where i is an integer from 1 to 10.

Embodiment 16 is the amphiphilic block copolymer of any one of embodiments 1 to 15, wherein the hydrophilic block comprises HEA, HEMAM, HPMA, PEG, or combinations thereof.

Embodiment 17 is the amphiphilic block copolymer of any one of embodiments 1 to 16, wherein the first hydrophilic monomer is HEA, HEMAM, HPMA or PEG.

Embodiment 18 is an amphiphilic block copolymer having any one of the formulas D-S—H, S(D)-H, S—H(D), D-S—H—S, D-S—H—S-D, S(D)-H—S, S(D)-H—S(D) or S—H(D)-S, wherein S is a hydrophilic block; H is a hydrophobic block; D is a drug molecule; ( ) denotes that D is bonded directly or indirectly as a side chain or as part of a side chain group to the adjacent S or H; and the hyphen, “-” (or sometimes “-”), denotes that each of the adjacent S, H or D are linked either directly to one another or indirectly to one another via a linker, additionally wherein the hydrophilic block comprises a first hydrophilic monomer and the hydrophobic block comprises a first hydrophobic monomer comprising at least one aromatic group.

Embodiment 19 is the amphiphilic block copolymer of embodiment 18, wherein the amphiphilic block copolymer comprises a second hydrophilic monomer present in the hydrophobic block, wherein the second hydrophilic monomer comprises (meth)acrylates or (meth)acrylamides, of the formula CH₂=CR₂—C(O)—R₁ (“Formula I”), or combinations thereof, wherein
each R₁ is independently —OR₃, —NHR₃ or —N(CH₃)R₃,
each R₂ is independently H or CH₃,
each R₃ is independently H (except for ORs), CH₃, CH₂CH₃, CH₂CH₂OH, CH₂(CH₂)₂OH, CH₂CH(OH)CH₃, CHCH₃CH₂OH, or (CH₂CH₂O)ᵢH, where i is an integer from 1 to 5.

Embodiment 20 is the amphiphilic block copolymer of embodiment 18 or 19, wherein the hydrophobic block comprises HEA, HEMAM, HPMA, PEG, or combinations thereof.

Embodiment 21 is the amphiphilic block copolymer of embodiment 19 or 20, wherein the second hydrophilic monomer is HEA, HEMAM, HPMA or PEG.

Embodiment 22 is the amphiphilic block copolymer of any one of embodiments 18 to 21, wherein the hydrophobic block further comprises a second hydrophobic monomer.

Embodiment 23 is the amphiphilic block copolymer of embodiment 22, wherein the second hydrophobic monomer is a temperature-responsive monomer.

Embodiment 24 is the amphiphilic block copolymer of embodiment 22 or 23, wherein the second hydrophobic monomer comprises (meth)acrylates or (meth)acrylamides of the formula CH₂=CR₁₂—C(O)—R₁₁ (“Formula IV”), or combinations thereof, wherein
each R₁₁ is independently —OR₁₃, —NHR₁₃ or —N(CH₃)R₁₃,
each R₁₂ is independently H or CH₃,
each R₁₃ is a hydrophobic substituent.

Embodiment 25 is the amphiphilic block copolymer of embodiment 24, wherein R₁₃ is an aliphatic group having three or more carbon atoms, which may be linear or branched or saturated or unsaturated, including linear chains such as —(CH₂)ₗCH₃, wherein l is an integer from 3 to 19; branched chains such as CH(CH₃)₂, (CH₂)ₗ_{^{*}}CH(CH₃)₂, wherein l* is an integer from 1 to 11; and cyclic rings, such as (CH₂)₁₊(C₅H₉), (CH₂)₁₊(C₆H₁₁), (CH₂)₁₊(C₇H₁₃) or (CH₂)₁₊(C₈H₁₅), wherein l⁺ is an integer from 0 to 6.

Embodiment 26 is the amphiphilic block copolymer of any one of embodiments 18 to 25, wherein the hydrophobic block comprises NIPMAM, NANPP, NVIBA, BEEP or TEGMA, or combinations thereof.

Embodiment 27 is the amphiphilic block copolymer of any one of embodiments 22 to 26, wherein the second hydrophobic monomer is NIPMAM, NANPP, NVIBA, BEEP or TEGMA.

Embodiment 28 is the amphiphilic block copolymer of any one of embodiments 18 to 27, wherein the first hydrophobic monomer comprises (meth)acrylates, or (meth)acrylamides of the formula CH₂=CR₁₂—C(O)—R₁₁ (“Formula IV”), or combinations thereof, wherein
each R₁₁ is independently —OR₁₃, —NHR₁₃ or —N(CH₃)R₁₃,
each R₁₂ is independently H or CH₃,
each R₁₃ is a hydrophobic substituent comprising at least one aromatic group.

Embodiment 29 is the amphiphilic block copolymer of any one of embodiments 18 to 28, wherein the aromatic group of the first hydrophobic monomer comprises phenyl, fused phenyl or heterocyclic aromatic groups, or combinations thereof.

Embodiment 30 is the amphiphilic block copolymer of any one of embodiments 18 to 29, wherein the hydrophobic block comprises BnMAM.

Embodiment 31 is the amphiphilic block copolymer of any one of embodiments 18 to 30, wherein the first hydrophobic monomer is BnMAM.

Embodiment 32 is the amphiphilic block copolymer of any one of embodiments 18 to 31, wherein the first hydrophobic monomer is BnMAM and the second hydrophobic monomer is NIPMAM, NANPP, NVIBA, BEEP or TEGMA.

Embodiment 33 is the amphiphilic block copolymer of any one of embodiments 18 to 32, wherein the hydrophobic block is comprised of 10 to 100 mol % of the first hydrophobic monomer.

Embodiment 34 is the amphiphilic block copolymer of any one of embodiments 18 to 33, wherein the hydrophobic block is comprised of 25 to 75 mol % of the first hydrophobic monomer.

Embodiment 35 is the amphiphilic block copolymer of any one of embodiments 18 to 34, wherein the first hydrophilic monomer comprises (meth)acrylates or (meth)acrylamides, of the formula CH₂=CR₂—C(O)—R₁ (“Formula I”), or combinations thereof, wherein
each R₁ is independently —OR₃, —NHR₃ or —N(CH₃)R₃,
each R₂ is independently H or CH₃,
each R₃ is independently H (except for OR₃), CH₃, CH₂CH₃, CH₂CH₂OH, CH₂(CH₂)₂OH, CH₂CH(OH)CH₃, CHCH₃CH₂OH, or (CH₂CH₂O)ᵢH, where i is an integer from 1 to 5.

Embodiment 36 is the amphiphilic block copolymer of any one of embodiments 18 to 35, wherein the hydrophilic block comprises HEA, HEMAM, HPMA, PEG, or combinations thereof.

Embodiment 37 is the amphiphilic block copolymer of any one of embodiments 18 to 36, wherein the first hydrophilic monomer is HEA, HEMAM, HPMA or PEG.

Embodiment 38 is an amphiphilic block copolymer having any one of the formulas D-S—H, S(D)-H, S—H(D), D-S—H—S, D-S—H—S-D, S(D)-H—S, S(D)-H—S(D) or S—H(D)-S, wherein S is a hydrophilic block; H is a hydrophobic block; D is a drug molecule; ( ) denotes that D is bonded directly or indirectly as a side chain or as part of a side chain group to the adjacent S or H; and the hyphen, “-” (or sometimes “-”), denotes that each of the adjacent S, H or D are linked either directly to one another or indirectly to one another via a linker, additionally wherein the hydrophilic block comprises a first hydrophilic monomer and the hydrophobic block comprises a first hydrophobic monomer and a second hydrophobic monomer, wherein the first hydrophobic monomer comprises temperature-responsive monomers and the second hydrophobic monomer comprises hydrophobic monomers comprising a fluorinated aromatic ring, or a fused aromatic ring, or combinations thereof.

Embodiment 39 is the amphiphilic block copolymer of embodiment 38, wherein the hydrophobic block comprises NIPMAM, NANPP, NVIBA, BEEP or TEGMA, or combinations thereof.

Embodiment 40 is the amphiphilic block copolymer of embodiment 38 or 39, wherein the first hydrophobic monomer is NIPMAM, NANPP, NVIBA, BEEP or TEGMA.

Embodiment 41 is the amphiphilic block copolymer of any one of embodiments 38 to 40, wherein the hydrophobic block comprises N-3,4,5-trifluorobenzyl methacrylamide, N-2,3,4,5,6 pentafluorobenzyl methacrylamide, N-trifluoromethylbenzyl methacrylamide and N-bitrifluoromethylbenzyl methacrylamide.

Embodiment 42 is the amphiphilic block copolymer of any one of embodiments 38 to 41, wherein the second hydrophobic monomer is chosen from N-3,4,5-trifluorobenzyl methacrylamide, N-2,3,4,5,6 pentafluorobenzyl methacrylamide, N-trifluoromethylbenzyl methacrylamide and N-bitrifluoromethylbenzyl methacrylamide.

Embodiment 43 is the amphiphilic block copolymer of any one of embodiments 38 to 42, wherein the first hydrophobic monomer is NIPMAM and the second hydrophobic monomer is N-3,4,5-trifluorobenzyl methacrylamide, N-2,3,4,5,6 pentafluorobenzyl methacrylamide, N-trifluoromethylbenzyl methacrylamide or N-bitrifluoromethylbenzyl methacrylamide.

Embodiment 44 is the amphiphilic block copolymer of any one of embodiments 38 to 43, wherein the hydrophobic block is comprised of 80 to 99 mol % of the first hydrophobic monomer and of 1 to 20 mol % of the second hydrophobic monomer.

Embodiment 45 is the amphiphilic block copolymer of any one of embodiments 38 to 44, wherein the hydrophobic block is comprised of 90 to 99 mol % of the first hydrophobic monomer and of 1 to 10 mol % of the second hydrophobic monomer.

Embodiment 46 is the amphiphilic block copolymer of any one of embodiments 38 to 45, wherein the hydrophilic block comprises HEA, HEMAM, HPMA, PEG, or combinations thereof.

Embodiment 47 is the amphiphilic block copolymer of any one of embodiments 38 to 46, wherein the first hydrophilic monomer is HEA, HEMAM, HPMA or PEG.

Embodiment 48 is the amphiphilic block copolymer of any one of embodiments 1 to 47, having a degree of polymerization block ratio of hydrophilic block to hydrophobic block of 0.5:1 to 4:1.

Embodiment 49 is the amphiphilic block copolymer of any one of embodiments 1 to 48, having a degree of polymerization block ratio of hydrophilic block to hydrophobic block of 0.75:1 to 3:1.

Embodiment 50 is the amphiphilic block copolymer of any one of embodiments 1 to 49, wherein the amphiphilic block copolymer has a molecular weight of about 5 kDa to about 60 kDa.

Embodiment 51 is the amphiphilic block copolymer of any one of embodiments 1 to 50, wherein the amphiphilic block copolymer has a molecular weight of about 15 kDa to about 50 kDa.

Embodiment 52 is the amphiphilic block copolymer of any one of embodiments 1 to 51, wherein the amphiphilic block copolymer has a molecular weight of about 25 kDa to about 45 kDa.

Embodiment 53 is the amphiphilic block copolymer of any one of embodiments 1 to 52, wherein D is chosen from ocular drugs, steroidal and nonsteroidal anti-inflammatory drugs, senolytic drugs or immunomodulatory drugs.

Embodiment 54 is the amphiphilic block copolymer of any one of embodiments 1 to 53, wherein D is linked directly or indirectly via a linker to an adjacent S or H.

Embodiment 55 is the amphiphilic block copolymer of any one of embodiments 1 to 54, wherein the amphiphilic block copolymer has the formula D-S—H, D-S—H—S or D-S—H—S-D, and D is linked to an end of the hydrophilic block of the amphiphilic block copolymer.

Embodiment 56 is the amphiphilic block copolymer of any one of embodiments 1 to 54, wherein the hydrophilic block further comprises a first reactive monomer that is distributed along the backbone of the hydrophilic block, and wherein the amphiphilic block copolymer has the formula S(D)-H, S(D)-H—S or S(D)-H—S(D) and D is linked to the amphiphilic block copolymer through the first reactive monomer.

Embodiment 57 is the amphiphilic block copolymer of any one of embodiments 1 to 56, wherein the hydrophilic block further comprises a first charged monomer.

Embodiment 58 is the amphiphilic block copolymer of any one of embodiments 1 to 57, wherein the hydrophilic block further comprises at least one negatively charged monomer.

Embodiment 59 is the amphiphilic block copolymer of any one of embodiments 57 or 58, wherein the first charged monomer is a (meth)acrylate or a (meth)acrylamide of the formula CH₂=CR₅—C(O)—R₄ (“Formula II”), wherein
R₄ is —OR₆, —NHR₆ or —N(CH₃)R₆,
R₅ is H or CH₃,
R₆ is OH, (CH₂)ⱼNH₂, (CH₂)ⱼCH(NH₂)COOH, (CH₂)ⱼCOOH, (CH₂)ⱼPO₃H₂, (CH₂)ⱼOPO₃H₂, (CH₂)ⱼSO₃H, (CH₂)ⱼOSO₃H, (CH₂)ⱼB(OH)₂, CH₂CH₂N(CH₃)₂, CH[CH₂N(CH₃)₂]₂, CH(COOH)CH—CH₂COOH, [CH₂CH(CH₃)O]₅PO₃H₂, (CH₂)₃CH(OPO₃H₂)(CH₂)₂CH(OPO₃H₂)(CH₂)₃CH₃, C(CH₃)₂CH₂SO₃H, and C₆H₄B(OH)₂, wherein each j is an integer from 1 to 6.

Embodiment 60 is the amphiphilic block copolymer of any one of embodiments 57 to 59, wherein the first charged monomer is wherein R₄=—OR₆, R₅=CH₃ and R₆=OH.

Embodiment 61 is the amphiphilic block copolymer of any one of embodiments 1 to 60, wherein the hydrophilic block further comprises a reactive monomer.

Embodiment 62 is the amphiphilic block copolymer of embodiment 61, wherein the reactive monomer comprises azide, alkyne, tetrazine, transcyclooctyne (TCO), protected hydrazine, ketone, aldehyde, hydroxyl, isocyanate, isothiocyanate, activated carboxylic acid, protected maleimide, thiol and/or amine groups.

Embodiment 63 is the amphiphilic block copolymer of embodiment 61 or 62, wherein the reactive monomer comprises a (meth)acrylate or a (meth)acrylamide of the formula CH₂=CR₈—C(O)—R₇ (“Formula III”), wherein,
R₇ is —OR₉, —NHR₉ or —N(CH₃)R₉,
R₈ is H or CH₃, and
R₉ is (CH₂)ₖR₁₀, (CH₂)ₖC(O)NHR₁₀, (CH₂CH₂O)ₖCH₂CH₂C(O)NHR₁₀, where k is an integer from 0 to 6, and
each R₁₀ is independently chosen from (CH₂)ₕ-FG, (CH₂CH₂O)ₕCH₂CH₂—FG or (CH₂CH₂O)ₕCH₂CH₂—FG, where h is an integer from 0 to 10, and FG is any functional group

Embodiment 64 is the amphiphilic block copolymer of embodiment 63, wherein FG is carboxylic acid, activated carboxylic acids, anhydride, amine, protected amines, OSi(CH₃), CCH, N₃, propargyl, halogen, an olefin, an endo cyclic olefins, CN, OH, or epoxy.

Embodiment 65 is the amphiphilic block copolymer of any one of embodiments 63 to 64, wherein in the compound of Formula III R₇ is NHR₉, R₈ is CH₃, R₉ is (CH₂)ₖC(O)NHR₁₀, k is equal to 2 and R₁₀ is propargyl.

Embodiment 66 is the amphiphilic block copolymer of any one of embodiments 1 to 65, wherein the hydrophilic block further comprises a reactive monomer linked to a CD22 agonist.

Embodiment 67 is the amphiphilic block copolymer of any one of embodiments 1 to 66, wherein the amphiphilic block copolymer exists as unimers at concentrations greater than 50 mg/mL in aqueous solutions, and wherein the amphiphilic block copolymer exists as particles at concentrations of less than or equal to 50 mg/mL.

Embodiment 68 is the amphiphilic block copolymer of any one of embodiments 1 to 67, wherein the amphiphilic block copolymer exists as unimers in aqueous solutions below a transition temperature, and wherein the amphiphilic block copolymer exists as particles in aqueous solutions above the transition temperature.

Embodiment 69 is the amphiphilic block copolymer of embodiment 68, wherein the transition temperature is 1° C. or more to 37° C. or lower.

Embodiment 70 is the amphiphilic block copolymer of embodiment 68 or 69, wherein the transition temperature is about 20° C. to about 34° C.

Embodiment 71 is the amphiphilic block copolymer of any one of embodiments 68 to 70, wherein the particles are about 20 nm to 200 nm in diameter.

Embodiment 72 is the amphiphilic block copolymer of any one of embodiments 68 to 71, wherein the particles are about 30 nm to 80 nm in diameter.

Embodiment 73 is the amphiphilic block copolymer of any one of embodiments 68 to 72, wherein the particles are about 30 nm to 60 nm in diameter.

Embodiment 74 is a solution comprising an aqueous solvent and unimers comprising the amphiphilic block copolymer of any one of embodiments 1 to 73.

Embodiment 75 is the solution of embodiment 74, wherein the concentration of unimers is greater than 50 mg/mL.

Embodiment 76 is the solution of embodiment 74, wherein the concentration of unimers is less than or equal to 50 mg/mL, and wherein the unimers form particles.

Embodiment 77 is the solution of any one of embodiments 74 to 76, wherein the temperature of the solution is not higher than a transition temperature.

Embodiment 78 is the solution of any one of embodiments 74 to 76, wherein the temperature of the solution is higher than a transition temperature, and wherein the unimers form particles.

Embodiment 79 is the solution of embodiment 77 or 78, wherein the transition temperature is 1° C. or more to 37° C. or lower.

Embodiment 80 is the solution of any one of embodiments 77 to 79, wherein the transition temperature is about 20° C. to about 34° C.

Embodiment 81 is a solution comprising an aqueous solvent and particles comprising the amphiphilic block copolymer of any one of embodiments 1 to 73.

Embodiment 82 is the solution of embodiment 81, having a concentration of unimers in the form of particles of less than or equal to 50 mg/mL.

Embodiment 83 is the solution of embodiment 81 or 82, wherein the solution temperature is above a transition temperature.

Embodiment 84 is the solution of embodiment 83, wherein the transition temperature is 1° C. or more to 37° C. or lower.

Embodiment 85 is the solution of embodiment 83 or 84, wherein the transition temperature is about 20° C. to about 34° C.

Embodiment 86 is the solution of any one of embodiments 76, and 78 to 85, wherein the particles have a diameter of about 20 nm to about 200 nm.

Embodiment 87 is the solution of any one of embodiments 76, and 78 to 86, wherein the particles have a diameter of about 30 nm to about 80 nm.

Embodiment 88 is the solution of any one of embodiments 76, and 78 to 87, wherein the particles have a diameter of about 30 nm to about 60 nm.

Embodiment 89 is a method of delivering a drug comprising administering the amphiphilic block copolymer of any one of embodiments 1 to 73 or the solution of any one of embodiments 74 to 88 to the subject.

Embodiment 90 is a method of delivering a drug to a subject in need of treatment comprising administering the amphiphilic block copolymer of any one of embodiments 1 to 73 or the solution of any one of embodiments 74 to 88 to the subject.

Embodiment 91 is the method of embodiment 90, wherein administering is an ocular, intravitreal, suprachoroidal, intrabursal, intraarticular, periarticular, intraperitoneal, intrapericardial, intraperipleural, intrathecal, intraventricular, intravenous, subcutaneous or intradermal injection.

Embodiment 92 is the method of embodiment 90 or 91, wherein the amphiphilic block copolymer or the solution is injected into a body cavity.

Embodiment 93 is the method of any one of embodiments 90 to 92, wherein the amphiphilic block copolymer or the solution is injected into the eye or the knee.

Embodiment 94 is the method of any one of embodiments 89 to 93, wherein the solution that is administered has a concentration of unimers greater than 50 mg/mL before administration.

Embodiment 95 is the method of any one of embodiments 89 to 94, wherein the solution that is administered has a concentration of unimers of 50 mg/mL or less after administration.

Embodiment 96 is the method of any one of embodiments 89 to 95, wherein the amphiphilic block copolymer or unimers exist in the form of particles after administration.

### BRIEF DESCRIPTION OF DRAWINGS

Embodiments of the present invention will be discussed with reference to the accompanying drawings wherein:

FIG. 1 shows the effect of varying (i) the mole-fraction (i.e., mol % or mole %) of a second hydrophobic monomer comprising an aromatic group (i.e., BnMAM) on the single hydrophobic block polymer transition temperature for methacrylamide or methacrylate polymers based on the p[(F1)_{f1}-co-(BnMAM)_{f2}] where F1 is any monomer that undergoes a transition from hydrophilic to hydrophobic. For all polymers shown the degree of polymerization (Xₙ) is between 100-200. For polymers composed of TEGMA or NIPMAM, co-polymerization with BnMAM reduces the transition temperature from above body temperature (>37° C.) to near room temperature (20° C.) to enable the hydrophobic single block polymer to have suitable thermoresponsive properties for a thermo-responsive transition between room temperature and body temperature.

FIG. 2 shows the effect that varying (i) the mole-fraction (i.e., mol %) of a second hydrophobic monomer comprising an aromatic group (i.e., BnMAM) and (ii) the length of the hydrophilic block have on the transition temperature of temperature-responsive amphiphilic block copolymers based on p[(NIPMAM)_{f1}-co-(BnMAM)_{f2}]-b-p(HPMA)ₐ, wherein the hydrophobic block number average molecular weight (Mn) is about 16 kDa, corresponding to a hydrophobic block degree of polymerization (Xₙ) of about 110-120, and the hydrophilic block Mn is varied between about 10-45 kDa corresponding to a hydrophilic block degree of polymerization between about 75-300. Transition temperature for p(NIPMAM) and p(NIPMAM-co-BnMAM) polymers without a hydrophilic block are shown as control groups. p(NIPMAM) has a transition temperature of 45° C. Increasing the BnMAM mole fraction up to 20 mol % reduces for p(NIPMAM-co-BnMAM) decreases the transition temperature to 19° C., while addition of a hydrophilic block is associated with an increase in the transition temperature in proportion to the length of the hydrophilic block, which can also be expressed as the degree of polymerization block ratio of hydrophilic block to hydrophobic block.

FIG. 3 is a different representation of the data shown in FIG. 2 wherein the degree of polymerization block ratio of hydrophilic block to hydrophobic block rather is shown on the x-axis instead of the hydrophilic block length. The data show that increasing block ratios are associated with increased transition temperature.

FIG. 4 shows the effect that the hydrophilic block length has on the hydrodynamic diameter of transition temperature of temperature-responsive amphiphilic block copolymers based on p[(NIPMAM)_{f1}-co-(BnMAM)_{f2}]-b-p(HPMA)ₐ, wherein the hydrophobic block number average molecular weight (Mn) is about 16 kDa, corresponding to a hydrophobic block degree of polymerization (Xₙ) of about 110-120, and the hydrophilic block Mn is varied between about 10-45 kDa corresponding to a hydrophilic block degree of polymerization between about 75-300. Temperature-responsive amphiphilic block copolymers were suspended at 5 mg/mL in PBS pH 7.4 at 37° C. and were evaluated using dynamic light scattering (DLS) to measure particle size (hydrodynamic diameter (D_{H}), nm). A plot of particle size versus hydrophilic block length is shown. While p[(NIPMAM)_{f1}-co-(BnMAM)_{f2}], i.e., the hydrophobic block alone, aggregated, all of the temperature-responsive amphiphilic block copolymers based on p[(NIPMAM)_{f1}-co-(BnMAM)_{f2}]-b-p(HPMA)a formed stable nanoparticle micelles, with those with block length greater than 10 kDa (corresponding to block ratios greater than 0.75) exhibiting the greatest hydrodynamic stability.

FIG. 5 is a different representation of the data shown in FIG. 4 wherein the degree of polymerization block ratio of hydrophilic block to hydrophobic block rather is shown on the x-axis instead of the hydrophilic block length.

FIG. 6 shows the impact that solution temperature and concentration have on the hydrodynamic behavior of a temperature-responsive amphiphilic diblock copolymer comprising CN-p[(NIPMAM)_{f1}-co-(BnMAM)_{f2}]-b-p(HPMA)ₐ-DTB (Compound 200) with a total number-average molecular weight (Mn) of about 40 kDa, hydrophilic to hydrophobic block ratio of 1.42 and 20 mol % BnMAM. Compound 200 was suspended in PBS, 150 mM, pH 7.4 at concentrations ranging from 0.8 to 200 mg/mL and particle size was assessed by DLS over temperatures ranging from between 20-37° C.

FIG. 7 shows viscosity measurements performed on a VROC Initium of polymer solutions in PBS including, thermo-responsive polymer samples of structure CN-p[(NIPMAM)f1-co-(BnMAM)f2]-b-p(HPMA)a-DTB with 20 mol % BnMAM (Compound 200) and 25 mol % BnMAM (Compound 209) and Star-polymer PAMAM(G5)-[p(HPMA30 kDa)]₂₇ (Compound 310).

FIG. 8 shows that a preferred embodiment of a temperature-responsive amphiphilic diblock copolymer, CN-p[(NIPMAM)_{f1}-co-(BnMAM)_{f2}]-b-p(HPMA)ₐ-N3 (i.e., Compound 229) has a stable micelle diameter in PBS, 150 mM, pH 7.4 at a concentration of 5 mg/mL over a time period of 90 days when incubated at 37° C.

FIG. 9 shows that a preferred embodiment of a temperature-responsive amphiphilic diblock copolymer p[(NIPMAM)_{f1}-co-(BnMAM)_{f2}]-b-p(HPMA)ₐ-N3 (i.e., Compound 229) has similar hydrodynamic behavior in rabbit vitreous and PBS pH 7.4 over a range of temperatures.

FIG. 10 shows the ability to conjugate the small molecule hydrophobic drug 2Bxy to hydrophobic block co-monomers to enable the polymer to be either a permanent micelle or a thermo-responsive diblock copolymer micelle. Shown are the thermo-responsive properties of the 2Bxy reacted version of p[(NIPMAM)_{f1}-co-(MA-b-Ala-TT)_{f2}]-b-p(HPMA)ₐ-DBCO (Compound 234) with different mole percentages of the hydrophobic block reacted with 2Bxy to yield p[(NIPMAM)_{f1}-co-(MA-b-Ala-2Bxy)_{f2}]-b-p(HPMA)ₐ-DBCO. Hydrophobic block modification with 10 and 20 mol % 2Bxy co-monomer (Compound 279 and Compound 280) yielded permanent micelles with no transition temperature between 4-37° C. Hydrophobic block modification with 5 mol % 2Bxy (Compound 281) yielded a thermo-responsive micelle with transition temperature of 33° C. Hydrophobic block modification with 2.5 mol % 2Bxy (Compound 282) yielded a thermo-responsive micelle with transition temperature of 46° C.

FIG. 11 shows the thermo-responsive properties of the 2BXy reacted version of Pg-p[(NIPMAM)_{f1}-co-(MA-b-Ala-TT)_{f2}]-DBCO (Compound 137) with different mole percentages of the hydrophobic block reacted with 2Bxy to yield Pg-p[(NIPMAM)_{f1}-co-(MA-b-Ala-2Bxy)_{f2}]-DBCO (Compounds 238-243).

FIG. 12 shows that temperature responsive amphiphilic diblock copolymers, e.g., p[(NIPMAM)_{f1}-co-(BnMAM)_{f2}]-b-p(HPMA)ₐ-N3 retains temperature responsiveness even after conjugation of large protein molecules to the hydrophilic terminus. Shown are the thermo-responsive properties of p[(NIPMAM)_{f1}-co-(BnMAM)_{f2}]-b-p(HPMA)ₐ-N3 (Compound 229) and p[(NIPMAM)_{f1}-co-(BnMAM)_{f2}]-b-p(HPMA)ₐ-PEG4-Ova (Compound 291) at a concentration of 5 mg/mL in PBS.

FIG. 13 shows that temperature responsive amphiphilic diblock copolymers with a large, globular hydrophilic molecule such as a protein conjugated to the hydrophilic terminus, e.g., p[(NIPMAM)_{f1}-co-(BnMAM)_{f2}]-b-p(HPMA)ₐ-PEG4-Ova (Compound 291), retain concentration responsive micellization properties. Shown are the thermo-responsive properties of p[(NIPMAM)_{f1}-co-(BnMAM)_{f2}]-b-p(HPMA)ₐ-PEG4-Ova (Compound 291) at a concentration of 5 mg/mL in PBS, at 200 mg/mL in PBS and at 5 mg/mL in PBS after dilution from 200 mg/mL. At 200 mg/mL total concentration, the protein concentration in the example shown was approximately 50 mg/mL, while the polymer concentration was approximately 150 mg/mL.

FIG. 14 shows the influence of the hydrophilic terminus small molecule on thermo-responsive behavior of the diblock copolymer micelle. Shown are polymers based on p[(NIPMAM)_{f1}-co-(BnMAM)_{f2}]-b-p(HPMA)ₐ-FG where the FG is either dithiobenzoate (DTB) from the chain transfer agent (terminated polymer Compound 200), azide (N3) (terminated polymer Compound 229), propargyl (terminated polymer Compound 230), dibenzocyclooctyne (DBCO, terminated polymer Compound 231) or small molecule hydrophobic drug 2Bxy (Compound 289).

FIG. 15 shows the influence of using alternative hydrophilic block co-monomers on thermo-responsive properties and micelle diameters. Shown are polymers p[(NIPMAM)_{f1}-co-(BnMAM)_{f2}]-b-p(HPMA)ₐ-DTB (Compound 200), p[(NIPMAM)_{f1}-co-(BnMAM)_{f2}]-b-p(HEMAM)ₐ-DTB (Compound 225) and p[(NIPMAM)_{f1}-co-(BnMAM)_{f2}]-b-p(HEA)ₐ-DTB (Compound 226) with similar hydrodynamic diameters and transition temperatures. For the structure shown, X may be either an —NH— group for methacrylamides (HPMA and HEMAM) or oxygen for acrylates (HEA) or methacrylates. For the structure shown, R is a methyl (—CH₃) for methacrylamides or methacrylates (HPMA and HEMAM) or hydrogen for acrylates (HEA). For the structure shown, R′ is a methyl (—CH₃) for HPMA or a hydrogen for HEMAM and HEA.

FIG. 16 shows the influence of conjugating a peptide (p2860, Compound 31) to the hydrophilic terminus of the p[(NIPMAM)_{f1}-co-(BnMAM)_{f2}]-b-p(HPMA)ₐ-DBCO (Compound 231) to yield p[(NIPMAM)_{f1}-co-(BnMAM)_{f2}]-b-p(HPMA)ₐ-p2860 (Compound 294). Conjugation of the peptide increased the transition temperature from approximately 23° C. to 25° C. Micelle diameter at 37° C. was not influenced.

FIG. 17 shows the influence of conjugating small molecule drug molecule (2Bxy) to the hydrophilic block co-monomers of CN-p[(NIPMAM)_{f1}-co-(BnMAM)_{f2}]-b-p[(HPMA)ₐ-co-(Ma-b-Ala-TT)ₑ]-DBCO (Compound 235). For comparison, the polymer without 2Bxy reacted containing 10 mol % MA-b-Ala-A2P is shown (Compound 244). The hydrophilic block modification with 2-6 mol % 2Bxy (Compounds 245, 246, 247, 248) and assessed for hydrodynamic number mean diameter between room temperature and 37° C.

FIG. 18 shows the influence of conjugating a small peptide (p2610, Compound 30) to the hydrophilic block co-monomers of CN-p[(NIPMAM)_{f1}-co-(BnMAM)_{f2}]-b-p[(HPMA)ₐ-co-(Ma-b-Ala-TT)ₑ]-DBCO (Compound 235) to yield CN-p[(NIPMAM)_{f1}-co-(BnMAM)_{f2}]-b-p[(HPMA)ₐ-co-(Ma-b-Ala-p2610)ₑ]-DBCO (Compound 249). The starting polymer had 10% mole fraction of the hydrophilic block as reactive co-monomer MA-b-Ala-TT, which was first reacted with a defined mole ratio of p2610 to yield fractionally modified hydrophilic block, followed by reaction with amino-2-propanol (A2P, CAS 78-96-6) to mimic HPMA co-monomers. For comparison, the polymer containing 10 mol % MA-b-Ala-A2P, unreacted with p2610, is shown (Compound 244). Hydrophilic block modification with 6 mol % p2610 (Compound 249) did not change the effective hydrodynamic diameter of the micelle at 37° C. or the effective transition temperature.

FIG. 19 shows the influence of synthesizing the thermo-responsive block either starting with the hydrophobic block or hydrophilic block to yield polymers of the same functional structure. Synthesizing the diblock thermo-responsive polymer by starting with the hydrophobic block yields a polymer structure CN-p[(NIPMAM)_{f1}-co-(BnMAM)_{f2}]-b-p(HPMA)ₐ-N3 (Compound 229) while synthesizing the polymer starting from the hydrophilic block yields a polymer structure N3-p(HPMA)ₐ-p[(NIPMAM)_{f1}-co-(BnMAM)_{f2}]-DTB (Compound 228).

FIG. 20 shows that the thermo-responsive polymer micelle in the absence of any drug molecule CN-p[(NIPMAM)_{f1}-co-(BnMAM)_{f2}]-b-p(HPMA)ₐ-N3 (Compound 229) does not negatively influence cell viability of human retinal pigmented epithelial cells (ARPE19). Also shown is negative control, branched polyethyleneimine 25 kDa (CAS 9002-98-6) at a concentration of 0.04 mg/mL in growth medium.

FIG. 21 shows that the thermo-responsive polymer micelle in the absence of any drug molecule CN-p[(NIPMAM)_{f1}-co-(BnMAM)_{f2}]-b-p(HPMA)ₐ-N3 (Compound 229) does not negatively influence cell viability of human monocyte cells (THP1). Also shown is negative control 2B (Compound 34) at a concentration 100 μM in cell growth media.

FIG. 22 shows levels of innate immune stimulation by NF-κB activation in THP1 cells by the thermo-responsive polymer micelle in the absence of any drug molecule CN-p[(NIPMAM)_{f1}-co-(BnMAM)_{f2}]-b-p(HPMA)ₐ-N3 (Compound 229) a positive control for NF-κB activation, small molecule immunostimulant TLR7/8 agonist 2B (Compound 34).

FIG. 23 shows a counter example of unsuitable hydrophobic co-monomers for the hydrophobic block of polymers with structure CN-p[(NIPMAM)_{f1}-co-(MA-b-Ala-TT)_{f2}]-b-p(HPMA)ₐ-Pg (Compound 233) with the MA-b-Ala-TT co-monomers (20% mole fraction) reacted with different hydrophobic amino ligands. Reaction with benzylamine to yield a polymer with 20% mole fraction MA-b-Ala-benzylamine (Compound 250) yielded a polymer that had a defined transition temperature of 23° C. and formed stable micelles of approximately 28 nm at 37° C. In contrast, polymers resulting from conjugation of hydrophobic alkyl ligands 1-octylamine (Compound 269) or 1-dodecylamine (Compound 272) to the MA-b-Ala-TT co-monomer yielded polymers without a defined transition temperature that did not yield stable micelles regardless of temperature.

FIG. 24 shows a counter example of a unsuitable hydrophobic co-monomers for the hydrophobic block of polymers with structure CN-p[(NIPMAM)_{f1}-co-(MA-b-Ala-TT)_{f2}]-b-p(HPMA)ₐ-Pg (Compound 233) with the MA-b-Ala-TT co-monomers reacted with 1-butylamine. Reaction with benzylamine to yield a polymer with 20% mole fraction MA-b-Ala-benzylamine (Compound 250) yielded a polymer that had a defined transition temperature of 23° C. and formed stable micelles of approximately 28 nm at 37° C. In contrast, reacting 1-butylamine with the starting polymer hydrophobic block co-monomer to yield polymer p[(NIPMAM)_{f1}-co-(MA-b-Ala-butylamine)₂]-b-p(HPMA)ₐ-Pg with either 20% (Compound 266) or 10% (Compound 267) mole fraction co-monomer creates polymers that have either an unstable hydrodynamic diameter, too high of a transition temperature or micelles outside of the optimal range of 30-60 nm.

FIG. 25 shows an example of additional suitable amino ligands that can be used with polymers of structure CN-p[(NIPMAM)_{f1}-co-(MA-b-Ala-TT)_{f2}]-b-p(HPMA)ₐ-Pg (Compound 233). Reaction with benzylamine to yield a polymer with 20% mole fraction MA-b-Ala-benzylamine (Compound 250) yielded a polymer that had a defined transition temperature of 23° C. and formed stable micelles of approximately 28 nm at 37° C. In contrast, reacting aniline at a 20% mole fraction to synthesize CN-p[(NIPMAM)_{f1}-co-(MA-b-Ala-aniline)_{f2}]-b-p(HPMA)ₐ-Pg (Compound 275) yielded a polymer with a transition temperature of approximately 35° C. with micelle diameter at 37° C. of approximately 40 nm. Reacting 3,5-dimethylaniline at a 20% mole fraction to synthesize CN-p[(NIPMAM)_{f1}-co-(MA-b-Ala-3,5-dimethylaniline)_{f2}]-b-p(HPMA)ₐ-Pg (Compound 277) yielded a polymer with an indistinct transition temperature, but a difference in diameter between 20° C. and 37° C. from approximately 10 nm to 20 nm.

FIG. 26 shows counter examples of unsuitable hydrophobic co-monomers for the hydrophobic block of polymers with structure CN-p[(NIPMAM)_{f1}-co-(MA-b-Ala-TT)_{f2}]-b-p(HPMA)ₐ-Pg (Compound 233) with the MA-b-Ala-TT co-monomers (20 mol %) reacted with fluorinated benzyl or aniline structures. Reaction with benzylamine to yield a polymer with 20% mole fraction MA-b-Ala-benzylamine (Compound 250) yielded a polymer that had a defined transition temperature of 23° C. and formed stable micelles of approximately 28 nm at 37° C. In contrast, reaction with 3,5-Bis(Trifluoromethyl)benzylamine to yield CN-p[(NIPMAM)_{f1}-co-(MA-b-Ala-3,5-Bis(Trifluoromethyl)benzylamine)_{f2}]-b-p(HPMA)ₐ-Pg at either 20% (Compound 263) or 10% (Compound 264) mole fractions yielded either a permanent micelle or a non-thermo-responsive polymer. Reaction with 3,5-Bis(Trifluoromethyl)aniline at 20% mole fraction to yield CN-p[(NIPMAM)_{f1}-co-(MA-b-Ala-3,5-Bis(Trifluoromethyl)aniline)_{f2}]-b-p(HPMA)ₐ-Pg (Compound 276) yielded a thermo-responsive polymer with a transition temperature of approximately 30° C. and a hydrodynamic diameter of approximately 125 nm at 37° C. Reaction with 3,5-difluoroaniline at 20% mole fraction to yield CN-p[(NIPMAM)_{f1}-co-(MA-b-Ala-3,5-Bis(Trifluoromethyl)aniline)_{f}2]-b-p(HPMA)ₐ-Pg (Compound 278) yielded a thermo-responsive polymer with an indistinct transition temperature of approximately 44° C. that formed unstable micelles.

FIG. 27 is a table defining the abbreviations used throughout.

### DESCRIPTION OF EMBODIMENTS

Details of terms and methods are given below to provide greater clarity concerning compounds, compositions, methods and the use(s) thereof for the purpose of guiding those of ordinary skill in the art in the practice of the present disclosure. The terminology in this disclosure is understood to be useful for the purpose of providing a better description of particular embodiments and should not be considered limiting.

About: In the context of the present disclosure, “about” when referring to a measurable value such as an amount, a temporal duration, and the like, is meant to encompass variations of ±20%, 10%, 5%, 1%, or +0.1% from the specified value, as such variations are appropriate to perform the disclosed methods.

Administration: To provide or give to a subject an agent, for example, formulations, e.g., aqueous solutions, comprising amphiphilic block copolymers as described herein, by any effective route. Exemplary routes of administration include, but are not limited to, oral, injection (such as ocular, intravitreal, suprachoroidal, intraarticular, periarticular, intrapericardial, intraperipleural, intrathecal, intraventricular, intrabursal, periarticular, subcutaneous, intramuscular, intradermal, intraperitoneal, and intravenous), transdermal (for example, topical), intranasal, vaginal, and inhalation routes.

“Administration of” and “administering a” compound should be understood to mean providing a compound, a prodrug of a compound, or an amphiphilic block copolymer composition as described herein. The compound or composition can be administered by another person to the subject or it can be self-administered by the subject.

Antigen-presenting cell (APC): Any cell that presents antigen bound to MHC class I or class II molecules to T cells, including but not limited to monocytes, macrophages, dendritic cells, B cells, T cells and Langerhans cells.

Antigen: Any molecule that contains an epitope that binds to a T cell or B cell receptor and can stimulate an immune response, in particular, a B cell response and/or a T cell response in a subject. The epitopes may be comprised of peptides, glycopeptides, lipids or any suitable molecules that contain an epitope that can interact with components of specific B cell or T cell receptors. Such interactions may generate a response by the immune cell. “Epitope” refers to the region of an antigen to which B and/or T cell proteins, i.e., B-cell receptors and T-cell receptors, interact.

Amphiphilic: The term “amphiphilic” is used herein to describe the properties of a substance containing both hydrophilic or polar (water-soluble) and hydrophobic or non-polar (water-insoluble) groups. Substances with amphiphilic properties may be referred to generically as amphiphiles. Amphiphiles include polymers that are comprised of both a hydrophilic region and a hydrophobic region, such as the amphiphilic block copolymers described herein that comprise hydrophilic blocks and hydrophobic blocks. Amphiphiles include materials that may only behave as amphiphiles at specific temperatures, displaying a change in their hydrophilicity or hydrophobicity with respect to temperature. Amphiphiles are referenced in formulae as any compound with a hydrophobic block (“S”) and a hydrophobic block (“H”), e.g., S—H.

Body temperature: Refers to normal body temperature in a healthy adult subject. Body temperature typically refers to the body temperature taken orally or over the temporal artery and is about 36 to 38° C. in a healthy adult subject. Body temperature as used herein may also refer to average temperature in certain tissues of the body, such as the vitreous of the eye, which is about 32 to 36° C.

Charge: A physical property of matter that affects its interactions with other atoms and molecules, including solutes and solvents. Charged matter experiences electrostatic force from other types of charged matter as well as molecules that do not hold a full integer value of charge, such as polar molecules. Two charged molecules of like charge repel each other, whereas two charged molecules of different charge attract each other. Charge is often described in positive or negative integer units.

Charged monomers: Refers to monomers that have one or more functional groups (FG) that are or can be (under certain conditions) positively or negatively charged. The functional groups comprising the charged monomers may be partial or full integer values of charge. A charged monomer may have a single charged functional group or multiple charged functional groups, which may be the same or different. Functional groups may be permanently charged or the functional groups comprising the charged molecule may have charge depending on the pH. The charged monomer may be comprised of positive functional groups, negative functional groups or both positive and negative functional groups. The net charge of the charged monomer may be positive, negative or neutral. The charge of a molecule, such as a charged monomer, can be readily estimated based on the molecule's Lewis structure and accepted methods known to those skilled in the art. Charge may result from inductive effects, e.g., atoms bonded together with differences in electron affinity may result in a polar covalent bond resulting in a partially negatively charged atom and a partially positively charged atom. For example, nitrogen bonded to hydrogen results in partial negative charge on nitrogen and a partial positive charge on the hydrogen atom. Alternatively, an atom may be considered to have a full integer value of charge when the number of electrons assigned to that atom is less than or equal to the atomic number of the atom. The charge of a functional group is determined by summing the charge of each atom comprising the functional group. The net charge of the charged monomer is determined by summing the charge of each atom comprising the molecule. Those skilled in the art are familiar with the process of estimating charge of a molecule, or individual functional groups, by summing the formal charge of each atom in a molecule or functional group, respectively.

Charged monomers may comprise negatively charged functional groups such as those that occur as the conjugate base of an acid at physiologic pH (e.g., functional groups with a pKa less than about 6.5), e.g., at a pH of about 7.4. These include but are not limited to molecules bearing carboxylates, sulfates, sulfonates, phosphates, phosphoramidates, and phosphonates. Charged monomers may comprise positively charged functional groups such as those that occur as the conjugate acid of a base at physiologic pH (e.g., functional groups wherein the pKa of the conjugate acid of a base is greater than about 8.5). These include but are not limited to molecules bearing primary, secondary and tertiary amines, as well as ammonium and guanidinium. Charged monomers may comprise functional groups with charge that is pH independent, including quaternary ammonium, phosphonium and sulfonium functional groups. Charged monomers may comprise zwitterions comprising both negative and positive functional groups. Charged monomers useful for the practice of the invention of the present disclosure are disclosed herein. Charged monomers on a copolymer are sometimes referred to as charged comonomers.

Chemotherapeutic: Refers to pharmaceutically active substances useful in the treatment of cancer and include growth inhibitory agents or other cytotoxic agents and include alkylating agents, anti-metabolites, anti-microtubule inhibitors, topoisomerase inhibitors, receptor tyrosine kinase inhibitors, angiogenesis inhibitors and the like. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-FU; folic acid analogues such as denopterin, methotrexate, pteropterin, trimetrexate; purine analogues such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; members of taxoid or taxane family, such as paclitaxel (TAXOL® docetaxel (TAXOTERE®) and analogues thereof; chlorambucil; gemcitabine; 6-thioguanine; mercaptopurine; methotrexate; platinum analogues such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; inhibitors of receptor tyrosine kinases and/or angiogenesis, including sorafenib (NEXAVAR®), sunitinib (SUTENT®), pazopanib (VOTRIENT™), toceranib (PALLADIA™), vandetanib (ZACTIMA™), cediranib (RECENTIN®), regorafenib (BAY 73-4506), axitinib (AG013736), lestaurtinib (CEP-701), erlotinib (TARCEVA®), gefitinib (IRESSA™), BIBW 2992 (TOVOK™), lapatinib (TYKERB®), neratinib (HKI-272), and the like, and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (FARESTON®); and anti-androgens such as flutamide, nilutamide, bicalutamide, leuprolide, and goserelin; and pharmaceutically acceptable salts, acids or derivatives of any of the above. Other conventional cytotoxic chemical compounds as those disclosed in in Medical Oncology (Calabresi et al, ISBN: 0071054081), are also suitable chemotherapeutic agents. Chemotherapeutics are a type of pharmaceutically active compound and chemotherapeutics and are referred to herein as drugs or drug molecules, or “D” in formulae.

Click chemistry reaction: A bio-orthogonal reaction that joins two compounds together under mild conditions in a high yield reaction that generates minimal, biocompatible and/or inoffensive byproducts. An exemplary click chemistry reaction used in the present disclosure is the reaction of an azide group with an alkyne to form a triazole through strain-promoted [3+2] azide-alkyne cyclo-addition.

Copolymer: A polymer derived from two (or more) different monomers, as opposed to a homopolymer where only one monomer is used. Since a copolymer includes at least two types of constituent units (also structural units), copolymers may be classified based on how these units are arranged along the chain. A copolymer may be a statistical copolymer wherein the two or monomer units are distributed randomly; or, the copolymer may be an alternating copolymer wherein the two or more monomer units are distributed in an alternating sequence. The term “block copolymer” refers generically to a polymer composed of two or more contiguous blocks of different constituent monomers or comonomers (if a block comprises two or more different monomers). Block copolymer may be used herein to refer to a copolymer that comprises two or more homopolymer subunits, two or more copolymer subunits or one or more homopolymer subunits and one or more copolymer subunits, wherein the subunits may be linked directly by covalent bonds, or the subunits may be linked indirectly an intermediate non-repeating subunit, such as a junction block or linker. Blocks may be based on linear and/or brush architectures. Block copolymers with two or three distinct blocks are referred to herein as “diblock copolymers” and “triblock copolymers,” respectively. Copolymers may be referred to generically as polymers, e.g., a statistical copolymer may be referred to as a polymer or copolymer. Similarly, a block copolymer may be referred to generically as a polymer.

Critical micelle concentration (CMC): Refers to the concentration of a material above which micelles spontaneously form to satisfy thermodynamic equilibrium.

Drug: Refers to any pharmaceutically active molecule—including, without limitation, proteins, peptides, sugars, saccharides, nucleosides, inorganic compounds, lipids, nucleic acids, small synthetic chemical compounds, macrocycles, etc. —that has a physiological effect when ingested or otherwise introduced into the body. Pharmaceutically active compounds can be selected from a variety of known classes of compounds, including, for example, analgesics, anesthetics, anti-inflammatory agents (including steroidal and nonsteroidal anti-inflammatory drugs), anthelmintics, anti-arrhythmic agents, antiasthma agents, antibiotics (including penicillins), anticancer agents (including Taxol), anticoagulants, antidepressants, antidiabetic agents, antiepileptics, antihistamines, antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives (hypnotics and neuroleptics), astringents, bacteriostatic agents, beta-adrenoceptor blocking agents, blood products and substitutes, bronchodilators, buffering agents, cardiac inotropic agents, chemotherapeutics, contrast media, corticosteroids, cough suppressants (expectorants and mucolytics), diagnostic agents, diagnostic imaging agents, diuretics, dopaminergics (antiparkinsonian agents), free radical scavenging agents, growth factors, haemostatics, immunological agents, lipid regulating agents, muscle relaxants, proteins, such as therapeutic antibodies and antibody fragments, MHC-peptide complexes, cytokines and growth factors, glycoproteins, peptides and polypeptides, parasympathomimetics, parathyroid calcitonin and biphosphonates, prostaglandins, radio-pharmaceuticals, hormones, sex hormones (including steroids), time release binders, anti-allergic agents, stimulants and anoretics, steroids, sympathomimetics, thyroid agents, vaccines, vasodilators, and xanthines. Drugs may also be referred to as pharmaceutically active agents, pharmaceutically active substances or biologically active compounds or bioactive molecules.

Graft polymer: Refers to a polymer that results from the linkage of a polymer of one composition to the side chains of a second polymer of a different composition. A first polymer linked through comonomers, e.g., reactive monomers, to a second polymer results in a graft copolymer. A first polymer linked through an end group to a second polymer may be described as a block polymer (e.g., A-B type diblock) or an end-grafted polymer (or end-grafted copolymer).

Hydrophilic: Refers to the tendency of a material to disperse freely in aqueous media. A material is considered hydrophilic if it prefers interacting with other hydrophilic material and avoids interacting with hydrophobic material. In some cases, hydrophilicity may be used as a relative term, e.g., the same molecule could be described as hydrophilic or not depending on what it is being compared to. Hydrophilic molecules are often polar and/or charged and have good water solubility, e.g., are soluble up to 0.1 mg/mL or more. Neutral hydrophilic monomers (sometimes referred to as “hydrophilic monomers”) are monomers that form water-soluble polymers. For example, a HPMA monomer may be referred to as a hydrophilic monomer because poly(HPMA) is a water-soluble polymer. Note: charged monomers may be hydrophilic but are typically charged at physiologic pH and so are referred to as charge monomers herein, whereas hydrophilic monomers that are not charged at physiologic pH are referred to as neutral hydrophilic monomers. Hydrophilic block refers to the portion of a block copolymer that is water soluble. A hydrophilic block may contain hydrophobic monomers dispersed through the block and yet remain water soluble, and therefore remain a hydrophilic block.

Hydrophobic: Refers to the tendency of a material to avoid contact with water. A material is considered hydrophobic if it prefers interacting with other hydrophobic material and avoids interacting with hydrophilic material. Hydrophobicity is a relative term; the same molecule could be described as hydrophobic or not depending on what it is being compared to. Hydrophobic molecules are often non-polar and non-charged and have poor water solubility, e.g., are insoluble down to 0.1 mg/mL or less. Hydrophobic monomers are monomers that form polymers that insoluble in water or insoluble in water at certain temperatures, pH and concentration. For example, a styrene monomer may be referred to as a hydrophobic monomer because poly(styrene) is water insoluble polymer. Hydrophobic block refers to the portion of a block copolymer that is insoluble in water at certain temperature, pH and concentrations. A hydrophobic block may contain hydrophilic monomers dispersed through the block and yet remain water insoluble, and therefore remain a hydrophobic block.

Immune response: A change in the activity of a cell of the immune system, such as a B cell, T cell, or monocyte, as a result of a stimulus, either directly or indirectly, such as through a cellular or cytokine intermediary. In one embodiment, the response is specific for a particular antigen (an “antigen-specific response”). In one embodiment, an immune response is a T cell response, such as a CD4 T cell response or a CD8 T cell response. In one embodiment, an immune response results in the production of additional T cell progeny. In one embodiment, an immune response results in the movement of T cells. In another embodiment, the response is a B cell response, and results in the production of specific antibodies or the production of additional B cell progeny. In other embodiments, the response is an antigen-presenting cell response. “Enhancing an immune response” refers to co-administration of an adjuvant and an immunogenic agent, such as a peptide antigen, as part of a peptide antigen conjugate, wherein the adjuvant increases the desired immune response to the immunogenic agent compared to administration of the immunogenic agent to the subject in the absence of the adjuvant. In some embodiments, an antigen is used to stimulate an immune response leading to the activation of cytotoxic T cells that kills virally infected cells or cancerous cells. In some embodiments, an antigen is used to induce tolerance or immune suppression. A tolerogenic response may result from the unresponsiveness of a T cell or B cell to an antigen. A suppressive immune response may result from the activation of regulatory cells, such as regulatory T cells that downregulate the immune response, i.e., dampen then immune, response. Antigens administered to a patient in the absence of an adjuvant are generally tolerogenic or suppressive and antigens administered with an adjuvant are generally stimulatory and lead to the recruitment, expansion and activation of immune cells.

Linked or coupled: The terms “linked” and “coupled” mean joined together, either directly or indirectly. A first moiety may be covalently or noncovalently linked to a second moiety. In some embodiments, a first molecule is linked by a covalent bond to another molecule. In some embodiments, a first molecule is linked by electrostatic attraction to another molecule. In some embodiments, a first molecule is linked by dipole-dipole forces (for example, hydrogen bonding) to another molecule. In some embodiments, a first molecule is linked by van der Waals forces (also known as London forces) to another molecule. A first molecule may be linked by any and all combinations of such couplings to another molecule. The molecules may be linked indirectly, such as by using a linker (sometimes referred to as linker molecule). The molecules may be linked indirectly by interposition of a component that binds non-covalently to both molecules independently.

As used herein, “linked” and variations thereof, refer to maintaining molecules in chemical or physical association, including after injection, at least until they contact a cell or release linked drug at a defined rate. In some embodiments, linked components are associated so that the components are not immediately freely dispersible from one another. For example, two components may be covalently linked to one another so that the two components are incapable of separately dispersing or diffusing.

Membrane: A spatially distinct collection of molecules that defines a 2-dimensional surface in 3-dimensional space, and thus separates one space from another in at least a local sense. A “bilayer membrane” or “bilayer(s)” is a self-assembled membrane of amphiphiles or super-amphiphiles in aqueous solutions.

Micelles: Spherical receptacles comprised of a single monolayer defining a closed compartment. Generally, amphiphilic molecules spontaneously form micellar structures in polar solvents. In contrast to liposome bilayers, micelles are “sided” in that they project a hydrophilic, polar outer surface and a hydrophobic interior.

Mol % or Mole %: Refers to the percentage of a particular type of monomeric unit (or “monomer”) that is present in copolymer (sometimes just referred to as a polymer). For example, a copolymer comprised of 100 monomeric units of A and B with a density (or “mol %”) of monomer A equal to 10 mol % would have 10 monomeric units of A, and the remaining 90 monomeric units (or “monomers”) may be monomer B or another monomer unless otherwise specified.

Monomeric unit: The term “monomeric unit” is used herein to mean a unit of polymer molecule containing the same or similar number of atoms as one of the monomers. Monomeric units, as used in this specification, may be of a single type (homogeneous) or a variety of types (heterogeneous). For example, poly(amino acids) are comprised of amino acid monomeric units. Monomeric units may also be referred to as monomers or monomer units or the like.

Net charge: The sum of electrostatic charges carried by a molecule or, if specified, a section of a molecule. As defined herein, the net charge of a molecule is the sum of the formal charge of each atom in the molecule.

Ocular drug(s): Refers to any drug molecule(s) possessing biological activity relevant for any disease affecting the eye, including the retina, choroid or vasculature of the eye, such as drugs used to treat macular degeneration, such as biologics targeting VEGF receptor in the treatment of wet age-related macular degeneration (AMD).

Particle: A nano- or micro-sized supramolecular structure comprised of an assembly of molecules. For example, in some embodiments, the amphiphilic block copolymer forms a particle, or exists as a particle, in aqueous solution. In some embodiments, particle formation by the amphiphilic block copolymer is dependent on pH or temperature. In some embodiments, the nanoparticles comprised of amphiphilic block copolymers have an average diameter between 5 nanometers (nm) to 500 nm. In some embodiments, the nanoparticles comprised of amphiphilic block copolymers form micelles and have an average diameter between 5 nanometers (nm) to 100 nm, or between 5 nm and 200 nm, or between 20 and 200 nm, or between 100 nm and 200 nm, or between 30 and 60 nm, or between 30 and 80 nm, or between 10 and 30 nm. In some embodiments, the nanoparticles comprised of amphiphilic block copolymers may be larger than 100 nm. Diameter as stated here refers to a number mean diameter acquired by dynamic light scattering at aqueous solution such as phosphate buffered saline (PBS).

Phosphate buffer saline: Refers to a physiologically equivalent buffer abbreviated PBS, containing approximately 15 mM phosphate buffer and 135 mM sodium chloride salt for 150 mM isotonic osmolarity. Unless otherwise specified pH of PBS was used at pH 7.4 and at a 1×concentration of 150 mM salt concentration.

Polar: A description of the properties of matter. Polar is a relative term, and may describe a molecule or a portion of a molecule that has partial charge that arises from differences in electronegativity between atoms bonded together in a molecule, such as the bond between nitrogen and hydrogen. Polar molecules prefer interacting with other polar molecules and typically do not associate with non-polar molecules. In specific, non-limiting cases, a polar group may contain a hydroxyl group, or an amino group, or a carboxyl group, or a charged group. In specific, non-limiting cases, a polar group may prefer interacting with a polar solvent such as water. In specific, non-limiting cases, introduction of additional polar groups may increase the solubility of a portion of a molecule.

Polymer: A molecule containing repeating structural units (i.e., monomers, sometimes referred to as monomer units). As described in greater detail throughout the disclosure, polymers may be used for any number of components of the amphiphilic block copolymer. Various compositions of polymers useful for the practice of the invention are discussed in greater detail throughout.

Polymerization: A chemical reaction, usually carried out with a catalyst, heat or light, in which monomers combine to form a chainlike, branched or cross-linked macromolecule (a polymer). The chains, branches or cross-linked macromolecules can be further modified by additional chemical synthesis using the appropriate substituent groups and chemical reactions. The monomers may contain reactive substances. Polymerization commonly occurs by addition or condensation. Addition polymerization occurs when an initiator, usually a free radical, reacts with a double bond in the monomer. The free radical adds to one side of the double bond, producing a free electron on the other side. This free electron then reacts with another monomer, and the chain becomes self-propagating, thus adding one monomer unit at a time to the end of a growing chain. Condensation polymerization involves the reaction of two monomers resulting in the splitting out of a water molecule. In other forms of polymerization, a monomer is added one at a time to a growing chain through the staged introduction of activated monomers, such as during solid phase peptide synthesis.

Polymersome: Refers to a vesicle that is assembled from synthetic block copolymers in aqueous solutions. Unlike liposomes, a polymersome does not include lipids or phospholipids as its majority component. Consequently, polymersomes can be thermally, mechanically, and chemically distinct and, in particular, more durable and resilient than the most stable of lipid vesicles. The polymersomes assemble during processes of lamellar swelling, e.g., by film or bulk rehydration or through an additional phoresis step, as described below, or by other known methods. Like liposomes, polymersomes form by “self-assembly,” a spontaneous, entropy-driven process of preparing a closed semi-permeable membrane.

Purified: A substance or composition that is relatively free of impurities or substances that adulterate or contaminate the substance or composition. The term purified is a relative term and does not require absolute purity. Substantial purification denotes purification from impurities. A substantially purified substance or composition is at least 60%, 70%, 80%, 90%, 95%, 98%, or 99% pure.

Soluble: Capable of becoming molecularly or ionically dispersed in a solvent to form a homogeneous solution. When referring to a polymer, a soluble polymer is understood to be a single molecule in solution that does not assemble into multimers or other supramolecular structures through hydrophobic or other non-covalent interactions. A soluble polymer is understood to be freely dispersed as single molecules in solution. Hydrophobic polymers described herein are insoluble in aqueous solutions down to about 0.1 mg/mL or less. Solubility can be determined by visual inspection, by turbidity measurements or by dynamic light scattering.

Subject and patient: These terms may be used interchangeably herein to refer to both human and non-human animals, including birds and non-human mammals, such as rodents (for example, mice and rats), non-human primates (for example, rhesus macaques), companion animals (for example domesticated dogs and cats), livestock (for example pigs, sheep, cows, llamas, and camels), as well as non-domesticated animals (for example big cats).

Physiologic: Refers to a condition or conditions that are representative of the conditions in a subject. A physiologic buffer refers to a buffer that has similar salt and pH to fluids in the body of a subject, such as serum. Physiologic pH is about pH 7.4.

Reactive: as used herein describes the stability of a molecule or functional group of a molecule and its propensity to undergo a chemical reaction in the presence of another functional group or molecule. For example, amines have the tendency to react with electrophiles under certain conditions, and therefore molecules comprising amines may be referred to as reactive. Reactive monomers refer to monomers with one or more functional groups that are reactive. Various examples of reactive monomers are described in greater detail elsewhere.

Room temperature: Refers to the average range of air temperatures that preferred in indoor settings. As used herein, room temperature may refer to temperatures between 16 to 26° C., typically about 20 to 22° C.

Telechelic: Is used to describe a polymer that has one or two reactive ends that may be the same or different. The word is derived from telos and chele, the Greek words for end and claw, respectively. A semi-telechelic polymer describes a polymer with only a single end group, such as a reactive functional group that may undergo additional reactions, such as polymerization. A hetero-telechelic polymer describes a polymer with two end groups, such as reactive functional groups, that have different reactive properties. Herein, polymer arms (A) with different linkers precursors at each end, i.e., X2 and Z1, are heterotelechelic polymers.

Treating, preventing, or ameliorating a disease: “Treating” refers to an intervention that reduces a sign or symptom or marker of a disease or pathological condition after it has begun to develop. For example, treating a disease may result in a reduction in tumor burden, meaning a decrease in the number or size of tumors and/or metastases, or treating a disease may result in immune tolerance that reduces systems associated with autoimmunity. “Preventing” a disease refers to inhibiting the full development of a disease. A disease may be prevented from developing at all. A disease may be prevented from developing in severity or extent or kind. “Ameliorating” refers to the reduction in the number or severity of signs or symptoms or marker of a disease, such as cancer.

Temperature-responsive: Refers to changes in properties of a material with changes in temperature. A material may be described as temperature-responsive if the properties of the material change with respect to changes in temperature. Temperature-responsive polymers are polymers that experience a change in physical properties in response to temperature, such as a change in solubility with changes temperature, which may be described by a lower critical solution temperature (LCST) or upper critical solution temperature (UCST). Preferred embodiments of the temperature-responsive polymers described herein have reduced solubility at elevated temperatures, which may be described by an LCST or transition temperature (Ttr), if the LCST is unknown. Transitions temperatures described herein are experimentally determined by dynamic light scattering or by turbidity, wherein particle size (or light scattering intensity) or absorbance are plotted against temperature to produce a sigmoidal curve. The transition temperature as used herein is the temperature (value on the x-axis) at which the y-value is 50% the maximum value of the sigmoidal curve. Temperature-responsive monomers are monomers that form temperature-responsive polymers. For example, a NIPAM monomer may be referred to as a temperature-responsive monomer because poly(NIPAM) is a temperature-responsive polymer. Note: temperature-responsive monomers are defined herein as a type of hydrophobic monomer that is hydrophobic at certain temperatures.

Unimer: Refers to a solo subunit of a particle, such as a micelle, polymersome, encapsulating membrane or other supramolecular structure. The amphiphilic block copolymers described herein exist as unimers in solutions of a certain concentration or in solutions of a certain temperature. A unimer is distinguishable from a monomer (e.g., BnMAM, or NIPMAM), which is the subunit of a polymer (e.g., an amphiphilic block copolymer).

A person of ordinary skill in the art would recognize that the definitions provided above are not intended to include impermissible substitution patterns (e.g., methyl substituted with 5 different groups, and the like). Such impermissible substitution patterns are easily recognized by a person of ordinary skill in the art. Any functional group disclosed herein and/or defined above can be substituted or unsubstituted, unless otherwise indicated herein. Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. The term “comprises” means “includes.” Therefore, comprising “A” or “B” refers to including A, including B, or including both A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described herein. In case of conflict, the present specification, including explanations of terms, will control. In addition, the materials, methods, and examples are illustrative only and not intended to be limiting

Provided herein is an amphiphilic block copolymer having any one of the formulas, S—H, D-S—H, S(D)-H, S—H(D), S—H—S, D-S—H—S, D-S—H—S-D, S(D)-H—S, S(D)-H—S(D) or S—H(D)-S; wherein S is a hydrophilic block; H is a hydrophobic block; D is a drug molecule; ( ) denotes that the group is bonded directly or indirectly as a side chain or as part of a side chain group to the adjacent group; and the hyphen, “-” (or sometimes “-”), denotes that each of the adjacent S, H or D are linked either directly to one another or indirectly to one another via a linker.

The amphiphilic block copolymers described herein have many uses but have particular utility in the formation of particles (e.g., micelles, polymersomes, encapsulating membranes or other types of supramolecular structures) to encapsulate or array one or more drug molecule(s) (D) and any other agents as required, such as dye molecules or radiotracers, which may be covalently linked to the amphiphilic block copolymers either directly or through a linker, or may be incorporated into the particles formed by the amphiphilic block copolymers through one or more different types of intermolecular interactions (e.g., electrostatic, hydrogen bonding, pi-stacking, hydrophobic, etc.). The “loaded” particles formed by amphiphilic block copolymers may be further used to retain an encapsulatable material (an “encapsulant”) at a site of injection when administered to a subject, or may be used to transport an encapsulatable material (an “encapsulant”) from a site of injection to another tissue; for example, the micelle or polymersome can be used to deliver a drug or therapeutic composition to a patient's tissue or through the blood stream.

Particles may be formed comprising a single composition of amphiphilic block copolymer, wherein the drug molecule, D, is non-covalently incorporated within the particle or directly linked to the particle through covalent attachment to the amphiphilic block copolymer:
S—H+D
D-S—H
S(D)-H
S—H(D)
S—H—S+D
S—H(D)-S
D-S—H—S
D-S—H—S-D
S(D)-H—S
S(D)-H—S(D)

Particles may be formed comprising an amphiphilic block copolymer and two or more different drug molecules, or two or more amphiphilic block copolymers comprising two or more different drug molecules. Non-limiting examples include:
a. S—H+D1+D2
b. S—H+D1+D2 . . . +Dn
c. D1-S—H+D2-S—H
d. D1-S—H+D2-S—H . . . +Dn-S—H
e. S(D1)-H(D2)
f. S(D1)-H+S(D2)-H
g. S(D1)-H+S(D2)-H . . . +S(Dn)
h. S—H(D1)+S—H(D2)
i. S—H(D1)+S—H(D2) . . . +S—H(Dn)
j. S—H—S+D1+D2
k. S—H—S+D1+D2 . . . +Dn
1. S—H(D1)-S+S—H(D2)-S
m. S—H(D1)-S+S—H(D2)-S . . . +S—H(Dn)-S
n. D1-S—H—S+D2-S—H—S
o. D1-S—H—S+D2-S—H—S . . . +Dn-S—H—S
p. D1-S—H—S-D2
q. S(D1)-H—S+S(D2)-H—S
r. S(D1)-H—S+S(D2)-H—S . . . +S(Dn)-H—S
s. S(D1)-H—S(D2)

In the above examples of particles comprising two or more drug molecules, the two or more drug molecules are indicated by Dn, where D is a drug molecule and n is any integer value. For example a particle comprising a first drug molecule and a second drug molecule may have the formula S—H+D1+D2. Particles comprising S—H+D1+D2 . . . +Dn indicate that there are an integer number, n, of different drug molecules. Throughout the specification, compositions of particles based on amphiphilic block copolymers are described with reference to a drug molecule, but it should be understood that compositions may include one, two, or more drug molecules, which may be the same or different.

Incorporation of certain drug molecules to particles based on amphiphilic block copolymers can be improved by, e.g., attachment of the drug molecule to a hydrophobic block, to yield D-H, which can be used in the preparation of mosaic particles comprising S—H+D-H or S—H—S+D-H.

For drug molecules that bind extracellular receptors, optimal array of the drug molecules on particles based on amphiphilic block copolymers can require modulation of the density to achieve an optimal effect. For such applications, density of the drug molecule arrayed on the surface of the particles based on amphiphilic block copolymers can be achieved through the use of mosaic particles comprising different ratios of D-S—H+S—H, D-S—H+S—H—S, D-S—H—S+S—H—S, S(D)-H+S—H, S(D)-H+S—H—S, S(D)-H—S+S—H—S, or S(D)-H—S(D)+S—H—S.

The amphiphilic block copolymers described herein can also be used to prepare “empty” micelles, polymersomes or other supramolecular structures.

The amphiphilic block copolymers described herein can also be used to control the release of an encapsulated material from a micelle, polymersome or other supramolecular structure by modulating and controlling the micelle or polymersome stability and surface properties.

In embodiments in which the drug molecule is linked to the amphiphilic block copolymer through a covalent bond, the rate of release of the drug molecule from the particle (micelle, polymersome or other supramolecular structure) may be modulated by varying the composition of the linker molecule.

The amphiphilic block copolymer can be a diblock, triblock, or other multi-block copolymer, which may each be referred generically as block copolymers. Each block serves to segregate the hydrophilic and hydrophobic characteristics to provide polarity to the amphiphile. The architecture of each block may be the same or different. In some embodiments, the amphiphilic block copolymer comprises of two or more linear blocks that are attached end-to-end either directly or through any suitable linker molecule. In other embodiments, a branched copolymer block is attached to a linear copolymer block. In other embodiments, a branched copolymer block is attached to a branched copolymer block. In some embodiments, the amphiphilic block copolymer is a brush copolymer, such as a brush copolymer formed by grafting multiple polymer arms to a linear copolymer. In other embodiments, the amphiphilic block copolymer comprises a linear or branched copolymer block linked to a brush copolymer block. In preferred embodiments, the amphiphilic block copolymer comprises linear hydrophilic block linked to a linear hydrophobic block.

### Hydrophilic and Hydrophobic Block Compositions

Amphiphilic diblock copolymers of the present disclosure comprise a hydrophilic block and a hydrophobic block.

The hydrophilic block (sometimes designated “S” in formulae) is a hydrophilic (i.e., water-soluble) polymer that is water soluble in aqueous solutions at pH 7.4 and at body temperature. A function of the hydrophilic block is to stabilize particles formed by amphiphilic block copolymers and prevent aggregation. Hydrophilic blocks with additional features and functions are described throughout the specification.

The hydrophobic block (sometimes designated “H” in formulae) is a hydrophobic polymer or oligomer with substantially limited water solubility, or is amphiphilic in properties, and capable of assembling into supramolecular structures, e.g., micellar, nano- or micro-particles in aqueous solutions at certain concentrations, temperatures and pH.

In certain embodiments, the hydrophobic block is insoluble, or forms particles, in aqueous solutions down to about 0.1 mg/mL or about 0.01 mg/mL or less. In some embodiments, the hydrophobic block is soluble at certain concentrations, temperatures and/or pH ranges but becomes insoluble in response to a change in concentration, temperature and/or pH. For instance, in some embodiments, the hydrophobic block is a hydrophobic polymer that is temperature-responsive, i.e., the hydrophobic polymer is soluble in aqueous solutions at temperatures below a transition temperature (Tₜᵣ) but becomes insoluble at temperatures above the transition temperature.

The hydrophilic and hydrophobic blocks can each independently comprise a linear, branched or brush polymer. The hydrophilic and hydrophobic blocks can each independently be a homopolymer or copolymer. The hydrophilic and hydrophobic blocks can each independently comprise one or many different types of monomer units. The hydrophilic and hydrophobic blocks can each independently be a statistical copolymer or alternating copolymer. The hydrophilic and hydrophobic blocks can each independently be a block copolymer, such as the A-B type, or the blocks can be comprised of a grafted copolymer, whereby two or more polymers are linked through polymer analogous reaction.

The hydrophilic block typically comprises a majority of monomer units selected from neutral hydrophilic monomers (sometimes designated “A” in formulae) and optionally a minority of monomer units selected from charged monomers (sometimes designated “C” in formulae) and/or reactive monomers (sometimes designated “E” in formulae). The hydrophobic block typically comprises a majority of monomer units selected from hydrophobic monomers (sometimes designated “F” in formulae) and optionally a minority of monomer units selected from charged monomers, reactive monomers and/or neutral hydrophilic monomers. In some embodiments, the hydrophobic block comprises a minority of monomer units selected from hydrophobic monomers and a majority of monomer units selected from neutral hydrophilic monomers.

In certain embodiments, the amphiphilic block copolymer comprises a hydrophilic and hydrophobic block, wherein the majority monomer units comprising the hydrophilic block are selected from neutral hydrophilic monomers and the majority of monomer units comprising the hydrophobic block are selected from hydrophobic monomers. In certain preferred embodiments, the amphiphilic block copolymer comprises a hydrophilic and hydrophobic block, wherein the majority monomer units comprising the hydrophilic block are selected from neutral hydrophilic monomers and the majority of monomer units comprising the hydrophobic block are selected from a 1^{st} hydrophobic monomer and a 2^{nd} hydrophobic monomer, wherein the 1^{st} hydrophobic monomer is selected from temperature-responsive monomers. In some embodiments, the amphiphilic block copolymer comprises a hydrophilic and hydrophobic block, wherein the majority monomer units comprising the hydrophilic block and hydrophobic block are selected from neutral hydrophilic monomers.

Monomers comprising the hydrophobic and hydrophilic blocks can be selected from acrylates, (meth)acrylates, acrylamides, (meth)acrylamides, allyl ethers, vinyl acetates, vinyl amides, substituted styrenes, amino acids, acrylonitrile, heterocyclic monomers (e.g., ethylene oxide), saccharides, phosphoesters, phosphonamides, sulfonate esters, sulfonamides, or combinations thereof. In some embodiments, monomers comprising the hydrophobic and hydrophobic blocks are selected from natural biopolymers. Alternatively, in some embodiments, the amphiphilic diblock copolymer may comprise naturally occurring monomers, non-natural monomers or combinations thereof. In some embodiments, natural biopolymers are selected from peptides (sometimes referred to as poly(amino acids)). In still other embodiments, natural biopolymers are selected from polysaccharides, such as glycogen, cellulose, dextran, alginate and chitosan, etc., including derivatives therefore, including alkylated and acetylate saccharides.

In some embodiments, the hydrophilic block and/or hydrophobic block comprise neutral hydrophilic monomers, which may be described generically as hydrophilic monomers. In some embodiments, neutral hydrophilic monomers (or hydrophilic monomers) are selected from (meth)acrylates or (meth)acrylamides of the chemical formula CH₂=CR₂—C(O)—R₁ (“Formula I”), wherein the acryl side group R₁ may be selected from one or more of the groups consisting of —OR₃, —NHR₃ or —N(CH₃)R₃, where R₂ can be H or CH₃, and R₃ is independently selected from any hydrophilic substituent. Non-limiting examples of R₃ include but are not limited to H (except for OR₃), CH₃, CH₂CH₃, CH₂CH₂OH, CH₂(CH₂)₂OH, CH₂CH(OH)CH₃, CHCH₃CH₂OH or (CH₂CH₂O)ᵢH, where i is an integer number of repeating units from, for example, 0 to 20, 0 to 6, or 1 to 11, or 1 to 10, or 1 to 6, or 1 to 5, or 2 to 15, or 2 to 10, or 3 to 19, or 3 to 15, or 3 to 10, or 1, 2, 3, 4, 5, 6, 7, 8, 9, 10, 11, 12, 13, 14, 15, 16, 17, 18, 19 or 20. Note the description (meth)acrylates or (meth)acrylamides is used herein to encompass acrylates, methacrylates, acrylamides and methacrylamides. In some embodiments R₃ is a linear or branched alkyl with from 2 to 6 carbon atoms, for example isopropyl.

A non-limiting example of a neutral hydrophilic monomer of Formula I wherein R₁=NHR₄, R₂=CH₃, and R₃=CH₂CH(OH)CHais N-2-hydroxypropyl(methacrylamide) (HPMA):

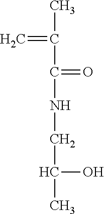

In some embodiments, the hydrophilic block and/or hydrophobic block comprise charged monomers that contain a functional group that either has a fixed charge or has net charge under certain physiological conditions. Non-limiting examples of charged monomers include (meth)acrylamides and (meth)acrylates that comprise amine, quaternary ammonium, sulfonic acid, sulfuric acid, sulfonium, phosphoric acid, phosphonic acid, phosphonium, carboxylic acid and/or boronic acid functional groups, as well as any composition of salts thereof. Non-limiting examples of salts of include e.g., positively charged functional groups, e.g., ammonium ions paired with halide (e.g., chloride) ions. Other non-limiting examples of suitable salts of charged amino acids include conjugate bases of carboxylic, sulfonic and phosphonic acids, paired with group 1 metals, such as sodium, or ammonium or guanidinium ions.

In some embodiments, charged monomers are selected from (meth)acrylates and (meth)acrylamides with chemical formula CH₂=CR₅—C(O)—R₄ (“Formula II”). The acryl side group R₄ may be selected from one or more of the groups consisting of —OR₆, —NHR₆ or —N(CH₃)R₆, where R₅ can be H or CH₃ and R₆ can be selected from, but is not limited to, OH, linear alkyl structures such as (CH₂)ⱼNH₂, (CH₂)ⱼCH(NH₂)COOH, (CH₂)ⱼCOOH, (CH₂)ⱼPO₃H₂, (CH₂)ⱼOPO₃H₂, (CH₂)ⱼSO₃H, (CH₂)ⱼOSO₃H, (CH₂)ⱼB(OH)₂, where j is an integer number of a repeating units, typically between 1 to 6, such as 1, 2, 3, 4, 5 or 6, and more versatile structures such as CH₂CH₂N(CH₃)₂, CH[CH₂N(CH₃)₂]₂, CH(COOH)CHCH₂COOH, [CH₂CH(CH₃)O]ₛPO₃H₂, (CH₂)₃CH(OPO₃H₂)(CH₂)₂CH(OPO₃H₂)(CH₂)₃CH₃, C(CH₃)₂CH₂SO₃H, and C₆H₄B(OH)₂.

A non-limiting example of a charged monomer of Formula II wherein R₄=—OR₆, Rₛ=CH₃ and R₈=OH is:

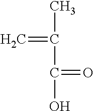

wherein in this example, the monomer would be expected to be deprotonated at physiologic pH (i.e., pH 7.4) and carry a negative charge.

In some embodiments, the hydrophilic block and/or hydrophobic block comprise a reactive monomer. Suitable reactive monomers include but are not limited to any monomer bearing a functional group suitable for attachment of drug molecules, including monomers with azide, alkyne, tetrazine, transcyclooctyne (TCO), protected hydrazine, ketone, aldehyde, certain hydroxyl groups, isocyanate, isothiocyanate, activated carboxylic acid, protected maleimide, thiol and/or amine groups. Suitable linker chemistries used to link drug molecules to the reactive monomer are discussed throughout the present specification. While reactive monomers may comprise functional groups that can impart charge and/or improve water solubility, such as carboxylic acid and hydroxyl groups, respectively, and may also therefore be classified as charged monomer or neutral hydrophilic monomers, the classification of a monomer as a reactive monomer is context-dependent and based on its intended use. For example, monomers comprising carboxylic acids would be considered charged monomers if the carboxylic acid is not used for drug attachment, whereas the same monomers linked to an amine bearing drug molecule, e.g., via an amide bind, would be considered a reactive monomer.

In some embodiments, the hydrophilic block and/or hydrophobic block comprise reactive monomers selected from (meth)acrylates and (meth)acrylamides of chemical formula CH₂=CR₈—C(O)—R₇ (“Formula III”). The acryl side group R₇ may be selected from one or more of the groups consisting of —OR₉, —NHR₉ or —N(CH₃)R₉, where R₃ can be H or CH₃ and R₉ can be independently selected, but is not limited to, linear alkyl structures such as (CH₂)ₖR₁₀, (CH₂)ₖC(O)NHR₁₀ or (CH₂CH₂O)ₖCH₂CH₂C(O)NHR₁₀, where k is an integer number of repeating units, typically between 0 to 6, and R₁₀ is independently selected from (CH₂)ₕ-FG, (CH₂CH₂O)ₕCH₂CH₂—FG or (CH₂CH₂O)ₕCH₂CH₂—FG, where h is an integer number of repeating units and FG is any functional group, which may be selected from, but not limited to, carboxylic acid and activated carboxylic acids (e.g., carbonylthiazolidine-2-thione, tert-butyl and/or nitrobenzyl protected carboxylic acid), anhydride, amine and protected amines (e.g., tert-butyloxycarbonyl protected amine), OSi(CH₃), CCH, N₃, propargyl, halogen (e.g., fluoride, chloride), olefins and endo cyclic olefins (e.g., allyl), CN, OH, and epoxy. In some embodiments h and k are an integer number of repeating units from, for example, 0 to 20, 0 to 6, or 1 to 11, or 1 to 10, or 1 to 6, or 1 to 5, or 2 to 15, or 2 to 10, or 3 to 19, or 3 to 15, or 3 to 10, or 1, 2, 3, 4, 5, 6, 7, 8, 9, 10, 11, 12, 13, 14, 15, 16, 17, 18, 19 or 20.

A non-limiting example of a reactive monomer of Formula III wherein R₇ is NHR₉, Rₐ is CH₃, R₉ is (CH₂)ₖC(O)NHR₁₀, k is equal to 2 and R₁₀ is propargyl is:

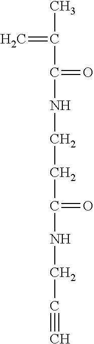

The hydrophobic block typically comprises a majority of monomer units selected from hydrophobic monomers, which may be the same or different.

Certain monomers described herein as hydrophobic monomers may be water soluble under certain conditions but are hydrophobic and water insoluble as amphiphilic block copolymer compositions at certain conditions. Non-limiting examples include temperature-responsive monomers, such as N-isopropylmethacrylamide (NIPMAM); a homopolymer comprised entirely of NIPMAM may be water soluble at room temperature but may become insoluble and form particles at elevated temperatures. Such distinctions are made to facilitate description of certain embodiments.

In some embodiments, the hydrophobic block may include monomers of (meth)acrylates, (meth)acrylamides, styryl and vinyl moieties. In some embodiments, hydrophobic monomers are selected from (meth)acrylates or (meth)acrylamides of chemical formula CH₂=CR₁₂—C(O)—R₁₁ (“Formula IV”), wherein the acryl side group R₁₁ may be selected from one or more of the groups consisting of —OR₁₃, —NHR₁₃ or —N(CH₃)R₁₃, where R₁₂ can be H or CH₃, and R₁₃ is independently selected from any hydrophobic substituent, sometimes referred to as a hydrophobic group. Non-limiting examples of R₁₃ include but are not limited to aliphatic groups often having three or more carbon atoms, which may be linear or branched or saturated or unsaturated, including linear chains such as —(CH₂)ₗCH₃, wherein l is an integer number greater than or equal to three; branched chains such as CH(CH₃)₂, (CH₂)ₗCH(CH₃)₂, wherein l is an integer number greater than or equal to one; and cyclic rings, such as (CH₂)ₗ(C₅H₉), (CH₂)ₗ(C₆H₁₁), (CH₂)ₗ(C₇H₁₃) or (CH₂)ₗ(C₈H₁₅), wherein l is an integer number greater than or equal to zero. In some embodiments l is an integer number of repeating units from, for example, 0 to 20, 0 to 6, or 1 to 11, or 1 to 10, or 1 to 6, or 1 to 5, or 2 to 15, or 2 to 10, or 3 to 19, or 3 to 15, or 3 to 10, or 1, 2, 3, 4, 5, 6, 7, 8, 9, 10, 11, 12, 13, 14, 15, 16, 17, 18, 19 or 20.

A non-limiting example of a hydrophobic monomer of Formula IV wherein R₁₁ is NHR₁₃, R₁₂ is CH₃ and R₁₃ is CH(CH₃)₂ is N-isopropylmethacrylamide (NIPMAM), which has the structure:

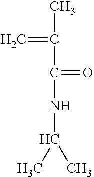

In certain preferred embodiments, the hydrophobic block comprises a majority of monomer units of NIPMAM. In other embodiments, the hydrophobic block comprises a majority of monomer units selected from N-isopropylacrylamide (NIPAM, or sometimes NIPAAM).

An additional non-limiting example of a hydrophobic monomer of Formula IV wherein R₁₁ is NHR₁₃, R₁₂ is CH₃, R₁₃ is —(CH₂)ₗCH₃ and l is equal to 3 is:

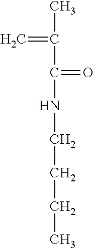

Additional non-limiting examples of R₁₃ include hydrophobic alcohols, such as —CH₂(CH₂)ₗOH, wherein l is an integer number greater than or equal to 3; amphiphilic ethers that are hydrophobic under certain conditions, such as (CH₂CH₂O)ₗCH₃, (CH₂CH₂O)₁CH₂CH₃, wherein l is an integer number greater than or equal to 2; and hydrophobic ethers, such as (CH₂CH₂CH₂O)₁H or (CH₂CH₂CH₂O)ₗCH₃ wherein l is an integer number greater than or equal to 2. In some embodiments l is an integer number of repeating units from, for example, 0 to 20, 0 to 6, or toll, or to 10, or to 6, or 1 to 5, or 2 to 15, or 2 to 10, or 3 to 19, or 3 to 15, or 3 to 10, or 1, 2, 3, 4, 5, 6, 7, 8, 9, 10, 11, 12, 13, 14, 15, 16, 17, 18, 19 or 20.

A non-limiting example of a hydrophobic monomer of Formula IV wherein R₁₁ is OR₁₃, R₁₂ is CH₃, R₁₃ is (CH₂CH₂O)ₗCH₃ and l is equal to 3 is triethylene glycol methyl ether methacrylate (TEGMA), which has the structure:

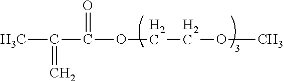

In certain preferred embodiments, the hydrophobic block comprises a majority of monomer units of TEGMA. In other embodiments, the hydrophobic block comprises a majority of monomer units selected from 2-(2-methoxyethoxy)ethyl methacrylate (DEGMA) which has the structure:

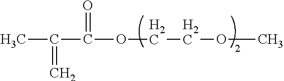

In certain preferred embodiments, the hydrophobic block comprises hydrophobic monomers further comprising aromatic groups. Exemplary aromatic groups (sometimes referred to as “aromatics” or “aromatic rings”) include but are not limited to benzene and fused benzene ring structures (also referred to as fused phenyl groups) or heterocyclic aromatic molecules. The following non-limiting examples of aromatic groups may be present in hydrophobic monomers:

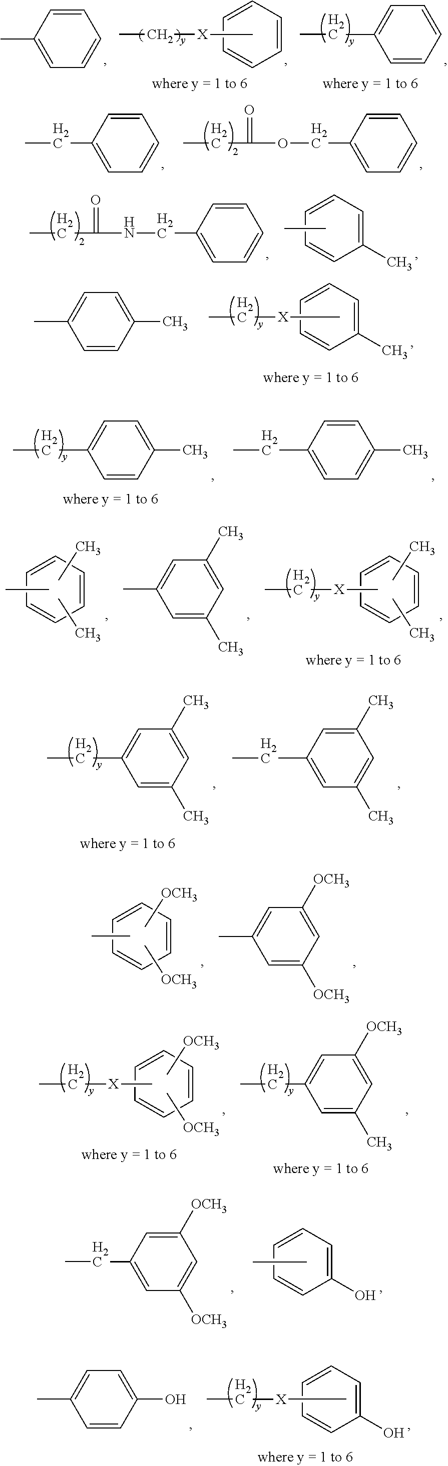

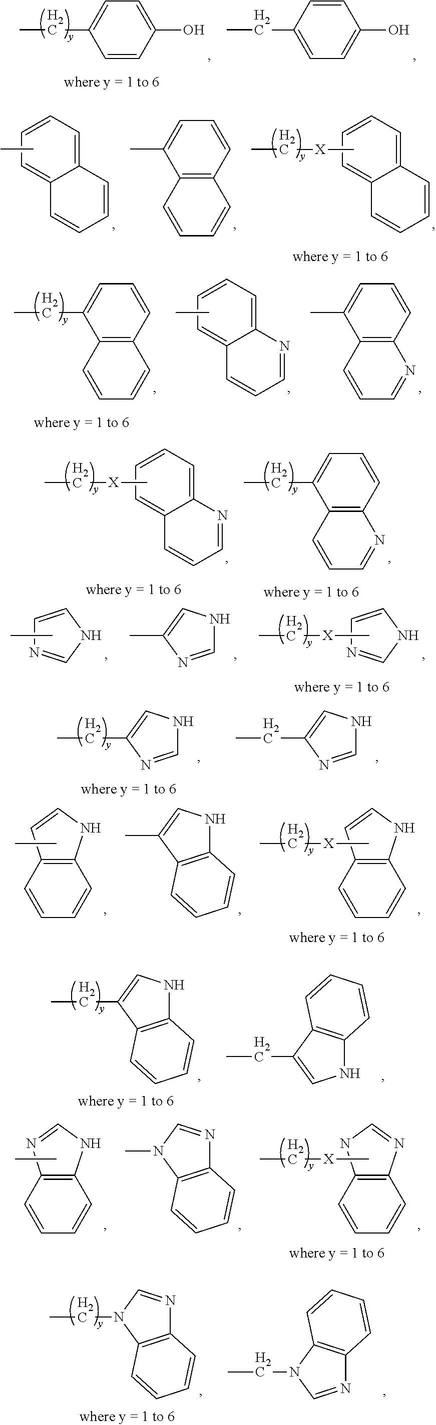

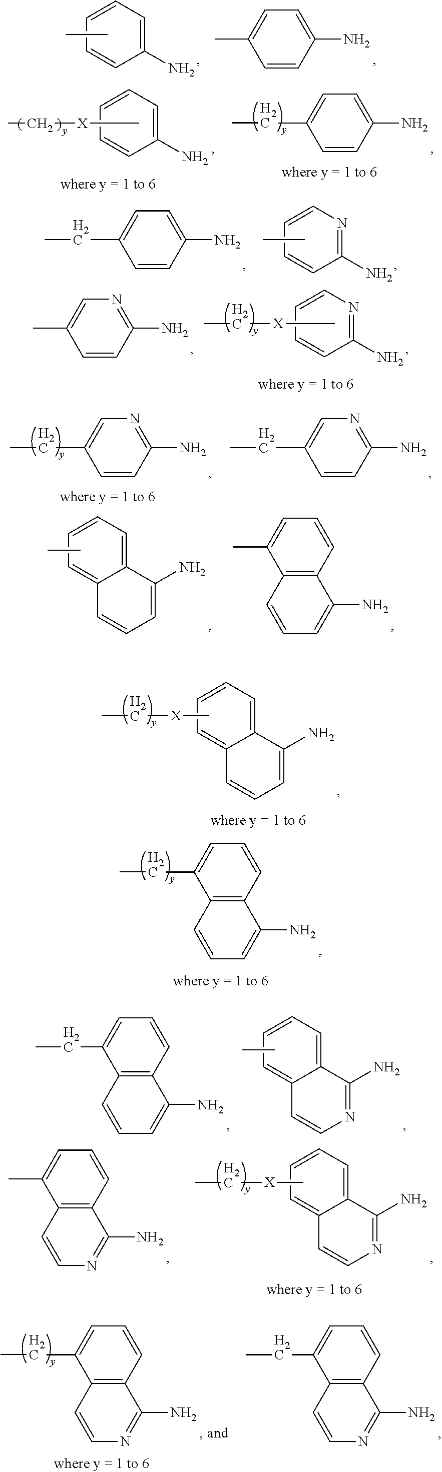

wherein X is any suitable linker molecule and y is an integer value, typically between 1 and 6.

In some embodiments, the hydrophobic monomer comprises fused aromatic ring groups (e.g., naphthalene) or fused heterocyclic aromatic groups (e.g., xanthene or quinoline). In some embodiments, the hydrophobic block comprises reactive monomers linked to hydrophobic drug molecules. In some embodiments, the hydrophobic drug molecules (e.g., imidazoquinolines) are aromatic and thus the reactive monomers linked to hydrophobic drug molecules comprising aromatic groups can also be considered hydrophobic monomers comprising aromatic groups.

A non-limiting example of a hydrophobic monomer of Formula IV wherein R₁₁ is NHR₁₃, R₁₂ is CH₃, and

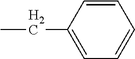

R₁₃ is benzyl methacrylamide (BnMAM), which has the structure:

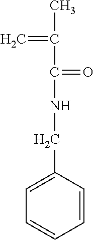

In some embodiments, the hydrophobic block comprises a majority of monomer units selected from BnMAM. In certain preferred embodiments, the hydrophobic block comprises a majority of monomer units selected from temperature-responsive monomers, such as NIPMAM, and a minority of monomer units selected from BnMAM.

In certain embodiments, the hydrophobic block comprises fluorinated aliphatic or aromatic groups, wherein one or more hydrogen atoms of the aforementioned aliphatic or aromatics groups comprising the hydrophobic monomer may be substituted for one or more fluorine atoms. The following non-limiting examples of fluorinated aromatic groups may be present in hydrophobic monomers:

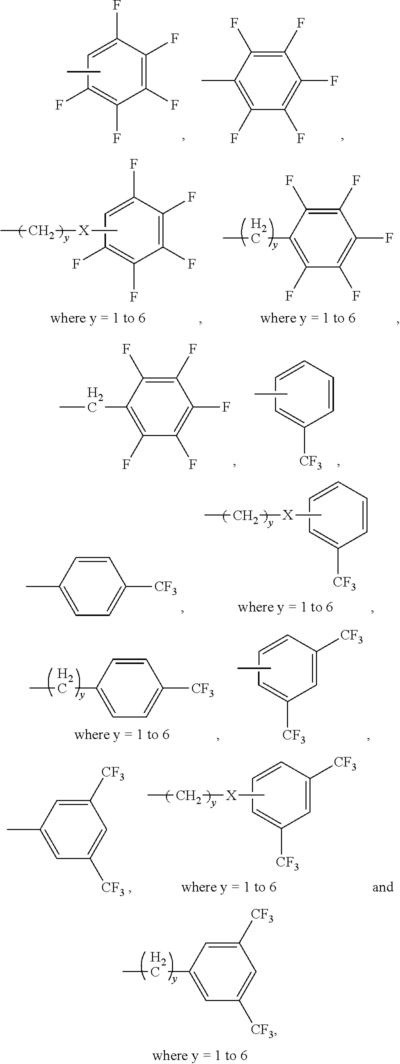

wherein X is any suitable linker molecule and y is an integer value, typically between 1 and 6.

In some embodiments, the hydrophobic block comprises hydrophobic monomers selected from naturally occurring or non-naturally occurring amino acids and/or sugars. Non-limiting examples of hydrophobic amino acids include leucine, isoleucine, norleucine, valine, tryptophan, phenylamine, tyrosine and methionine, as well as hydrophilic amino acids that have been modified, such as by acetylation or benzoylation to have hydrophobic characteristics. Similarly, hydrophilic polysaccharides, which may include but are not limited to glycogen, cellulose, dextran, alginate and chitosan can be modified chemically via acetylation or benzoylation to render the resulting modified polysaccharide water insoluble. In still further embodiments the hydrophobic block comprises monomers selected from lactic acid and/or glycolic acid.

In some embodiments, the hydrophobic block comprises a majority of monomer units selected from hydrophobic monomers that are temperature-responsive (sometimes referred to as “temperature-responsive monomers”), such as NIPAM, NIPMAM, N,N′-diethylacrylamide (DEAAM), N-(L)-(1-hydroxymethyl)propyl methacrylamide (HMPMAM), N,N′-dimethylaminoethylmethacrylate (DMAEMA), N—(N-ethylcarbamido)propylmethacrylamide, N-vinylisobutyramide (PNVIBA), N-vinyl-n-butyramide (PNVBA), N-acryloyl-N-propylpiperazine (PNANPP), N-vinylcaprolacta (PVCa), DEGMA, TEGMA, or poly(amino acids) or γ-(2-methoxyethoxy)esteryl-L-glutamate. The LCST of homopolymers formed by temperature-responsive monomers is provided for reference (Table 1), wherein monomers may be grouped into one of four different classes, as designated herein, based on experimentally determined transition temperature or LCST.

TABLE 1
Classes of temperature-responsive monomers based on experimentally determined LCST or Tₜᵣ.

| Class of | | |
|---|---|---|
| temperature- | | |
| responsive | | LCST or |
| monomer | Polymer composition | Tₜᵣ (° C.) |
| Class I: | Poly(N-isopropylacrylamide) p(NIPAM) | 32 |
| LCST or Tₜᵣ: 25 to | Poly(N,N-diethylacrylamide) p(DEAM) | 33 |
| 35° C. | Poly(N-(1-hydroxymethyl)propylmethacrylamide) | 30 (L-iso); |
| | p(HMPMA) | 34 (DL-mix) |
| | Poly(N-vinyl-n-butyramide) p(NVBA) | 32 |
| | Poly(N-vinylcaprolactam) p(VCa) | 32 |
| | Poly(N-vinylpyrrolidone) p(VPy) | 30 |
| | Poly(2-(2-methoxyethoxy)ethyl methacrylate) | 26 |
| | p(MEO2MA), sometimes referred to as DEGMA | |
| | Poly(endo,exo-bicyclo[2.2.1]hept-5-ene-2,3-dicarboxylic | 25 |
| | acid, bis[2-[2-(2-ethoxyethoxy)ethoxy]ethyl] ester) | |
| | Oligo(ethylene oxide)-grafted polylactide | 27 |
| | P(Val-Pro-Gly-Val-Gly)(SEQ ID NO: 3) | 27 |
| | Val-Pro-Gly-Val-Gly (SEQ ID NO: 3) and oligo(ethylene | 16-30 |
| | glycol) grafted polynorbornene | |
| Class II: | Poly(N-vinylisobutyramide) p(NVIBA) | 39 |
| LCST or Tₜᵣ: 36 to | Poly(N-acryloyl-N0-propylpiperazine) p(NANPP) | 37 |
| 45° C. | Poly(4-vinylbenzyl methoxytetrakis(oxyethylene)ether) | 39 |
| Slightly Above | Poly[bis((ethoxyethoxy)ethoxy)phosphazene] p(BEEP) | 38 |
| Body Temperature | Poly[bis(2,3-bis(2-methoxyethoxy)propanoxy) | 38 |
| | phosphazene] p(BBMEPP) | |
| | Poly(2-(2-ethoxy)ethoxyethyl vinyl ether) p(EOEOVE) | 41 |
| | Poly(2-isopropyl-2-oxazoline) p(iPOx) | 36 |
| | Poly(methyl vinyl ether) p(MVE) | 35-36 |
| | Poly(methyl vinyl ether) p(MVE) | 35-36 |
| | Poly(2-n-propyl-2-oxazoline) p(nPOx) | 36 |
| | Poly(N-isopropylmethacrylamide) p(NIPMAM) | ~45 |
| Class III: | Poly(N-cyclopropylacrylamide) p(NCPAM) | 53 |
| LCST or Tₜᵣ: 45 to | Poly(N-(N′-ethylcarbamido)propyl methacrylamide) | 49.5-56.5 |
| 60° C. | p(iBuCPMA) | |
| | Poly(N-(2-ethoxy-1,3-dioxan-5-yl) methacrylamide) | 52 |
| | p(NEM) | |
| | Poly[N-(2-methacryloyloxyethyl) pyrrolidone] p(NMP) | 51.9 |
| | Poly(N-acryloylpyrrolidine) p(APR) | 51 |
| | Poly(2-[2-(2-methoxyethoxy)ethoxy]ethyl methacrylate) | 52 |
| | p(MEO3MA), sometimes referred to as TEGMA | |
| | Poly(oligo(ethylene glycol) methacrylate p(OEGMA) | 60-90 |
| | Poly(2-ethyl-2-oxazine) (PEtOZI) | 56 |
| | | (n > 100) |
| Class IV: | Copolymer of N-isopropylmethacrylamide and a | 13-44 |
| Broadly observed | methacrylamide monomer with labile hydrazone linkages | |
| Tₜᵣ | Poly(ethoxyethyl glycidal ether) | 29.6-40.4 |
| | Poly([N-(2,2-dimethyl-1,3-dioxolane)methyl] acrylamide- | 23-49 |
| | co-[N-(2,3-dihydroxyl-n-propyl)] acrylamide) | |
| | Poly(dimethylaminoethyl methacrylate) (PDMAEMA) | 14-50 |
| | Poly[(di(ethylene glycol) ethyl ether acrylate)-co- | 15-90 |
| | (oligoethylene glycol acrylate)] p(DEGA-co-OEGA) | |
| | Val-Pro-Gly-Val-Gly (SEQ ID NO: 3) derived | 15-55 |
| | polymethacrylate | |
| | Poly(2-hydroxypropylacrylate) p(HPA) | 30-60 |
| | Poly([oligo(2-ethyl-2-oxazoline) methacrylate]-co-(methyl | 35-80 |
| | methacrylate)) | |

In some embodiments, the hydrophobic block may be comprised of two monomers referred to as “F1” and “F2” in the formula p[(F1)_{f1}-co-(F2)_{f2}] where subscripts f1 and f2 refer to the number of monomer units of monomers F1 and F2 respectively. In preferred embodiments, the first hydrophobic monomer referred to as “F1” is selected non-exhaustively from thermo-responsive monomers included in Table 1 that undergo a transition from a hydrophilic to hydrophobic state in response to an increase in temperature. The change in hydrophobicity of these monomers may be attributed to an entropic effect associated with increased water entropy at higher temperature. F1 monomers may contribute to thermo-responsivity of the hydrophobic block enabling the block polymer to undergo a transition from water soluble in a unimer state at temperatures below to the transition temperature to hydrophobic and aggregated at temperatures above the transition temperature.

In some embodiments, the thermo-responsivity associated with the hydrophobic block comprised of monomers F1 and F2 may enable the block polymer to aggregate from a unimer state to an aggregate state above the thermo-responsive transition temperature with the hydrophobic block aggregating with itself. In other embodiments, the thermo-responsivity associated with the hydrophobic block comprised of monomers F1 and F2 may enable the block polymer to aggregate with other hydrophobic molecules in the aqueous environment. In other embodiments, the thermo-responsivity associated with the hydrophobic block comprised of monomers F1 and F2 may enable the block polymer to aggregate with other unimers in a manner that forms size stable nanoparticles.

In preferred embodiments, the hydrophobic block comprises a majority of monomer units selected from temperature-responsive monomers, typically selected from temperature-responsive monomers selected from Class II temperature-responsive monomers and a minority of monomer units selected from hydrophobic monomers that comprise an aromatic group.

### Selection of Hydrophobic Block Compositions

The propensity of amphiphilic block copolymers to undergo particle formation in aqueous solutions is driven by the composition of the hydrophobic block. Hydrophobic blocks may be water insoluble at any concentration, pH or temperature, or may be insoluble only at certain concentrations, pH or temperature. Disclosed herein are amphiphilic block copolymer compositions that form particles in aqueous solutions over a broad range of concentrations, temperatures and pH, and amphiphilic block copolymer compositions that undergo particle formation only at certain concentrations, temperatures and pH. In certain preferred compositions of drug carriers, the amphiphilic block copolymers comprise a temperature-responsive hydrophobic block and exist as soluble single molecules in aqueous solutions below body temperature, but undergo particle assembly when dispersed in aqueous solutions, including buffered solutions, at concentrations less than 100 mg/mL, typically less than 50 mg/mL and at temperatures greater than or equal to body temperature.

A major limitation of temperature-responsive amphiphilic block copolymers that have been used as drug carriers to-date is that they are insufficiently hydrophobic and/or insufficiently stable in vivo and tend to fall apart and rapidly disperse following administration to a subject. For instance, temperature-responsive amphiphilic block copolymers based on PEG-b-PPG, pHPMA-b-pNIPAM and pHPMA-b-pDEGMA diblock polymers have been used for drug delivery but comprise hydrophobic blocks, i.e., PPG, pNIPAM and pDEGMA, respectively, that are insufficiently hydrophobic and yield micelles that tend to rapidly dissociate when diluted. PLGA-based temperature-responsive amphiphilic block copolymers, such as certain PEG-PLGA compositions, have also been thoroughly investigated for drug delivery applications, but are comprised of ester-based backbones that readily fall apart in vivo.

An unexpected finding disclosed herein is that the stability of particles formed by temperature-responsive amphiphilic block copolymers with hydrophobic blocks comprising a majority of monomer units selected from a first monomer that is temperature-responsive can be improved by incorporating a second hydrophobic monomer into the hydrophobic block. For such embodiments, it was observed that the second hydrophobic monomer can reduce the transition temperature of the temperature-responsive amphiphilic block copolymer in proportion to the density and hydrophobicity of the second monomer. Based on the findings disclosed herein, hydrophobic monomers can be grouped into one of four classes of monomers from least to most impactful on transition temperature: those comprising lower alkyl, aromatic, higher alkyl or fluorinated groups, which cause about a 10-20° C. decrease in transition temperature for every 10-30 mol %, 5-15 mol %, 1-10 mol % and 1-10 mol % density, respectively, added into the hydrophobic block of a temperature-responsive amphiphilic block copolymer. Hydrophobic blocks and amphiphilic block copolymers comprising different classes of hydrophobic monomers, as well as their impact on transition of temperature-responsive polymers, are described throughout. Note: an additional finding disclosed herein is that hydrophobic monomers comprising fused aromatic rings (e.g., naphthalene), which may be heterocyclic (e.g., quinoline), have a larger effect on the transition temperature of temperature-responsive polymers than hydrophobic monomers comprising aromatic groups with a single ring (e.g., benzyl group), with the lowering effect on transition temperature approximately proportional to the total number of carbons. For example, 20 mol % hydrophobic monomer comprising a benzyl group would be expected to have a similar effect on the lowering of the transition temperature of a temperature-responsive polymer than 10 mol % hydrophobic monomer comprising a naphthyl group.

Notably, in addition to modulating the transition temperature, hydrophobic monomers comprising aromatic groups were also found to enhance the stability of particles formed by temperature-responsive amphiphilic block copolymers. Specifically, increasing the density of aromatic hydrocarbon monomers resulted in a greater proportion of amphiphilic block copolymers existing in the micellar state as opposed to monomeric state in aqueous solutions at temperatures above the transition temperature. A non-limiting explanation for this observation is that aromatic hydrophobic monomers undergo pi-stacking and provide greater stabilization of the particles, thereby reducing the propensity of the individual polymer chains to exist in the monomeric state.

While it was observed that incorporation of hydrophobic monomers comprising aromatic groups in the hydrophobic block can improve the stability of particles formed by temperature-responsive amphiphilic block copolymers, it was also observed that the composition of monomers comprising the hydrophobic block must be carefully selected to ensure that the transition temperature and size of particles formed are operable for the intended application(s) of such compositions. For instance, temperature-responsive amphiphilic block copolymers comprising drug molecules used as injectable drug products should have transition temperatures between about 20 to 34° C., such as 20, 21, 22, 23, 24, 25, 26, 27, 28, 29, 30, 31, 32, 33 or 34° C., though, more preferably between about more preferably between about 22 to 32° C. The results herein show that >10 mol % hydrophobic monomers comprising aromatic groups stabilize particles but also typically result in a >15° C. decrease in transition temperature (as compared with the homopolymers). Based on these unexpected results, temperature-responsive monomers should be selected from temperature-responsive monomers that have transition temperatures (or LCST) between about 30 to 50° C. as homopolymers, such as certain Class I, Class II or Class Ill temperature-responsive monomers, though, more preferably between about 35 to 45° C. (e.g., Class II monomers, Table 1), and can thus accommodate 10 mol % or higher densities of hydrophobic monomers comprising aromatic groups. Notably, certain embodiments of temperature-responsive amphiphilic block copolymers were also observed to have concentration-responsive properties and exist as soluble single molecules at concentrations greater than or equal to 50 mg/mL. For such temperature-responsive amphiphilic block copolymers that are also concentration-responsive and exist as soluble single molecules at concentrations above 50 mg/mL but assemble into particles in aqueous solutions at concentrations less than 50 mg/mL when the temperature of the solution is greater than transition temperature, lower transition temperatures can be tolerated. More specifically, for temperature-responsive amphiphilic block copolymers that are also concentration-responsive, suitable transition temperatures are those that are less than 34° C.

In some embodiments, the amphiphilic block copolymers described herein exist as unimers in an aqueous solvent. In some embodiments, the concentration of unimers is greater than 50 mg/mL. In some embodiments, the temperature of the solution is below the transition temperature. In some embodiments, the amphiphilic block copolymers described herein exist as particles in an aqueous solvent. In some embodiments, the concentration of unimers is less than or equal to 50 mg/mL. In some embodiments, the temperature of the solution is at the transition temperature or is higher than the transition temperature. In some embodiments, the transition temperature is 1° C. or more to 37° C. or lower. In some embodiments, the transition temperature is about 20° C. to about 34° C. In some embodiments, the transition temperature is body temperature

In certain preferred embodiments of temperature-responsive amphiphilic block copolymers, the hydrophobic block comprises a first hydrophobic monomer comprising a temperature-responsive monomer selected from NIPMAM, NANPP, NVIBA, BEEP, or TEGMA and a second hydrophobic monomer selected from hydrophobic monomers comprising non-fluorinated aromatic groups (e.g., BnMAM), wherein the density of the 1^{st} hydrophobic monomer is between 50 to 95 mol %, such as 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94 or 95 mol %, and the density of the 2^{nd} monomer is between 5 to 50 mol %, such as 5, 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mol %. In certain preferred embodiments, a first hydrophobic monomer is selected from NIPMAM and is distributed along the hydrophobic block at a density of between 50 to 85 mol %, such as between 70 and 85 mol %, and a second hydrophobic monomer is selected from BnMAM and is distributed along the hydrophobic block at a density of between 15 mol % and 50 mol %, such as between 15 and 30 mol %.

In some embodiments of amphiphilic block copolymers, the hydrophobic block comprises a hydrophobic monomer selected from hydrophobic monomers of Formula IV, preferably a hydrophobic monomer selected from monomers of Formula IV comprising an aromatic group, such as BnMAM, and a hydrophilic monomer selected from hydrophilic monomers of Formula I, preferably HPMA; and, the hydrophilic monomer is distributed along the hydrophobic block at a density of between about 10-90 mol %, preferably about 75 mol % or less, and the hydrophobic monomer is distributed along the hydrophobic block at a density of between about 10-90 mol %, preferably about 25 mol % or higher; though, in some embodiments the hydrophilic monomer is absent and the hydrophobic monomer is distributed along the hydrophobic block at a density of 100 mol %.

Selection of hydrophilic block compositions

Hydrophilic blocks function to stabilize particles formed amphiphilic block copolymers in aqueous solutions. Hydrophilic blocks stabilize particles by creating a hydrated shell that prevents particle aggregation and/or by providing countervailing forces, such as electrostatic charge that provides a repulsive force between particles. In addition to impacting solubility and particle stability, hydrophilic blocks can also be selected to modulate pharmacokinetics, biodistribution, cell interactions, cell uptake, intracellular processing, as well as interactions with blood components, such as antibodies and complement. Thus, hydrophilic blocks must be selected to meet the specific demands of the intended application.

In certain embodiments of amphiphilic block copolymers of the present disclosure, the hydrophilic block is neutral and comprises a majority of monomer units selected from neutral, hydrophilic monomers, such as PEG, neutral hydrophilic polymers of Formula I (e.g., HPMA, HEMAM or HEA) or neutral saccharides, e.g., dextran. In preferred embodiments of amphiphilic block copolymers, the hydrophilic block comprises monomers selected from neutral hydrophilic polymers of Formula I, preferably HPMA. HPMA may be selected as the majority monomer unit comprising hydrophilic blocks based on its high solubility in aqueous solutions that promotes micellization and biocompatibility that minimizes interactions with cells and blood components.

For some applications, hydrophilic block compositions are selected to promote interactions with cells or blood components. For instance, amphiphilic block copolymers used to stimulate immune responses may comprise hydrophilic blocks further comprising charged monomers or reactive monomers linked to saccharides that promote interactions with immune cells. A non-limiting explanation is that the positive charge promoted interactions with and uptake by immune cells that promote T cell immunity. For certain compositions of amphiphilic diblock copolymers used for promoting tolerance, negatively charged monomers and/or sugars that bind to antigen-presenting cell scavenger receptors were incorporated into the hydrophilic block and associated with improved tolerance induction. A non-limiting explanation is that the negative charge and/or sugar residues that bind to scavenger receptors led to improved uptake by antigen-presenting cells that promote tolerance.

### Selection of Amphiphilic Diblock Copolymer Compositions for Use as Drug Carriers

There is a need for particle drug delivery systems that are a uniform size between about 20 to 200 nm diameter, particularly between about 30 to 80 nm diameter, more preferably between 30 to 60 nm, which are needed for various medical applications, such as for vitreous drug delivery that requires particles of sufficiently large size (>20 nm diameter, preferably >30 nm diameter) to keep drug molecules retained in the vitreous but not too large (<100 nm, preferably <80 nm diameter, more preferably <60 nm) to appreciably scatter light. The present disclosure addresses this need by disclosing compositions of amphiphilic block copolymers that unexpectedly lead to particles within a narrow size range between 20 to 200 nm diameter, such as between 30 to 80 nm diameter, need for certain medical applications, such as vitreous drug delivery.

In some embodiments, an amphiphilic block copolymer as described herein comprises a hydrophobic block, wherein the hydrophobic block is comprised of 50 to 95 mol % of a first hydrophobic monomer and of 5 to 50 mol % of a second hydrophobic monomer. In some embodiments, the hydrophobic block is comprised of 70 to 85 mol % of the first hydrophobic monomer and of 15 to 30 mol % of the second hydrophobic monomer.

In some embodiments, an amphiphilic block copolymer as described herein comprises a hydrophobic block, wherein the hydrophobic block is comprised of 80 to 99 mol % of a first hydrophobic monomer and of 1 to 20 mol % of a second hydrophobic monomer. In some embodiments, the hydrophobic block is comprised of 90 to 99 mol % of the first hydrophobic monomer and of 1 to 10 mol % of the second hydrophobic monomer.

In some embodiments, an amphiphilic block copolymer as described herein comprises a hydrophobic block, wherein the hydrophobic block is comprised of 50 to 95 mol % of a first hydrophobic monomer, and of 5-45 mol % of a hydrophilic monomer. In some embodiments, the hydrophobic block is comprised of 70 to 90 mol % of the first hydrophobic monomer, and of 10 to 30 mol % of the hydrophilic hydrophobic monomer.

In some embodiments, an amphiphilic block copolymer as described herein comprises a hydrophobic block, wherein the hydrophobic block is comprised of 50 to 95 mol % of a first hydrophobic monomer, and of 5 to 45 mol % of a second hydrophobic monomer. In some embodiments, the hydrophobic block is comprised of 70 to 95 mol % of the first hydrophobic monomer, and of 5 to 30 mol % of the second hydrophobic monomer.

In some embodiments, an amphiphilic block copolymer as described herein comprises a hydrophobic block, wherein the hydrophobic block is comprised of 50 to 95 mol % of a first hydrophobic monomer, and of 5 to 45 mol % of a second hydrophobic monomer, and of 5 to 45 mol % of a hydrophilic monomer. In some embodiments, the hydrophobic block is comprised of 70 to 85 mol % of the first hydrophobic monomer, and of 5 to 25 mol % of the second hydrophobic monomer, and of 5 to 25 mol % of the hydrophilic hydrophobic monomer.

A key parameter impacting the size of particles formed by the amphiphilic diblock copolymers disclosed herein is the ratio (“block ratio”) of the hydrophilic and hydrophobic block lengths, which may be expressed as the ratio of molecular weight of the hydrophilic and hydrophobic blocks or the ratio of the degree of polymerization of the hydrophilic and hydrophobic blocks. Note: block ratios based on the degree of polymerization are sometimes referred to as a degree of polymerization block ratio, whereas block ratios based on molecular weight are referred as a weight block ratio. For example, an amphiphilic diblock copolymer comprised of a hydrophilic block with 150 HPMA (MW=143.2 g/mol) monomer units and a hydrophobic block with 100 NIPMAM (MW=127.2 g/mol) monomers units has a degree of polymerization block ratio of 1.5 to 1 and a weight block ratio of 1.68 to 1. Unless otherwise specified, block ratio used herein refers to the degree of polymerization block ratio of the hydrophilic block to hydrophobic block.

In some embodiments, an amphiphilic block copolymer as described herein comprises a hydrophilic block and hydrophobic block and has a degree of polymerization block ratio of hydrophilic block to hydrophobic block of 0.5:1 to 4:1. In some embodiments, the degree of polymerization block ratio of hydrophilic block to hydrophobic block of 0.75:1 to 3:1.

An unexpected finding was that amphiphilic diblock copolymers of formula S—H, optionally comprising a drug molecule (e.g., D-S—H, S(D)-H, S—H(D), D-S—H—S, D-S—H—S-D, S(D)-H—S, S(D)-H—S(D) or S—H(D)-S), with block ratios of greater than or equal to 0.5 to 1 (0.5:1), preferably greater than 0.75 to 1 (0.75:1), tended to form nanoparticles with stable hydrodynamic size, whereas those with lower block ratios tended to aggregate. While increasing the hydrophilic block length was found to have a stabilizing effect, it was also observed that an upper limit for hydrophilic to hydrophobic block ratio is preferred for biological activity, and thus a specific range of block ratios is preferred to balance particle stability with biological activity. Thus, in preferred embodiments of amphiphilic diblock copolymers of formula S—H optionally comprising a drug molecule (e.g., D-S—H, S(D)-H, S—H(D), D-S—H—S, D-S—H—S-D, S(D)-H—S, S(D)-H—S(D) or S—H(D)-S), the block ratio is typically greater than or equal to 0.5:1, such as between about 0.5:1 to 4:1, such as 0.5:1, 0.6:1, 0.7:1, 0.8:1, 0.9:1, 1.0:1, 1.1:1, 1.2:1, 1.3:1, 1.4:1, 1.5:1, 1.6:1, 1.7:1, 1.8:1, 1.9:1, 2.0:1, 2.1:1, 2.2:1, 2.3:1, 2.4:1, 2.5:1, 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1 or 4:1, though, more preferably between about 0.75:1 and 3:1, such as 0.75:1, 0.8:1, 0.9:1, 1.1:1, 1.2:1, 1.3:1, 1.4:1, 1.5:1, 1.6:1, 1.7:1, 1.8:1, 1.9:1, 2.0:1, 2.1:1, 2.2:1, 2.3:1, 2.4:1, 2.5:1, 2.6:1, 2.7:1, 2.8:1, 2.9:1 and 3:1.

The specific composition of the hydrophobic block, including the composition and density of monomers, were also found to impact the size and stability of particles formed by amphiphilic diblock copolymers of formula S—H, optionally comprising a drug molecule (e.g., D-S—H, S(D)-H, S—H(D), D-S—H—S, D-S—H—S-D, S(D)-H—S, S(D)-H—S(D) or S—H(D)-S), and led to the identification of specific compositions of amphiphilic diblock copolymers that assembled to 30 to 80 nm diameter particles that were stable for 1 or more days under physiological conditions, e.g., in aqueous buffer at physiological pH (˜pH 7.4) and at body temperature.

A non-limiting example of an amphiphilic diblock copolymer that assembles to 20 to 200 nm diameter particles, such as between 30 to 80 nm dimeter particles, is a temperature-responsive amphiphilic diblock copolymer of formula S—H, optionally comprising a drug molecule (e.g., D-S—H, S(D)-H, S—H(D), D-S—H—S, D-S—H—S-D, S(D)-H—S, S(D)-H—S(D) or S—H(D)-S), that has a block ratio between about 0.5:1 and 4:1, more preferably between about 0.75:1 and 3:1, wherein the hydrophilic block comprises monomers selected from hydrophilic monomers, and optionally charged monomers and/or reactive monomers; the hydrophobic block comprises a first hydrophobic monomer, a second hydrophobic monomer, and optionally charged monomers and/or reactive monomers; the first hydrophobic monomer is selected from hydrophobic monomers that are temperature-responsive, such as monomers of Formula IV that exhibit LCST transition temperatures between about 35 to 45° C. (e.g., Class I temperature-responsive monomers, e.g., NIPMAM); the second hydrophobic monomer is selected from hydrophobic monomers comprising an aromatic group, such as monomers of Formula IV comprising an aromatic group (e.g., BnMAM); and, the first monomer is distributed along the hydrophobic block at a density of between about 50 and 85 mol %, more preferably between about 70 and 85 mol %, and the second monomer is distributed along the hydrophobic block at a density of between about 15 mol % and 50 mol %, more preferably between about 15 and 30 mol %.

An additional finding disclosed herein is that hydrophilic drug molecules, particularly hydrophilic biomolecules, such as hydrophilic glycans, peptides, glycopeptides, proteins or glycoproteins, linked to the hydrophilic block of amphiphilic block copolymers, stabilized particles formed by amphiphilic block copolymers. For instance, in some embodiments of amphiphilic block copolymers of formula D-S—H with a hydrophilic to hydrophobic block ratio of less than or equal to 0.75:1 (note: unless otherwise specified the drug is not included in the calculation of the hydrophilic to hydrophobic block ratios described herein), such as between about 0.25:1 and 0.75:1, wherein the drug molecule is a hydrophilic protein, the amphiphilic block copolymer alone, S—H, has a tendency to aggregate, but the amphiphilic block copolymer linked to a hydrophilic protein (i.e., D-S—H) formed stable nanoparticle micelles. Therefore, in preferred embodiments of amphiphilic block copolymers of formula D-S—H, wherein the drug molecule is a hydrophilic biomolecule, such as a hydrophilic protein, the hydrophilic to hydrophobic block ratio is between about 0.25 and 3:1, such as 0.25:1, 0.3:1, 0.4:1, 0.5:1, 0.6:1, 0.7:1, 0.8:1, 0.9:1, 1.0:1, 1.1:1, 1.2:1, 1.3:1, 1.4:1, 1.5:1, 1.6:1, 1.7:1, 1.8:1, 1.9:1, 2.0:1, 2.1:1, 2.2:1, 2.3:1, 2.4:1, 2.5:1, 2.6:1, 2.7:1, 2.8:1, 2.9:1 and 3:1. In some embodiments wherein the drug molecule is a highly hydrophilic polysaccharide, protein, glycoprotein or proteoglycan, the preferred block ratio may be less than 1:1 or the drug molecule may be attached directly to the hydrophobic block.

In addition to particle size, increasing block ratio and/or attachment of hydrophilic biomolecules, e.g., hydrophilic proteins, were also found to lower the transition temperature of temperature-responsive amphiphilic block copolymers. For instance, increasing the hydrophilic block to hydrophobic block ratio from 0.1:1 to 1:1 typically increased the transition temperature of temperature-responsive amphiphilic block copolymers by about 5-7° C.; increasing the block ratio from about 1:1 to about 2.5:1 resulted in a further increase in transition temperature of about 3-5° C.; and, increasing the block ratio further, from about 2.5:1 to about 4:1 resulted in a further increase in transition temperature of about 2-3° C. Similarly, attachment of biomolecules to temperature-responsive amphiphilic block copolymers also increased the transition temperature in proportion to the size (i.e., molecular weight) and hydrophilicity of the biomolecule. Based on these unexpected findings, preferred hydrophobic block compositions for a given block ratio and/or biomolecule conjugate were identified that provide amphiphilic block copolymer-drug conjugate (e.g., D-S—H) compositions with transition temperatures between about 20 to 34° C. and form stable nanoparticle micelles at body temperature.

For instance, for temperature-responsive amphiphilic block copolymers of formula D-S—H comprising a hydrophilic block comprising a first hydrophilic monomer selected from monomers of Formula I (e.g., HPMA) and a hydrophobic block comprising a first hydrophobic monomer selected from temperature-responsive monomers of Class II (e.g., NIPMAM) and a second hydrophobic monomer selected from hydrophobic monomers comprising non-fluorinated aromatic groups (e.g., BnMAM), wherein the total molecular weight of the amphiphilic diblock copolymer (excluding the drug molecule) is between about 15 to 45 kDa, non-limiting exemplary combinations of block ratio and hydrophobic block monomer density for different drug molecules are:
0.75:1 to 3:1 block ratio and densities for the first hydrophobic monomer and second hydrophobic monomer of between about 75 and 85 mol % and 15 and 25 mol %, respectively, for D-S—H wherein the drug molecule is a hydrophilic small molecule, peptide or glycopeptide <10 kDa molecular weight;
0.75:1 to 3:1 block ratio and densities for the first hydrophobic monomer and second hydrophobic monomer of between about 70 and 80 mol % and 20 and 30 mol %, respectively, for D-S—H wherein the drug molecule is a protein or glycoprotein >10 kDa but ≤40 kDa;
0.75:1 to 3:1 block ratio and densities for the first hydrophobic monomer and second hydrophobic monomer of between about 50 and 80 mol % and 20 and 50 mol %, respectively, for D-S—H wherein the drug molecule is a protein or glycoprotein with >40 kDa molecular weight; or,
0.25:1 to 2.5:1 block ratio and densities for the first hydrophobic monomer and second hydrophobic monomer of between about 70 and 80 mol % and 20 and 30 mol %, respectively, for D-S—H wherein the drug molecule is a protein or glycoprotein with >40 kDa molecular weight.

While specific block ratios and hydrophobic block monomer densities were found to be preferred for specific drug molecules, in general, temperature-responsive amphiphilic block copolymers of formula D-S—H comprising a hydrophilic block comprising a first hydrophilic monomer selected from monomers of Formula I (e.g., HPMA) and a hydrophobic block comprising a first hydrophobic monomer selected from temperature-responsive monomers of Class II (e.g., NIPMAM) and a second hydrophobic monomer selected from hydrophobic monomers comprising non-fluorinated aromatic groups (e.g., BnMAM), wherein the total molecular weight of the amphiphilic diblock copolymer (excluding the drug molecule) is between about 15 to 45 kDa; the block ratio is between 0.5:1 to 4:1, more preferably between about 0.75:1 and 3:1; the density of the first hydrophobic monomer is between about 50 and 85 mol %, typically between about 70 and 85 mol %; the density of the second hydrophobic monomer is between about 15 and 50 mol %, typically between about 15 and 30 mol %, were found to be effective for accommodating drug molecules with diverse physical and chemical properties.

In preferred embodiments of temperature-responsive amphiphilic diblock copolymers of formula S—H, optionally comprising a drug molecule (e.g., D-S—H, S(D)-H, S—H(D), D-S—H—S, D-S—H—S-D, S(D)-H—S, S(D)-H—S(D) or S—H(D)-S), the block ratio is between about 0.75:1 to 3:1, the hydrophilic block comprises a majority of monomer units selected from hydrophilic monomers of Formula I, preferably HPMA; the hydrophobic block comprises a first hydrophobic monomer selected from NIPMAM, NANPP, NVIBA, BEEP or TEGMA and a second hydrophobic monomer selected from monomers of Formula IV comprising an aromatic group, preferably BnMAM; and, the first monomer is distributed along the hydrophobic block at a density of between about 70-85 mol % and the second monomer is distributed along the hydrophobic block at a density of between about 15 mol % to about 30 mol %. A non-limiting example of an amphiphilic block copolymer that has these characteristics is provided here for clarity:

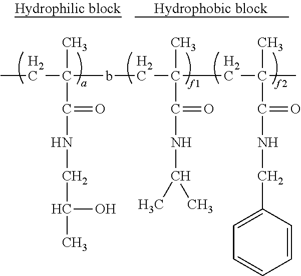

For this example, p(HPMA)ₐ-b-p[(NIPMAM)_{f1}-co-(BnMAM)_{f2}] would describe its structure wherein the symbol -b- delineates the two blocks, -co- denotes a random co-polymer, and subscripts a, f1 and f2 indicate that there are an integer number of repeating units of the hydrophilic monomer, first hydrophobic monomer and second hydrophobic monomer, respectively. The sum of monomer units can be between about 50 to 500, but is preferably between about 100 to 400 monomer units, and more preferably between about 150 to 350 monomer units; the ratio of hydrophilic block monomer units to hydrophobic block monomer units is typically between about 0.5:1 to about 4:1, and more preferably between 0.75:1 and 3:1; and, f2 is present in the hydrophobic block at a density of about 15 mol % to about 30 mol %.

In the above example, drug molecules may be attached through any suitable means to either end of the amphiphilic diblock copolymer; or, when reactive monomers are present, drug molecules can be linked to reactive monomers distributed along either the hydrophilic or hydrophobic blocks. A non-limiting example of drug molecules linked to the ends of an amphiphilic diblock copolymer is shown here for clarity:

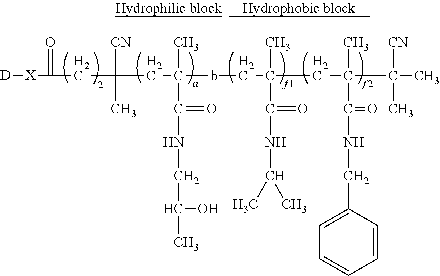

Wherein X is any suitable linker and D is a drug molecule.

An additional non-limiting example of drug molecules linked to reactive monomers distributed along the hydrophilic block of an amphiphilic diblock copolymer is shown here for clarity:

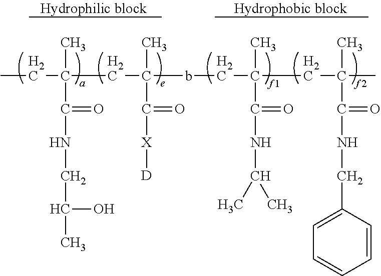

Wherein X is any suitable linker, D is a drug molecule linked to a co-monomer of the hydrophilic block via suitable linker X, and subscript e refers to the integer number of monomer units of the co-monomer to which the drug molecule is linked.

An additional non-limiting example of an amphiphilic diblock copolymer that assembles to 20 to 200 nm diameter particles, such as between 30 to 80 nm diameter particles, is an amphiphilic diblock copolymer of formula S—H, optionally comprising a drug molecule (e.g., D-S—H, S(D)-H, S—H(D), D-S—H—S, D-S—H—S-D, S(D)-H—S, S(D)-H—S(D) or S—H(D)-S), that has a block ratio greater than or equal to 0.5:1, preferably between about 0.5:1 and 4:1 and more preferably between 0.75:1 to 3:1, wherein the hydrophilic block comprises monomers selected from hydrophilic monomers, and optionally charged monomers and/or reactive monomers; the hydrophobic block comprises a hydrophobic monomer, a hydrophilic monomer and optionally charged monomers and/or reactive monomers; the hydrophobic monomer is selected from hydrophobic monomers comprising an aromatic group, such as monomers of Formula IV comprising an aromatic group (e.g., BnMAM), and the hydrophilic monomer of the hydrophobic block is selected from hydrophilic monomers of Formula I; the hydrophobic monomer is distributed along the hydrophobic block at a density of between about 10 mol % to about 100 mol % and the hydrophilic monomer of the hydrophobic block is distributed along the hydrophobic block at a density of between about 0 to 90 mol %.

In certain preferred embodiments of amphiphilic diblock copolymers of formula S—H, optionally comprising a drug molecule (e.g., D-S—H, S(D)-H, S—H(D), D-S—H—S, D-S—H—S-D, S(D)-H—S, S(D)-H—S(D) or S—H(D)-S), the block ratio is between about 0.75:1 and 3:1, the hydrophilic block comprises a majority of monomer units selected from hydrophilic monomers of Formula I, preferably HPMA; the hydrophobic block comprises a hydrophobic monomer selected from monomers of Formula IV comprising an aromatic group, preferably BnMAM and hydrophilic monomers selected from hydrophilic monomers of Formula I, preferably HPMA; and, the hydrophilic monomer of the hydrophobic block is distributed along the hydrophobic block at a density of between about 0 to 90 mol %, preferably about 75 mol % or less, and the hydrophobic monomer is distributed along the hydrophobic block at a density of between about 10 to 100 mol %, preferably about 25 mol % or higher. A non-limiting example of an amphiphilic block copolymer that has these characteristics is provided here for clarity:

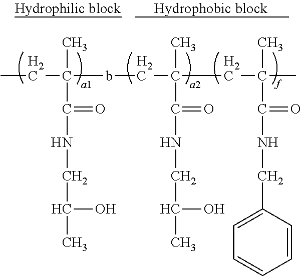

Wherein the symbol -b- delineates the two blocks, and a1, a2 and f indicate that there are an integer number of repeating units of the first hydrophilic monomer, second hydrophilic monomer and hydrophobic monomer, respectively. The sum of monomer units can be between about 50 to 500, but is preferably between about 100 to 400 monomer units, and more preferably between about 150 to 350 monomer units; the ratio of hydrophilic block monomer units to hydrophobic block monomer units is typically between about 0.5:1 to about 4:1, and more preferably between 0.75:1 and 3:1; and, f is present in the hydrophobic block at a density between 10-100 mol %, preferably about 25 mol % or higher.

Another current challenge is that concentration and/or viscosity can be limiting to the amount of drug encapsulated within particles based on amphiphilic block copolymers that can be administered per site and injection. For instance, high concentrations of drug molecules must be administered to enable prolonged drug activity in specific tissues, such as intracranial, intrathecal, intraocular, intrabursal, intraarticular, periarticular, etc.; however, many particle delivery systems become unstable at too high concentrations and/or viscosity of highly concentrated particles can become too high to make injection feasible. Therefore, there is a need for particle drug delivery systems that can be highly concentrated but are sufficiently low viscosity to make administration via needle injection feasible.

An unexpected finding disclosed herein is that certain compositions of amphiphilic block copolymers form particles in aqueous solutions at concentrations of less than 50 mg/mL (for example between about 0.01 mg/mL and 50 mg/mL) but become soluble when concentrated above about 50 mg/mL or 100 mg/mL in aqueous solutions. The ability of certain amphiphilic block copolymers to undergo concentration-dependent particle formation enabled the manufacture of highly concentrated drug products, which were able to be administered into tissues at low volumes. A non-binding explanation is that because viscosity is proportional to the size and concentration of solutes, amphiphilic block copolymers that exist as soluble single molecules when highly concentrated (e.g., at concentrations >100 mg/mL) are less viscous than those that are assembled to particles. Thus, in preferred embodiments of amphiphilic block copolymers used as drug carriers for injection to cranial, thecal, ocular, articular or bursal spaces, amphiphilic block copolymers that form particles in aqueous solutions at concentrations of less than 50 mg/mL but become soluble when concentrated above 50 mg/mL in aqueous solutions are preferred.

A non-limiting example of an amphiphilic block copolymer that forms particles in aqueous solutions at concentrations of less than 50 mg/mL but becomes soluble when concentrated above about 50 mg/mL, such as above 100 mg/mL in aqueous solutions, is an amphiphilic block copolymer comprising a hydrophilic block comprising a majority of monomer units selected from hydrophilic monomers of Formula I, preferably HPMA, and a hydrophobic block comprising a majority of monomer units selected from either a hydrophilic monomer of Formula I, preferably HPMA, or a hydrophobic monomer of Formula IV that is temperature-response, preferably NIPMAM, and a minority of monomer units selected from hydrophobic monomers of Formula IV that comprise an aromatic group, such as BnMAM.

The length of the amphiphilic block copolymer may either be expressed as the degree of polymerization of the amphiphilic block copolymer, degree of polymerization of individual blocks, molecular weight of the amphiphilic block copolymer or molecular weight of individual blocks. Wherein the amphiphilic block copolymer comprises a hydrocarbon-based backbone, the length of the amphiphilic block copolymer comprising a hydrocarbon-based backbone is such that the molecular weight is close to the renal excretion limit for poly(HPMA), i.e., approximately 50 kDa or 350 monomer units in length. Note that the degree of polymerization of the amphiphilic block copolymer can be calculated by dividing the molecular weight of the amphiphilic block copolymer by the average molecular weight of the monomer unit(s) comprising the amphiphilic block copolymer. Similarly, the degree of polymerization of an individual block can be calculated by dividing the molecular weight of the individual block by the average molecular weight of the monomer unit(s) comprising the individual block. For such calculations, the number-average molecular weight, abbreviated Mn, is preferred for calculating the degree of polymerization. As a non-limiting example, an amphiphilic block copolymer with a Mn of 25 kDa and an average monomer molecular weight of 250 g/mol would have a degree of polymerization of 100. The molecular weight can also be calculated by multiplying the degree of polymerization by the average monomer molecular weight. Note: unless otherwise specified, the molecular weight of the amphiphilic block copolymers described herein does not include the molecular of any drug molecules linked to the block copolymers.

In preferred embodiments of amphiphilic block copolymers (excluding any drug molecules linked to the amphiphilic block copolymer), the number-average molecular weight, Mn, is preferably about 5 kDa to about 60 kDa, such as 5 kDa, 6 kDa, 7 kDa, 8 kDa, 9 kDa, 10 kDa, 11 kDa, 12 kDa, 13, kDa, 14 kDa, 15 kDa, 16 kDa, 17 kDa, 18 kDa, 19 kDa, 20 kDa, 21 kDa, 22 kDa, 23 kDa, 24 kDa, 25 kDa, 26 kDa, 27 kDa, 28 kDa, 29 kDa, 30 kDa, 31 kDa, 32 kDa, 33 kDa, 34 kDa, 35 kDa, 36 kDa, 37 kDa, 38 kDa, 39 kDa, 40 kDa, 41 kDa, 42 kDa, 43 kDa, 44 kDa, 45 kDa, 46 kDa, 47 kDa, 48 kDa, 49 kDa, 50 kDa, 51 kDa, 52 kDa, 53 kDa, 54 kDa, 55 kDa, 56 kDa, 57 kDa, 58 kDa, 59 kDa or 60 kDa, and more preferably about 15 to about 45 kDa, such as 15 kDa, 16 kDa, 17 kDa, 18 kDa, 19 kDa, 20 kDa, 21 kDa, 22 kDa, 23 kDa, 24 kDa, 25 kDa, 26 kDa, 27 kDa, 28 kDa, 29 kDa, 30 kDa, 31 kDa, 32 kDa, 33 kDa, 34 kDa, 35 kDa, 36 kDa, 37 kDa, 38 kDa, 39 kDa, 40 kDa, 41 kDa, 42 kDa, 43 kDa, 44 kDa and 45 kDa. In certain embodiments, the amphiphilic block copolymer has a Mn between about 5 kDa and about 60 kDa or about 10 kDa to about 50 kDa, or about 15 kDa to about 50 kDa, or about 25 kDa to about 45 kDa. In preferred embodiments, the amphiphilic block copolymers have a Mn of about 15 to 45 kDa. In some preferred embodiments, the amphiphilic block copolymers have a Mn of about 15 to 45 kDa and the hydrophobic block and hydrophilic block are at least 5 kDa and 10 kDa in molecular weight, respectively.

Amphiphilic block copolymers describe herein can be synthesized by any suitable means and should preferably have low polydispersity, which may be calculated by dividing the weight-average molecular weight, Mw, by Mn, i.e., polydispersity index (PDI)=Mw/Mn. Therefore, in preferred embodiments, living polymerization, e.g., RAFT polymerization, is used to synthesize amphiphilic block copolymers with PDI less than 2.0, typically between about 1.01 and 1.2. While the amphiphilic block copolymers described herein may be synthesized starting from the hydrophilic block or hydrophobic block, it was unexpectedly observed that forming the hydrophobic block first, and then using the hydrophobic block terminated with a chain transfer agent as a macro CTA to polymerize the hydrophobic block to form the amphiphilic block copolymers led to better conversion and lower polydispersity than starting from the hydrophilic block. Thus, in preferred methods of manufacturing the amphiphilic block copolymers described herein, the hydrophobic block is synthesized first and then used as a macro CTA to synthesize the hydrophobic block from the macro CTA to yield amphiphilic block copolymers.

### Linkers

Linkers (sometimes referred to as “linker molecules”) generally refer to any molecules that join together any two or more different molecules comprising amphiphilic block copolymers, which may additionally perform any one or more of the following functions: i) increase or decrease water solubility; ii) increase distance between any two components, i.e., different molecules, of the amphiphilic block copolymers; iii) impart rigidity or flexibility; or iv) control/modulate the rate of degradation/hydrolysis of the link between any two or more different molecules.

In certain embodiments, a linker may join, i.e., link, any two components of an amphiphilic block copolymer through a covalent bond. Covalent bonds are the preferred linkages used to join any two components of amphiphilic block copolymers and ensure that no component, e.g., drug molecule, is able to immediately disperse from the other components following administration to a subject. Moreover, covalent linkages typically provide greater stability over non-covalent linkages and help to ensure that each component of the amphiphilic block copolymers are co-delivered to specific tissues and/or cells at or near the proportions of each component that was administered.

In a non-limiting example of a covalent linkage, a click chemistry reaction may result in a triazole that links, i.e., joins together, any two components of an amphiphilic block copolymer. In certain embodiments, the click chemistry reaction is a strain-promoted [3+2] azide-alkyne cyclo-addition reaction. An alkyne group and an azide group may be provided on respective molecules comprising to be linked by “click chemistry”. In some embodiments, a drug molecule that bearing an azide functional group is reacted with an alkyne (e.g., acetylene) or a strained-alkyne (e.g., dibenzylcyclooctyne (DBCO)) present on an amphiphilic block copolymer.

In some embodiments, a molecule bearing a thiol functional group is linked to an amphiphilic block copolymer through an appropriate reactive group such as an alkyne, alkene or maleimide, resulting in a thioether bond, or the thiol may be reacted with a pyridyl disulfide, e.g., resulting in a disulfide linkage.

In some embodiments, an amine is provided on one molecule and may be linked to another molecule by reacting the amine with any suitable electrophilic group such as carboxylic acids, acid chlorides or activated esters (for example, NHS ester), which results in an amide bond, or the amine may be reacted with alkenes (via Michael addition), aldehydes, and ketones (via Schiff base).

Linkers minimally join two components of amphiphilic block copolymers but may also increase distance between components and or impart hydrophobic or hydrophilic properties. There are many suitable linkers that are well known to those of skill in the art and include, but are not limited to, straight or branched-chain carbon linkers, heterocyclic carbon linkers, rigid aromatic linkers, flexible ethylene oxide linkers, peptide linkers, or a combination thereof.

In some embodiments, the carbon linker can include a C1-C18 alkane linker, such as a lower alkyl C4; the alkane linkers can serve to increase the space between two or more molecules, i.e., different components, comprising amphiphilic block copolymers, while longer chain alkane linkers can be used to impart hydrophobic characteristics and increase space between different components. In some embodiments, where the linker comprises a carbon chain, the linker may comprise a chain of between about 1 or 2 and about 18 carbon atoms, such as 1, 2, 3, 4, 5, 6, 7, 8, 9, 10, 11, 12, 13, 14, 15, 16, 17, 18 carbon atoms, or more. In some embodiments, wherein the linker comprises a carbon chain, the linker may comprise a chain of between about 12 and about 20 carbon atoms. In preferred embodiments, drug molecules are linked to reactive monomers through short alkane linkers, typically no more than 6 carbon atoms in length. In other embodiments, the linker can be an aromatic compound, or poly(aromatic) compound that imparts rigidity.

Alternatively, hydrophilic linkers, such as ethylene oxide linkers, may be used in place of alkane linkers to increase the space between any two or more molecules and increase water solubility. In some embodiments, the linker may comprise poly(ethylene glycol) (PEG). The length of the linker depends on the purpose of the linker. For example, the length of the linker, such as a PEG linker, can be increased to separate components, for example, to reduce steric hindrance, or in the case of a hydrophilic PEG linker can be used to improve water solubility. PEG linkers are typically between about 2 and about 24 monomers in length, such as 2, 3, 4, 5, 6, 7, 8, 9, 10, 11, 12, 13, 14, 15, 16, 17, 18, 19, 20, 21, 22, 23, 24 monomers in length.

In preferred embodiments of amphiphilic block copolymers linked to drug molecules that bind to extracellular receptors, the drug molecules are linked to the amphiphilic block copolymers through a PEG linker. In some embodiments, drug molecules than bind to extracellular receptors are linked to reactive co-monomers distributed along the backbone of hydrophilic blocks of amphiphilic block copolymers via a PEG linker. In other embodiments, drug molecules that bind to extracellular receptors are linked to the ends of amphiphilic block copolymers indirectly via a PEG linker.

Degradable linkers may also be used to control the rate of release of different components comprising amphiphilic block copolymers, optionally further comprising drug molecules. In some embodiments, the linker is cleavable under intracellular conditions, such that cleavage of the linker results in the release of any component linked to the linker, for example, an immunostimulant or chemotherapeutic drug.

In several embodiments, the linker includes a degradable peptide sequence that is cleavable by an intracellular enzyme (such as a cathepsin or the immuno-proteasome).

For example, the linker can be cleavable by enzymes localized in intracellular vesicles (for example, within a lysosome or endosome or caveolae) or by enzymes in the cytosol, such as the proteasome, or immuno-proteasome. The linker can be, for example, a peptide linker that is cleaved by protease enzymes, including, but not limited to proteases that are localized in intracellular vesicles, such as cathepsins in the lysosomal or endosomal compartment. The peptide linker is typically between 1-6 amino acids, such as 1, 2, 3, 4, 5 or 6 amino acids.

Certain dipeptides are known to be hydrolyzed by proteases that include cathepsins, such as cathepsins B and D and plasmin, (see, e.g., Dubowchik, G. M. et al. Pharmacology & Therapeutics, 1999, 83 (2), 67-123). For example, a peptide linker that is cleavable by the thiol-dependent protease cathepsin-B can be used (for example, a Phe-Leu or a Gly-Phe-Leu-Gly (SEQ ID NO: 1) linker). Other examples of such linkers described, for example, in U.S. Pat. No. 6,214,345, are incorporated herein by reference. In a specific embodiment, the peptide linker cleavable by an intracellular protease is a Val-Cit linker or a Phe-Lys linker (see, for example, U.S. Pat. No. 6,214,345, which describes the synthesis of doxorubicin with the Val-Cit linker).

Particular sequences for the cleavable peptide in the linker can be used to promote processing by immune cells following intracellular uptake. For example, embodiments of amphiphilic block copolymers used to stimulate innate or adaptive immune responses are internalized by immune cells, such as antigen-presenting cells (e.g., dendritic cells). The cleavable peptide linker can be selected to promote processing (i.e., hydrolysis) of the peptide linker following intracellular uptake by the immune cells. The sequence of the cleavable peptide linker can be selected to promote processing by intracellular proteases, such as cathepsins in intracellular vesicles or the proteasome or immuno-proteasome in the cytosolic space.

In several embodiments, cleavable peptide linkers of the formula Pn . . . P4-P3-P2-P1 are used to promote recognition by cathepsins, wherein P1 is selected from arginine, lysine, citrulline, glutamine, threonine, leucine, norleucine, or methionine; P2 is selected from glycine, leucine, valine or isoleucine; P3 is selected rom glycine, serine, alanine, proline or leucine; and P4 is selected from glycine, serine, arginine, lysine aspartic acid or glutamic acid. In a non-limiting example, a tetrapeptide cleavable peptide linker of the formula P4-P3-P2-P1 has the sequence Lys-Pro-Leu-Arg (SEQ ID NO: 2). For clarity, the amino acid residues (Pn) are numbered from proximal to distal from the site of cleavage, which is C-terminal to the P1 residue, for example, the amide bond between P1-P1′ is hydrolyzed. Peptide sequences recognized by endosomal and lysosomal proteases, such as cathepsin, that are suitable for use as cleavable peptide linkers are well described in the literature (see: Choe, Y. et al. J. Biol. Chem. 2006, 281 (18), 12824-12832).

In several embodiments, linkers comprised of peptide sequences are selected to promote recognition by the proteasome or immuno-proteasome. Peptide sequences of the formula Pn . . . P4-P3-P2-P1 are selected to promote recognition by proteasome or immuno-proteasome, wherein P1 is selected from basic residues and hydrophobic, branched residues, such as arginine, lysine, leucine, isoleucine and valine; and, P2, P3 and P4 are optionally selected from leucine, isoleucine, valine, lysine and tyrosine. In a non-limiting example, a cleavable peptide linker of the formula P4-P3-P2-P1 that is recognized by the proteasome has the sequence Tyr-Leu-Leu-Leu (SEQ ID NO:5). Sequences that promote degradation by the proteasome or immuno-proteasome may be used alone or in combination with cathepsin cleavable linkers. In some embodiments, amino acids that promote immuno-proteasome processing are linked to linkers that promote processing by endosomal proteases. A number of suitable sequences to promote cleavage by the immuno-proteasome are well described in the literature (see: Kloetzel, P.-M. et al. Nat. Rev. Mol. Cell Biol., 2001, 2, 179-187; Huber, E. M. et al. Cell, 2012, 148 (4), 727-738, and Harris, J. L. et al. Chem. Biol., 2001, 8 (12) 1131-1141).

In some embodiments, the cleavable peptide linker requires enzymatic processing before it can be cleaved. A non-limiting example is a cleavable peptide linker that comprises acetylated lysine and a cathepsin cleavage site; in this example, the lysine may require deacetylation prior to cleavage by the cathepsin.

In other embodiments, any two or more components of the amphiphilic block copolymers may be joined together through a pH-sensitive linker that is sensitive to hydrolysis under acidic conditions. A number of pH-sensitive linkages are familiar to those skilled in the art and include for example, a hydrazone, carboxyhydrazone, semicarbazone, thiosemicarbazone, cis-aconitic amide, orthoester, acetal, ketal, or the like (see, for example, U.S. Pat. Nos. 5,122,368; 5,824,805; 5,622,929; Dubowchik, G. M. et al. Pharmacology & Therapeutics, 1999, 83 (2), 67-123; Neville D. M. et al. Biol. Chem., 1989, 264, 14653-14661). In certain embodiments, the linkage is stable at physiologic pH, e.g., at a pH of about 7.4, but undergoes hydrolysis at lysosomal pH, ˜pH 5-6.5. In some embodiments, chemotherapeutic and/or immunostimulatory drugs, such as TLR-7/8 agonists, are linked to reactive monomers through a functional group that forms a pH-sensitive bond, such as the reaction between a ketone and a hydrazine to form a pH labile hydrazone bond. A pH-sensitive linkage, such as a hydrazone, provides the advantage that the bond is stable at physiologic pH, at about pH 7.4, but is hydrolyzed at lower pH values, such as the pH of intracellular vesicles.

In other embodiments, the linker comprises a linkage that is cleavable under reducing conditions, such as a reducible disulfide bond. Many different linkers used to introduce disulfide linkages are known in the art (see, for example, Thorpe, P. E. et al. Cancer Res., 1987, 47, 5924-5931; Wawrzynczak et al., In Immunoconjugates: Antibody Conjugates in Radioimagery and Therapy of Cancer (C. W. Vogel ed., Oxford U. Press, 1987); Phillips, G. D. L et al., Cancer Res., 2008, 68 (22), 9280-9290. See also U.S. Pat. No. 4,880,935).

In yet additional embodiments the linkage between any two components of the amphiphilic block copolymers can be formed by an enzymatic reaction, such as expressed protein ligation or by sortase (see: Fierer, J. O. et al. Proc. Natl. Acad. Sci., 2014, 111 (13), 1176-1181 and Theile, C. S. et al. Nat. Protoc., 2013, 8 (9), 1800-1807) chemo-enzymatic reactions (Smith, E. L. et al. Bioconjug. Chem., 2014, 25 (4), 788-795) or non-covalent high affinity interactions, such as, for example, biotin-avidin and coiled-coil interactions (Pechar, M. et al. Biotechnol. Adv., 2013, 31 (1), 90-96) or any suitable means that are known to those skilled in the art (see: Chalker, J. M. et al. Acc. Chem. Res., 2011, 44 (9), 730-741, Dumas, A. et al. Angew Chem. Int. Ed. Engl., 2013, 52 (14), 3916-3921).

### Amphiphilic Block Copolymer Compositions for Array of Drug Molecules

Amphiphilic block copolymers disclosed herein can be used to densely array drug molecules onto the surfaces of particles formed by the amphiphilic block copolymers. For instance, amphiphilic block copolymers comprising drug molecules linked to the hydrophilic block (e.g., D-S—H, S(D)-H, D-S—H—S, D-S—H—S-D, S(D)-H—S or S(D)-H—S(D)) assemble into particles in aqueous solutions at certain concentration, temperature and pH. Drug molecules linked to the hydrophilic block either at the end (e.g., D-S—H) or linked to reactive monomers distributed along the backbone (e.g., S(D)-H) may be solvent exposed and therefore presented in a multivalent array on the surface of the particles formed by the amphiphilic block copolymers. As disclosed herein, multivalent array of certain drug molecules is critical to the activity of certain drug molecules that are otherwise inactive when not surface accessible.

Therefore, in preferred embodiments of amphiphilic block copolymers comprising one or more drug molecules, which may be the same or different, that act extracellularly, such as by binding to or associating with soluble (non-cell associated) or cell surface associated (e.g., membrane bound) extracellular receptors, the drug molecules are arrayed on the amphiphilic block copolymers in a manner that enables accessibility of the extracellular receptors.

Drug molecules that bind extracellular receptors may be selected from synthetic or naturally occurring compounds, including protein, peptide, polysaccharide, glycopeptide, glycoprotein, lipid, or lipopeptide-based compounds. Examples of proteins include naturally occurring proteins, as well as antibodies or antibody fragments that are agonists or antagonists of extracellular receptors. The antibody may be engineered or naturally occurring, i.e., derived from an organism, or a combination thereof, e.g., a partially engineered antibody or antibody fragment, which may be modified chemically or otherwise. Other examples include synthetic, such as non-naturally occurring heterocycles that bind to extracellular receptors, or macrocycles.

The present inventors have unexpectedly found that the array of drug molecules that bind extracellular receptors on particles based on amphiphilic block copolymers improved receptor binding as well as enhanced biological activity as compared with that observed with drug molecules arrayed on linear co-polymers, or delivered on conventional particle delivery systems based on liposomes.

Advantageously, amphiphilic block copolymers of the present disclosure can be modulated to optimize the pharmacokinetics and pharmacodynamics of a range of different drug molecules. The amphiphilic block copolymers of the present disclosure can be used to display drug molecules and modulate the pharmacokinetics of the drug molecules.

In certain embodiments, the drug molecule has a molecular weight of from about 250 to about 10,000 Da. Note: molecular weight expressed in Daltons (Da) is the equivalent value as molecular weight expressed in g/mol; e.g., a molecule with a molecular weight of 250 Da can also be described as having a molecular weight of 250 g/mol. Drug molecules with relatively low molecular weight, e.g., less than about 10,000 Da can typically be accessed synthetically and are often suitable for use in organic solvents during manufacturing. In certain other embodiments, the drug molecule has a molecular weight of greater than about 10,000 Da. Drug molecules comprising peptides with relatively high molecular weight, e.g., greater than about 10,000 Da, are typically accessed by producing the drug molecule recombinantly using an expression system and are often not suitable for use in organic solvents during the manufacturing of the amphiphilic block copolymers.

An unexpected finding disclosed herein is that, for relatively high molecular weight drug molecules, i.e., drug molecules with molecular weight greater than 10,000 Da, linking relatively higher molecular weight drug molecules to amphiphilic block copolymers and then inducing particle assembly in aqueous solutions led to more densely arrayed drug molecules than when the same drug molecules were covalently conjugated to preformed particles. A non-binding explanation is that covalent attachment of relatively high molecular weight drug molecules is limited by steric hindrance, whereas the self-assembly of amphiphilic block copolymers linked to relatively high molecular weight drugs is more energetically favoured and therefore results in the more densely arrayed drug molecules than is possible with modification of preformed particles. Thus, amphiphilic block copolymers provide key advantages for arraying relatively large molecules, particularly biomolecules acting as drug molecules that bind to extracellular receptors.

Suitable biomolecules as drug molecules include therapeutic antibodies or antibody fragments useful for the treatment of a disease. Suitable antibodies include those that can modify disease, including the prevention, mitigation or reversal of disease, such as antibodies directed against beta-amyloid, sclerostin, IL-6, TNF-alpha, VEGF, VEGFR, IL-5, IL-12, IL-23, Kallikrein, PCSK9, BAFF, CD125 or similar such targets of antibodies. Therapeutic antibody molecules include antibodies directed against pathogens, cancer cells, soluble host proteins, toxins, as well as extracellular receptors and ion channels that may be blocked or stimulated to modulate signalling within the cell.

Suitable antibodies directed against tumor antigens. Non-limiting examples of antibodies directed against tumor antigens include antibodies directed against CD19, CD20, CD22, CD30, CD33, CD38, CD51, EGFR, PDGF-R, VEGFR, SLAMF7, integrin avP3, carbonic anhydrase 9, HER2, GD2 ganglioside, mesothelin, TAG-72. Suitable antibodies include antibodies against immune checkpoint molecules that can be used to reverse or modulate immune suppression. Non-limiting examples include PD1, PD-L1, OX-40, CTLA-4, 41BB. Suitable antibodies include agonists of the immune response, including but not limited to antibodies directed against CD40.

In some embodiments, the drug molecule arrayed on the amphiphilic block copolymers is a peptide-MHC complex, e.g., a complex of a CD8 or CD4 T cell epitope bound to an MHC-I or MHC-II, respectively, which may be used for inducing tolerance, when not provided with an additional immune stimulus, or may be used for activating and/or expanding T cells when used in combination with an immunostimulatory molecule.

In certain embodiments, a drug molecule that binds extracellular receptors is selected from molecules that bind to checkpoint molecules, such as PD1, PD-L1 and CTLA-4, such as antagonists of checkpoint molecules. Non-limiting examples of peptide check-point inhibitors include peptide-57, peptide-71 and other peptides (disclosed USPTO #20140294898, WO2014151634, WO2016039749, WO2016057624 A1, WO2016077518 A1, WO2016100285 A1, WO2016100608 A1, WO2016126646 A1 and WO2016149351 A1), D-peptides (Chang, H. -N. et al. Angewandte Chemie, 2015, 4, 11760-11764) and peptides identified via phage display (Liu, H. et al. Journal for ImmunoTherapy of Cancer, 2019, 7 (270), 1-14).

In some embodiments, the drug molecule is a peptide that binds to VEGF receptors, such as peptide-based antagonists of VEGF receptors or pathways associated with VEGF including biomimetic peptides such as AXT107 (Silva, R. L. E. et al. Science Translational Medicine, 2017, 9 (373), 1-11) or peptides identified via computational modelling or phage display such as HRHTKQRHTALH (SEQ ID NO: 4)(Zhang, Y. et al. Signal Transduction and Targeted Therapy, 2017, 2, 1-7).

In certain embodiments, the drug molecule is a protein or peptide that binds to B cell receptors and encompasses a full immunogen, or an epitope(s) derived from an immunogen(s) isolated from infectious organisms or cancer cells. In other embodiments, the drug molecule is a full immunogen or a peptide comprises T cell epitopes that bind to T cell receptors and encompasses an epitope(s) derived from immunogen(s) isolated from infectious organisms or cancer cells. In still other embodiments, the drug molecule is a protein or peptide that comprises T cell epitopes derived from a self-protein that bind to T cell receptors. The protein or peptide comprising an epitope(s) from infectious organisms may be from any infectious organism, such as a protein or glycoprotein derived from a fungus, bacterium, protozoan or virus. Alternatively, the protein or peptide-based drug molecule(s) comprises an epitope from a tumor-associated antigen including self-antigens or tumor-specific neoantigens. A broad variety of protein and peptide-based antigens are known in the art and are incorporated herein by reference: WO 2018/187515.

In certain embodiments, the drug molecule that binds to extracellular receptors is a saccharide that binds to lectin receptors, such as CD22. In other embodiments, the drug molecule that bind to extracellular receptors are selected from synthetic or naturally occurring agonists of extracellular pattern recognition receptors (PRRs) and have immunostimulatory properties, such as agonists of Toll-like receptor-1 (TLR-1), TLR-2, TLR-4, TLR-5 and TLR-6; and, agonists of C-type lectin receptors.

In some embodiments, drug molecules that binds to C-type lectin receptors (CLRs) are used to promote uptake by certain antigen presenting cells (APCs). In several embodiments, the drug molecule that binds to CLRs is a modified mannose and has the structure:

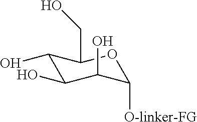

wherein the “linker” is any suitable linker molecule and FG is any suitable functional group that can be used to attach the linker modified mannose to a reactive monomer or either or both ends of the amphiphilic block copolymer. In some embodiments, the linker is PEG and FG is an azide.

In other embodiments, the drug molecule that binds to CLRs is a tetrasaccharide that binds to DC-SIGN and has the structure:

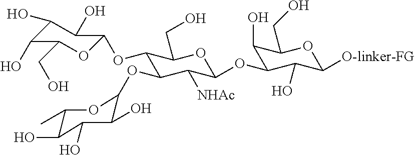

wherein the “linker” is any suitable linker molecule and FG is any suitable functional group that can be used to attach the linker modified tetrasaccharide to a reactive monomer or either or both ends of the amphiphilic block copolymer. In some embodiments, the linker is PEG, and FG is an azide.

Disclosed herein is the finding that multivalent array of certain drug molecules, particularly drug molecules that bind extracellular receptors, can improve biological activity. A non-binding explanation is that increased density of drug molecules that bind extracellular receptors leads to increased avidity for receptor binding. Therefore, in preferred embodiments of particles delivering drug molecules that bind extracellular receptors, the drug molecules that bind extracellular receptors are densely arrayed on the surface of particles based on amphiphilic block copolymers. A non-limiting example is an amphiphilic diblock copolymer of the formula D-S—H, wherein a drug molecule that binds extracellular receptors is covalently linked to the end of a hydrophilic block linked to a hydrophobic block, wherein the hydrophilic block comprises a majority of monomer units selected from hydrophilic monomers and the hydrophobic block comprises a majority of monomer units selected from hydrophobic monomers, preferably hydrophobic monomers that are temperature-responsive, additionally wherein the ratio of hydrophilic to hydrophobic block lengths is between about 0.5:1 to 4:1, preferably about 0.75:1 to 3:1.

Compositions of Amphiphilic Diblock Copolymers that Mitigate Antibodies Against Drug Molecules

When the amphiphilic block copolymers of the present disclosure are used for applications other than for inducing an antibody response, it may be beneficial to prevent anti-drug molecule antibodies (ADAs). In some embodiments, the agonist that binds to CD22 is a trisaccharide that has the structure:

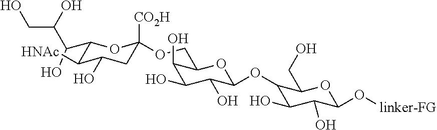

wherein the “linker” is any suitable linker molecule and FG is any suitable functional group that can be used to attach the linker modified trisaccharide to a reactive monomer or either or both ends of the amphiphilic block copolymer. In some embodiments, the linker is PEG, and FG is an azide or an amine.

The CD22 agonist may also be incorporated into a polymer via reaction with any suitable reactive monomer or via monomer functionalization prior to polymerization, shown here as a non-limiting CD22a co-monomer where X is any suitable linker and where R₂ can be H or CH₃.

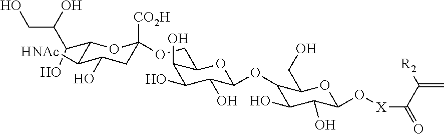

ADAs can have a deleterious impact on the activity of amphiphilic block copolymers comprising drug molecules. Therefore, in certain embodiments of amphiphilic block copolymers of the present disclosure, the hydrophilic blocks comprise anionic charged monomers and/or reactive comonomers linked to an agonist that binds CD22 to prevent antibody responses generated against the drug molecules.

A non-limiting example of an amphiphilic diblock copolymer comprised of monomers selected from (meth)acrylates or (meth)acrylamides, wherein a drug molecule is inked to the end of the hydrophilic block of the amphiphilic diblock copolymer and CD22a molecules are linked to reactive monomer units distributed along the hydrophilic block of the amphiphilic block copolymer is provided here for clarity:

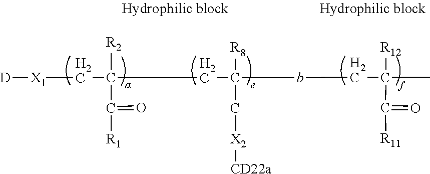

Wherein the symbol b delineates the two blocks; a, e and f denote an integer number of repeating units of hydrophilic monomers, reactive monomers and hydrophobic monomers, respectively; R₁ can be any suitable hydrophilic group; R₁₁ can be any suitable hydrophobic group; R₂, R₃ and R₁₂ can be independently selected from H or CH₃; X₁ and X₂ are any suitable linkers molecule; D denotes any suitable drug molecule; the density of the reactive monomer is typically between about 1 to 50 mol % of the hydrophilic block, preferably between about 3 to 30 mol % and more preferably about 10 mol %; the degree of polymerization block ratio of the hydrophilic block to hydrophobic block is typically between about 0.5:1 and 4:1, more preferably between about 0.75:1 and 3:1; and, the molecular weight is between about 5 to 60 kDa, more preferably between about 15 to 45 kDa. In preferred embodiments, the hydrophilic monomer is HPMA; and, the hydrophobic block comprises a first hydrophobic monomer and a second hydrophobic monomer selected from NIPMAM and BnMAM distributed along the hydrophobic block at a density of between about 70 and 85 mol % and 15 and 30 mol %, respectively; for example:

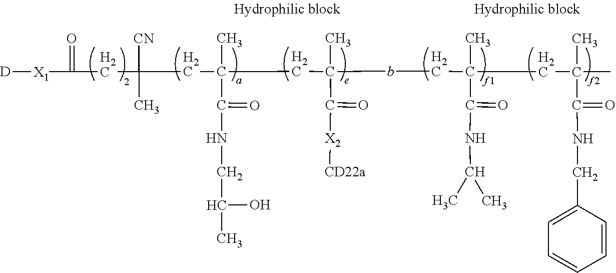

A non-limiting example of an amphiphilic diblock copolymer comprised of monomers selected from (meth)acrylates or (meth)acrylamides, wherein a CD22a agonist is inked to the end of the hydrophilic block of the amphiphilic diblock copolymer and drug molecules are linked to reactive monomer units distributed along the hydrophilic block of the amphiphilic block copolymer is provided here for clarity:

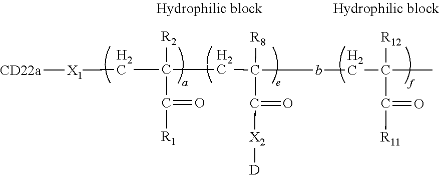

Wherein the symbol b delineates the two blocks; a, e and f denote an integer number of repeating units of hydrophilic monomers, reactive monomers and hydrophobic monomers, respectively; R₁ can be any suitable hydrophilic group; R₁₁ can be any suitable hydrophobic group; R₂, R₃ and R₁₂ can be independently selected from H or CH₃; X₁ and X₂ are any suitable linkers molecule; D denotes any suitable drug molecule; the density of the reactive monomer is typically between about 1 to 50 mol % of the hydrophilic block, preferably between about 3 to 30 mol % and more preferably about 10 mol %; the degree of polymerization block ratio of the hydrophilic block to hydrophobic block is typically between about 0.5:1 and 4:1, more preferably between about 0.75:1 and 3:1; and, the molecular weight is between about 5 to 60 kDa, more preferably between about 15 to 45 kDa. In preferred embodiments, the hydrophilic monomer is HPMA; and, the hydrophobic block comprises a first hydrophobic monomer and a second hydrophobic monomer selected from NIPMAM and BnMAM distributed along the hydrophobic block at a density of between about 70-85 mol % and 15 to 30 mol %, respectively; for example:

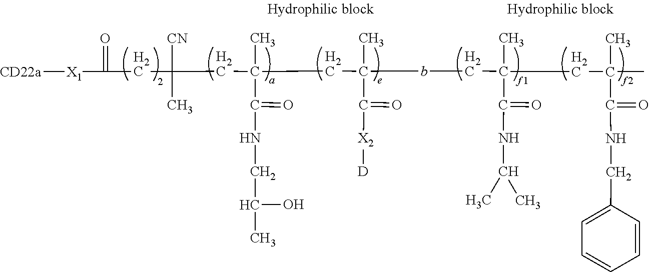

A non-limiting example of an amphiphilic diblock copolymer comprised of monomers selected from (meth)acrylates or (meth)acrylamides, wherein a drug molecule is linked to the end of the hydrophilic block of the amphiphilic diblock copolymer and charged monomer units are distributed along the hydrophilic block of the amphiphilic block copolymer is provided here for clarity:

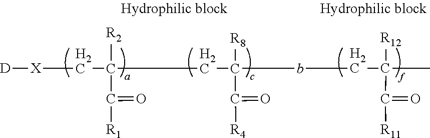

Wherein the symbol b delineates the two blocks; a, c and f denote an integer number of repeating units of hydrophilic monomers, charged monomers and hydrophobic monomers, respectively; R₁ can be any suitable hydrophilic group; R₄ can be any suitable charged molecule, preferably comprising a negatively charged functional group; R₁₁ can be any suitable hydrophobic group; R₂, R₅ and R₁₂ can be independently selected from H or CH₃; X is any suitable linker molecule; D denotes any suitable drug molecule; the density of the charged monomer is typically between about 1 to 50 mol % of the hydrophilic block, preferably between about 10 to 30 mol % and more preferably about 20 mol %; the degree of polymerization block ratio of the hydrophilic block to hydrophobic block is typically between about 0.5:1 and 4:1, more preferably between about 0.75:1 and 3:1; and, the molecular weight is between about 5 to 60 kDa, more preferably between about 15 to 45 kDa. In preferred embodiments, the hydrophilic monomer is HPMA and the charged monomer if methacrylic acid; and, the hydrophobic block comprises a first hydrophobic monomer and a second hydrophobic monomer selected from NIPMAM and BnMAM distributed along the hydrophobic block at a density of between about 70 and 85 mol % and 15 and 30 mol %, respectively; for example:

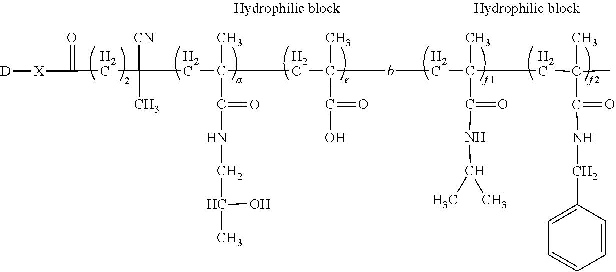

A non-limiting example of an amphiphilic diblock copolymer comprised of monomers selected from (meth)acrylates or (meth)acrylamides, wherein both reactive monomers linked to drug molecules and charged monomer are distributed along the hydrophilic block of the amphiphilic block copolymer is provided here for clarity:

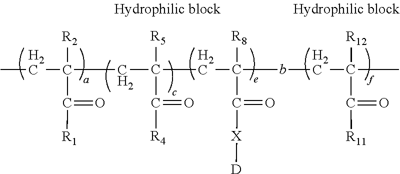

Wherein the symbol b delineates the two blocks; a, c, e and f denote an integer number of repeating units of hydrophilic monomers, charged monomers, reactive monomers and hydrophobic monomers, respectively; R₁ can be any suitable hydrophilic group; R₄ can be any suitable charged molecule, preferably comprising a negatively charged functional group; R₁₁ can be any suitable hydrophobic group; R₂, R₅, R₃ and R₁₂ can be independently selected from H or CH₃; X is any suitable linker molecule; D denotes any suitable drug molecule; the density of the charged monomer is typically between about 1 to 50 mol % of the hydrophilic block, preferably between about 10 to 30 mol % and more preferably about 20 mol %; the degree of polymerization block ratio of the hydrophilic block to hydrophobic block is typically between about 0.5:1 and 4:1, more preferably between about 0.75:1 and 3:1; and, the molecular weight is between about 5 to 60 kDa, more preferably between about 15 to 45 kDa. In preferred embodiments, the hydrophilic monomer is HPMA and the charged monomer is methacrylic acid; and, the hydrophobic block comprises a first hydrophobic monomer selected from temperature-responsive hydrophobic monomers (e.g., NIPMAM) and a second hydrophobic monomer selected from hydrophobic monomers comprising aromatic groups (e.g., BnMAM) distributed along the hydrophobic block at a density of between about 70 and 85 mol % and 15 and 30 mol %, respectively.

In some embodiments of particles comprising drug molecules, the particle comprises two or more compositions of amphiphilic block copolymers wherein a first amphiphilic block copolymer comprises drug molecules and a second amphiphilic block copolymer comprises CD22a linked to reactive monomers of the second amphiphilic block copolymer, for example:

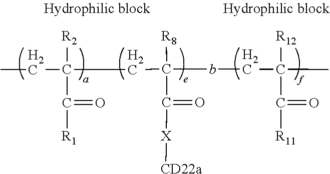

Wherein the symbol b delineates the two blocks; a, e and f denote an integer number of repeating units of hydrophilic monomers, reactive monomers and hydrophobic monomers, respectively; R₁ can be any suitable hydrophilic group; R₁₁ can be any suitable hydrophobic group; R₂, R₈ and R₁₂ can be independently selected from H or CH₃; X is any suitable linker molecule; CD22a is any suitable CD22a, such as the trisaccharide CD22a; the density of the reactive monomer is typically between about 1 to 50 mol % of the hydrophilic block, preferably between about 3 to 30 mol % and more preferably about 10 mol %; the degree of polymerization block ratio of the hydrophilic block to hydrophobic block is typically between about 0.5:1 and 4:1, more preferably between about 0.75:1 and 3:1; and, the molecular weight is between about 5 to 60 kDa, more preferably between about 15 to 45 kDa. In preferred embodiments, the hydrophilic monomer is HPMA; and, the hydrophobic block comprises a first hydrophobic monomer selected from temperature-responsive hydrophobic monomers (e.g., NIPMAM) and a second hydrophobic monomer selected from hydrophobic monomers comprising aromatic groups (e.g., BnMAM) distributed along the hydrophobic block at a density of between about 70 and 85 mol % and 15 and 30 mol %, respectively; for example:

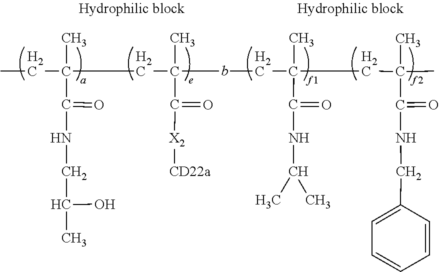

In some embodiments of particles comprising drug molecules, the particle comprises two or more compositions of amphiphilic block copolymers wherein a first amphiphilic block copolymer comprises CD22a linked to the end of a first amphiphilic block copolymer, for example:

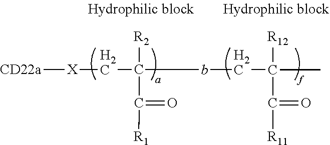

Wherein the symbol b delineates the two blocks; a and f denote an integer number of repeating units of hydrophilic monomers and hydrophobic monomers, respectively; R₁ can be any suitable hydrophilic group; R₁₁ can be any suitable hydrophobic group; R₂ and R₁₂ can be independently selected from H or CH₃; X is any suitable linker molecule; CD22a is any suitable CD22a, such as the trisaccharide CD22a; the degree of polymerization block ratio of the hydrophilic block to hydrophobic block is typically between about 0.5:1 and 4:1, more preferably between about 0.75:1 and 3:1; and, the molecular weight is between about 5 to 60 kDa, more preferably between about 15 to 45 kDa. In preferred embodiments, the hydrophilic monomer is HPMA; and, the hydrophobic block comprises a first hydrophobic monomer selected from temperature-responsive hydrophobic monomers (e.g., NIPMAM) and a second hydrophobic monomer selected from hydrophobic monomers comprising aromatic groups (e.g., BnMAM) distributed along the hydrophobic block at a density of between about 70 and 85 mol % and 15 and 30 mol %, respectively; for example:

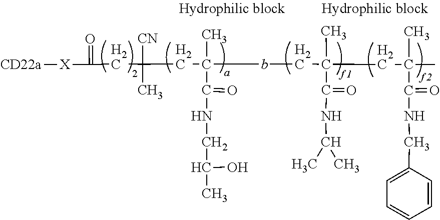

### Compositions of Amphiphilic Block Copolymers for Drug Delivery to Tumors

In addition to the surface array drug molecules that bind extracellular receptors, amphiphilic block copolymers of the present disclosure may also be used for the delivery of drugs that bind to intracellular targets, such as small molecule immunostimulants and chemotherapeutics used for cancer treatment. For such applications, the drug molecules may be arrayed on the surface of particles based on amphiphilic block copolymers, or the drug molecules more preferably may be incorporated within the core of particles formed by amphiphilic block copolymers. In preferred embodiments, of amphiphilic block copolymers comprising drug immunostimulatory or chemotherapeutic drug molecules, the drug molecules are linked to the hydrophobic block (e.g., S—H(D), S—H-D or S—H(D)-S) or are encapsulated within the hydrophobic core when admixed with the amphiphilic block copolymers (e.g., S—H+D or S—H—S+D), or may be linked to a hydrophobic block (e.g., D-H or H(D)) incorporated within particles formed by amphiphilic block copolymers (e.g., S—H+D-H, S—H+H(D), S—H—S+D-H or S—H—S+H(D)).

In some embodiments, immunogenic compositions of particles based on amphiphilic block copolymers comprise immunostimulatory drug molecules that bind to PRRs and induce anticancer immunity. While any class of PRR agonist molecule could potentially be used as an immunostimulant, it was found, unexpectedly, that certain classes of immunostimulants lead to unexpectedly enhanced tumor clearance as compared with other classes of immunostimulants. Herein, it is disclosed that preferred immunostimulants are those that induce the production of specific cytokines, i.e., interferons (IFNs) and/or IL-12. Thus, in preferred embodiments for cancer treatment, amphiphilic block copolymers include immunostimulants selected from agonists of Stimulator of Interferon Genes (STING), TLR-3, TLR-4, TLR-7, TLR-8, TLR-7/8 and TLR-9. For clarity, since TLR-4 is surface expressed (i.e., extracellular) and present within endosomes intracellularly, so can be classified as both intracellularly and extracellularly acting drug molecule.

Non-limiting examples of TLR-3 agonists include dsRNA, such as Polyl:C, and nucleotide base analogs; TLR-4 agonists include lipopolysaccharide (LPS) derivatives, for example, monophosphoryl lipid A (MPL) small molecules such as pyrimidoindole; TLR-7 & -8 agonists include ssRNA and nucleotide base analogs, including derivatives of imidazoquinolines, hydroxy-adenine, benzonapthyridine and loxoribine; TLR-9 agonists include unmethylated CpG and small molecules that bind to TLR-9; STING agonists include cyclic dinucleotides, and synthetic small molecules, such as alpha-mangostin and its derivatives as well as linked amidobenzimidazole (“diABZI”) and related molecules (see: Ramanjulu, J. M. et al. Nature, 2018, 564, 439-443).

In several embodiments, the compositions of amphiphilic block copolymers for cancer treatment comprise small molecule drugs selected from imidazoquinoline-based agonists of TLR-7, TLR-8 and/or TLR-7 & -8. Numerous such agonists are known, including many different imidazoquinoline compounds.

Imidazoquinolines are of use as small molecule immunostimulatory drugs (D) used in star polymers found in immunogenic composition used for vaccination and/or for treating cancer or infectious diseases in the absence of a co-administered antigen. Imidazoquinolines are synthetic immunomodulatory compounds that act by binding Toll-like receptors 7 and 8 (TLR-7/TLR-8) on antigen presenting cells (e.g., dendritic cells), structurally mimicking these receptors' natural ligand, viral single-stranded RNA. Imidazoquinolines are heterocyclic compounds comprising a fused quinoline-imidazole skeleton. Derivatives, salts (including hydrates, solvates, and N-oxides), and prodrugs thereof also are contemplated by the present disclosure. Particular imidazoquinoline compounds are known in the art, see for example, U.S. Pat. Nos. 6,518,265; and 4,689,338. In some non-limiting embodiments, the imidazoquinoline compound is not imiquimod and/or is not resiquimod.

In some embodiments, the drugs molecules that bind to TLR-7 or TLR-8 can be selected from a small molecule having a 2-aminopyridine fused to a five membered nitrogen-containing heterocyclic ring, including but not limited to imidazoquinoline amines and substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, hydroxylamine substituted imidazoquinoline amines, oxime substituted imidazoquinoline amines, 6-, 7-, 8-, or 9-aryl, heteroaryl, aryloxy or arylalkyleneoxy substituted imidazoquinoline amines, and imidazoquinoline diamines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, hydroxylamine substituted tetrahydroimidazoquinoline amines, oxime substituted tetrahydroimidazoquinoline amines, and tetrahydroimidazoquinoline diamines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; pyrazolopyridine amines; pyrazoloquinoline amines; tetrahydropyrazoloquinoline amines; pyrazolonaphthyridine amines; tetrahydropyrazolonaphthyridine amines; and 1H-imidazo dimers fused to pyridine amines, quinoline amines, tetrahydroquinoline amines, naphthyridine amines, or tetrahydronaphthyridine amines.

In some embodiments, the drug molecule is an imidazoquinoline with the formula:

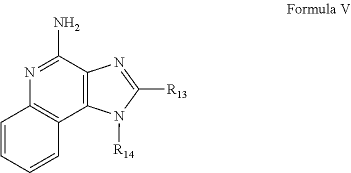

In Formula V, R₁₃ is selected from one of hydrogen, optionally-substituted lower alkyl, or optionally-substituted lower ether; and R₁₄ is selected from one of optionally substituted arylamine, or optionally substituted lower alkylamine. R₁₃ may be optionally substituted to a linker that links to a polymer.

In some embodiments, the R₁₃ included in Formula V can be selected from hydrogen,

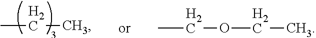

In some embodiments, R₁₄ can be selected from,

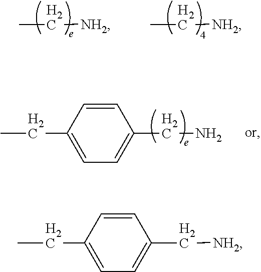

wherein e denotes the number of methylene unites is an integer from 1 to 4.

In some embodiments, R₁₄ can be

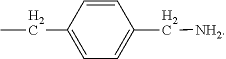

In some embodiments, R₁₄ can be 4

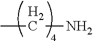

In some embodiments, R₁₃ can be

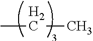

### and R14 can be

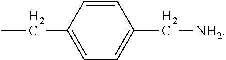

In certain embodiments for cancer treatment, particles based on amphiphilic block copolymer further comprise drug molecules selected from chemotherapeutic agents (or “chemotherapeutic molecules”). Chemotherapeutic agents include, without limitation, alkylating agents (cisplatin, cyclophosphamide & temozolomide as an example), topoisomerase inhibitors (Topoisomerase I inhibitors and Topoisomerase II inhibitors), mitotic inhibitors (taxanes and Vinca alkaloids as an example), antimetabolites (5-fluorouracil, capecitabine & methotrexate as an example), and anti-tumor antibiotics (anthracycline family, actinomycin-D and bleomycin as an example). Also, included in this definition are receptor tyrosine kinase inhibitors, differentiating agents, histone deacetylase inhibitors, angiogenesis inhibitors, steroids and anti-hormonal agents, among others.

In a non-limiting example, the anthracycline is doxorubicin and has the structure:

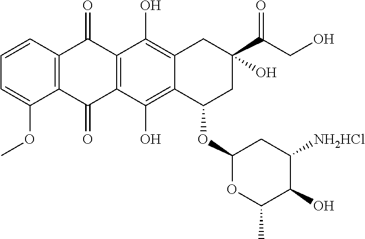

wherein the doxorubicin molecule may be linked to amphiphilic block copolymers through the amine or ketone position via an amide or hydrazone bond, respectively.

Drug molecules used for cancer treatment, such as immunostimulatory drugs or chemotherapeutic agents, may be attached to any suitable functional group on the amphiphilic block copolymers of the present disclosure through any suitable means, though, preferably via reactive monomers distributed along the hydrophobic block. The inventors' results show that high loading of immunostimulatory drugs or chemotherapeutic agents is fundamental to achieving high levels of efficacy and that maximal drug molecule loading is achieved when the drug molecules are arrayed along the hydrophobic block of the amphiphilic block copolymers. Therefore, in preferred embodiments of amphiphilic block copolymers used for cancer treatment, immunostimulatory drugs or chemotherapeutic agents are linked to reactive monomers distributed along the hydrophobic block. Though, as certain drug molecules may require solvent exposure for activity and/or release, in certain embodiments of amphiphilic block copolymers used for cancer treatment, immunostimulatory drugs or chemotherapeutic agents are linked to reactive monomers distributed along the hydrophilic block.

### EMBODIMENTS

Additional non-limiting exemplary embodiments include the following.

In a first aspect, provided herein is an amphiphilic block copolymer having any one of the formulas D-S—H, S(D)-H, S—H(D), D-S—H—S, D-S—H—S-D, S(D)-H—S, S(D)-H—S(D) or S—H(D)-S, wherein S is a hydrophilic block; H is a hydrophobic block; D is a drug molecule; ( ) denotes that the group is bonded directly or indirectly as a side chain or as part of a side chain group to the adjacent group; and the hyphen, “-” (or sometimes “-”), denotes that each of the adjacent S, H or D are linked either directly to one another or indirectly to one another via a linker, additionally wherein the hydrophilic block comprises a first hydrophilic monomer and the hydrophobic block comprises a first hydrophobic monomer and a second hydrophobic monomer.

In certain embodiments, the first hydrophobic monomer comprises at least one temperature-responsive monomer and the second hydrophobic monomer comprises at least one hydrophobic monomer comprising an aromatic group. In certain specific embodiments, the first hydrophobic monomer is selected from NIPMAM, NANPP, NVIBA, BEEP or TEGMA. In certain specific embodiments, the first hydrophobic monomer is NIPMAM and the second hydrophobic monomer is BnMAM. The first hydrophobic monomer may comprise between 50 and 95 mol % of the hydrophobic block and the second hydrophobic monomer may comprise between 5 and 50 mol % of the hydrophobic block. For example, the first hydrophobic monomer may comprise 70 and 85 mol % of the hydrophobic block and the second hydrophobic monomer may comprise between 15 to 30 mol % of the hydrophobic block.

In certain embodiments, the first hydrophilic monomer is selected from (meth)acrylates or (meth)acrylamides of the chemical formula CH₂=CR₂—C(O)—R₁ (“Formula I”), wherein the acryl side group R₁ may be selected from one or more of the groups consisting of —OR₃, —NHR₃ or —N(CH₃)R₃, where R₂ can be H or CH₃, and R₃ is independently selected from H (except for OR₃), CH₃, CH₂CH₃, CH₂CH₂OH, CH₂(CH₂)₂OH, CH₂CH(OH)CH₃ or CHCH₃CH₂OH or (CH₂CH₂O)ᵢH, where i is an integer number of repeating units. In certain specific embodiments, the first hydrophilic monomer is selected from HEA, HEMAM, HPMA, PEG or combinations thereof.

In certain embodiments, the amphiphilic block copolymer has a degree of polymerization block ratio of hydrophilic block to hydrophobic block of 0.5:1 to 4:1, such as a degree of polymerization block ratio of hydrophilic block to hydrophobic block of 0.75:1 to 3:1.

In certain embodiments, the amphiphilic block copolymer has a molecular weight of between about 5 to 60 kDa.

The amphiphilic block copolymer may be linked to the drug molecule (D) either directly or indirectly through a linker.

In certain embodiments, the drug molecule (D) is selected from ocular drugs, steroidal or nonsteroidal anti-inflammatory drugs.

In certain embodiments, the amphiphilic block copolymer has the formula D-S—H, D-S—H—S or D-S—H—S-D and the drug molecule is linked to the end of the hydrophilic block of the amphiphilic block copolymer. In certain other embodiments, the amphiphilic block copolymer has the formula S(D)-H, S(D)-H—S or S(D)-H—S(D) and the drug molecule is linked to the amphiphilic block copolymer through a first reactive monomer that is distributed along the backbone of the hydrophilic block.

In certain embodiments, the hydrophilic block further comprises a first charged monomer comprising a negatively charged monomer.

In certain embodiments, the hydrophilic block further comprises a reactive monomer linked to a CD22 agonist.

In certain embodiments, the amphiphilic block copolymer exists as unimers in aqueous solutions below a transition temperature but forms particles in aqueous solutions above the transition temperature. The transition temperature may be below about 37° C. and may be between about 20° C. and about 34° C.

In certain embodiments, the amphiphilic block copolymer assembles to particles between about 20 to 200 nm in diameter in aqueous solutions above the transition temperature. In certain specific embodiments, the amphiphilic block copolymer assembles to particles between about 20 to 200 nm in diameter in aqueous solutions above the transition temperature, such as about 30 to 80 nm in diameter in aqueous solutions above the transition temperature.

In a second aspect, provided herein is a method of treating a disease, the method comprising providing a composition comprising the amphiphilic block copolymer of the first aspect in an aqueous solution at a concentration greater than 50 mg/mL and introducing the solution into a body cavity. The composition may be introduced by ocular, intravitreal, suprachoroidal, intrabursal, intrarticular, periarticular, intraperitoneal, intrapericardial, intraperipleural, intrathecal or intraventricular injection.

In a third aspect, provided herein is an amphiphilic block copolymer having any one of the formulas D-S—H, S(D)-H, S—H(D), D-S—H—S, D-S—H—S-D, S(D)-H—S, S(D)-H—S(D) or S—H(D)-S, wherein S is a hydrophilic block; H is a hydrophobic block; D is a drug molecule; ( ) denotes that the group is bonded directly or indirectly as a side chain or as part of a side chain group to the adjacent group; and the hyphen, “-” (or sometimes “-”), denotes that each of the adjacent S, H or D are linked either directly to one another or indirectly to one another via a linker, additionally wherein the hydrophilic block comprises a first hydrophilic monomer and the hydrophobic block comprises a first hydrophobic monomer selected from hydrophobic monomers comprising an aromatic group.

In certain embodiments of the third aspect, the amphiphilic block copolymer comprises a second hydrophilic monomer present on the hydrophobic block, wherein the second hydrophilic monomer is selected from (meth)acrylates or (meth)acrylamides of the chemical formula CH₂=CR₂—C(O)—R₁ (“Formula I”), wherein the acryl side group R₁ may be selected from one or more of the groups consisting of —OR₃, —NHR₃ or —N(CH₃)R₃, where R₂ can be H or CH₃, and R₃ is independently selected from H (except for OR₃), CH₃, CH₂CH₃, CH₂CH₂OH, CH₂(CH₂)₂OH, CH₂CH(OH)CH₃ or CHCH₃CH₂OH or (CH₂CH₂O)ᵢH, and where i is an integer number of repeating units.

In certain embodiments of the third aspect, the amphiphilic block copolymer comprises a second hydrophobic monomer present on the hydrophobic block, wherein the second hydrophobic monomer is selected from temperature-responsive monomers selected from NIPMAM, NANPP, NVIBA, BEEP or TEGMA.

In certain embodiments of the third aspect, the first hydrophobic monomer is BnMAM.

In certain embodiments of the third aspect, the first hydrophobic monomer comprises between 10 to 100 mol % of the hydrophobic block. In certain specific embodiments of the third aspect, the first hydrophobic monomer comprises between 25 to 75 mol % of the hydrophobic block.

In certain embodiments of the third aspect, the amphiphilic block copolymer exists as unimers at concentrations greater than 50 mg/mL in aqueous solutions but assembles into particles at concentrations of less than about 50 mg/mL.

In certain embodiments of the third aspect, the first hydrophilic monomer is selected from (meth)acrylates or (meth)acrylamides of the chemical formula CH₂=CR₂—C(O)—R₁ (“Formula I”), wherein the acryl side group R₁ may be selected from one or more of the groups consisting of —OR₃, —NHR₃ or —N(CH₃)R₃, where R₂ can be H or CH₃, and R₃ is independently selected from H (except for OR₃), CH₃, CH₂CH₃, CH₂CH₂OH, CH₂(CH₂)₂OH, CH₂CH(OH)CH₃, CHCH₃CH₂OH or (CH₂CH₂O)ᵢH, where i is an integer number of repeating units. In certain specific embodiments of the third aspect, the first hydrophilic monomer is selected from HEA, HEMAM, HPMA, PEG or combinations thereof.

In certain embodiments of the third aspect, the amphiphilic block copolymer has a degree of polymerization block ratio of hydrophilic block to hydrophobic block of 0.5:1 to 4:1, such as a degree of polymerization block ratio of hydrophilic block to hydrophobic block of 0.75:1 to 3:1.

In certain embodiments of the third aspect, the amphiphilic block copolymer has a molecular weight of between about 5 to 60 kDa.

In certain embodiments of the third aspect, the amphiphilic block copolymer is linked to the drug molecule (D) either directly or indirectly through a linker.

In certain embodiments of the third aspect, the drug molecule is selected from ocular drugs, steroidal or nonsteroidal anti-inflammatory drugs.

In certain embodiments of the third aspect, the amphiphilic block copolymer has the formula D-S—H, D-S—H—S or D-S—H—S-D and the drug molecule is linked to the end of the hydrophilic block of the amphiphilic block copolymer. In certain other embodiments of the third aspect, the amphiphilic block copolymer has the formula S(D)-H, S(D)-H—S or S(D)-H—S(D) and the drug molecule is linked to the amphiphilic block copolymer through a first reactive monomer that is distributed along the backbone of the hydrophilic block.

In certain embodiments of the third aspect, the hydrophilic block further comprises a first charged monomer selected from negatively charged monomers.

In certain embodiments of the third aspect, the hydrophilic block further comprises a reactive monomer linked to a CD22 agonist.

In certain embodiments of the third aspect, the amphiphilic block copolymer assembles to particles between about 20 to 200 nm in diameter in aqueous solutions at concentrations of less than about 50 mg/mL. In certain specific embodiments of the third aspect, the amphiphilic block copolymer assembles to particles between about 30 to 80 nm in diameter in aqueous solutions at concentrations of less than about 50 mg/mL.

In a fourth aspect, provided herein is a method of treating a disease, the method comprising providing a composition comprising the amphiphilic block copolymer of the third aspect in an aqueous solution at a concentration greater than 50 mg/mL and introducing the solution into a body cavity.

In certain embodiments of the fourth aspect, the composition may be introduced by ocular, intravitreal, suprachoroidal, intrabursal, intrarticular, periarticular, intraperitoneal, intrapericardial, intraperipleural, intrathecal or intraventricular injection.

In a fifth aspect, provided herein is a formulation comprising a first amphiphilic block copolymer and a second amphiphilic block copolymer having any one of the formulas D-S—H, S(D)-H, S—H(D), D-S—H—S, D-S—H—S-D, S(D)-H—S, S(D)-H—S(D) or S—H(D)-S, wherein S is a hydrophilic block; H is a hydrophobic block; D is a drug molecule; ( ) denotes that the group is bonded directly or indirectly as a side chain or as part of a side chain group to the adjacent group; and the hyphen, “-” (or sometimes “-”), denotes that each of the adjacent S, H or D are linked either directly to one another or indirectly to one another via a linker, additionally wherein the first amphiphilic block copolymer comprises a drug molecule (D) selected from agonists of CD22a linked to the end(s) of the first amphiphilic block copolymer or linked to a first reactive monomer distributed along the backbone of the hydrophilic block of the first amphiphilic block copolymer.

In certain embodiments of the fifth aspect, the hydrophilic block comprises a first hydrophilic monomer and the hydrophobic block comprises a first hydrophobic monomer and a second hydrophobic monomer, wherein the first hydrophobic monomer is selected from temperature-responsive monomers selected from NIPMAM, NANPP, NVIBA, BEEP or TEGMA, and the second hydrophobic monomer is selected from hydrophobic monomers comprising an aromatic group, additionally wherein the first hydrophobic monomer comprises between 70 and 85 mol % of the hydrophobic block and the second hydrophobic monomer comprises between 15 to 30 mol % of the hydrophobic block.

In certain embodiments of the fifth aspect, the first hydrophilic monomer is HPMA, the first hydrophobic monomer is NIPMAM and the second hydrophobic monomer is BnMAM.

In certain embodiments of the fifth aspect, the hydrophilic block comprises a first hydrophilic monomer and the hydrophobic block comprises a first hydrophobic monomer and a second hydrophilic monomer, wherein the first hydrophobic monomer comprises an aromatic group, and the second hydrophilic monomer is selected from (meth)acrylates or (meth)acrylamides of the chemical formula CH₂=CR₂—C(O)—R₁ (“Formula I”), wherein the acryl side group R₁ may be selected from one or more of the groups consisting of —OR₃, —NHR₃ or —N(CH₃)R₃, where R₂ can be H or CH₃, and R₃ is independently selected from H (except for OR₃), CH₃, CH₂CH₃, CH₂CH₂OH, CH₂(CH₂)₂OH, CH₂CH(OH)CH₃ or CHCH₃CH₂OH or (CH₂CH₂O)ᵢH, and where i is an integer number of repeating units.

In certain embodiments of the fifth aspect, the first hydrophobic monomer is BnMAM.

In certain embodiments of the fifth aspect, the first hydrophobic monomer comprises between 25 to 75 mol % of the hydrophobic block.

In a sixth aspect, provided herein is an amphiphilic block copolymer having any one of the formulas D-S—H, S(D)-H, S—H(D), D-S—H—S, D-S—H—S-D, S(D)-H—S, S(D)-H—S(D) or S—H(D)-S, wherein S is a hydrophilic block; H is a hydrophobic block; D is a drug molecule; ( ) denotes that the group is bonded directly or indirectly as a side chain or as part of a side chain group to the adjacent group; and the hyphen, “-” (or sometimes “-”), denotes that each of the adjacent S, H or D are linked either directly to one another or indirectly to one another via a linker, additionally wherein the hydrophilic block comprises a first hydrophilic monomer and the hydrophobic block comprises a first hydrophobic monomer and a second hydrophobic monomer, wherein the first hydrophobic monomer is selected from temperature-responsive monomers and the second hydrophobic monomer is selected from hydrophobic monomers comprising a fluorinated aromatic ring or fused aromatic ring group.

In certain embodiments of the sixth aspect, the first hydrophobic monomer is selected from NIPMAM, NANPP, NVIBA, BEEP or TEGMA. In certain specific embodiments, the first hydrophobic monomer is NIPMAM.

In certain embodiments of the sixth aspect, the second hydrophobic monomer is selected from N-3,4,5-trifluorobenzyl methacrylamide, N-2,3,4,5,6 pentafluorobenzyl methacrylamide, N-trifluoromethylbenzyl methacrylamide or N-bitrifluoromethylbenzyl methacrylamide.

In certain embodiments of the sixth aspect, the first hydrophobic monomer comprises between 80 and 99 mol % of the hydrophobic block and the second hydrophobic monomer comprises between 1 and 20 mol % of the hydrophobic block, such as the first hydrophobic monomer comprises between 90 and 99 mol % of the hydrophobic block and the second hydrophobic monomer comprises between 1 and 10 mol % of the hydrophobic block.

In certain embodiments of the sixth aspect, the first hydrophilic monomer is selected from (meth)acrylates or (meth)acrylamides of the chemical formula CH₂=CR₂—C(O)—R₁ (“Formula I”), wherein the acryl side group R₁ may be selected from one or more of the groups consisting of —OR₃, —NHR₃ or —N(CH₃)R₃, where R₂ can be H or CH₃, and R₃ is independently selected from H (except for OR₃), CH₃, CH₂CH₃, CH₂CH₂OH, CH₂(CH₂)₂OH, CH₂CH(OH)CH₃ or CHCH₃CH₂OH or (CH₂CH₂O)ᵢH, where i is an integer number of repeating units. In certain specific embodiments, the first hydrophilic monomer is selected from HEA, HEMAM, HPMA or PEG.

In certain embodiments of the sixth aspect, the amphiphilic block copolymer has a degree of polymerization block ratio of hydrophilic block to hydrophobic block of 0.5:1 to 4:1, such as 0.75:1 to 3:1.

In certain embodiments of the sixth aspect, the amphiphilic block copolymer has a molecular weight of between about 5 to 60 kDa.

In certain embodiments of the sixth aspect, the amphiphilic block copolymer is linked to the drug molecule (D) either directly or indirectly through a linker.

In certain embodiments of the sixth aspect, the drug molecule is selected from ocular drugs, steroidal or nonsteroidal anti-inflammatory drugs.

In certain embodiments of the sixth aspect, the amphiphilic block copolymer has the formula D-S—H, D-S—H—S or D-S—H—S-D and the drug molecule is linked to the end of the hydrophilic block of the amphiphilic block copolymer.

In certain embodiments of the sixth aspect, the amphiphilic block copolymer has the formula S(D)-H, S(D)-H—S or S(D)-H—S(D) and the drug molecule is linked to the amphiphilic block copolymer through a first reactive monomer that is distributed along the backbone of the hydrophilic block.

In certain embodiments of the sixth aspect, the hydrophilic block further comprises a first charged monomer selected from negatively charged monomers.

In certain embodiments of the sixth aspect, the hydrophilic block further comprises a reactive monomer linked to a CD22 agonist.

In certain embodiments of the sixth aspect, the amphiphilic block copolymer exists as unimers in aqueous solutions below a transition temperature but forms particles in aqueous solutions above the transition temperature. In certain embodiments, the transition temperature is below 37° C. In certain specific embodiments, the transition temperature is between about 20° C. and 34° C.

In certain embodiments of the sixth aspect, the amphiphilic block copolymer assembles to particles between about 20 to 200 nm in diameter in aqueous solutions above the transition temperature, such as between about 30 to 80 nm in diameter in aqueous solutions above the transition temperature.

In a seventh aspect, provided herein is a method of treating a disease, the method comprising formulating the amphiphilic block copolymer of the sixth aspect in an aqueous solution at a concentration greater than 50 mg/mL and injecting the solution into a body cavity.

In certain embodiments of the seventh aspect, the injection is selected from any of ocular, intravitreal, suprachoroidal, intrabursal, intrarticular, periarticular, intraperitoneal, intrapericardial, intraperipleural, intrathecal and intraventricular routes of injection.

### EXAMPLES

The following preparations of compounds and intermediates are given to enable those skilled in the art to more clearly understand and to practice the present disclosure. They should not be considered as limiting the scope of the disclosure, but merely as being illustrative and representative thereof.

The starting materials and reagents used in preparing these compounds are either available from commercial suppliers such as Aldrich Chemical Co., (Milwaukee, Wis.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art having referred to this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., or from about 0° C. to about 125° C. or at about room (or ambient) temperature, e.g., about 20° C.

Compounds of Formula (I) and subformulae and species described herein, including those where the substituent groups as defined herein, can be prepared as illustrated and described below.

In therapeutic applications described herein, the compounds can be formulated using techniques and formulations generally may be found in Remington, The Science and Practice of Pharmacy, (20th ed. 2000). For injection, the compounds may be formulated and diluted in aqueous solutions, such as in physiologically compatible buffers such as Hank's solution, Ringer's solution, or physiological saline buffer.

The following abbreviations are used in the text: CTA=chain transfer agent, NHS=N-hydroxysuccinimide, Et₂O=diethylether, ESI-MS=electrospray ionization mass spectrometry, APCI=Atmospheric pressure chemical ionization, min=minute, r.t.=room temperature, h=hour, EtOAc=ethyl acetate, EDC=1-Ethyl-3-(3-dimethylaminopropyl)carbodiimide, HATU=1-[Bis(dinethylamino)methylene]-1H-1,2,3-triazolo[4,5-b]pyridinium 3-oxide hexafluorophosphate, or Hexafluorophosphate Azabenzotriazole Tetramethyl Uronium, DMF=N, N-dimethylacetamide, DMAP=4-Dimethylaminopyridine, DIC=N, N-Diisopropylcarbodiimide, MeOH=methanol, DCM=dichloromethane, GPC=gel permeation chromatography, MW=molecular weight, Mn number average molecular weight, Mw=weight average molecular weight, PDI=polydispersity index, PBS=phosphate buffered saline, D_{H}=hydrodynamic diameter equivalent to dynamic light scattering assessed number mean diameter, tBuOH=tertiary butyl alcohol, AIBN=azobisisobutyronitrile, GPC-MALS=Gel Permeation Chromatography (GPC) with Multi-Angle Light Scattering Detection (MALS), RAFT=reversible addition-fragmentation chain-transfer, DMSO=dimethyl sulfoxide, DMAc=Dimethylacetamide, HPLC=high pressure liquid chromatography, wt=weight, DLS=dynamic light scattering, Tₜᵣ=thermo-responsive transition temperature, M=molar, SEC=size exclusion chromatography, rcf=relative centrifugal force (equivalent to times force of gravity or *g), L=Liter.

### Example 1—Synthesis of Monomers, Initiators, CTAs and Drugs

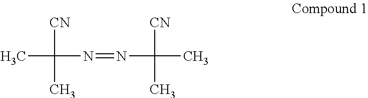

Compound 1. Initiator 2,2′-azo-bis-isobutyrylnitrile or 2,2′-Azobis(2-methylpropionitrile) (AIBN) was purchased from Sigma Aldrich (St Louis, MO) and recrystallized in methanol prior to use.

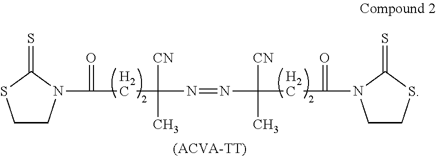

Compound 2. 2-[1-Cyano-1-methyl-4-oxo-4-(2-thioxo-thiazolidin-3-yl)-butylazo]-2-methyl-5-oxo-5-(2-thioxothiazolidin-3-yl)-pentanenitrile (ACVA-TT) was synthesized by activating the carboxylic acids in 4,4′-azobis(4-cyanovaleric acid) (ACVA-COOH) (CAS 2638-94-0) with 2-thiazoline-2-thiol via N,N′-diisopropylcarbodiimide (DIC) coupling reaction. To a 20 mL scintillation vial, ACVA-COOH (501.5 mg, 1.79 mmol), 2-thiazoline-2-thiol (411.8 mg, 3.46 mmol), 4-(dimethylamino)pyridine (DMAP, 10.6 mg, 0.087 mmol), and 15 mL of DCM were added. The mixture was stirred vigorously in an ice-bath for 15 min before DIC (497.1 mg, 3.94 mmol) was added. The mixture was allowed to slowly warm up to r.t. and react for another 15 min before it was washed with saturated solution of NaHCO₃ (20 mL×2), DI water (20 mL×1). The organic phase was then dried over MgSO₄ and evaporated to yield dry product, which was purified by recrystallizing from DCM/Et₂O at −20° C. After decanting the solvent, bright yellow powder was obtained (658.3 mg, 76.2%). ESI-MS: m/z=483.07 (M-H)⁺.

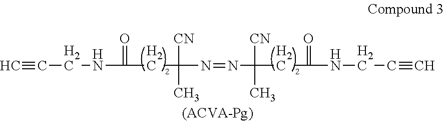

Compound 3. 4-Cyano-4-(1-cyano-3-ethynylcarbamoyl-1-methylpropylazo)-N-ethynyl-4-methylbutyramide (ACVA-Pg) was synthesized by reacting ACVA-TT with 3-amino-1-propyne. To a 20 mL scintillation vial, ACVA-TT (329.7 mg, 0.684 mmol), 3-amino-1-propyne (99.76 mg, 1.81 mmol), and 10 mL of DCM were added. Triethylamine (253 μL, 1.82 mmol) was then added to the mixture. The reaction was allowed to proceed for another 1 h at r.t. before solvent was removed. The crude product was purified via flash C-18 column. Product fractions were pool and dried to yield white solid (190.3 mg, 78.5%). ESI-MS: m/z=355.18 (M-H)⁺.

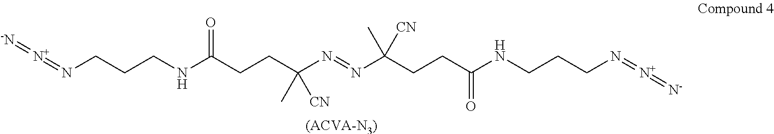

Compound 4. ACVA-N₃ was synthesized by reacting ACVA with 1-azido-3-propanamine. To a 20 mL scintillation vial, ACVA (250.0 mg, 0.893 mmol), 1-azido-3-propanamine (187.7 mg, 1.87 mmol), and 5 mL of DCM were added. EDC (375.2, 1.96 mmol) was then added to the mixture over 20 min. The reaction was allowed to proceed for another 1 h at r.t. before solvent was removed. The crude product was recrystallized from EtOAc/Et₂O to yield white solid (130.0 mg, 32.8%). ESI-MS: m/z=445.2 (M-H)⁺.

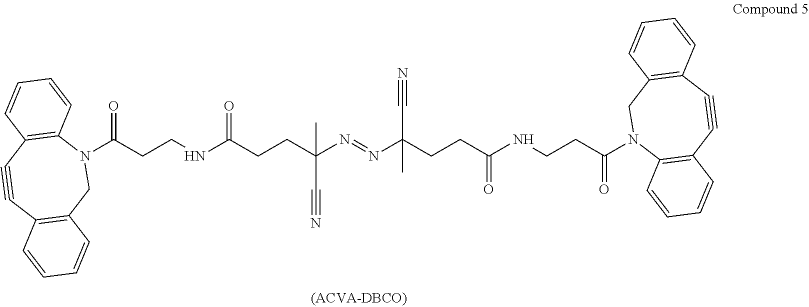 [SMILES: CC(C#N)(CCC(=O)NCCC(=O)N1Cc2ccccc2C#Cc2ccccc21)/N=N/C(C)(C#N)CCC(=O)NCCC(=O)N1Cc2ccccc2C#Cc2ccccc21]

Compound 5. ACVA-DBCO was synthesized by reacting DBCO-amine (CAS 1255942-06-3) with ACVA-TT (Compound 2). To a 20 mL scintillation vial, ACVA-TT (900 mg, 1.865 mmol), DBCO-amine (1033.3 mg, 3.739 mmol), and 5 mL of DCM were added. The reaction was allowed to proceed for 1 h at r.t. before solvent was removed. The product was purified using flash chromatography (100 g column) using a ramp from 0-5% (v/v) MeOH in DCM and monitoring at 305 nm. Product eluted from the column at approximately 3% (v/v) MeOH, which was then confirmed to be >95% purity by HPLC, dried under vacuum and stored dry at −20° C. (Yield 1128.6 mg, 75.8%). ESI-MS: m/z=797.35 (M-H)⁺.

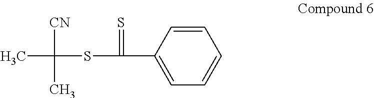

Compound 6. 2-Cyano-2-propyl benzodithioate (CPDB) (CAS 201611-85-0) was purchased from Sigma Aldrich and used without further purification.

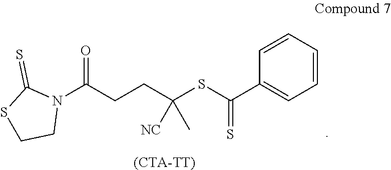

Compound 7. Dithiobenzoic acid 1-cyano-1-methyl-4-oxo-4-(2-thioxothiazolidin-3-yl)butyl ester (CTA-TT) was synthesized by activating the carboxylic acid in 4-cyano-4-(phenylcarbonothioylthio)pentanoic acid (CTA-COOH) with 2-thiazoline-2-thiol. To a 20 mL scintillation vial, CTA-COOH (499.8 mg, 1.79 mmol), 2-thiazoline-2-thiol (196.5 mg, 1.65 mmol), DMAP (8 mg, 0.065 mmol), and 10 mL of DCM were added. The mixture was stirred vigorously in an ice-bath for 15 min before EDC (446.2 mg, 2.33 mmol) was added. The mixture was allowed to slowly warm up to r.t. and react for another 15 min before it was washed with saturated solution of NaHCO₃ (10 mL×2) and DI water (10 mL×2). The organic phase was then dried over MgSO₄ and evaporated to yield dry product, which was purified on a preparatory HPLC system using a gradient of 58-78% (v/v) acetonitrile/H₂O (0.05% (v/v) TFA) over 12 minutes on an Agilent Prep C-18 column, 30×100 mm, 5 μm. The product eluted at 6.5 minutes and the product fractions were pooled and lyophilized yielding red viscous liquid (400.0 mg, 63.8%). ESI-MS: m/z=381.0 (M-H)⁺.

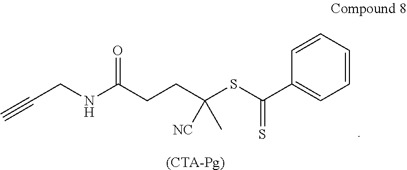

Compound 8. Dithiobenzoic acid 1-cyano-1-methyl-3-prop-2-ynylcarbamoylpropyl ester (CTA-Pg) was synthesized by reacting CTA-COOH with 3-amino-1-propyne. To a 20 mL scintillation vial, CTA-COOH (100.0 mg, 0.358 mmol), 3-amino-1-propyne (21.69 mg, 0.394 mmol), HATU (272.2 mg, 0.716 mmol), DIEA (185.0 mg, 1.432 mmol), and 4 mL of DMF were added. The mixture was stirred at r.t. for 2 h before it was washed with saturated solution of NaHCO₃ (10 mL×2) and brine (10 mL×1). The organic phase was then dried over MgSO₄ and evaporated to yield dry product, which was purified on a preparatory HPLC system using a gradient of 40-70% (v/v) acetonitrile/H₂O (0.05% (v/v) TFA) over 12 minutes on an Agilent Prep C-18 column, 50×100 mm, 5 μm. The product eluted at 8.5 minutes and the product fractions were pooled and lyophilized yielding red viscous liquid (54.0 mg, 47.7%). ESI-MS: m/z=317.07 (M-H)⁺.

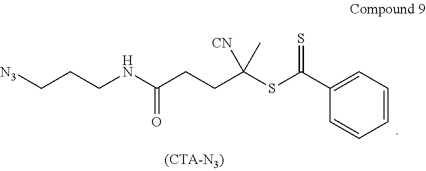

Compound 9. 5-((3-azidopropyl)amino)-2-cyano-5-oxopentan-2-yl benzodithioate (CTA-N₃) was prepared similarly to Compound 7, but with 3-azidoporpylamine substituted for 3-amino-1-propyne. ESI-MS: m/z=361.10 (M-H)⁺.

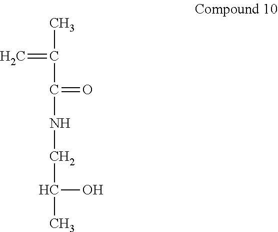

Compound 10. N-(2-Hydroxypropyl)methacrylamide (HPMA) (CAS 21442-01-3) was synthesized by reacting 1-amino-2-propanol with methacryloyl chloride. To a 1 L round-bottom flask equipped with magnetic stir bar, 1-amino-2-propanol (60.0 mL, 0.777 mol), sodium bicarbonate (60.27 g, 0.717 mol), 4-methoxyphenol (1.00 g, 8.1 mmol), and 200 mL of dichloromethane (DCM) were added. The flask was immersed in an acetone-dry ice bath for 15 min with vigorous stirring. Methacryloyl chloride (70.0 mL, 0.723 mol) dissolved in 80 mL of DCM was added dropwise under Ar (g) over 3 h. The reaction was allowed to proceed at r.t. for another 30 min. After removing the salt, crude product was purified via flash column (Biotage SNAP ultra 100g), using gradient eluent DCM/MeOH with MeOH increased from 0 to 10% (v/v). The solid thus obtained after solvent removal was then recrystallized from acetone to yield HPMA as white crystal (22.4 g, 21.6%). ESI-MS: m/z=144.09 (M-H)⁺.

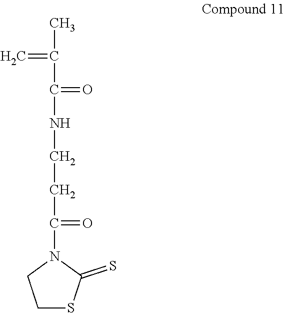 [SMILES: C=C(C)C(=O)NCCC(=O)N1CCSC1=S]

Compound 11. N-Methacryloyl-3-aminopropanoic acid-thiazolidine-2-thione (MA-b-Ala-TT) was prepared in two-step synthesis. First, N-methacryloyl-3-aminopropanoic acid (MA-b-Ala-COOH) was synthesized by reacting beta-alanine (15.07 g, 169.2 mmol) to methacrylic anhydride (28.60 g, 185.5 mmol) in the presence of 4-methoxyphenol (0.218 g, 1.76 mmol) in a 100 mL round bottom flask at r.t. over weekend. The mixture was passed through flash column (Biotage SNAP ultra 100g), using gradient eluent DCM/MeOH with MeOH increased from 0 to 10% (v/v). After combining fractions and removing solvent, product was recrystallized from EtOAc/Et₂O (1/1 v/v) at −20° C., yielding a white crystal. MA-b-Ala-COOH (5.17 g, 33 mmol), 1,3-thiazolidine-2-thione (4.30 g, 36 mmol), EDC (7.93 g, 41 mmol), DMAP (0.39 g, 3 mmol), and 100 mL DCM were mixed in a 250 mL round bottom flask. It was allowed to react 1 h before the product was washed by 1 M HCl (2×) and DI water (1×). Upon solvent removal, yellow solid product was collected.

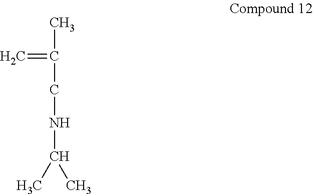

Compound 12. N-isopropylmethacrylamide (NIPMAM) (CAS 13749-61-6) was purchased from Sigma Aldrich and further purified by monomer recrystallization from 60/40 v/v toluene/hexane to remove impurities.

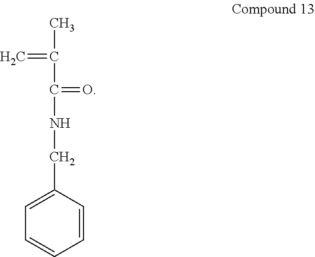

Compound 13. Benzyl methacryalmide (BnMAM) (CAS 3219-55-4) was purchased from Polysciences (Warrington, PA) and used without further purification.

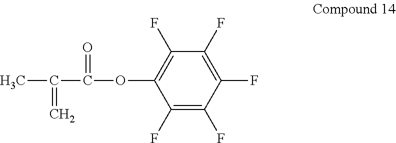 [SMILES: C=C(C)C(=O)Oc1c(F)c(F)c(F)c(F)c1F]

Compound 14. Perfluorophenyl methacrylate (MA-PFP) (CAS 13642-97-2) was purchased from Tokyo Chemical Industry (Tokyo, Japan) and used without further purification.

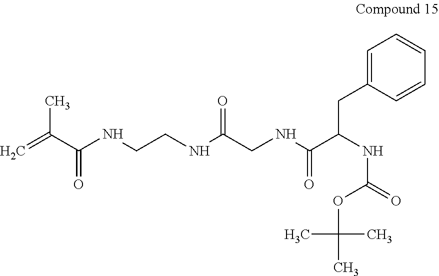 [SMILES: C=C(C)C(=O)NCCNC(=O)CNC(=O)C(Cc1ccccc1)NC(=O)OC(C)(C)C]

Compound 15. tert-butyl (1-((1-((2-((2-methylacryloyl)amino)ethyl)amino)-1-oxo-3-phenylpropan-2-yl)amino)-1-oxo-3-phenylpropan-2-yl)carbamate (MA-EDA-Phe-Phe-Boc) was synthesized by reacting 2-aminoethylmethacrylamide hydrochloride (AEMA⁺Cl⁻, 76259-32-0, Polysciences, PA, USA) to Boc-Phe-Phe-OH (13122-90-2, Bachem, Switzerland) in the presence of COMU (1075198-30-9, Merck, NJ, USA) and triethylamine (121-44-8, Merck, NJ, USA) as follows: A mixture of AEMA⁺Cl⁻ (0.16 g, 1.0 mmol), Boc-Phe-Phe-OH (0.41 g, 1.0 mmol), COMU (0.51 g, 1.2 mmol) and triethylamine (0.53 mL, 4.0 mmol) was dissolved in DMF (8 mL) and stirred at r.t. for 30 min. The reaction mixture was then diluted with DCM (50 mL) and washed sequentially with aqueous HCl (1 M, 1×50 mL) and saturated aqueous NaHCO₃ (1×50 mL) and NaCl (1×50 mL). The organic phase was dried over MgSO₄, concentrated in vacuo and passed through a silica gel column, using DCM/MeOH/NH₄OH (90:10:1) mixture as the mobile phase (R_{f}=0.1). Upon solvent removal, white amorphous solid product (0.36 g) was obtained.

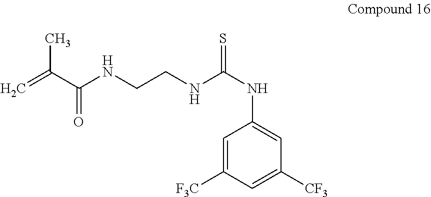 [SMILES: C=C(C)C(=O)NCCNC(=S)Nc1cc(C(F)(F)F)cc(C(F)(F)F)c1]

Compound 16. N-(2-(((3,5-bis(trifluoromethyl)phenyl)carbamothioyl)amino)ethyl)-2-methacrylamide (MA-EDA-BTFMP) was synthesized by reacting 2-aminoethylmethacrylamide hydrochloride (AEMA⁺Cl⁻, 76259-32-0, Polysciences, PA, USA) to 3,5-bis(trifuoromethyl)phenyl isothiocyanate (BTFMPI, 23165-29-9, TCI, Japan) in the presence of triethylamine (121-44-8, Merck, NJ, USA) as follows: A mixture of AEMA⁺Cl⁻ (0.44 g, 6.7 mmol), BTFPI (0.73 g, 6.7 mmol) and triethylamine (1.45 mL, 10.0 mmol) was dissolved in DCM (10 mL) and stirred at r.t. for 30 min. The reaction mixture was then washed sequentially with saturated aqueous KHSO₄ (1×10 mL) and NaCl (1×10 mL). The organic phase was dried over MgSO₄ and the solvent was evaporated to dryness, yielding white amorphous solid product (1.10 g).

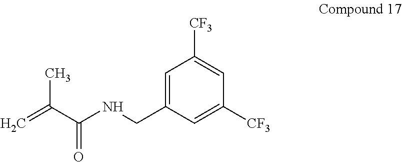 [SMILES: C=C(C)C(=O)NCc1cc(C(F)(F)F)cc(C(F)(F)F)c1]

Compound 17. N-(3,5-bis(trifluoromethyl)benzyl)-2-methacrylamide (MA-BTFMB) was synthesized by reacting methacrylic acid (MA, 79-41-4, TCI, Japan) to 3,5-bis(trifluoromethyl)benzylamine (BTFMB, 85068-29-7, TCI, Japan) in the presence of EDC (25952-53-8, TCI, Japan) and DMAP (5683-33-0, TCI, Japan) as follows: A mixture of MA (0.20 g, 2.32 mmol) and BTFMB (0.57 g, 2.32 mmol) was dissolved in DCM (12 mL). Solid EDC (0.49 g, 2.56 mmol) and a catalytic amount of DMAP were added and the reaction mixture was stirred at r.t. for 30 min. The solution was concentrated in vacuo and passed through a silica gel column, using gradient elution hexane with ethyl acetate increased from 50 to 100% (v/v). Upon solvent removal, white amorphous solid product (0.61 g) was obtained.

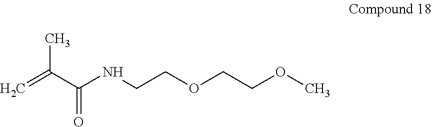

Compound 18. N-(2-(2-methoxyethoxy)ethyl)-2-methacrylamid (DEGMAM) was prepared in a three-step synthesis by reacting 1-bromo-2-(2-methoxyethoxy)ethane (54149-17-6, TCI, Japan) with sodium azide (26628-22-8, TCI, Japan) followed by reduction of the formed 1-azido-2-(2-methoxyethoxy)ethane with triphenylphosphine (603-35-0, TCI, Japan) and subsequent condensation of the generated 2-(2-methoxyethoxy)ethanamine with methacrylic acid (MA, 79-41-4, TCI, Japan) in the presence of EDC (25952-53-8, TCI, Japan) and DMAP (5683-33-0, TCI, Japan) as follows: First, a mixture of 1-bromo-2-(2-methoxyethoxy)ethane (1.0 g, 5.5 mmol) and NaN₃ was dissolved in DMF (7 mL), solid KI (0.23 g, 1.4 mmol) was added and the solution was stirred at 60° C. overnight. The solvent was removed under reduced pressure and the product was obtained by extracting the residue into diethyl ether. Upon solvent removal, oily 1-azido-2-(2-methoxyethoxy)ethane (0.62 g, 4.4 mmol) was dissolved in THF (5 mL), the solution was cooled to 0° C. and triphenylphosphine (1.23 g, 4.7 mmol) was slowly added. The reaction mixture was then freely warmed to r.t. and allowed to react for three hours. After that, water (1.5 mL, 94.0 mmol) was added and the solution was stirred at r.t. overnight. The solution was concentrated in vacuo and passed through a silica gel column, using DCM/MeOH/NH₄OH (90:10:1 v/v) mixture as the mobile phase (R_{f}=0.05). Upon solvent removal, liquid 2-(2-methoxyethoxy)ethanamine (0.42 g, 3.5 mmol) was dissolved in DCM (7 mL) and mixed with MA (0.30 g, 3.5 mmol). Solid EDC (0.74 g, 3.9 mmol) and a catalytic amount of DMAP were added and the reaction mixture was stirred at r.t. for three hours. The solution was concentrated in vacuo and passed through a silica gel column, using ethyl acetate as the mobile phase (R_{f}=0.3). Upon solvent removal, colourless liquid product (0.49 g) was obtained.

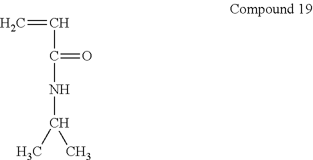

Compound 19. N-isopropylacrylamide (NIPAM) (CAS 2210-25-5) was purchased from Sigma Aldrich and used without further purification.

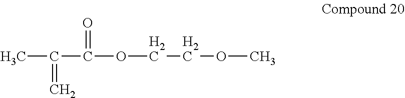

Compound 20. Methyl ether methacrylate (MEGMA) (CAS 6976-93-8) was purchased from Sigma Aldrich and used without further purification.

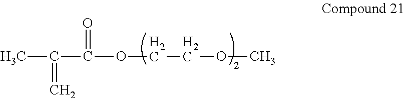

Compound 21. Di(ethylene glycol) methyl ether methacrylate (DEGMA) (CAS 45103-58-0) was purchased from Tokyo Chemical Inventory and passed through a spin column containing potassium carbonate (K₂CO₃) and Brockmann number #1 basic alumina to remove inhibitor 4-methoxyphenol.

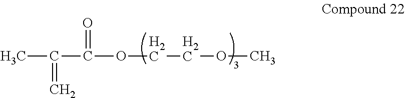

Compound 22. Triethylene glycol methyl ether methacrylate (TEGMA) (CAS 24493-59-2) was purchased from Sigma Aldrich and passed through a spin column containing potassium carbonate (K₂CO₃) and Brockmann number #1 basic alumina to remove inhibitor 4-methoxyphenol.

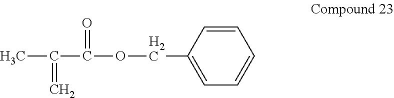

Compound 23. Benzyl methacrylate (BnMA) (CAS 2495-37-6) was purchased from Sigma Aldrich and passed through a spin column containing potassium carbonate (K₂CO₃) and Brockmann number #1 basic alumina to remove inhibitor 4-methoxyphenol prior to use.

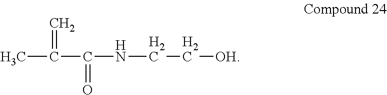

Compound 24. N-(2-Hydroxyethyl)methacrylamide (HEMAM) (CAS 5238-56-2) was purchased from Astatech and passed through a spin column containing potassium carbonate (K₂CO₃) and Brockmann number #1 basic alumina to remove inhibitors prior to use.

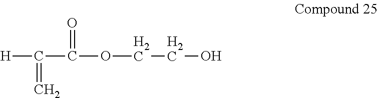

Compound 25. 2-hydroxyethyl acrylate (HEA) (CAS 818-61-1) was purchased from Sigma Aldrich and used without further purification.

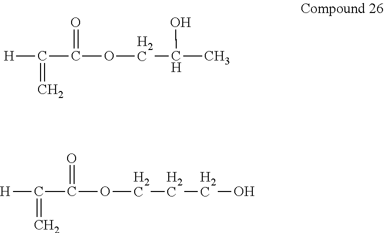

Compound 26. Hydroxypropyl acrylate (HPA) (CAS 25584-83-2) as a mixture of isomers was purchased from Sigma Aldrich and used without further purification.

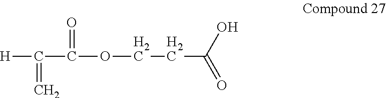

Compound 27. 2-carboxyethyl acrylate (CEA) (CAS 24615-84-7) was purchased from Sigma Aldrich and used without further purification.

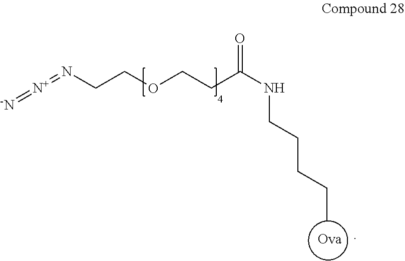

Compound 28. Ovalbumin-PEG4-azide (Ova-N3) was synthesized by reacting ovalbumin protein (vac-stova) purchased from Invivogen (Carlsbad, CA) with azido-PEG4-NHS ester (CAS 944251-24-5) purchased from Broadpharm (San Diego, CA). Ovalbumin was dissolved in conjugation buffer (150 mM PBS, pH 7.4) and reacted with azido-PEG4-NHS ester at a molar ratio of 1:3 (protein to NHS) for 16 hours at room temperature. The resulting Ova-N3 functionalized drug was purified via centrifugal filter separation using 3 washes with a 10 kDa molecular weight cutoff filter and protein concentration determined using absorbance measurements at 280 nm.

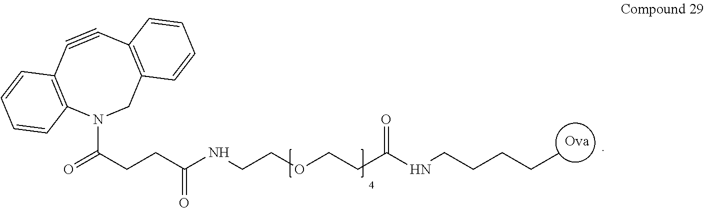

Compound 29. Ovalbumin-PEG4-DBCO (Ova-DBCO) was synthesized by reacting ovalbumin protein (vac-stova) purchased from Invivogen (Carlsbad, CA) with DBCO-PEG4-NHS ester (CAS 1427004-19-0) purchased from Click Chemistry Tools. Ovalbumin was dissolved in conjugation buffer (150 mM PBS, pH 7.4) and reacted with DBCO-PEG4-NHS ester at a molar ratio of 1:3 (protein to NHS) for 16 hours at room temperature. The resulting Ova-DBCO functionalized drug was purified via centrifugal filter separation using 3 washes with a 10 kDa molecular weight cutoff filter and protein concentration determined using absorbance measurements at 280 nm.

Compound 30. Peptide p2610 was synthesized by Genscript from peptide sequence requested: NH2-LSPRTLNAW (SEQ ID NO: 6) with C-terminal amidation.

Compound 31. Peptide p2860 was synthesized by Genscript from peptide sequence requested: Azide-PEG12-TESNKKFLPFQQFGRDIA (SEQ ID NO: 7) with C-terminal amidation.

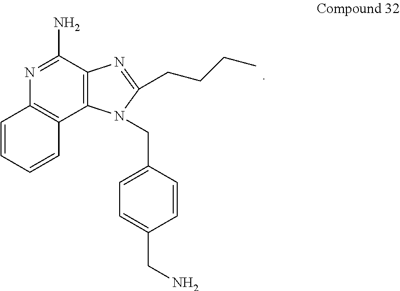 [SMILES: CCCCc1nc2c(N)nc3ccccc3c2n1Cc1ccc(CN)cc1]

Compound 32. 1-(4-(aminomethyl)benzyl)-2-butyl-1H-imidazo[4,5-c]quinolin-4-amine, referred to as 2Bxy, was previously described (see: Lynn, G. M. et al. Nat. Biotechnol., 2015, 33 (11), 1201-1210 and Shukla, N. M. et al. Bioorg Med Chem Lett 2010, 20 (22), 6384-6386). ¹H NMR (400 MHz, DMSO-d6) δ 7.77 (dd, J=8.4, 1.4 Hz, 1H), 7.55 (dd, J=8.4, 1.2 Hz, 1H), 7.35-7.28 (m, 1H), 7.25 (d, J=7.9 Hz, 2H), 7.06-6.98 (m, 1H), 6.94 (d, J=7.9 Hz, 2H), 6.50 (s, 2H), 5.81 (s, 2H), 3.64 (s, 2H), 2.92-2.84 (m, 2H), 2.15 (s, 2H), 1.71 (q, J=7.5 Hz, 2H), 1.36 (q, J=7.4 Hz, 2H), 0.85 (t, J=7.4 Hz, 3H). MS (APCI) calculated for C₂₂H₂₅N₅ m/z 359.2, found 360.3

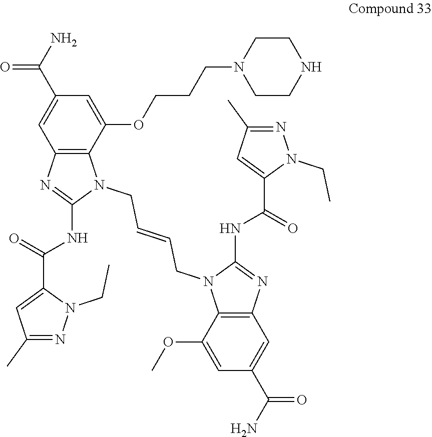 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCNCC3)c21]

Compound 33. E)-1-(4-(5-carbamoyl-2-(1-ethyl-3-methyl-1H-pyrazole-5-carboxamido)-7-(3-(piperazin-1-yl)propoxy)-1H-benzo[d]imidazol-1-yl)but-2-en-1-yl)-2-(1-ethyl-3-methyl-1H-pyrazole-5-carboxamido)-7-methoxy-1H-benzo[d]imidazole-5-carboxamide referred to as diABZI-piperidine or diABZI-pip was synthesized by WuXi Apptec and confirmed by LC-MS to have m/z 848.42 Da. diABZI-pip was adapted from the amidobenzimidazole disclosed with a morpholino group replaced with a piperidine (Ramanjulu, J. M. et al. Nature, 2018, 564, 439-443).

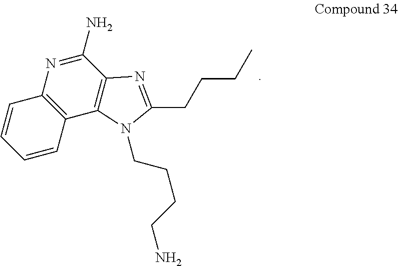 [SMILES: CCCCc1nc2c(N)nc3ccccc3c2n1CCCCN]

Compound 34. Sometimes referred to as “2B,” 1-(4-aminobutyl)-2-butyl-1H-imidazo[4,5-c]quinolin-4-amine, is a TLR-7/8 agonist that was synthesized as previously described (Lynn G M, et al., Nat Biotechnol 38(3):320-332, 2020). Note: the butyl amine group provided a reactive handle for attachment to star polymers either directly or through a linker. ¹H NMR (400 MHZ, DMSO-d6) δ 8.03 (d, J=8.1 HZ, 1H), 7.59 (d, J=8.1 Hz, 1H), 7.41 (t, J=7.41 Hz, 1H), 7.25 (t, J=7.4 Hz, 1H), 6.47 (s, 2H), 4.49 (t, J=7.4 Hz, 2H), 2.91 (t, J=7.78 Hz, 2H), 2.57 (t, J=6.64 Hz, 1H), 1.80 (m, 4H), 1.46 (sep, J=7.75 Hz, 4H), 0.96 (t, J=7.4 Hz, 3H). MS (ESI) calculated for C₁₈H₂₅N₅, m/z 311.21, found 312.3.

### Example 2—Synthesis of Single Block Polymers

The following section details the methods used in the preparation of single and di-block polymers useful in the practice of the invention(s) described herein as well as single-block polymer controls. Synthetic procedures are described in detail for each unique polymer composition. Unless otherwise specified, a standard workflow of platform purification and analytical methods were used in the purification and characterization of the polymers and is summarized here:

Purification procedures. Unless otherwise specified, polymers were purified from the crude reaction mixture by either size exclusion chromatography (SEC) using a column packed with Sephadex LH-20 (Sigma) as the stationary phase and methanol as the mobile phase, or by precipitation.

SEC purification. Sephadex LH-20 was used to separate polymers from low molecular weight impurities when precipitation was not possible. The LH-20 resin was solvated with anhydrous methanol and packed into a vertical column. Following column equilibration with 2 or more column volumes of methanol, crude samples were diluted with anhydrous methanol added to directly to the LH-20 column and the eluted with methanol as the mobile phase. All fractions were assessed by UV; polymer-containing fractions were combined and then solvent was removed under vacuum to yield purified polymer.

Precipitation. Precipitation was typically employed when a polymer was not soluble in certain solvents or solvent systems, but impurities were soluble. In brief, crude samples, e.g., reaction mixtures were diluted with anhydrous methanol and then added dropwise to between 5-10 fold-volumes of the specified precipitation solvent. The solution was mixed thoroughly and then the precipitated polymer was collected either via centrifugation (4000 rcf, 10 minutes), or by vacuum filtration using a Büchner funnel. The collected polymer was then redissolved in a minimal volume of anhydrous methanol and precipitated a second time and the solid was collected by centrifugation or filtration. The solid was dried under vacuum to remove any residual precipitation solvent.

Analytical Methods.

Molecular weights (Mw and Mn) and polydispersity of polymers and co-polymers were measured by gel permeation chromatography using a high-performance liquid chromatography (HPLC) system (Agilent, USA) equipped with a UV-Vis photodiode array (PDA, Agilent) detector, refractive index (RI) Optilab T-rEX and multiangle light scattering (MALS, Wyatt, Santa Barbara, CA) DAWN HELEOS-II detectors (Wyatt Technology). A set of TSK-Gel SuperAW3000 and SuperAW4000 columns (Tosoh Bioscience, Japan) were connected in series and used with isocratic mobile phase composed of either (A) methanol-acetate GPC buffer composed of 80% methanol-20% sodium acetate buffer (0.3 M, pH 6.5) or (2) DMF GPC buffer composed of DMF with 10 mM LiBr at a flow rate of 0.5 mL/min. The specific refractive index increment (dn/dc) of copolymers was determined using a known total injected mass with an assumption of 100% recovery. Degree of polymerization (Xₙ) was estimated based on GPC determined number average molecular weight (Mn) and average monomer MW (i.e., Mn/average monomer molecular weight=degree of polymerization). Polymer purity following purification was estimated based on the % area-under-the-peak at 220 nm; all polymers had 85% purity (220 nm).

Unless otherwise indicated, the hydrodynamic diameter (D_{H}), also referred to as simply the diameter or as the number average diameter or number mean diameter, of the polymers and copolymers were measured using dynamic light scattering (DLS) at a scattering angle θ=90° using a Zetasizer Ultra (Malvern Panalytical, Malvern, England) equipped with a 633 nm laser, capillary cell (ZSU1002) and ZS Xplorer software. DLS measurement of diameter of nanoparticles may be assessed as a number average, equivalent to number mean, intensity mean or volume mean diameter. For measurements presented, number mean diameter was used for assessment. Volume mean diameter and intensity mean diameter may measure a larger diameter for some formulations due to the manner by which nanoparticle materials scatter light proportionally to the hydrodynamic radius of the material in solution. Additionally, DLS can be performed at multiple scattering angles (0) including backscatter angles of 173°, 175°, 187°, or forward scatter angles including 15°. Scattering angle may influence the sensitivity of the DLS instrument to specific diameter measurements. DLS instruments may also a laser of wavelengths other than 633 nm, which may similarly change the sensitivity to nanoparticles depending on the material of the nanoparticles. For transition temperature (Tₜᵣ) characterization, DLS measurements were performed for temperature sweeps over a maximum range of 4-50° C. at a concentration of 0.5 mg/mL, 5 mg/mL or 200 mg/mL in PBS (150 mM NaCl, pH 7.4). At each step, measurements were performed after reaching steady-state temperature conditions. Characterization of the polymer chain conformational changes was evaluated from the temperature dependence of the hydrodynamic diameter (D_{H}); the Tₜᵣ value was determined from the intersection point of two lines formed by the linear regression of a lower horizontal asymptote and a vertical section of the S-shaped curve (sigmoidal curve) fit.

Absorbance measurements (UV-Vis) of polymers diluted into anhydrous methanol were used to determine end group functionalization and mole fraction (i.e., mol %) of monomers with appreciable absorbance above 254 nm. In brief, the end group functionalization and/or mol % of monomers can be experimentally determined using the extinction coefficient of the end group or monomer and experimentally determined absorbance of a sample at a known concentration. For example: UV-Vis can be used to estimate the agonist density (mol %) of a monomer y, for a statistical copolymer comprised of monomers x and y using the following relationship:

$$mol {\%}_{y} = ( \frac{1}{1 + ( \frac{ρ \times ε}{A \times M {w}_{x}} - \frac{M {w}_{y}}{M w x} )} ) * 100$$

Where, mol %_{y} (agonist density)=percentage of copolymer that is y (e.g., TLR-7/8a containing monomer), for copolymer comprised of x and y monomers; ρ=volumetric mass density (mg/mL) of copolymer during UV-Vis measurement; ε=molar extinction coefficient for monomer y (e.g., for TLR-7/8a=5,012); and, A=Absorbance; Mwₓ=molecular weight (g/mol) of majority monomer; and, Mw_{y}=molecular weight (g/mol) of minority monomer.

Example calculation: For a polymer comprised HPMA (MW_{HPMA}=143.2) as the majority monomer and MA-Ahx-PEG4-7/8a (MW_{MA-PEG47/8a}=741.9) as a minority monomer that is suspended in methanol at 0.1 mg/mL and has an average absorbance of 0.25 at 325 nm, the mol % of the minority unit, MA-PEG4-7/8a is:

$$mol {\%}_{M A - A h x - P E G 4 - 7 / 8 a} = ( \frac{1}{1 + ( \frac{0.1 \times 5012}{0.25 \times 143.2} - \frac{741.9}{143.2} )} ) * 100 = 10.2 \%$$

Chromophore wavelength and extinction coefficients used include: (a) dithiobenzoate (DTB), 305 nm, ε=12,606 L mol⁻1 cm⁻¹, (b) activated carboxylic acid/thiazolidine-2-thione (TT), 305 nm, ε=10,300 L mol⁻1 cm⁻¹, (c) dibenzocyclooctyne (DBCO), 292 nm, ε=13,000 L mol⁻1 cm⁻¹.

The following sections describe the synthesis of single block polymers and copolymers and multiblock polymers and copolymers.

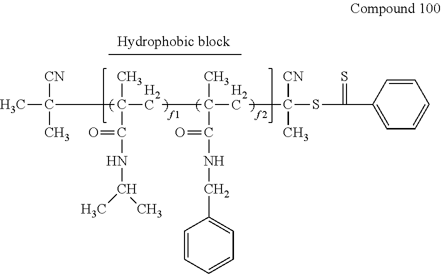

Compound 100. CN-p[(NIPMAM)_{f1}-co-(BnMAM)_{f2}]-DTB were synthesized via RAFT polymerization of NIPMAM (Compound 12) and BnMAM (Compound 13) using CPDB (Compound 6) as a chain transfer agent and AIBN (Compound 1) as an initiator in tert-butanol (tBuOH) with 10% dimethylacetamide (DMAc) as cosolvent. The initial combined monomer concentration [NIPMAM+BnMAM]₀ was 2 mol/L; the molar ratio of chain transfer agent to initiator, [CPDB]₀:[AIBN]₀, was 1:0.05 (i.e., 2:1); and [NIPMAM+BnMAM]₀:[CTA]₀ was 148. to target a number average molecular weight of 16 kDa. NIPMAM (814.0 mg, 6.40 mmol) was dissolved in 3.6 mL of tBuOH. BnMAM (280.2 mg, 1.6 mmol) was dissolved in 0.4 mL of anhydrous DMAc. CPDB (12 mg, 0.0542 mmol) and AIBN (4.45 mg, 0.0271 mmol) were dissolved in anhydrous DMSO before mixing with the monomer solution. The mixture was transferred to a 5 mL ampule, which was sealed with a rubber septum and sparged with Ar (g) at r.t. for 20 min. The flask was then immersed in a water circulator preheated to 70° C. and polymerized for 16 h. The polymer was purified by separation through LH-20 using methanol as the mobile phase. Methanol was removed by rotary evaporation and the polymer was dried under high vacuum overnight to remove residual solvent, yielding a light pink viscous gel (383 mg, 35% yield). Number-average (Mn) and weight-average molecular weight (M_{w}) were 16.2 kDa and 16.5 kDa, respectively, and polydispersity (PDI) was 1.016 measured by GPC-MALS.

Compounds 101-110, p(NIPMAM)_{f1}-DTB, p[(NIPMAM)_{f1}-co-(BnMAM)_{f1}]-DTB with varying mol % of NIPMAM and BnMAM and p(BnMAM)-DTB were synthesized similarly to as described for Compound 33. In brief, [NIPMAM+BnMAM]₀ was between 1-2 mol/L; and, the molar ratio of chain transfer agent to initiator, [CPDB]₀:[AIBN]₀, was 1:0.05 (i.e., 2:1). The [NIPMAM+BnMAM]₀:[CTA]₀ and monomer mole fraction of [BnMAM]₀ defined as [BnMAM]₀/([NIPMAM]₀+[BnMAM]₀) and solvent system are provided in Table 2 and were varied to obtain polymers of varying length and mol % BnMAM monomer composition. All polymers were prepared using CPDB (Compound 6) as a chain transfer agent and AIBN (Compound 1) as an initiator in the specified solvent system (see Table 2) at 70° C. for 16 hours. Solvent systems are reported as the ratio of solvents used, e.g., tBuOH/DMAc (9/1) refers to a 90% volume solution of tert-butanol with 10% volume dimethylacetamide. A [monomer]:[CTA] ratio of [125] refers to a molar ratio of 125 to 1 molar ratio of monomer to chain transfer agent. Monomer molar ratio refers to the ratio of molar amounts of the monomers; e.g., a monomer molar ratio of NIPMAM/BnMAM (9/1) refers to a 90 mol % NIPMAM and 10 mol % BnMAM monomers of total monomer in the reaction, which would be expected to lead to a copolymer with 90 mol % NIPMAM and 10 mol % BnMAM monomers. The two purification procedures (LH-20 SEC and precipitation) are described elsewhere. Number-average (Mₙ), weight-average (M_{w}) molecular weights and PDI measured by GPC-MALS are listed in Table 7.

TABLE 2
Synthesis conditions of single block temperature-responsive p[(NIPMAM)_{f1}-co-(BnMAM)_{f2}] polymers.

| | | Monomer | | |
|---|---|---|---|---|
| | | concentration, | Monomer | |
| Compound | Polymer | solvent system, and | molar ratio | Purification |
| # | structure | [monomer:CTA] ratio | (f1/f2) | procedure |
| 101 | CN- | 2M, tBuOH/DMAc | N.A. | purification using |
| | p(NIPMAM)_{f1}- | (9/1), [125:1] | | LH-20 SEC |
| | DTB | | | |
| 102 | CN- | 1M, tBuOH/DMAc | NIPMAM/ | precipitation into |
| | p[(NIPMAM)_{f1}- | (9/1), [231:1] | BnMAM | Et₂O/hexane (3:1), |
| | co-(BnMAM)_{f2}]- | | (95/5) | purification using |
| | DTB | | | LH-20 SEC |
| 103 | CN- | 1M, tBuOH/DMAc | NIPMAM/ | precipitation into |
| | p[(NIPMAM)_{f1}- | (9/1), [227:1] | BnMAM | Et₂O/hexane |
| | co-(BnMAM)_{f2}]- | | (90/10) | (3:1), purification |
| | DTB | | | using LH-20 SEC |
| 104 | CN- | 1M, tBuOH/DMAc | NIPMAM/ | precipitation into |
| | p[(NIPMAM)_{f1}- | (9/1), [447:1] | BnMAM | Et₂O/hexane |
| | co-(BnMAM)_{f2}]- | | (85/15) | (3:1), purification |
| | DTB | | | using LH-20 SEC |
| 105 | CN- | 1M, tBuOH/DMAc | NIPMAM/ | precipitation into |
| | p[(NIPMAM)_{f1}- | (9/1), [270:1] | BnMAM | Et₂O/hexane |
| | co-(BnMAM)_{f2}]- | | (80/20) | (3:1), purification |
| | DTB | | | using LH-20 SEC |
| 106 | CN- | 2M, tBuOH/DMAc | NIPMAM/ | purification using |
| | p[(NIPMAM)_{f1}- | (9/1), [232:1] | BnMAM | LH-20 SEC |
| | co-(BnMAM)_{f2}]- | | (75/25) | |
| | DTB | | | |
| 107 | CN- | 2M, tBuOH/DMAc | NIPMAM/ | purification using |
| | p[(NIPMAM)_{f1}- | (85/15), [247:1] | BnMAM | LH-20 SEC |
| | co-(BnMAM)_{f2}]- | | (70/30) | |
| | DTB | | | |
| 108 | CN- | 2M, tBuOH/DMAc | NIPMAM/ | purification using |
| | p[(NIPMAM)_{f1}- | (8/2), [258:1] | BnMAM | LH-20 SEC |
| | co-(BnMAM)_{f2}]- | | (50/50) | |
| | DTB | | | |
| 109 | CN-p(BnMAM)_{f1}- | 1M, Anisole/MeOH | N.A. | purification using |
| | DTB | (4/1), [240:1] | | LH-20 SEC |
| 110 | CN-p(BnMAM)_{f1}- | 1M, Anisole/MeOH | N.A. | purification using |
| | DTB | (4/1), [120:1] | | LH-20 SEC |

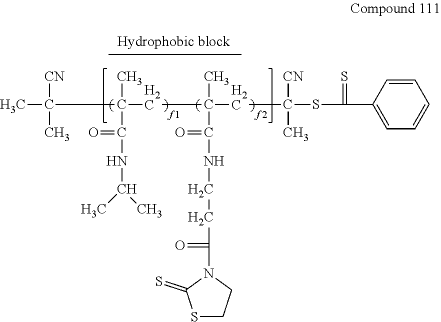

Compound 111. CN-p[(NIPMAM)_{f1}-co-(MA-b-Ala-TT)_{f2}]-DTB. A temperature-responsive single block co-polymer of NIPMAM (Compound 12) with an amine reactive comonomer (MA-b-Ala-TT, Compound 11) was synthesized via RAFT polymerization similarly as described for Compound 33 using CPDB (Compound 6) as a chain transfer agent and AIBN (Compound 1) as an initiator in tert-butanol (tBuOH) with 25% v/v dimethylacetamide co-solvent at 70° C. for 16 h. The initial combined monomer concentration [NIPMAM+MA-b-Ala-TT]₀=2 mol/L, the molar ratio [CPDB]₀:[AIBN]₀=1:0.5, and [NIPMAM+MA-b-Ala-TT]₀:[CTA]₀=275. The monomer mole fraction of [MA-b-Ala-TT]₀ defined as [MA-b-Ala-TT]₀/([NIPMAM]₀+[MA-b-Ala-TT]₀) was set to be 20% molar fraction. The following procedure was employed for a typical polymerization to produce p[(NIPMAM)-co-(MA-b-Ala-TT)]-DTB targeting a molecular weight of 20 kDa: NIPMAM (2032.4 mg, 16 mmol) was dissolved in 7.5 mL of tBuOH. MA-b-Ala-TT (1030.4 mg, 4 mmol) was dissolved in 2.5 mL of anhydrous DMAc. CPDB (16.1 mg, 0.0727 mmol) and AIBN (5.97 mg, 0.0364 mmol) were dissolved in anhydrous DMSO before mixing with the monomer solution. The mixture was transferred to a 20 mL ampule, which was sealed with a rubber septum and sparged with Ar (g) at r.t. for 20 min. The flask was then immersed in a water circulator preheated to 70° C. and polymerized for 16 h. The polymer was purified by separation through LH-20 lipophilic-Sephadex resin in methanol. Methanol was removed by rotary evaporation and the polymer was dried under vacuum overnight to remove residual solvent, yielding a light pink viscous gel (951 mg, 30.9% yield). Number-average (Mₙ) and weight-average molecular weight (M_{w}) were 21.7 kDa and 21.75 kDa, respectively, and polydispersity (PDI) was 1.01 measured by GPC-MALS with a dn/dc value used of 0.167 mL/g in methanol-acetate GPC solvent.

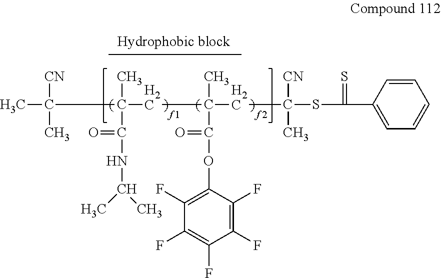

Compound 112. CN-p[(NIPMAM)_{f1}-co-(MA-PFP)_{f2}]-DTB. A temperature-responsive single block co-polymer of NIPMAM (Compound 12) with an amine reactive comonomer (MA-PFP, Compound 14) was synthesized via RAFT polymerization similarly as described for Compound 100 using CPDB (Compound 6) as a chain transfer agent and AIBN (Compound 1) as an initiator in 1,4-dioxane at 70° C. for 16 h. The initial combined monomer concentration [NIPMAM+MA-PFP]₀=4 mol/L, the molar ratio [CPDB]₀:[AIBN]₀=1:0.5, and [NIPMAM+MA-PFP]₀:[CTA]₀=169. The monomer mole fraction of [MA-PFP]₀ defined as [MA-PFP]₀/([NIPMAM]₀+[MA-PFP]₀) was set to be 20% molar fraction. The monomer mole fraction of [MA-PFP]₀ defined as [MA-b-PFP]₀ o/([NIPMAM]₀+[MA-PFP]₀) was set to be 20% molar fraction. The RAFT polymerization mixture was prepared as described for Compound 100, and the polymer was isolated by precipitation with a 10-fold volume of diethyl ether/hexane 1/3 v/v mixture. The polymer was dried under vacuum overnight to remove residual solvent, yielding a light pink viscous gel. Number-average (Mₙ) and weight-average molecular weight (M_{w}) were 21 kDa and 21.8 kDa, respectively, and polydispersity (PDI) was 1.04 measured by GPC-MALS.

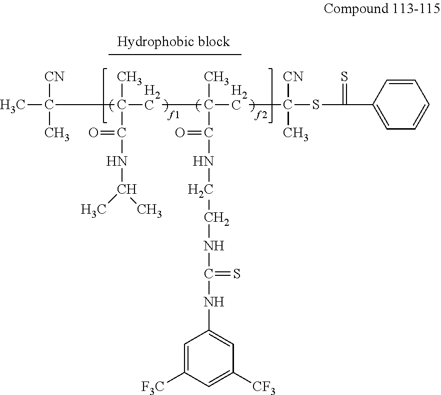

Compound 113-115. CN-p[(NIPMAM)_{f1}-co-(MA-EDA-BTFMP)_{f2}]-DTB. Temperature-responsive single block co-polymers of NIPMAM (Compound 12) with fluorinated monomer MA-EDA-BTFMP (Compound 16) were synthesized via RAFT polymerization similarly as described for Compound 100 using CPDB (Compound 6) as a chain transfer agent and AIBN (Compound 1) as an initiator in tert-butanol (tBuOH) with 10% v/v dimethylacetamide co-solvent at 70° C. for 16 h. The initial combined monomer concentration [NIPMAM+MA-EDA-BTFMP]₀=1 mol/L, the molar ratio [CPDB]₀:[AIBN]₀=1:0.5, and [NIPMAM+MA-EDA-BTFMP]₀:[CTA]₀ as defined in Table 3. The monomer mole fraction of [MA-EDA-BTFMP]₀ defined as [MA-EDA-BTFMP]₀/([NIPMAM]₀+[MA-EDA-BTFMP]₀) was set to be 1, 2 or 5 mol %. The RAFT polymerization mixture was prepared as described for Compound 100, and the polymer was isolated by precipitation followed by isolation via LH-20 in methanol as described in Table 3. Each polymer was dried under vacuum overnight to remove residual solvent, yielding a light pink viscous gel. Number-average (Mₙ), weight-average (M_{w}) molecular weights and PDI measured by GPC-MALS are listed in Table 7.

TABLE 3
CN-p[(NIPMAM)_{f1}-co-(Ma-EDA-BTFMP)_{f2}]-DTB synthesis conditions.

| | | Monomer | | |
|---|---|---|---|---|
| | | concentration, | Monomer | |
| Compound | Polymer | solvent system, and | molar ratio | Purification |
| # | structure | [monomer:CTA] ratio | (f1/f2) | procedure |
| 113 | CN- | 1M, tBuOH/DMAc | NIPMAM/ | Precipitation into |
| | p[(NIPMAM)_{f1}-co- | (9/1), [230:1] | MA-EDA- | Et₂O/Hexane |
| | (Ma-EDA- | | BTFMP | (1:3), purification |
| | BTFMP)_{f2}]-DTB | | (99/1) | using LH-20 SEC |
| 114 | CN- | 1M, tBuOH/DMAc | NIPMAM/ | Precipitation into |
| | p[(NIPMAM)_{f1}-co- | (9/1), [225:1] | MA-EDA- | Et₂O/Hexane |
| | (Ma-EDA- | | BTFMP | (1:3), purification |
| | BTFMP)_{f2}]-DTB | | (98/2) | using LH-20 SEC |
| 115 | CN- | 2M, tBuOH/DMAc | NIPMAM/ | Precipitation into |
| | p[(NIPMAM)_{f1}-co- | (9/1), [210:1] | MA-EDA- | hexane, |
| | (Ma-EDA- | | BTFMP | purification using |
| | BTFMP)_{f2}]-DTB | | (95/5) | LH-20 SEC |

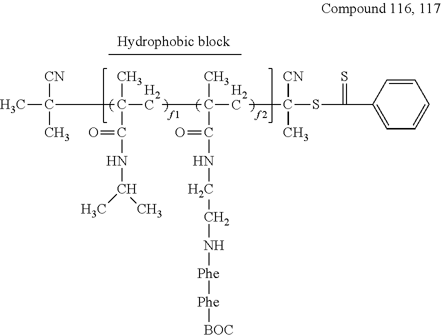

Compound 116, 117. CN-p[(NIPMAM)-co-(Ma-EDA-Phe-Phe-Boc)]-DTB. Temperature-responsive single block co-polymers of NIPMAM (Compound 12) with monomer MA-EDA-Phe-Phe-Boc (Compound 15) were synthesized via RAFT polymerization similarly as described for Compound 100 using CPDB (Compound 6) as a chain transfer agent and AIBN (Compound 1) as an initiator in tert-butanol (tBuOH) with 10% v/v dimethylacetamide co-solvent at 70° C. for 16 h. The initial combined monomer concentration [NIPMAM+MA-EDA-Phe-Phe-Boc]₀=1 mol/L, the molar ratio [CPDB]₀:[AIBN]₀=1:0.5, and [NIPMAM+MA-EDA-Phe-Phe-Boc]₀:[CTA]₀ as defined in Table 4. The monomer mole fraction of [MA-EDA-Phe-Phe-Boc]₀ defined as [MA-EDA-Phe-Phe-Boc]₀/([NIPMAM]₀+[MA-EDA-Phe-Phe-Boc]₀) was set to be 5 or 10% molar fraction. The RAFT polymerization mixture was prepared as described for Compound 100, and the polymer was isolated by precipitation followed by isolation via LH-20 in methanol as described in Table 4. Each polymer was dried under vacuum overnight to remove residual solvent, yielding a light pink viscous gel. Number-average (MK), weight-average (M_{w}) molecular weights and PDI measured by GPC-MALS are listed in Table 7.

TABLE 4
CN-p[(NIPMAM)_{f1}-co-(Ma-EDA-Phe-Phe-Boc)_{f2}]-DTB synthesis conditions.

| | | Monomer | | |
|---|---|---|---|---|
| | | concentration, solvent | Monomer | |
| Compound | Polymer | system, and | molar ratio | Purification |
| # | structure | [monomer:CTA] ratio | (f1/f2) | procedure |
| 116 | CN-p[(NIPMAM)_{f1}- | 1M, tBuOH/DMAc (9/1), | NIPMAM/ | Precipitation |
| | co-(Ma-EDA-Phe- | [204:1] | MA-EDA- | into Et₂O/ |
| | Phe-Boc)_{f2}]-DTB | | Phe-Phe-Boc | hexane (1:1), |
| | | | (95/5) | purification |
| | | | | using LH-20 |
| | | | | SEC |
| 117 | CN-p[(NIPMAM)_{f1}- | 1M, tBuOH/DMAc (9/1), | NIPMAM/ | Precipitation |
| | co-(Ma-EDA-Phe- | [180:1] | MA-EDA- | into Et₂O/ |
| | Phe-Boc)_{f2}]-DTB | | Phe-Phe-Boc | hexane (1:1), |
| | | | (9/1) | purification |
| | | | | using LH-20 |
| | | | | SEC |

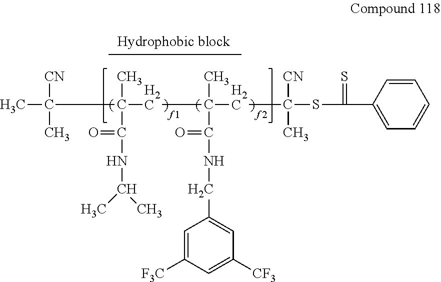

Compound 118. CN-p[(NIPMAM)_{f1}-co-(MA-BTFMB)_{f2}]-DTB. Temperature-responsive single block co-polymers of NIPMAM (Compound 12) with monomer MA-BTFMB (Compound 17) were synthesized via RAFT polymerization similarly as described for Compound 100 using CPDB (Compound 6) as a chain transfer agent and AIBN (Compound 1) as an initiator in tert-butanol (tBuOH) with 10% v/v dimethylacetamide co-solvent at 70° C. for 16 h. The initial combined monomer concentration [NIPMAM+MA-BTFMB]₀=1 mol/L, the molar ratio [CPDB]₀:[AIBN]₀=1:0.5, and [NIPMAM+MA-BTFMB]₀:[CTA]₀ was 220. The monomer mole fraction of [MA-BTFMB]₀ defined as [MA-BTFMB]₀/([NIPMAM]₀+[MA-BTFMB]₀) was set to be 5 mol %. The RAFT polymerization mixture was prepared as described for Compound 100, and the polymer was isolated by precipitation into a 1:3 mixture of diethyl ether/hexane followed by isolation via LH-20 in methanol. The polymer was dried under vacuum overnight to remove residual solvent, yielding a light pink viscous gel. Number-average (Mₙ), weight-average (M_{w}) molecular weights and PDI measured by GPC-MALS are listed in Table 7.

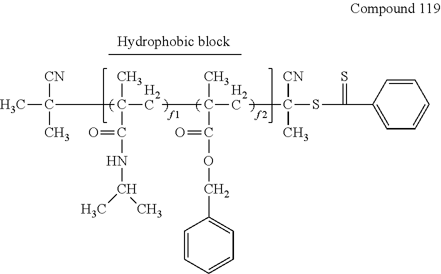

Compound 119. CN-p[(NIPMAM)_{f1}-co-(BnMA)_{f2}]-DTB. Temperature-responsive single block co-polymers of NIPMAM (Compound 12) with monomer BnMA (Compound 23) were synthesized via RAFT polymerization similarly as described for Compound 100 using CPDB (Compound 6) as a chain transfer agent and AIBN (Compound 1) as an initiator in tert-butanol (tBuOH) with 10% v/v dimethylacetamide co-solvent at 70° C. for 16 h. The initial combined monomer concentration [NIPMAM+BnMA]₀=1 mol/L, the molar ratio [CPDB]₀:[AIBN]₀=1:0.5, and [NIPMAM+BnMA]₀:[CTA]₀ was set to 149. The monomer mole fraction of [BnMA]₀ defined as [BnMA]₀/([NIPMAM]₀+[BnMA]₀) was set to be 20 mol %. The RAFT polymerization mixture was prepared as described for Compound 100, and the polymer was isolated via SEC using LH-20 in methanol. The polymer was dried under vacuum overnight to remove residual solvent, yielding a light pink viscous gel. Number-average (Mₙ), weight-average (M_{w}) molecular weights and PDI measured by GPC-MALS are listed in Table 7.

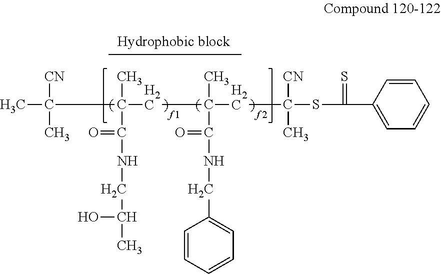

Compound 120-122. CN-p[(HPMA)_{f1}-co-(BnMAM)_{f2}]-DTB. Single block co-polymers of HPMA (Compound 10) with monomer BnMAM (Compound 13) were synthesized via RAFT polymerization similarly as described for Compound 100 using CPDB (Compound 6) as a chain transfer agent and AIBN (Compound 1) as an initiator in tert-butanol (tBuOH) with 10% v/v dimethylacetamide co-solvent at 70° C. for 16 h. The initial combined monomer concentration [HPMA+BnMAM]₀=1 mol/L, the molar ratio [CPDB]₀:[AIBN]₀=1:0.5, and [HPMA+BnMAM]₀:[CTA]₀ as defined in Table 5. The monomer mole fraction of [BnMAM]₀ defined as [BnMAM]₀/([HPMA]₀+[BnMAM]₀) was set to be 20, 30 or 50 mol %. The RAFT polymerization mixture was prepared as described for Compound 100, and the polymer was isolated by precipitation or via SEC using LH-20 in methanol as described in Table 5. Each polymer was dried under vacuum overnight to remove residual solvent, yielding a light pink viscous gel. Number-average (Mₙ), weight-average (M_{w}) molecular weights and PDI measured by GPC-MALS are listed in Table 7.

TABLE 5
CN-p[(HPMA)_{f1}-co-(BnMAM)_{f2}]-DTB synthesis conditions.

| | | Monomer | | |
|---|---|---|---|---|
| | | concentration, | | |
| | | solvent system, | Monomer | |
| | | and | molar | |
| Compound | Polymer | [monomer:CTA] | ratio | Purification |
| # | structure | ratio | (f1/f2) | procedure |
| 120 | CN-p[(HPMA)_{f1}- | 1M, tBuOH/ | HPMA/ | precipitation |
| | co-(BnMAM)_{f2}]- | DMAc | BnMAM | into Et₂O |
| | DTB | (9/1), [290:1] | (80/20) | |
| 121 | CN-p[(HPMA)_{f1}- | 1M, tBuOH/ | HPMA/ | purification |
| | co-(BnMAM)_{f2}]- | DMAc | BnMAM | using LH-20 |
| | DTB | (65/35), [331:1] | (70/30) | SEC |
| 122 | CN-p[(HPMA)_{f1}- | 1M, tBuOH/ | HPMA/ | purification |
| | co-(BnMAM)_{f2}]- | DMAc | BnMAM | using LH-20 |
| | DTB | (65/35), [331:1] | (50/50) | SEC |

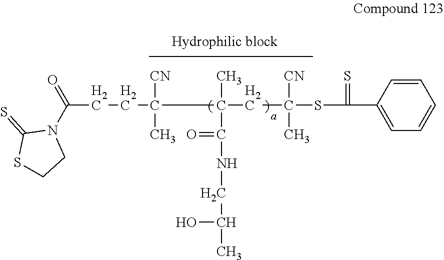

Compound 123. TT-functionalized poly[N-(2-hydroxypropyl)methacrylamide] (TT-p(HPMA)ₐ-DTB) was synthesized via the RAFT polymerization of HPMA (Compound 10) using CTA-TT (Compound 7) as a chain transfer agent and ACVA-TT (Compound 2) as an initiator in tert-butanol (tBuOH) at 70° C. for 16 h. The initial monomer concentration [HPMA]₀=1 mol/L, the molar ratio [CTA-TT]₀:[ACVA-TT]₀=1:0.5, and [HPMA]₀:[CTA-TT]₀ varied to obtain polymers with different chain lengths. The following procedure was employed for a typical polymerization to produce TT-p(HPMA)-DTB targeting a molecular weight of 30 kDa: HPMA (1144.0 mg, 8.00 mmol) was dissolved in 7.2 mL of tBuOH. CTA-TT (14.5 mg, 0.0381 mmol) and ACVA-TT (9.19 mg, 0.019 mmol) were dissolved in anhydrous DMSO before mixing with the monomer solution. Dimethylacetamide (DMAc), 0.8 mL, was added to 10% v/v as a cosolvent to increase solubility of polymer product. The mixture was transferred to a 20 mL ampule, which was sealed with a rubber septum and sparged with Ar (g) at r.t. for 20 min. The flask was then immersed in a water circulator preheated to 70° C. and polymerize for 16 h. The polymer was purified by precipitating against a 2:1 v/v acetone/ether counter-solvent twice. After drying under vacuum overnight, light pink powder was obtained (928.2 mg, 78.5% yield). Number-average (Mₙ) and weight-average molecular weight (M_{w}) were 30.98 kDa and 31.29 kDa, respectively, and polydispersity (PDI) was 1.01 measured by GPC-MALS. The chain end functionalities measured by UV-Vis spectroscopy [ε₃₀₅ (TT)=10300 L/(mol·cm), ε₃₀₅ (DTB)=12600 L/(mol·cm)] showed that (TT+DTB) %=96.6%.

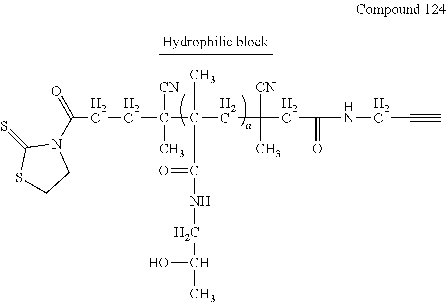

Compound 124. Pg-functionalized TT-poly[N-(2-hydroxypropyl)methacrylamide](TT-p(HPMA)ₐ-Pg) was prepared by reacting TT-p(HPMA)-DTB (Compound 123) with 20 molar equivalents of ACVA-Pg (Compound 3). Example of reaction: Dry polymer TT-p(HPMA)-DTB (293.6 mg, 9.48 μmol) and ACVA-Pg (67.19 mg, 189.6 μmol) were dissolved in 3 mL of anhydrous DMSO. The solution was transferred to a 5 mL ampule, which was sealed with a rubber septum and sparged with Ar (g) at r.t. for 20 min. The flask was then immersed in a water circulator preheated to 80° C. and reacted for 2 h. The polymer was purified by precipitating against 2:1 v/v acetone/ether for 2 times. After drying under vacuum overnight, off-white powder was obtained. Mₙ and M_{w} were 31.3 kDa and 32.08 kDa, respectively, and PDI was 1.025 measured by GPC-MALS.

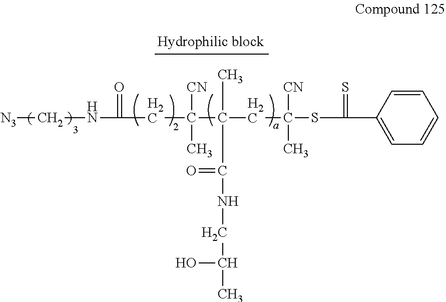

Compound 125. N3-functionalized poly[N-(2-hydroxypropyl)methacrylamide] (N3-p(HPMA)ₐ-DTB) was synthesized via the RAFT polymerization of HPMA (Compound 10) using CTA-N3 (Compound 9) as a chain transfer agent and ACVA-N3 (Compound 4) as an initiator in tert-butanol (tBuOH) at 70° C. for 16 h. The initial monomer concentration [HPMA]₀=1 mol/L, the molar ratio [CTA-N3]₀:[ACVA-N3]₀=1:0.5, and [HPMA]₀:[CTA-N3]₀ varied to obtain polymers with different chain lengths. The following procedure was employed for a typical polymerization to produce N3-p(HPMA)-DTB targeting a molecular weight of 25 kDa: HPMA (143.1 mg, 1.00 mmol) was dissolved in 1 mL of tBuOH. CTA-N3 (2.1 mg, 0.0057 mmol) and ACVA-N3 (1.27 mg, 0.0029 mmol) were dissolved in anhydrous DMSO before mixing with the monomer solution. The mixture was transferred to a 2 mL ampule, which was sealed with a rubber septum and sparged with Ar (g) at r.t. for 20 min. The flask was then immersed in a water circulator preheated to 70° C. and polymerize for 16 h. The polymer was purified by precipitating against a 2:1 v/v acetone/ether counter-solvent twice. After drying under vacuum overnight, light pink powder was obtained (145.3 mg, 97% yield). Number-average (Mₙ) and weight-average molecular weight (M_{w}) were 22.86 kDa and 24.17 kDa, respectively, and polydispersity (PDI) was 1.057 measured by GPC-MALS.

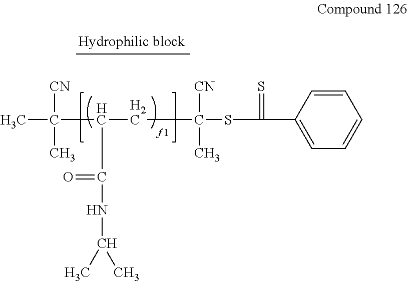

Compound 126. CN-p(NIPAM)_{f1}-DTB. Temperature-responsive single block polymers of NIPAM (Compound 19) were synthesized via RAFT polymerization similarly as described for Compound 100 using CPDB (Compound 6) as a chain transfer agent and AIBN (Compound 1) as an initiator in tert-butanol (tBuOH) with 10% v/v dimethylacetamide co-solvent at 70° C. for 16 h. The initial combined monomer concentration [NIPAM]₀=2 mol/L, the molar ratio [CPDB]₀:[AIBN]₀=1:0.5, and [NIPMAM]₀:[CTA]₀ was 120. The RAFT polymerization mixture was prepared as described for Compound 100, and the polymer was isolated by SEC using LH-20 in methanol. The polymer was dried under vacuum overnight to remove residual solvent, yielding a light pink viscous gel. Number-average (Mₙ), weight-average (M_{w}) molecular weights and PDI measured by GPC-MALS are listed in Table 7.

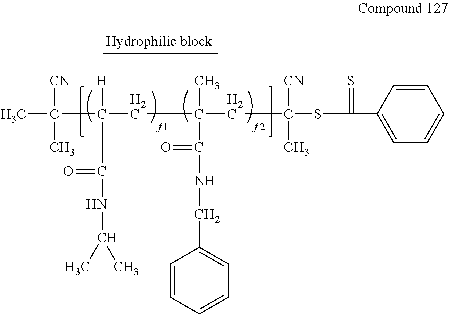

Compound 127. CN-p[(NIPAM)_{f1}-co-(BnMAM)_{f2}]-DTB. Temperature-responsive single block co-polymers of NIPAM (Compound 19) and BnMAM (Compound 13) were synthesized via RAFT polymerization similarly as described for Compound 100 using CPDB (Compound 6) as a chain transfer agent and AIBN (Compound 1) as an initiator in tert-butanol (tBuOH) with 10% v/v dimethylacetamide co-solvent at 70° C. for 16 h. The initial combined monomer concentration [NIPAM]₀=2 mol/L, the molar ratio [CPDB]₀:[AIBN]₀=1:0.5, and [NIPMAM]₀:[CTA]₀ was 120. The RAFT polymerization mixture was prepared as described for Compound 100, and the polymer was isolated by SEC using LH-20 in methanol. The polymer was dried under vacuum overnight to remove residual solvent, yielding a light pink viscous gel. Number-average (Mₙ), weight-average (M_{w}) molecular weights and PDI measured by GPC-MALS are listed in Table 7.

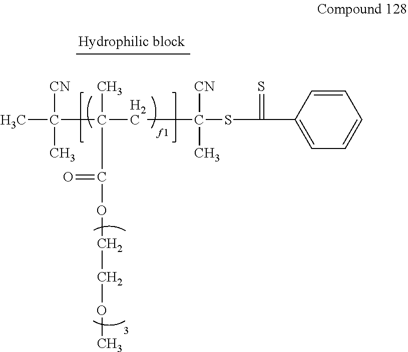

Compound 128. CN-p(TEGMA)_{f1}-DTB. Temperature-responsive single block polymers of TEGMA (Compound 22) were synthesized via RAFT polymerization similarly as described for Compound 100 using CPDB (Compound 6) as a chain transfer agent and AIBN (Compound 1) as an initiator in 1,4-dioxane at 70° C. for 16 h. The initial monomer concentration [TEGMA]₀=2 mol/L, the molar ratio [CPDB]₀:[AIBN]₀=1:0.5, and [TEGMA]₀:[CTA]₀ was 110. The RAFT polymerization mixture was prepared as described for Compound 100, and the polymer was isolated by precipitation in diethyl ether. The polymer was dried under vacuum overnight to remove residual solvent, yielding a light pink viscous gel. Number-average (Mₙ), weight-average (M_{w}) molecular weights and PDI measured by GPC-MALS are listed in Table 7.

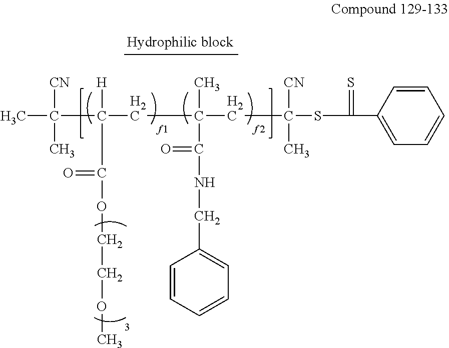

Compound 129-133. CN-p[(TEGMA)_{f1}-co-(BnMAM)_{f1}]-DTB. Temperature-responsive single block co-polymers of TEGMA (Compound 22) and BnMAM (Compound 13) were synthesized via RAFT polymerization similarly as described for Compound 100 using CPDB (Compound 6) as a chain transfer agent and AIBN (Compound 1) as an initiator in 1,4-dioxane at 70° C. for 16 h. The initial combined monomer concentration ([TEGMA]₀+[BnMAM]₀)=2 mol/L, the molar ratio [CPDB]₀:[AIBN]₀=1:0.5, and [TEGMA]₀:[CTA]₀ was 116. The RAFT polymerization mixture was prepared as described for Compound 100, and each polymer was isolated by precipitation in hexane. The polymer was dried under vacuum overnight to remove residual solvent, yielding a light pink viscous gel. Number-average (Mₙ), weight-average (M_{w}) molecular weights and PDI measured by GPC-MALS are listed in Table 7.

TABLE 6
CN-p[(TEGMA)_{f1}-co-(BnMAM)_{f2}]-DTB synthesis conditions.

| | | Monomer | | |
|---|---|---|---|---|
| | | concentration, | | |
| | | solvent system, | Monomer | |
| | | and | molar | |
| Compound | Polymer | [monomer:CTA] | ratio | Purification |
| # | structure | ratio | (f1/f2) | procedure |
| 129 | CN-p[(TEGMA)_{f1}- | 2M, tBuOH/ | TEGMA/ | Precipitation |
| | co-(BnMAM)_{f2}]- | DMAc | BnMAM | into hexane |
| | DTB | (95/5), [110:1] | (95/5) | |
| 130 | CN-p[(TEGMA)_{f1}- | 2M, tBuOH/ | TEGMA/ | Precipitation |
| | co-(BnMAM)_{f2}]- | DMAc | BnMAM | into hexane |
| | DTB | (90/10), [110:1] | (90/10) | |
| 131 | CN-p[(TEGMA)_{f1}- | 2M, tBuOH/ | TEGMA/ | Precipitation |
| | co-(BnMAM)_{f2}]- | DMAc | BnMAM | into hexane |
| | DTB | (85/15), [110:1] | (85/15) | |
| 132 | CN-p[(TEGMA)_{f1}- | 2M, tBuOH/ | TEGMA/ | Precipitation |
| | co-(BnMAM)_{f2}]- | DMAc | BnMAM | into hexane |
| | DTB | (80/20), [110:1] | (80/20) | |
| 133 | CN-p[(TEGMA)_{f1}- | 2M, tBuOH/ | TEGMA/ | Precipitation |
| | co-(BnMAM)_{f2}]- | DMAc | BnMAM | into hexane |
| | DTB | (70/30), [110:1] | (70/30) | |

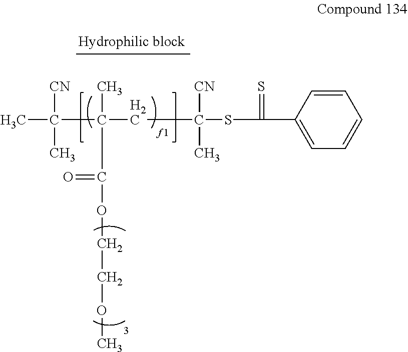

Compound 134. CN-p(DEGMA)_{f1}-DTB. Temperature-responsive single block polymers of DEGMA (Compound 21) were synthesized via RAFT polymerization similarly as described for Compound 100 using CPDB (Compound 6) as a chain transfer agent and AIBN (Compound 1) as an initiator in 1,4-dioxane at 70° C. for 16 h. The initial monomer concentration [DEGMA]₀=2 mol/L, the molar ratio [CPDB]₀:[AIBN]₀=1:0.5, and [DEGMA]₀:[CTA]₀ was 100. The RAFT polymerization mixture was prepared as described for Compound 100, and the polymer was isolated by precipitation in hexane. The polymer was dried under vacuum overnight to remove residual solvent, yielding a light pink viscous gel. Number-average (Mₙ), weight-average (M_{w}) molecular weights and PDI measured by GPC-MALS are listed in Table 7.

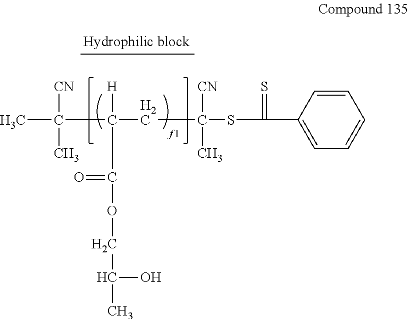

Compound 135. CN-p(HPA)_{f1}-DTB. Temperature-responsive single block polymers of HPA (Compound 26) were synthesized via RAFT polymerization similarly as described for Compound 100 using CPDB (Compound 6) as a chain transfer agent and AIBN (Compound 1) as an initiator in 1,4-dioxane at 70° C. for 16 h. The initial monomer concentration [HPA]₀=2 mol/L, the molar ratio [CPDB]₀:[AIBN]₀=1:0.5, and [HPA]₀:[CTA]₀ was 100. The RAFT polymerization mixture was prepared as described for Compound 100, and the polymer was isolated by precipitation in hexane. The polymer was dried under vacuum overnight to remove residual solvent, yielding a light pink viscous gel. Number-average (Mₙ), weight-average (M_{w}) molecular weights and PDI measured by GPC-MALS are listed in Table 7.

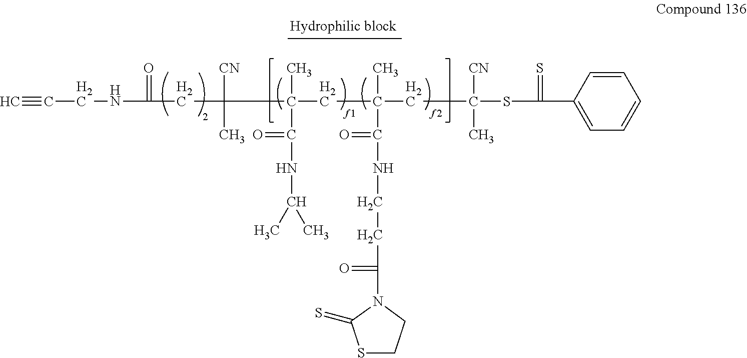

Compound 136. Pg-p[(NIPMAM)_{f1}-co-(MA-b-Ala-TT)_{f2}]-DTB. A temperature-responsive single block co-polymer of NIPMAM (Compound 12) with an amine reactive comonomer (MA-b-Ala-TT, Compound 11) was synthesized via RAFT polymerization similarly as described for Compound 33 using CTA-Pg (Compound 8) as a chain transfer agent and ACVA-Pg (Compound 3) as an initiator in tert-butanol (tBuOH) with 10% v/v dimethylacetamide co-solvent at 70° C. for 16 h. The initial combined monomer concentration [NIPMAM+MA-b-Ala-TT]₀=2 mol/L, the molar ratio [CPDB]₀:[AIBN]₀=1:0.5, and [NIPMAM+MA-b-Ala-TT]₀:[CTA]₀=275. The monomer mole fraction of [MA-b-Ala-TT]₀ defined as [MA-b-Ala-TT]₀/([NIPMAM]₀+[MA-b-Ala-TT]₀) was set to be 10% molar fraction. The following procedure was employed for a typical polymerization to produce p[(NIPMAM)-co-(MA-b-Ala-TT)]-DTB targeting a molecular weight of 30 kDa: NIPMAM (915.6 mg, 7.2 mmol) was dissolved in 3.6 mL of tBuOH. MA-b-Ala-TT (206.9 mg, 0.8 mmol) was dissolved in 0.4 mL of anhydrous DMAc. CTA-Pg (4.2 mg, 0.0291 mmol) and ACVA-Pg (5.16 mg, 0.0145 mmol) were dissolved in anhydrous DMSO before mixing with the monomer solution. The mixture was transferred to a 20 mL ampule, which was sealed with a rubber septum and sparged with Ar (g) at r.t. for 20 min. The flask was then immersed in a water circulator preheated to 70° C. and polymerized for 16 h. The polymer was purified by separation through LH-20 lipophilic-Sephadex resin in methanol. Methanol was removed by rotary evaporation and the polymer was dried under vacuum overnight to remove residual solvent, yielding a light pink viscous gel (380.7 mg, 34% yield). Number-average (Mₙ) and weight-average molecular weight (M_{w}) were 34.7 kDa and 35.1 kDa, respectively, and polydispersity (PDI) was 1.013 measured by GPC-MALS with a dn/dc value used of 0.167 mL/g in methanol-acetate GPC solvent.

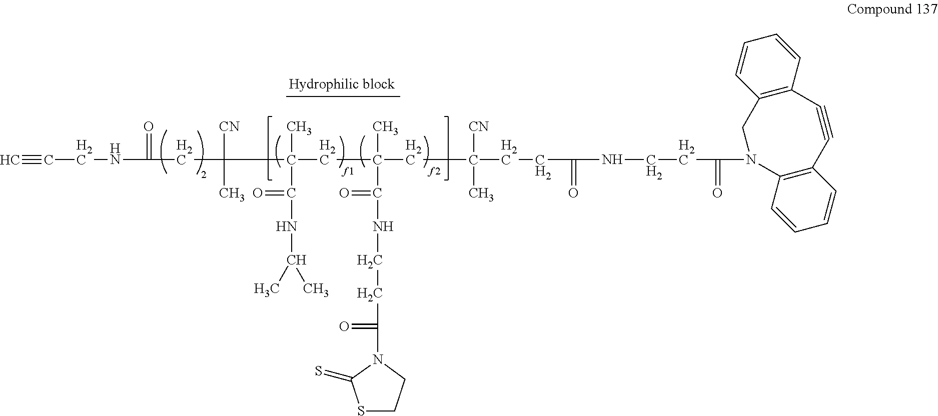

Compound 137. Pg-p[(NIPMAM)_{f1}-co-(MA-b-Ala-TT)_{f2}]-DBCO. DBCO functionalized polymer was prepared by reacting Pg-p[(NIPMAM)_{f1}-co-(MA-b-Ala-TT)_{f2}]-DTB (Compound 136) with 20 molar equivalents of ACVA-DBCO (Compound 5). Example of reaction: Dry polymer Pg-p[(NIPMAM)_{f1}-co-(MA-b-Ala-TT)_{f2}]-DTB (101.6 mg, 2.93 μmol) and ACVA-DBCO (46.72 mg, 58.63 μmol) were dissolved in 1.48 mL of anhydrous DMSO. The solution was transferred to a 5 mL ampule, which was sealed with a rubber septum and sparged with Ar (g) at r.t. for 20 min. The flask was then immersed in a water circulator preheated to 80° C. and reacted for 2 h. The polymer was purified by precipitating against 1:1 v/v acetone/ether for 2 times. After drying under vacuum overnight, off-white powder was obtained. Mn and Mw were 31.3 kDa and 32.08 kDa, respectively, and PDI was 1.025 measured by GPC-MALS.

TABLE 7
Results summary of single block polymer (Compounds 100-136)

| | | | | | | Mol % | |
|---|---|---|---|---|---|---|---|
| Cmpd | | % | Mn | | | F2 or | Tₜᵣ |
| # | Structure | conv. | (kDa) | PDI | Xₙ | E1 | (° C.) |
| 100 | CN-p[(NIPMAM)_{f1}-co- | 44.6 | 16.0 | 1.06 | 117 | 20 | 20 |
| | (BnMAM)_{f2}]-DTB, 20 mol % | | | | | | |
| | hydrophobic block BnMAM | | | | | | |
| 101 | N3-p(NIPMAM)f1-DTB | 89.6 | 14.3 | 1.04 | 112 | 0 | 45 |
| 102 | CN-p[(NIPMAM)_{f1}-co- | 35.8 | 15.8 | 1.10 | 122 | 5 | 35 |
| | (BnMAM)_{f1}]-DTB, 5 mol % | | | | | | |
| | hydrophobic block BnMAM | | | | | | |
| 103 | CN-p[(NIPMAM)_{f1}-co- | 30.7 | 16.1 | 1.06 | 122 | 10 | 28 |
| | (BnMAM)_{f1}]-DTB, 10 mol % | | | | | | |
| | hydrophobic block BnMAM | | | | | | |
| 104 | CN-p[(NIPMAM)_{f1}-co- | 21.2 | 16.0 | 1.06 | 119 | 15 | 23 |
| | (BnMAM)_{f1}]-DTB, 15 mol % | | | | | | |
| | hydrophobic block BnMAM | | | | | | |
| 105 | CN-p[(NIPMAM)_{f1}-co- | 44.6 | 16.0 | 1.06 | 117 | 20 | 20 |
| | (BnMAM)_{f1}]-DTB, 20 mol % | | | | | | |
| | hydrophobic block BnMAM | | | | | | |
| 106 | CN-p[(NIPMAM)_{f1}-co- | 43.4 | 16.8 | 1.02 | 114 | 25 | 14 |
| | (BnMAM)_{f1}]-DTB, 25 mol % | | | | | | |
| | hydrophobic block BnMAM | | | | | | |
| 107 | CN-p[(NIPMAM)_{f1}-co- | 35.7 | 12.7 | 1.01 | 88 | 30 | 12 |
| | (BnMAM)_{f1}]-DTB, 30 mol % | | | | | | |
| | hydrophobic block BnMAM | | | | | | |
| 108 | CN-p[(NIPMAM)_{f1}-co- | 38.0 | 15.0 | 1.01 | 98 | 50 | 10 |
| | (BnMAM)_{f1}]-DTB, 50 mol % | | | | | | |
| | hydrophobic block BnMAM | | | | | | |
| 109 | CN-p(BnMAM)_{f1}-DTB, 100 mol % | 56.6 | 23.8 | 1.02 | 136 | 100 | Agg. |
| | hydrophobic block BnMAM | | | | | | |
| 110 | CN-p(BnMAM)_{f1}-DTB, 100 mol % | 66.7 | 14.0 | 1.01 | 80 | 100 | Agg. |
| | hydrophobic block BnMAM | | | | | | |
| 111 | CN-p[(NIPMAM)_{f1}-co-(MA-b-Ala- | 50.9 | 21.7 | 1.01 | 140 | 20 | N.D. |
| | TT)ₑ]-DTB, 20 mol % | | | | | | |
| | hydrophobic block MA-b-Ala-TT | | | | | | |
| 112 | CN-p[(NIPMAM)_{f1}-co-(MA- | 49.5 | 21.0 | 1.04 | 138 | 20 | Agg. |
| | PFP)ₑ]-DTB, 20 mol % | | | | | | |
| | hydrophobic block MA-PFP | | | | | | |
| 113 | CN-p[(NIPMAM)_{f1}-co-(MA-EDA- | 71.0 | 8.9 | 1.08 | 69 | 1 | 32 |
| | BTFMP)_{f2}]-DTB, 1 mol % | | | | | | |
| | hydrophobic block MA-EDA- | | | | | | |
| | BTFMP | | | | | | |
| 114 | CN-p[(NIPMAM)_{f1}-co-(MA-EDA- | 84.6 | 8.4 | 1.08 | 63 | 2 | 23 |
| | BTFMP)12]-DTB, 2 mol % | | | | | | |
| | hydrophobic block MA-EDA- | | | | | | |
| | BTFMP | | | | | | |
| 115 | CN-p[(NIPMAM)_{f1}-co-(MA-EDA- | 76.0 | 8.9 | 1.08 | 63 | 5 | Agg. |
| | BTFMP)_{f2}]-DTB, 5 mol % | | | | | | |
| | hydrophobic block MA-EDA- | | | | | | |
| | BTFMP | | | | | | |
| 116 | CN-p[(NIPMAM)_{f1}-co-(MA-EDA- | 30.6 | 12.5 | 1.09 | 85 | 5 | N.D. |
| | Phe-Phe-Boc)_{f2}]-DTB, 5 mol % | | | | | | |
| | hydrophobic block MA-EDA- | | | | | | |
| | Phe-Phe-Boc | | | | | | |
| 117 | CN-p[(NIPMAM)_{f1}-co-(MA-EDA- | 27.5 | 12.1 | 1.07 | 73 | 10 | N.D. |
| | Phe-Phe-Boc)_{f2}]-DTB, 10 mol % | | | | | | |
| | hydrophobic block MA-EDA- | | | | | | |
| | Phe-Phe-Boc | | | | | | |
| 118 | CN-p[(NIPMAM)_{f1}-co-(MA- | 41.4 | 15.8 | 1.11 | 123 | 5 | 30 |
| | BTFMB)_{f2}]-DTB, 5 mol % | | | | | | |
| | hydrophobic block MA-BTFMB | | | | | | |
| 119 | CN-p[(NIPMAM)_{f1}-co-(BnMA)_{f2}]- | 92.4 | 19.7 | 1.03 | 142 | 20 | N.D. |
| | DTB, 5 mol % hydrophobic block | | | | | | |
| | BnMA | | | | | | |
| 120 | CN-p[(HPMA)ₐ-Co-(BnMAM)_{f2}]- | 25.9 | 11.5 | 1.09 | 75 | 20 | None |
| | DTB, 20 mol % hydrophobic | | | | | | |
| | block BnMAM | | | | | | |
| 121 | CN-p[(HPMA)ₐ-co-(BnMAM)_{f2}]- | 31.4 | 18.7 | 1.12 | 121 | 30 | None |
| | DTB, 30 mol % hydrophobic | | | | | | |
| | block BnMAM | | | | | | |
| 122 | CN-p[(HPMA)ₐ-co-(BnMAM)_{f2}]- | 28.8 | 23.9 | 1.19 | 149 | 50 | None |
| | DTB, 50 mol % hydrophobic | | | | | | |
| | block BnMAM | | | | | | |
| 123 | TT-p(HPMA)ₐ-DTB | 102.8 | 31.0 | 1.01 | 217 | 0 | None |
| 124 | TT-p(HPMA)ₐ-Pg | N.A. | N.D. | N.D. | N.D. | 0 | None |
| 125 | N3-p(HPMA)ₐ-DTB | 89.9 | 22.9 | 1.06 | 157.3 | NA | None |
| 126 | CN-p(NIPAM)_{f1}-N3 | 173.0 | 23.8 | 1.03 | 208 | 0 | 32 |
| 127 | CN-p[(NIPAM)_{f1}-co-(BnMAM)_{f2}]- | 122.1 | 40.3 | 1.08 | 335.9 | 10 | 14 |
| | DTB, 20 mol % hydrophobic | | | | | | |
| | block BnMAM | | | | | | |
| 128 | CN-p(TEGMA)_{f1}-DTB | 164.3 | 42.2 | 2.38 | 180.7 | NA | 40 |
| 129 | CN-p[(TEGMA)_{f1}-co-(BnMAM)_{f2}]- | 167.0 | 42.4 | 1.97 | 183.7 | 5 | 36 |
| | DTB, 5 mol % hydrophobic block | | | | | | |
| | TEGMA | | | | | | |
| 130 | CN-p[(TEGMA)_{f1}-co-(BnMAM)_{f2}]- | 132.5 | 33.2 | 1.65 | 145.7 | 10 | 37 |
| | DTB, 10 mol % hydrophobic | | | | | | |
| | block TEGMA | | | | | | |
| 131 | CN-p[(TEGMA)_{f1}-co-(BnMAM)_{f2}]- | 121.6 | 30.1 | 1.48 | 133.8 | 15 | 27 |
| | DTB 15 mol % hydrophobic block | | | | | | |
| | TEGMA | | | | | | |
| 132 | CN-p[(TEGMA)_{f1}-co-(BnMAM)_{f2}]- | 116.2 | 28.4 | 1.40 | 127.8 | 20 | <4 |
| | DTB, 20 mol % hydrophobic | | | | | | |
| | block TEGMA | | | | | | |
| 133 | CN-p[(TEGMA)_{f1}-co-(BnMAM)_{f2}]- | 104.8 | 25 | 1.23 | 115.3 | 30 | <4 |
| | DTB, 30 mol % hydrophobic | | | | | | |
| | block TEGMA | | | | | | |
| 134 | CN-p(DEGMA)_{f1}-DTB | 89.9 | 17.1 | 1.09 | 89.9 | NA | 24 |
| 135 | CN-p(HPA)_{f1}-DTB | 96.9 | 12.8 | 1.13 | 96.9 | NA | 16 |
| 136 | Pg-p[(NIPMAM)f-co-(Ma-b-Ala- | 89.0 | 34.66 | 1.01 | 244.8 | 10 | N.D. |
| | TT)_{f2}]-DTB, 10 mo% | | | | | | |
| | hydrophobic block MA-b-Ala-TT | | | | | | |
| 137 | Pg-p[(NIPMAM)_{f1}-co-(Ma-b-Ala- | 89.0 | N.D. | N.D. | N.D. | 10 | N.D. |
| | TT)_{f2}]-DBCO, 10 mo% | | | | | | |
| | hydrophobic block MA-b-Ala-TT | | | | | | |

characterization by GPC-MALS and Tₜᵣ determined using DLS. % conversion (abbreviated % conv.) refers to the isolated polymer Xₙ divided by input [monomer]ₒ : [CTA]ₒ. Mol % F2 or E1 refers to the mol % of either the second hydrophobic monomer or first reactive monomer. Aggregate (abbreviated Agg.) denotes polymers that were insoluble in PBS at 0.5 mg/mL at temperatures between 4-50° C. None denotes polymers that were completely soluble in PBS at 0.5 mg/mL at temperatures between 4-50° C. Polymers not tested for temperature-responsiveness are listed as (N.D.) for no data.

### Example 3—Synthesis of Di-Block Copolymers

The following section details the methods used in the preparation of block copolymers (e.g., diblock copolymers) useful in the practice of the invention(s) described herein as well as block copolymer controls. Unless otherwise specified, the methods used in the purification and characterization of the block copolymers is the same as the methods used in the purification and characterization of the single block polymers.

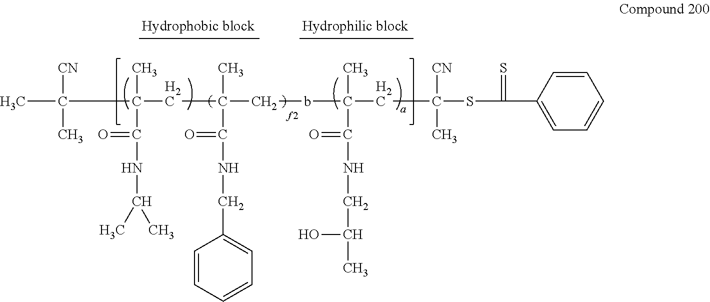

Compound 200. Example of the synthesis of a temperature-responsive amphiphilic block copolymer: CN-p[(NIPMAM)_{f1}-co-(BnMAM)_{f2}]-b-p(HPMA)ₐ-DTB via RAFT polymerization of HPMA (Compound 10) using a macro-chain transfer agent CN-[(NIPMAM)-co-(BnMAM)]-DTB (Compound 100) and AIBN (Compound 1) as an initiator targeting a molecular weight of 16-b-30 kDa (hydrophobic block-b-hydrophilic block) and degree of polymerization block ratio of hydrophobic block to hydrophilic block of 117-b-200 (which could be written 200:117 as the degree of polymerization block ratio of hydrophilic block to hydrophobic block): HPMA (71 mg, 0.5 mmol) (Compound 10) was dissolved in 0.3 mL of tBuOH. The macro-CTA (Compound 100), CN-p[(NIPMAM)-co-(BnMAM)]-DTB (36 mg, 0.0022 mmol) was dissolved in 0.2 mL of DMAc. AIBN was pre-dissolved in DMSO. Monomer, macro-CTA and initiator were then combined and transferred to a 2 mL ampule, which was sealed with a rubber septum and sparged with Ar (g) at r.t. for 20 min. The flask was then immersed in a water circulator preheated to 70° C. and polymerized for 16 h. The polymer was purified by precipitation in 10×volume of diethyl ether. The polymer was then dried under high vacuum overnight to remove residual solvent, yielding a light pink powder (35.6 mg, 33% yield).

Compounds 201-215. Temperature-responsive amphiphilic block copolymers with the composition CN-p[(NIPMAM)-co-(BnMAM)]-b-p(HPMA)-DTB and amphiphilic block copolymers with the composition CN-p(BnMAM)-b-p(HPMA)-DTB were synthesized using a similar procedure as that described for Compound 200, except varying macro-CTAs composed of varying mole percentages (mol %) of NIPMAM and #nMAM, and varying [monomer:macro-CTA] and solvent systems were used, as summarized in Table 8, below. In brief, the molar ratio [macro-CTA]₀:[AIBN]₀ was fixed at 1:0.5, but the [HPMA]₀:[macro-CTA]₀ ratio as well as the specific macro-CTA used were varied to obtain polymers with different chain lengths and block ratios. HPMA was dissolved in tBuOH at [HPMA]0=1 mol/L, the macro-CTA was dissolved in DMAc and AIPN was pre-dissolved in DMSO. The macro-CTA, initiator and monomer solutions were combined, sparged with Argon (g) and then reacted at 70° C. for 16 hours in the specified solvent system, followed by precipitation in diethyl ether for purification.

TABLE 8
Synthesis conditions for compounds 201-215.

| | | | Monomer concentration, |
|---|---|---|---|
| | | Macro | solvent system, and |
| Cmpd | | CTA | [monomer:macro-CTA] |
| # | Polymer structure | cmpd # | ratio |
| 201 | CN-p[(NIPMAM)_{f1}-co-(BnMAM)_{f2}]-b- | 103 | 1M, tBuOH/DMAc (9/1), |
| | p(HPMA)ₐ-DTB, 10 mol % hydrophobic | | [223:1] |
| | block BnMAM | | |
| 202 | CN-p[(NIPMAM)_{f1}-co-(BnMAM)_{f2}]-b- | 103 | 1M, tBuOH/DMAc (9/1), |
| | p(HPMA)ₐ-DTB, 10 mol % hydrophobic | | [219:1] |
| | block BnMAM | | |
| 203 | CN-p[(NIPMAM)_{f1}-co-(BnMAM)_{f2}]-b- | 103 | 1M, BuOH/DMAc (9/1), |
| | p(HPMA)ₐ-DTB, 10 mol % hydrophobic | | [223:1] |
| | block BnMAM | | |
| 204 | CN-p[(NIPMAM)_{f1}-co-(BnMAM)_{f2}]-b- | 104 | 1M, tBuOH/DMAc (9/1), |
| | p(HPMA)ₐ-DTB, 15 mol % hydrophobic | | [223:1] |
| | block BnMAM | | |
| 205 | CN-p[(NIPMAM)_{f1}-co-(BnMAM)_{f2}]-b- | 104 | 1M, tBuOH/DMAc (9/1), |
| | p(HPMA)ₐ-DTB, 15 mol % hydrophobic | | [670:1] |
| | block BnMAM | | |
| 206 | CN-p[(NIPMAM)_{f1}-co-(BnMAM)_{f2}]-b- | 105 | 1M, tBuOH/DMAc (9/1), |
| | p(HPMA)ₐ-DTB, 20 mol % hydrophobic | | [223:1] |
| | block BnMAM | | |
| 207 | CN-p[(NIPMAM)_{f1}-co-(BnMAM)_{f2}]-b- | 105 | 1M, tBuOH/DMAc (9/1), |
| | p(HPMA)ₐ-DTB, 20 mol % hydrophobic | | [447:1] |
| | block BnMAM | | |
| 208 | CN-p[(NIPMAM)_{f1}-co-(BnMAM)_{f2}]-b- | 105 | 1M, tBuOH/DMAc (9/1), |
| | p(HPMA)ₐ-DTB, 20 mol % hydrophobic | | [670:1] |
| | block BnMAM | | |
| 209 | CN-p[(NIPMAM)_{f1}-co-(BnMAM)_{f2}]-b- | 106 | 1M, tBuOH/DMAc (8/2), |
| | p(HPMA)ₐ-DTB, 25 mol % hydrophobic | | [261:1] |
| | block BnMAM | | |
| 210 | CN-p[(NIPMAM)_{f1}-co-(BnMAM)_{f2}]-b- | 107 | 1M, tBuOH/DMAc (7/3), |
| | p(HPMA)ₐ-DTB, 30 mol % hydrophobic | | [244:1] |
| | block BnMAM | | |
| 211 | CN-p[(NIPMAM)_{f1}-co-(BnMAM)_{f2}]-b- | 107 | 1M, tBuOH/DMAc (3/2), |
| | p(HPMA)ₐ-DTB, 30 mol % hydrophobic | | [100:1] |
| | block BnMAM | | |
| 212 | CN-p[(NIPMAM)_{f1}-co-(BnMAM)_{f2}]-b- | 108 | 1M, tBuOH/DMAc (7/3), |
| | p(HPMA)ₐ-DTB, 50 mol % hydrophobic | | [256:1] |
| | block BnMAM | | |
| 213 | CN-p[(NIPMAM)_{f1}-co-(BnMAM)_{f2}]-b- | 108 | 1M, tBuOH/DMAc (3/2), |
| | p(HPMA)ₐ-DTB, 50 mol % hydrophobic | | [100:1] |
| | block BnMAM | | |
| 214 | CN-p(BnMAM)_{f1}-b-p(HPMA)ₐ-DTB, | 109 | 1M, tBuOH/DMAc (3/2), |
| | 100 mol % hydrophobic block BnMAM | | [270:1] |
| 215 | CN-p(BnMAM)_{f1}-b-p(HPMA)ₐ-DTB, | 110 | 1M, tBuOH/DMAc (3/2), |
| | 100 mol % hydrophobic block BnMAM | | [225:1] |

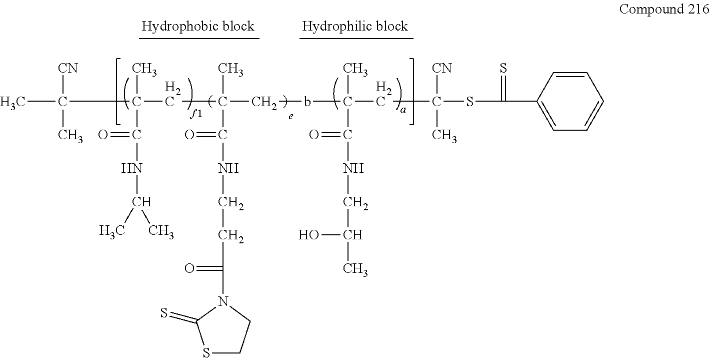

Compound 216. CN-p[(NIPMAM)_{f1}-co-(MA-b-Ala-TT)ₑ]-b-p(HPMA)ₐ-DTB. The hydrophilic p(HPMA) block of di-block copolymers of structure of CN-p[(NIPMAM)-co-(MA-b-Ala-TT)]-b-p(HPMA)-DTB was synthesized via RAFT polymerization of HPMA (Compound 10) using a macro-chain transfer agent CN-p[(NIPMAM)_{f1}-co-(MA-b-Ala-TT)ₑ]-DTB (Compound 111) and AIBN (Compound 1) as an initiator. For polymerization, the macro-CTA hydrophobic polymer block was dissolved in DMAc, AIBN was pre-dissolved in DMSO and HPMA monomer was dissolved in tBuOH at concentrations to enable [HPMA]₀=1 mol/L. The molar ratio [macro-CTA]₀:[AIBN]₀=1:0.5, and [HPMA]₀:[macro-CTA]₀ set to 225. The following procedure was employed for a typical polymerization to produce CN-p[(NIPMAM)_{f1}-co-(MA-b-Ala-TT)ₑ]-b-p(HPMA)ₐ-DTB targeting a p(HPMA) molecular weight of 25 kDa: HPMA (214.6 mg, 1.5 mmol) (Compound 10) was dissolved in 0.75 mL of tBuOH. The macro-CTA, CN-p[(NIPMAM)-co-(MA-b-Ala-TT)]-DTB (144.7 mg, 0.0067 mmol) was dissolved in 0.72 mL of DMAc. AIBN was pre-dissolved in DMSO. Monomer, macro-CTA and initiator were then combined and transferred to a 2 mL ampule, which was sealed with a rubber septum and sparged with Ar (g) at r.t. for 20 min. The flask was then immersed in a water circulator preheated to 70° C. and polymerized for 16 h. The polymer was purified by precipitation in 10×polymer volume of diethyl ether counter-solvent twice. The polymer was then dried under vacuum overnight to remove residual solvent, yielding a light pink powder gel (209.5 mg, 57.7% yield, 49.3% conversion). Results from GPC-MALS and DLS are presented in Table 12 and Table 17 respectively.

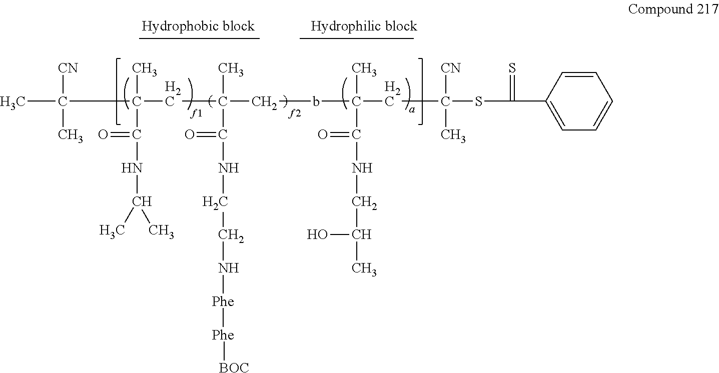

Compound 217. CN-p[(NIPMAM)_{f1}-co-(MA-EDA-Phe-Phe-Boc)_{f2}]-b-p(HPMA)ₐ-DTB was synthesized via RAFT polymerization of HPMA (Compound 10) using CN-[(NIPMAM)_{f1}-co-(MA-EDA-Phe-Phe-Boc)_{f2}]-DTB (Compound 116) as a macro-chain transfer agent and AIBN (Compound 1) as an initiator. For polymerization, the macro-CTA hydrophobic polymer block was dissolved in DMAc, AIBN was pre-dissolved in DMSO and HPMA monomer was dissolved in tBuOH at concentrations to enable [HPMA]₀=1 mol/L. The molar ratio [macro-CTA]₀:[AIBN]₀=1:0.5, and [HPMA]₀:[macro-CTA]₀ set to 149. A procedure similar to the preparation of Compound 200 was used for RAFT polymerization. Following polymerization of p(HPMA), the di-block copolymer was precipitated into cold acetone/Et₂O(1:1) followed by additional purification using LH-20 in methanol. Results from GPC-MALS and DLS are presented in Table 12 and Table 17 respectively.

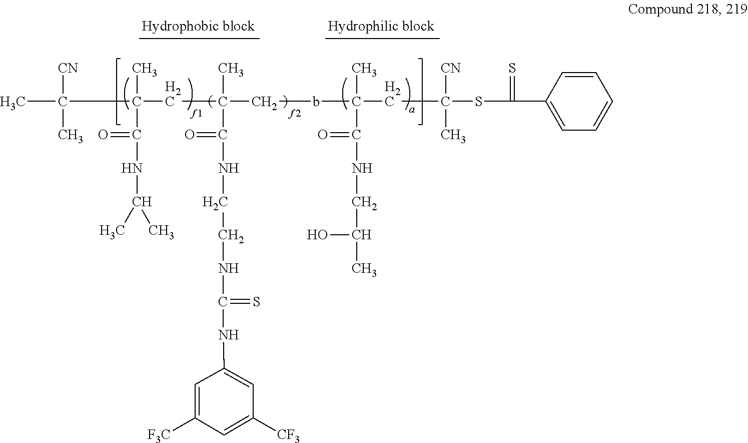

Compound 218, 219. CN-p[(NIPMAM)_{f1}-co-(MA-BTFMB)_{f2}]-b-p(HPMA)ₐ-DTB was synthesized via RAFT polymerization of HPMA (Compound 10) using CN-p[(NIPMAM)_{f1}-co-(MA-BTFMB)_{f2}]-DTB (Compound 115) as a macro-chain transfer agent and AIBN (Compound 1) as initiator. For polymerization, the macro-CTA hydrophobic polymer block was dissolved in DMAc, AIBN was pre-dissolved in DMSO and HPMA monomer was dissolved in tBuOH at [HPMA]₀=1 mol/L. The molar ratio [macro-CTA]₀:[AIBN]₀ was 1:0.5, and [HPMA]₀:[macro-CTA]₀ was set as listed in Table 8. A procedure similar to the preparation of Compound 200 was used for RAFT polymerization to obtain the diblock copolymer. Following polymerization of p(HPMA), the di-block copolymer was precipitated into cold acetone/Et₂O(1:1) followed by additional purification using LH-20 in methanol. Results from GPC-MALS and DLS are presented in Table 12 and Table 17 respectively.

TABLE 9
Compound 218 and 219 synthesis conditions.

| | | | Monomer |
|---|---|---|---|
| | | Macro-CTA | concentration, solvent |
| Cmpd | | compound | system, and |
| # | Polymer structure | number | [monomer]:[CTA] ratio |
| 218 | CN-p[(NIPMAM)_{f1}-co-(MA-BTFMB)_{f2}]- | 115 | 1M, tBuOH/DMAc (9/1), |
| | b-p(HPMA)ₐ-DTB | | [116:1] |
| 219 | CN-p[(NIPMAM)_{f1}-co-(MA-BTFMB)_{f2}]- | 115 | 1M, tBuOH/DMAc (9/1), |
| | b-p(HPMA)ₐ-DTB | | [464:1] |

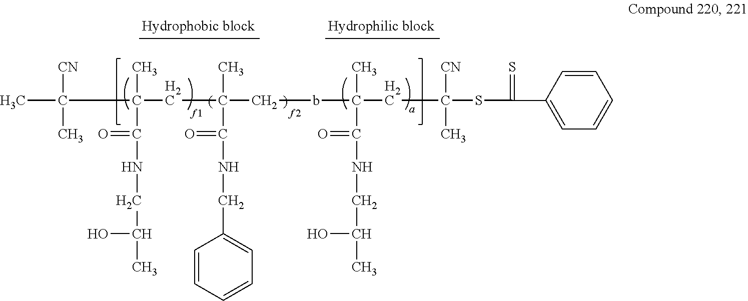

Compound 220-222. CN-p[(HPMA)ₐ₁-co-(BnMAM)_{f1}]-b-p(HPMA)ₐ₂-DTB wherein subscripts a1 refers to an integer number of hydrophilic co-monomers of the hydrophobic block, f1 refers to an integer number of hydrophobic co-monomers of the hydrophobic block and a2 refers to an integer number of hydrophilic monomers of the hydrophilic block. Polymers were synthesized via RAFT polymerization of HPMA (Compound 10) using macro-CTAs of structure CN-[p[(HPMA)ₐ₁-co-(BnMAM)_{f1}]-DTB with varying mol % BnMAM co-monomer and AIBN (Compound 1) as initiator. For polymerization, the macro-CTA hydrophobic polymer block was dissolved in DMAc, AIBN was pre-dissolved in DMSO and HPMA monomer was dissolved in tBuOH at [HPMA]₀=1 mol/L. The molar ratio [macro-CTA]₀:[AIBN]₀ was 1:0.5, and [HPMA]₀:[macro-CTA]₀ is defined in Table 10. A procedure similar to the preparation of Compound 200 was used for RAFT polymerization to obtain the diblock copolymer. Following polymerization of p(HPMA), the di-block copolymers were precipitated into Et₂O. Results from GPC-MALS and DLS are presented in Table 12 and Table 17 respectively.

TABLE 10
Compound 220-222 synthesis conditions.

| | | | Monomer |
|---|---|---|---|
| | | Macro-CTA | concentration, solvent |
| Cmpd | | compound | system, and |
| # | Polymer structure | number | [monomer]:[CTA] ratio |
| 220 | CN-p[(HPMA)ₐ₁-co-(BnMAM)_{f1}]-b- | 120 | 1M, tBuOH/DMAc |
| | p(HPMA)ₐ-DTB, 20 mol % hydrophobic | | (80/20), [188:1] |
| | block BnMAM | | |
| 221 | CN-p[(HPMA)ₐ₁-co-(BnMAM)_{f1}]-b- | 121 | 1M, tBuOH/DMAc |
| | p(HPMA)ₐ-DTB, 30 mol % hydrophobic | | (80/20), [257:1] |
| | block BnMAM | | |
| 222 | CN-p[(HPMA)ₐ₁-co-(BnMAM)_{f1}]-b- | 122 | 1M, tBuOH/DMAc |
| | p(HPMA)ₐ-DTB, 50 mol % hydrophobic | | (70/30), [257:1] |
| | block BnMAM | | |

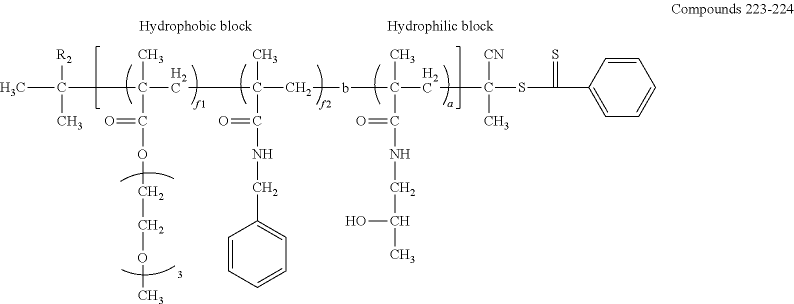

Compounds 223-224. CN-p[(TEGMA)_{f1}-co-(BnMAM)_{f2}]-b-p(HPMA)ₐ-DTB. Polymers were synthesized via RAFT polymerization of HPMA (Compound 10) using macro-CTAs of structure CN-[p[(TEGMA)_{f1}-co-(BnMAM)_{f2}]-DTB with varying mol % BnMAM co-monomer and AIBN (Compound 1) as initiator. For polymerization, the macro-CTA hydrophobic polymer block was dissolved in DMAc, AIBN was pre-dissolved in DMSO and HPMA monomer was dissolved in tBuOH at [HPMA]₀=1 mol/L. The molar ratio [macro-CTA]₀:[AIBN]₀ was 1:0.5, and [HPMA]₀:[macro-CTA]₀ is defined in Table 11. A procedure similar to the preparation of Compound 200 was used for RAFT polymerization to obtain the diblock copolymer. Following polymerization of p(HPMA), the di-block copolymers were precipitated into Et2O. Results from GPC-MALS and DLS are presented in Table 12 and Table 17 respectively.

TABLE 11
Compound 223-224 synthesis conditions.

| | | | Monomer |
|---|---|---|---|
| | | Macro-CTA | concentration, solvent |
| Cmpd | | compound | system, and |
| # | Polymer structure | number | [monomer]:[CTA] ratio |
| 223 | CN-p[(TEGMA)_{f1}-co-(BnMAM)_{f2}]-b- | 131 | 2M, tBuOH/DMAc |
| | p(HPMA)ₐ-DTB, 15 mol % hydrophobic | | (80/20), [150:1] |
| | block BnMAM | | |
| 224 | CN-p[(TEGMA)_{f1}-co-(BnMAM)_{f2}]-b- | 132 | 2M, tBuOH/DMAc |
| | p(HPMA)ₐ-DTB, 20 mol % hydrophobic | | (80/20), [150:1] |
| | block BnMAM | | |

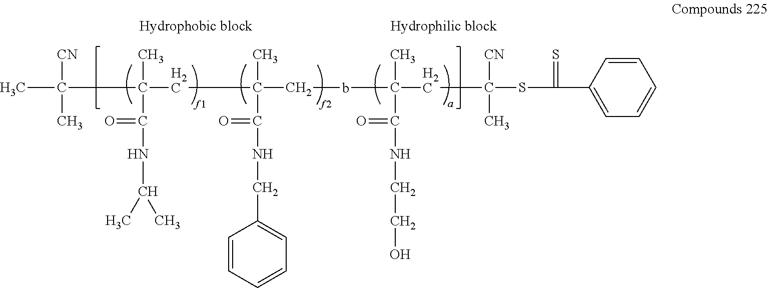

Compound 225. CN-p[(NIPMAM)_{f1}-co-(BnMAM)_{f2}]-b-p(HEMAM)ₐ-DTB. Polymer was synthesized via RAFT polymerization of HEMAM (Compound 24) using CN-p[(NIPMAM)_{f1}-co-(BnMAM)_{f2}]-DTB (Compound 100) as a macro-chain transfer agent and AIBN (Compound 1) as initiator. For polymerization, the macro-CTA hydrophobic polymer block was dissolved in DMAc, AIBN was pre-dissolved in DMSO and HEMAM monomer was dissolved in MeOH at [HEMAM]₀=1 mol/L. The molar ratio [macro-CTA]₀:[AIBN]₀ was 1:0.5, and [HEMAM]₀:[macro-CTA]₀ was set to 244. A procedure similar to the preparation of Compound 200 was used for RAFT polymerization to obtain the diblock copolymer. Following polymerization of p(HEMAM), the di-block copolymer was precipitated into cold acetone/Et2O (2:1) and dried under vacuum. Results from GPC-MALS and DLS are presented in Table 12 and Table 17 respectively.

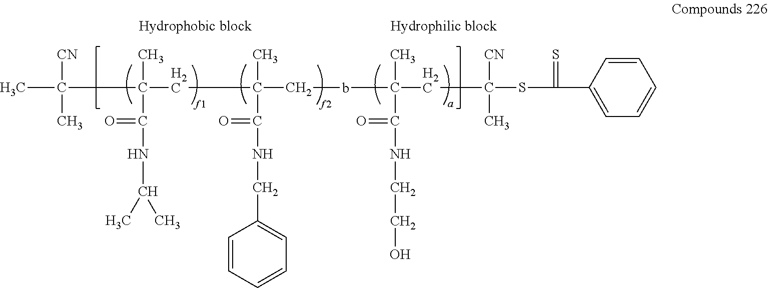

Compound 226. CN-p[(NIPMAM)_{f1}-co-(BnMAM)_{f2}]-b-p(HEA)ₐ-DTB. Polymer was synthesized via RAFT polymerization of HEA (Compound 25) using CN-p[(NIPMAM)_{f1}-co-(BnMAM)_{f2}]-DTB (Compound 100) as a macro-chain transfer agent and AIBN (Compound 1) as initiator. For polymerization, the macro-CTA hydrophobic polymer block was dissolved in DMAc, AIBN was pre-dissolved in DMSO and HEMAM monomer was dissolved in dioxane at [HEA]₀=2 mol/L. The molar ratio [macro-CTA]₀:[AIBN]₀ was 1:0.5, and [HEA]₀:[macro-CTA]₀ was set to 180. A procedure similar to the preparation of Compound 200 was used for RAFT polymerization to obtain the diblock copolymer. Following polymerization of p(HEMAM), the di-block copolymer was precipitated into cold acetone/Et₂O(2:1) and dried under vacuum. Results from GPC-MALS and DLS are presented in Table 12 and Table 17 respectively.

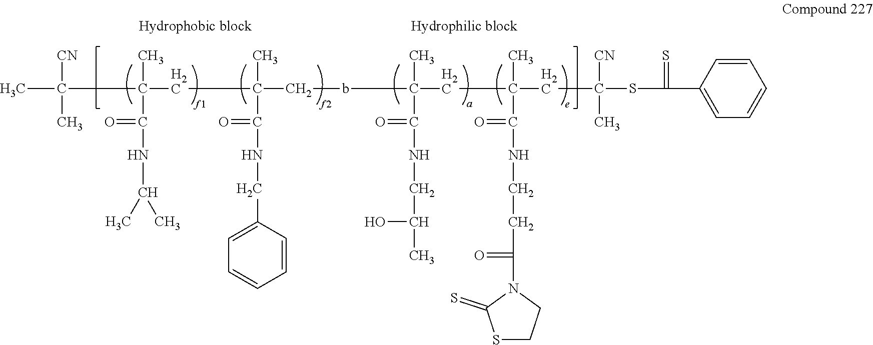

Compound 227. CN-p[(NIPMAM)_{f1}-co-(BnMAM)_{f2}]-b-p(HEA)ₐ-DTB wherein subscripts f1 and f2 refer to integer numbers of hydrophobic block co-monomers, a refers to an integer number of hydrophilic block hydrophilic co-monomers and e refers to an integer number of hydrophilic block reactive co-monomers of MA-b-Ala-TT (Compound 11). Polymer was synthesized via RAFT polymerization of HPMA 90 mol % (Compound 12) and MA-b-Ala-TT 10 mol % (Compound 11) using CN-p[(NIPMAM)_{f1}-co-(BnMAM)_{f2}]-DTB (Compound 100) as a macro-chain transfer agent and AIBN (Compound 1) as initiator. For polymerization, the macro-CTA hydrophobic polymer block was dissolved in DMAc, AIBN was pre-dissolved in DMSO and HPMA and MA-b-Ala-TT monomers were dissolved in tBuOH and DMAc respectively at a total concentration ([HPMA]₀+[MA-b-Ala-TT]₀)=1 mol/L. The molar ratio [macro-CTA]₀:[AIBN]₀ was 1:0.5, and ([HPMA]₀+[MA-b-Ala-TT]₀):[macro-CTA]₀ was set to 200. A procedure similar to the preparation of Compound 200 was used for RAFT polymerization to obtain the diblock copolymer. Following polymerization, the di-block copolymer was precipitated into cold acetone/Et₂O(2:1) and dried under vacuum. Results from GPC-MALS and DLS are presented in Table 12 and Table 17 respectively.

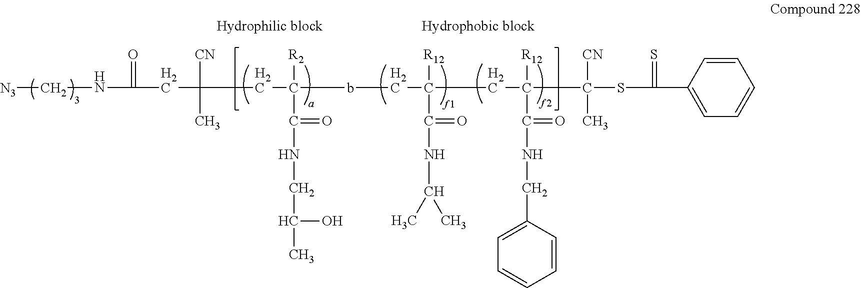

Compound 228. N3-p(HPMA)ₐ-b-p[(NIPMAM)_{f1}-co-(BnMAM)_{f2}]-DTB. Diblock co-polymer was synthesized in reverse direction using a hydrophilic block as a macro-CTA to polymerize NIPMAM 80 mol % (Compound 12) and BnMAM 20 mol % (Compound 13) via RAFT polymerization. N3-p(HPMA)ₐ-DTB (Compound 125) was used as a macro-chain transfer agent and AIBN (Compound 1) as initiator. For polymerization, the macro-CTA hydrophobic polymer block was dissolved in DMAc, AIBN was pre-dissolved in DMSO, NIPMAM and BnMAM monomers were dissolved in tBuOH and DMAc respectively at a total concentration ([NIPMAM]₀+[BnMAM]₀)=2 mol/L. The molar ratio [macro-CTA]₀:[AIBN]₀ was 1:0.5, and ([NIPMAM]₀+[BnMAM]₀):[macro-CTA]₀ was set to 250. A procedure similar to the preparation of Compound 200 was used for RAFT polymerization to obtain the diblock copolymer. Following polymerization, the di-block copolymer was precipitated into cold acetone/Et₂O(2:1) and dried under vacuum. Results from GPC-MALS and DLS are presented in Table 12 and Table 17 respectively.

TABLE 12
Compounds 200-228 GPC-MALS characterization results. Note: block 1 refers to the first block
synthesized, i.e., the macro-CTA, whereas block 2 refers to the second block.

| | Overall Mn | | Block 1 | Block 2 | Block 2 Degree |
|---|---|---|---|---|---|
| Cmpd # | (kDa) | PDI | Mn (kDa)/Xₙ | Mn (kDa)/Xₙ | of Conversion |
| 200 | 44.9 | 1.07 | 16/117 | 28.9/202 | 89.2 |
| 201 | 26.8 | 1.07 | 16.1/122 | 10.7/75 | 32.6 |
| 202 | 42.5 | 1.10 | 16.1/122 | 26.4/185 | 33.9 |
| 203 | 58.9 | 1.23 | 16.1/122 | 42.8/299 | 60.0 |
| 204 | 26.6 | 1.06 | 16/119 | 10.6/74 | 69.6 |
| 205 | 41.3 | 1.21 | 16/119 | 25.3/177 | 43.7 |
| 206 | 28.3 | 1.06 | 16/117 | 12.3/86 | 45.8 |
| 207 | 39.7 | 1.15 | 16/117 | 23.7/166 | 46.9 |
| 208 | 45.7 | 1.23 | 16/117 | 29.7/208 | 52.9 |
| 209 | 45.6 | 1.08 | 16.8/119 | 29.4/201 | 77.0 |
| 210 | 47.8 | 1.05 | 12.7/88 | 35.1/246 | 100.8 |
| 211 | 24.1 | 1.01 | 12.7/88 | 11.4/80 | 79.6 |
| 212 | 49.3 | 1.03 | 15/98 | 34.2/239 | 93.3 |
| 213 | 24.1 | 1.01 | 15/98 | 9.1/64 | 63.5 |
| 214 | 45.5 | 1.08 | 23.8/136 | 21.7/152 | 56.2 |
| 215 | 35.0 | 1.10 | 14/80 | 21/147 | 65.3 |
| 216 | 37.6 | 1.02 | 21.7/140 | 15.9/111 | 49.3 |
| 217 | 38.3 | 1.27 | 12.5/85 | 25.8/180 | 72.5 |
| 218 | 22.7 | 1.17 | 15.8/123 | 6.9/48 | 41.4 |
| 219 | 81.6 | 1.05 | 15.8/123 | 65.8/460 | 36.6 |
| 220 | 35.5 | 1.42 | 11.5/75 | 24/168 | 89.2 |
| 221 | 39.0 | 1.35 | 18.7/121 | 20.3/142 | 55.1 |
| 222 | 26.8 | 1.08 | 23.9/149 | 2.9/21 | 8.0 |
| 223 | 35.7 | 1.58 | 30.1/134 | 5.6/39 | 26.1 |
| 224 | 36.4 | 1.52 | 28.4/128 | 8/56 | 37.4 |
| 225 | 48.7 | 1.11 | 16/117 | 32.7/253 | 95.9 |
| 226 | 41.4 | 1.06 | 21.7/157 | 19.7/170 | 0.9 |
| 227 | 46.7 | 1.06 | 8.8/63 | 37.8/245 | 122.3 |
| 228 | 39.1 | 1.30 | 22.9/157 | 16.2/119 | 47.5 |

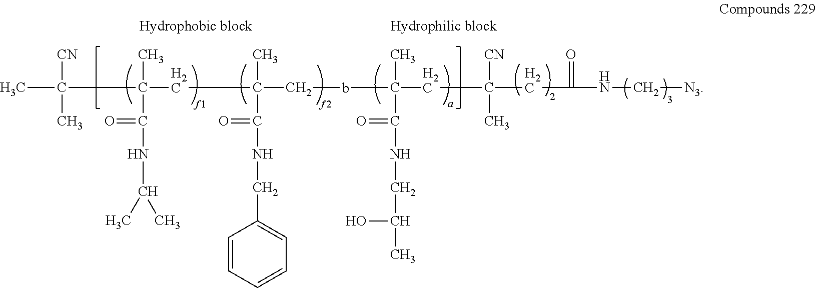

Compound 229. CN-p[(NIPMAM)_{f1}-co-(BnMAM)_{f2}]-b-p(HPMA)ₐ-N3. Azide-functionalized di-block copolymers were prepared by reacting DTB terminated polymer with 20 molar equivalents of ACVA-N3 (Compound 4). Example of reaction: Dry polymer CN-p[(NIPMAM)_{f1}-co-(BnMAM)_{f2}]-b-p(HPMA)ₐ-DTB (Compound 200) (50 mg, 1.25 μmol) and ACVA-N3 (11.11 mg, 25 μmol) were dissolved in 0.5 mL of anhydrous DMSO. The solution was transferred to a 2 mL ampule, which was sealed with a rubber septum and sparged with Ar (g) at r.t. for 20 min. The flask was then immersed in a water circulator preheated to 80° C. and reacted for 2 h. The polymer was purified by precipitating against diethyl ether for 2 times. After drying under vacuum overnight, off-white powder was obtained.

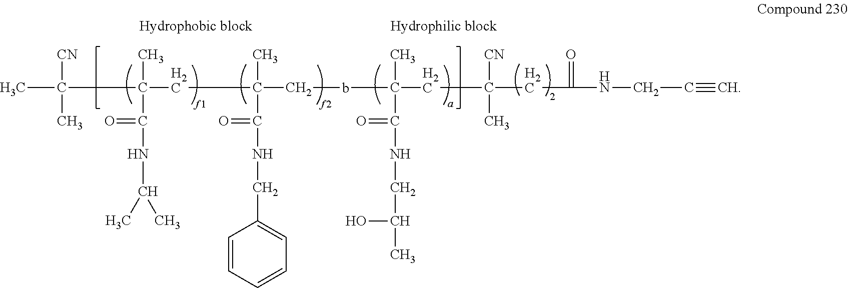

Compound 230. CN-p[(NIPMAM)_{f1}-co-(BnMAM)_{f2}]-b-p(HPMA)ₐ-Pg. Propargyl-functionalized di-block copolymers were prepared by reacting DTB terminated polymer with 20 molar equivalents of ACVA-Pg (Compound 3). Example of reaction: Dry polymer CN-p[(NIPMAM)_{f1}-co-(BnMAM)_{f2}]-b-p(HPMA)ₐ-DTB (Compound 200) (50 mg, 1.25 μmol) and ACVA-Pg (8.86 mg, 25 μmol) were dissolved in 0.5 mL of anhydrous DMSO. The solution was transferred to a 2 mL ampule, which was sealed with a rubber septum and sparged with Ar (g) at r.t. for 20 min. The flask was then immersed in a water circulator preheated to 80° C. and reacted for 2 h. The polymer was purified by precipitating against diethyl ether for 2 times. After drying under vacuum overnight, off-white powder was obtained.

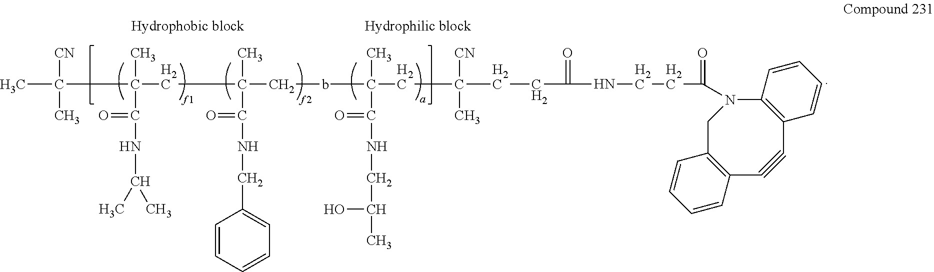

Compound 231. CN-p[(NIPMAM)_{f1}-co-(BnMAM)_{f2}]-b-p(HPMA)ₐ-DBCO. DBCO-functionalized di-block copolymers were prepared by reacting DTB terminated polymer with 20 molar equivalents of ACVA-DBCO (Compound 5). Example of reaction: Dry polymer CN-p[(NIPMAM)_{f1}-co-(BnMAM)_{f2}]-b-p(HPMA)ₐ-DTB (Compound 200) (50 mg, 1.25 μmol) and ACVA-DBCO (19.92 mg, 25 μmol) were dissolved in 0.7 mL of anhydrous DMSO. The solution was transferred to a 2 mL ampule, which was sealed with a rubber septum and sparged with Ar (g) at r.t. for 20 min. The flask was then immersed in a water circulator preheated to 80° C. and reacted for 2 h. The polymer was purified by precipitating against diethyl ether for 2 times. After drying under vacuum overnight, off-white powder was obtained.

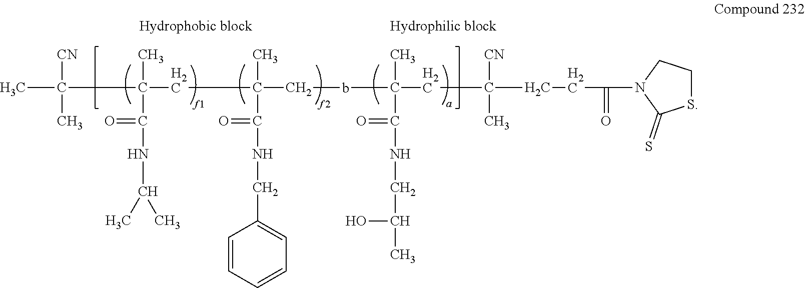

Compound 232. CN-p[(NIPMAM)_{f1}-co-(BnMAM)_{f2}]-b-p(HPMA)ₐ-TT. TT-functionalized di-block copolymers were prepared by reacting DTB terminated polymer with 20 molar equivalents of ACVA-TT (Compound 5). Example of reaction: Dry polymer CN-p[(NIPMAM)_{f1}-co-(BnMAM)_{f2}]-b-p(HPMA)ₐ-DTB (Compound 200) (50 mg, 1.25 μmol) and ACVA-TT (12.1 mg, 25 μmol) were dissolved in 0.6 mL of anhydrous DMSO. The solution was transferred to a 2 mL ampule, which was sealed with a rubber septum and sparged with Ar (g) at r.t. for 20 min. The flask was then immersed in a water circulator preheated to 80° C. and reacted for 2 h. The polymer was purified by precipitating against diethyl ether for 2 times. After drying under vacuum overnight, off-white powder was obtained.

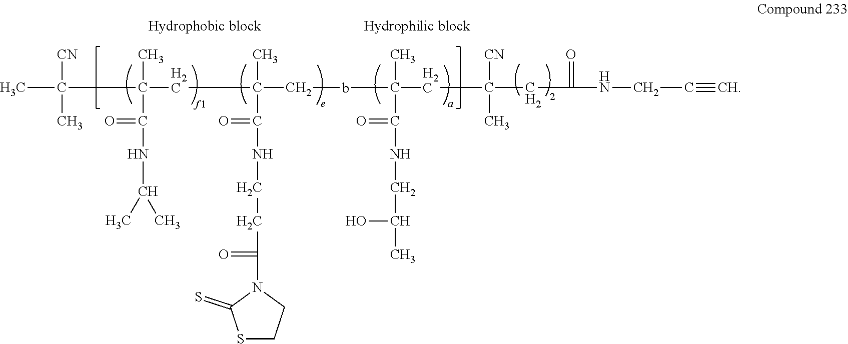

Compound 233. CN-p[(NIPMAM)_{f1}-co-(MA-b-Ala-TT)ₑ]-b-p(HPMA)ₐ-Pg. Propargyl-functionalized di-block copolymers were prepared by reacting DTB terminated polymer with 20 molar equivalents of ACVA-Pg (Compound 3). Example of reaction: Dry polymer CN-p[(NIPMAM)_{f1}-co-(MA-b-Ala-TT)ₑ]-b-p(HPMA)ₐ-DTB (Compound 216) (100 mg, 2.66 μmol) and ACVA-Pg (18.9 mg, 53.2 μmol) were dissolved in 1.2 mL of anhydrous DMSO. The solution was transferred to a 2 mL ampule, which was sealed with a rubber septum and sparged with Ar (g) at r.t. for 20 min. The flask was then immersed in a water circulator preheated to 80° C. and reacted for 2 h. The polymer was purified by precipitating against diethyl ether for 2 times. After drying under vacuum overnight, off-white powder was obtained.

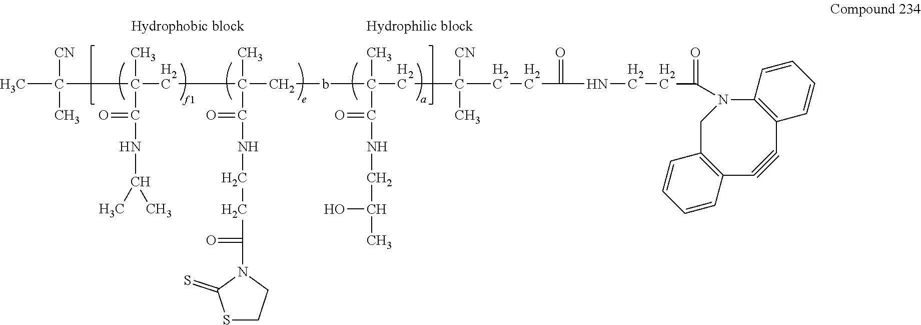

Compound 234. CN-p[(NIPMAM)_{f1}-co-(MA-b-Ala-TT)ₑ]-b-p(HPMA)ₐ-DBCO. Propargyl-functionalized di-block copolymers were prepared by reacting DTB terminated polymer with 20 molar equivalents of ACVA-DBCO (Compound 5). Example of reaction: Dry polymer CN-p[(NIPMAM)_{f1}-co-(MA-b-Ala-TT)ₑ]-b-p(HPMA)ₐ-DTB (Compound 216) (100 mg, 2.66 μmol) and ACVA-DBCO (42.4 mg, 53.19 μmol) were dissolved in 1.3 mL of anhydrous DMSO. The solution was transferred to a 2 mL ampule, which was sealed with a rubber septum and sparged with Ar (g) at r.t. for 20 min. The flask was then immersed in a water circulator preheated to 80° C. and reacted for 2 h. The polymer was purified by precipitating against diethyl ether for 2 times. After drying under vacuum overnight, off-white powder was obtained.

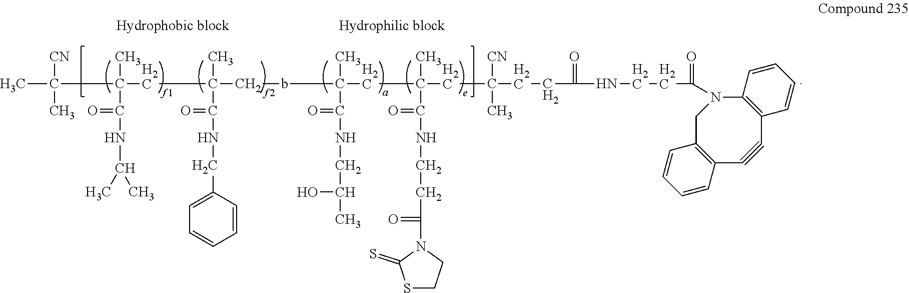

Compound 235. CN-p[(NIPMAM)_{f1}-co-(BnMAM)_{f2}]-b-p[(HPMA)ₐ-co-(MA-b-Ala-TT)ₑ]-DBCO. Propargyl-functionalized di-block copolymers were prepared by reacting DTB terminated polymer with 20 molar equivalents of ACVA-DBCO (Compound 5). Example of reaction: Dry polymer CN-p[(NIPMAM)_{f1}-co-(BnMAM)_{f2}]-b-p[(HPMA)ₐ-co-(MA-b-Ala-TT)ₑ]-DTB (Compound 227) (100 mg, 2.14 μmol) and ACVA-DBCO (34.13 mg, 42.83 μmol) were dissolved in 1.35 mL of anhydrous DMSO. The solution was transferred to a 2 mL ampule, which was sealed with a rubber septum and sparged with Ar (g) at r.t. for 20 min. The flask was then immersed in a water circulator preheated to 80° C. and reacted for 2 h. The polymer was purified by precipitating against diethyl ether for 2 times. After drying under vacuum overnight, off-white powder was obtained.

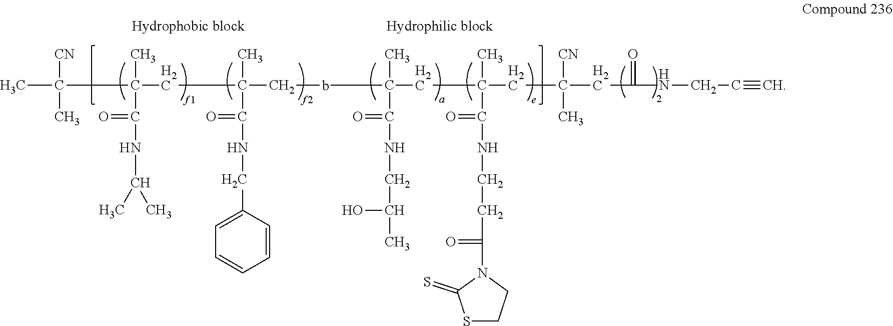

Compound 236. CN-p[(NIPMAM)_{f1}-co-(BnMAM)_{f2}]-b-p[(HPMA)ₐ-co-(MA-b-Ala-TT)ₑ]-Pg. Propargyl-functionalized di-block copolymers were prepared by reacting DTB terminated polymer with 20 molar equivalents of ACVA-Pg (Compound 3). Example of reaction: Dry polymer CN-p[(NIPMAM)_{f1}-co-(BnMAM)_{f2}]-b-p[(HPMA)ₐ-co-(MA-b-Ala-TT)ₑ]-DTB (Compound 227) (Compound 227) (100 mg, 2.14 μmol) and ACVA-Pg (15.18 mg, 42.8 μmol) were dissolved in 1.2 mL of anhydrous DMSO. The solution was transferred to a 2 mL ampule, which was sealed with a rubber septum and sparged with Ar (g) at r.t. for 20 min. The flask was then immersed in a water circulator preheated to 80° C. and reacted for 2 h. The polymer was purified by precipitating against diethyl ether for 2 times. After drying under vacuum overnight, off-white powder was obtained.

### Example 4—Polymer Functionalization Via Side Chain MA-b-Ala-TT Reaction

Polymers with reactive co-monomers including MA-b-Ala-TT (Compound 11) or MA-PFP (Compound 14) in either the hydrophobic or hydrophilic block of the polymer were reacted with amino ligands at defined molar ratios to functionalize the side chain monomers and change the overall property of the polymers. These experiments were performed to identify suitable hydrophobic co-monomer mol %, conjugate drug molecules and functionalize polymers with ligands including peptides. Polymers with reactive co-monomers used include the following listed in Table 13. The following procedure was employed for a typical reaction to react to activated carboxylic acids (e.g., carbonylthiazolidine-2-thione of MA-b-Ala-TT co-monomer). Polymer was dissolved in DMSO to a concentration of 200 mg/mL and known mass of amino ligand pre-dissolved in DMSO was added at the molar ratio to MA-b-Ala-TT co-monomer concentration stated in Table 14. Triethylamine was then added (10 equivalents to MA-b-Ala-TT co-monomer concentration) and the reaction was accelerated by heating to 40° C. for four hours. At four hours, a portion of the reaction mixture was sampled and analyzed by HPLC (Agilent, C-18 column with water/ACN gradient with 0.05% v/v TFA from 5-95% v/v ACN over 6 minutes with diode array detector monitoring absorbance). HPLC enabled reaction efficacy to be monitored by quantifying the amount of amino ligand remaining unreacted (if absorbance was detectable) and the amount of 2-thiazoline-2-thione released (at 280 nm and specific elution time) relative to the theoretical amount of 2-thiazoline-2-thione that should be released. After confirmation of reaction efficacy, any remaining unreacted MA-b-Ala-TT groups on the polymer were quenched by addition of 2 equivalents of either isopropylamine (CAS: 75-31-0) for hydrophobic block MA-b-Ala-TT co-monomers to mimic NIPMAM or amino-2-propanol (CAS: 78-96-6) to mimic HPMA. Fully reacted polymers were then purified using dialysis with a 10 kDa molecular weight cutoff (MWCO) regenerated cellulose dialysis tubing against 10% v/v DMSO in MeOH followed by dialysis against MeOH and drying under vacuum. Polymer yield was then determined and the functionalized polymer was then dissolved into DMSO at 100 mg/mL.

TABLE 13
Polymers used for co-monomer side-chain activated carboxylic acid reaction with amino ligands.

| Cmpd | | Mol % MA-b-Ala-TT | # MA-b-Ala-TT |
|---|---|---|---|
| # | Structure | of polymer block | Co-monomers (e) |
| 137 | Pg-p[(NIPMAM)_{f1}-co-(Ma-b-Ala- | 10 | 25 |
| | TT)ₑ]-DBCO | | |
| 233 | CN-p[(NIPMAM)_{f1}-co-(MA-b- | 20 | 22 |
| | Ala-TT)ₑ]-b-p(HPMA)ₐ-Pg | | |
| 234 | CN-p[(NIPMAM)_{f1}-co-(MA-b- | 20 | 22 |
| | Ala-TT)ₑ]-b-p(HPMA)ₐ-DBCO | | |
| 235 | CN-p[(NIPMAM)_{f1}-co- | 10 | 24 |
| | (BnMAM)_{f2}]-b-p[(HPMA)ₐ-co- | | |
| | (Ma-b-Ala-TT)ₑ]-DBCO | | |
| 236 | CN-p[(NIPMAM)_{f1}-co- | 10 | 24 |
| | (BnMAM)_{f2}]-b-p[(HPMA)ₐ-co- | | |
| | (Ma-b-Ala-TT)ₑ]-Pg | | |

TABLE 14
Side-chain MA-b-Ala-TT co-monomer reactions with amino ligands. Pre-rxn cmpd # refers to
the compound number of the polymer with MA-b-Ala-TT co-monomers listed in Table 13.
Post-rxn Cmpd # refers to the new compound number of the polymer after reaction with the
specified amino ligand at the specified mole ratio. Mole ratio to MA-b-Ala-TT co-monomers
in polymer refers to the molar ratio of the ligand equivalents to MA-b-Ala-TT co-monomer
concentration. Finally, # of Amino Ligands reacted per polymer refers to the number of amino
ligand molecules (or drug molecules) reacted to each polymer molecule and confirmed by HPLC.

| | | | | Mol % of | |
|---|---|---|---|---|---|
| | | | Mole ratio to | polymer | # of Amino |
| | | | MA-b-Ala-TT | block | Ligands |
| Pre-rxn | Post-rxn | | co-monomers | ligand | reacted per |
| Cmpd # | Cmpd # | Amino Ligand | in polymer | occupied | polymer |
| 137 | 237 | No ligand; | 0.00 | 0 | 0.0 |
| | | isopropylamine only | | | |
| | | (CAS: 75-31-0) | | | |
| 137 | 238 | 2BXy (Compound 32) | 0.10 | 1 | 2.7 |
| 137 | 239 | 2BXy (Compound 32) | 0.20 | 2 | 5.3 |
| 137 | 240 | 2BXy (Compound 32) | 0.30 | 3 | 8.0 |
| 137 | 241 | 2BXy (Compound 32) | 0.40 | 4 | 10.7 |
| 137 | 242 | 2BXy (Compound 32) | 0.60 | 6 | 16.0 |
| 137 | 243 | 2BXy (Compound 32) | 0.80 | 8 | 21.4 |
| 236 | 244 | No ligand; amino-2- | 0.00 | 0 | 0.0 |
| | | propanol only (CAS: | | | |
| | | 78-96-6) | | | |
| 236 | 245 | 2BXy (Compound 32) | 0.20 | 2 | 5 |
| 236 | 246 | 2BXy (Compound 32) | 0.30 | 3 | 7 |
| 236 | 247 | 2BXy (Compound 32) | 0.40 | 4 | 10 |
| 236 | 248 | 2BXy (Compound 32) | 0.50 | 5 | 12 |
| 236 | 249 | p2610 (Compound 30) | 0.6 | 6 | 14 |
| 233 | 250 | Benzylamine (CAS: | 2.00 | 20 | 22 |
| | | 100-46-9) | | | |
| 233 | 251 | 4-Phenylbutylamine | 2.00 | 20 | 22 |
| | | (CAS: 131214-66-9) | | | |
| 233 | 252 | 4-Phenylbutylamine | 0.50 | 10 | 11 |
| | | (CAS: 131214-66-9) | | | |
| 233 | 253 | 4-Phenylbutylamine | 0.25 | 5 | 6 |
| | | (CAS: 131214-66-9) | | | |
| 233 | 254 | 2,4-Dimethoxy- | 2.00 | 20 | 22 |
| | | benzylamine (CAS: | | | |
| | | 20781-20-8) | | | |
| 233 | 255 | 2,4-Dimethoxy- | 0.50 | 10 | 11 |
| | | benzylamine (CAS: | | | |
| | | 20781-20-8) | | | |
| 233 | 256 | 2,4-Dimethoxy- | 0.25 | 5 | 6 |
| | | benzylamine (CAS: | | | |
| | | 20781-20-8) | | | |
| 233 | 257 | Cyclohexanemethylamine | 2.00 | 20 | 22 |
| | | (CAS: 3218--02-8) | | | |
| 233 | 258 | Cyclohexanemethylamine | 0.50 | 10 | 11 |
| | | (CAS: 3218--02-8) | | | |
| 233 | 259 | Cyclohexanemethylamine | 0.25 | 5 | 6 |
| | | (CAS: 3218--02-8) | | | |
| 233 | 260 | 4-(Trifluoromethyl) | 2.00 | 20 | 22 |
| | | benzylamine | | | |
| | | (CAS: 3300-51-4) | | | |
| 233 | 261 | 4-(Trifluoromethyl) | 0.50 | 10 | 11 |
| | | benzylamine | | | |
| | | (CAS: 3300-51-4) | | | |
| 233 | 262 | 4-(Trifluoromethyl) | 0.25 | 5 | 6 |
| | | benzylamine | | | |
| | | (CAS: 3300-51-4) | | | |
| 233 | 263 | 3,5- | 2.00 | 20 | 22 |
| | | Bis(Trifluoromethyl) | | | |
| | | benzylamine | | | |
| | | (CAS: 85068-29-7) | | | |
| 233 | 264 | 3,5- | 0.50 | 10 | 11 |
| | | Bis(Trifluoromethyl) | | | |
| | | benzylamine | | | |
| | | (CAS: 85068-29-7) | | | |
| 233 | 265 | 3,5- | 0.25 | 5 | 6 |
| | | Bis(Trifluoromethyl) | | | |
| | | benzylamine | | | |
| | | (CAS: 85068-29-7) | | | |
| 233 | 266 | Butylamine | 2.00 | 20 | 22 |
| | | (CAS: 109-73-9) | | | |
| 233 | 267 | Butylamine | 0.50 | 10 | 11 |
| | | (CAS: 109-73-9) | | | |
| 233 | 268 | Butylamine | 0.25 | 5 | 6 |
| | | (CAS: 109-73-9) | | | |
| 233 | 269 | 1-Octylamine | 0.50 | 10 | 11 |
| | | (CAS: 111-86-4) | | | |
| 233 | 270 | 1-Octylamine | 0.25 | 5 | 6 |
| | | (CAS: 111-86-4) | | | |
| 233 | 271 | 1-Octylamine | 0.13 | 2.5 | 3 |
| | | (CAS: 111-86-4) | | | |
| 233 | 272 | 1-Dodecylamine | 0.50 | 10 | 11 |
| | | (CAS: 124-22-1) | | | |
| 233 | 273 | 1-Dodecylamine | 0.25 | 5 | 6 |
| | | (CAS: 124-22-1) | | | |
| 233 | 274 | 1-Dodecylamine | 0.13 | 2.5 | 3 |
| | | (CAS: 124-22-1) | | | |
| 233 | 275 | Aniline (CAS: 62-53-3) | 2.00 | 20 | 22 |
| 233 | 276 | 3,5-Bis | 2.00 | 20 | 22 |
| | | (Trifluoromethyl)aniline | | | |
| | | (CAS: 328-74-5) | | | |
| 233 | 277 | 3,5-Dimethylaniline | 2.00 | 20 | 22 |
| | | (CAS: 108-69-0) | | | |
| 233 | 278 | 3,5-Difluoroanaline | 2.00 | 20 | 22 |
| | | (CAS: 372-39-4) | | | |
| 234 | 279 | 2BXy (Compound 32) | 2.00 | 20 | 22 |
| 234 | 280 | 2BXy (Compound 32) | 0.50 | 10 | 11 |
| 234 | 281 | 2BXy (Compound 32) | 0.23 | 5 | 5 |
| 234 | 282 | 2BXy (Compound 32) | 0.125 | 2.5 | 3 |
| 234 | 283 | diABZI-pip (Compound | 1.00 | 20 | 22 |
| | | 33) | | | |
| 234 | 284 | diABZI-pip (Compound | 0.50 | 10 | 11 |
| | | 33) | | | |
| 234 | 285 | diABZI-pip (Compound | 0.25 | 5 | 6 |
| | | 33) | | | |
| 234 | 286 | diABZI-pip (Compound | 0.125 | 2.5 | 3 |
| | | 33) | | | |

TABLE 15
Thermo-responsive properties of polymers prepared by reaction to MA-b-Ala-TT co-monomers.
Aggregate (Agg.) denotes polymer was aggregated and not accurate for diameter measurement
by DLS at specified temperature.

| | | Measured | | |
|---|---|---|---|---|
| | | Transition | | |
| Post-rxn | | Temperature | Diameter at | Diameter at |
| Cmpd # | Amino Ligand | (° C.) | 20° C. | 37° C. |
| 237 | No ligand; isopropylamine | 52 | 6.3 | 6.1 |
| | only (CAS: 75-31-0) | | | |
| 238 | 2BXy (Compound 32) | 42 | 10.8 | 8.6 |
| 239 | 2BXy (Compound 32) | 35 | 10.1 | 17.3 |
| 240 | 2BXy (Compound 32) | 32 | 12.5 | 32.5 |
| 241 | 2BXy (Compound 32) | 30 | 10.6 | Agg. |
| 242 | 2BXy (Compound 32) | 25 | 14.8 | Agg |
| 243 | 2BXy (Compound 32) | 24 | 22.8 | Agg. |
| 244 | No ligand; amino-2-propanol | 32 | 9.5 | 37.7 |
| | only (CAS: 78-96-6) | | | |
| 245 | 2BXy (Compound 32) | 32 | 9.9 | 31.5 |
| 246 | 2BXy (Compound 32) | 25 | 6.8 | 33.2 |
| 247 | 2BXy (Compound 32) | 26 | 6.2 | 33.8 |
| 248 | 2BXy (Compound 32) | 24 | 7.0 | 31.0 |
| 249 | p2610 (Compound 30) | 29 | 10.5 | 19.8 |
| 250 | Benzylamine (CAS: 100-46-9) | 24 | 7.8 | 24.7 |
| 251 | 4-Phenylbutylamine (CAS: | 13 | 18.3 | 22.3 |
| | 131214-66-9) | | | |
| 252 | 4-Phenylbutylamine (CAS: | 16 | 15.3 | 18.9 |
| | 131214-66-9) | | | |
| 253 | 4-Phenylbutylamine (CAS: | 33 | 11.8 | 18.7 |
| | 131214-66-9) | | | |
| 254 | 2,4-Dimethoxy-benzylamine | 13 | 19.1 | 28.5 |
| | (CAS: 20781-20-8) | | | |
| 255 | 2,4-Dimethoxy-benzylamine | 24 | 14.3 | 26.4 |
| | (CAS: 20781-20-8) | | | |
| 256 | 2,4-Dimethoxy-benzylamine | 32 | 5.9 | 29.1 |
| | (CAS: 20781-20-8) | | | |
| 257 | Cyclohexanemethylamine | 24 | 5.9 | 29.1 |
| | (CAS: 3218--02-8) | | | |
| 258 | Cyclohexanemethylamine | 35 | 7.5 | 19.6 |
| | (CAS: 3218--02-8) | | | |
| 259 | Cyclohexanemethylamine | 46 | 8.5 | 5.7 |
| | (CAS: 3218--02-8) | | | |
| 260 | 4-(Trifluoromethyl) | <4 | 33.2 | 31.3 |
| | benzylamine | | | |
| | (CAS: 3300-51-4) | | | |
| 261 | 4-(Trifluoromethyl) | 36 | 7.9 | 13.8 |
| | benzylamine | | | |
| | (CAS: 3300-51-4) | | | |
| 262 | 4-(Trifluoromethyl) | 44 | 6.7 | 6.5 |
| | benzylamine | | | |
| | (CAS: 3300-51-4) | | | |
| 263 | 3,5-Bis(Trifluoromethyl) | <4° C. | 38.6 | 34.9 |
| | benzylamine | | | |
| | (CAS: 85068-29-7) | | | |
| 264 | 3,5-Bis(Trifluoromethyl) | 28 | 10.4 | 16.1 |
| | benzylamine | | | |
| | (CAS: 85068-29-7) | | | |
| 265 | 3,5-Bis(Trifluoromethyl) | 43 | 5.3 | 9.8 |
| | benzylamine | | | |
| | (CAS: 85068-29-7) | | | |
| 266 | Butylamine (CAS: 109-73-9) | 34 | 6.6 | 100.7 |
| 267 | Butylamine (CAS: 109-73-9) | 47 | 6.1 | 7.5 |
| 268 | Butylamine (CAS: 109-73-9) | None 4-37° C. | 7.3 | 6.7 |
| 269 | 1-Octylamine (CAS: | Inconsistent | 8.2 | 22.2 |
| | 111-86-4) | | | |
| 270 | 1-Octylamine (CAS: | Inconsistent | 7.3 | 6.3 |
| | 111-86-4) | | | |
| 271 | 1-Octylamine (CAS: | None 4-60° C. | 6.8 | 9.1 |
| | 111-86-4) | | | |
| 272 | 1-Dodecylamine (CAS: | Inconsistent | 15.2 | 12.1 |
| | 124-22-1) | | | |
| 273 | 1-Dodecylamine (CAS: | None 4-60° C. | 7.2 | 8.1 |
| | 124-22-1) | | | |
| 274 | 1-Dodecylamine (CAS: | None 4-60° C. | 6.6 | 9.2 |
| | 124-22-1) | | | |
| 275 | Aniline (CAS: 62-53-3) | 34 | 9.0 | 35.4 |
| 276 | 3,5-Bis(Trifluoromethyl) | 30 | 9.1 | 127.9 |
| | aniline (CAS: 328-74-5) | | | |
| 277 | 3,5-Dimethylaniline | 29 | 11.5 | 19.1 |
| | (CAS: 108-69-0) | | | |
| 278 | 3,5-Difluoroanaline | 44 | 7.3 | 7.2 |
| | (CAS: 372-39-4) | | | |
| 279 | 2BXy (Compound 32) | <4 | 35.2 | 30.6 |
| 280 | 2BXy (Compound 32) | <4 | 34.1 | 33.3 |
| 281 | 2BXy (Compound 32) | 35 | 7.1 | 29.0 |
| 282 | 2BXy (Compound 32) | 46 | 13.2 | 8.9 |
| 283 | diABZI-pip (Compound 33) | <4 | 35.1 | 34.5 |
| 284 | diABZI-pip (Compound 33) | <4 | 32.1 | 32.4 |
| 285 | diABZI-pip (Compound 33) | <4 | 26.5 | 23.9 |
| 286 | diABZI-pip (Compound 33) | 9 | 18.6 | 22.1 |

### Example 5—Polymer Functionalization Via Hydrophilic Terminal Functional Group Reaction

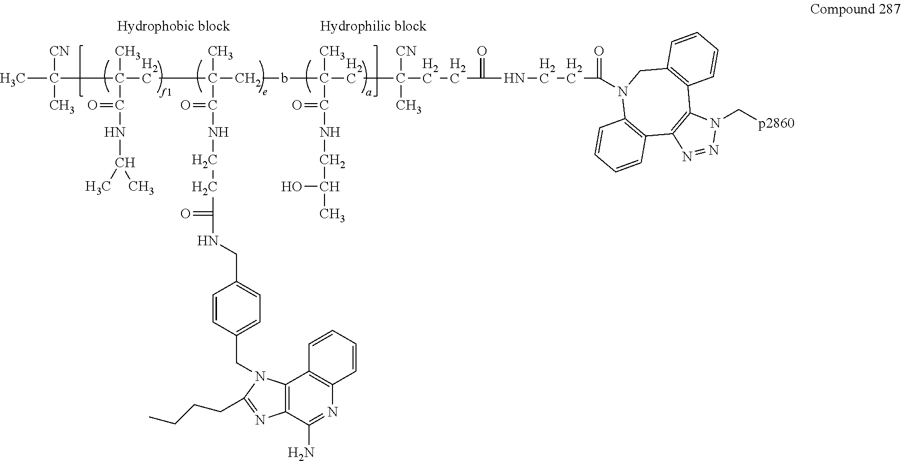

Compound 287. CN-p[(NIPMAM)_{f1}-co-(MA-b-Ala-2BXy)ₑ]-b-p(HPMA)ₐ-p2860. Hydrophilic terminus peptide conjugated polymer with 20 mol % hydrophobic block 2BXy co-monomer was prepared by reacting CN-p[(NIPMAM)_{f1}-co-(MA-b-Ala-2BXy)ₑ]-b-p(HPMA)ₐ-DBCO (Compound 279) with peptide drug molecule p2860 (Compound 31) using strain promoted alkyne azide cycloaddition. The polymer terminal DBCO group containing a strained alkyne was reacted with the azide residue of p2860. This reaction was performed by dissolving polymer in DMSO to a concentration of 200 mg/mL and mixing with a known mass of peptide p2860 pre-dissolved in DMSO at a 2:1 molar ratio to the terminal polymer DBCO concentration. The reaction was then accelerated by heating to 40° C. for four hours. At four hours, a portion of the reaction mixture was sampled and analyzed by HPLC (Agilent, C-18 column with water/ACN gradient with 0.05% TFA from 5-95% v/v ACN over 6 minutes with diode array detector monitoring absorbance). HPLC enabled reaction efficacy to be monitored by quantifying the amount of p2860 remaining unreacted. After confirmation of reaction efficacy, polymer was then purified from free peptide using dialysis with a 10 kDa molecular weight cutoff (MWCO) regenerated cellulose dialysis tubing against 10% v/v DMSO in MeOH followed by dialysis against MeOH and drying under vacuum. Polymer yield was then determined and the functionalized polymer was then dissolved into DMSO at 100 mg/mL. Thermo-responsive and hydrodynamic diameter properties are presented in Table 16.

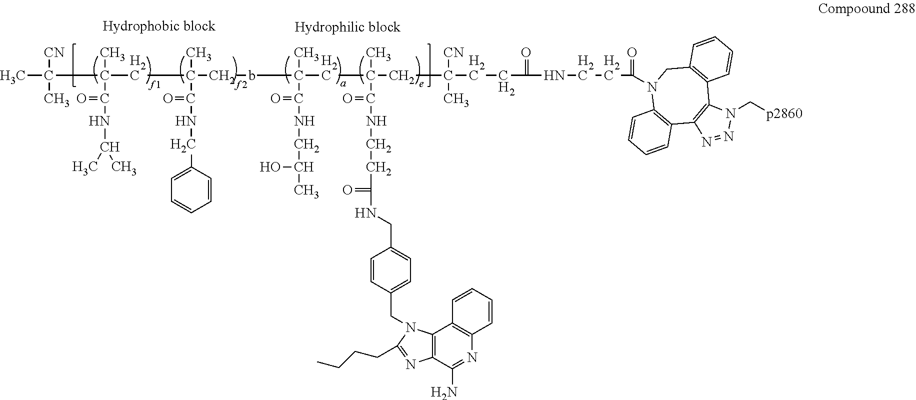

Compound 288. CN-p[(NIPMAM)_{f1}-co-(BnMAM)_{f2}]-b-p(HPMA)ₐ-co-(MA-b-Ala-2BXy)ₐ]-p2860. Hydrophilic terminus peptide conjugated polymer with 20 mol % hydrophobic block 2BXy co-monomer was prepared by reacting CN-p[(NIPMAM)_{f1}-co-(BnMAM)_{f2}]-b-p[(HPMA)ₐ-co-(MA-b-Ala-2BXy)ₐ]-DBCO (Compound 248) with peptide drug molecule p2860 (Compound 31) using strain promoted alkyne azide cycloaddition. The polymer terminal DBCO group containing a strained alkyne was reacted with the azide residue of p2860. This reaction was performed by dissolving polymer in DMSO to a concentration of 200 mg/mL and mixing with a known mass of peptide p2860 pre-dissolved in DMSO at a 2:1 molar ratio to the terminal polymer DBCO concentration. The reaction was then accelerated by heating to 40° C. for four hours. At four hours, a portion of the reaction mixture was sampled and analyzed by HPLC (Agilent, C-18 column with water/ACN gradient with 0.05% v/v TFA from 5-95% v/v ACN over 6 minutes with diode array detector monitoring absorbance). HPLC enabled reaction efficacy to be monitored by quantifying the amount of p2860 remaining unreacted. After confirmation of reaction efficacy, polymer was then purified from free peptide using dialysis with a 10 kDa molecular weight cutoff (MWCO) regenerated cellulose dialysis tubing against 10% DMSO in MeOH followed by dialysis against MeOH and drying under vacuum. Polymer yield was then determined and the functionalized polymer was then dissolved into DMSO at 100 mg/mL. Thermo-responsive and hydrodynamic diameter properties are presented in Table 16.

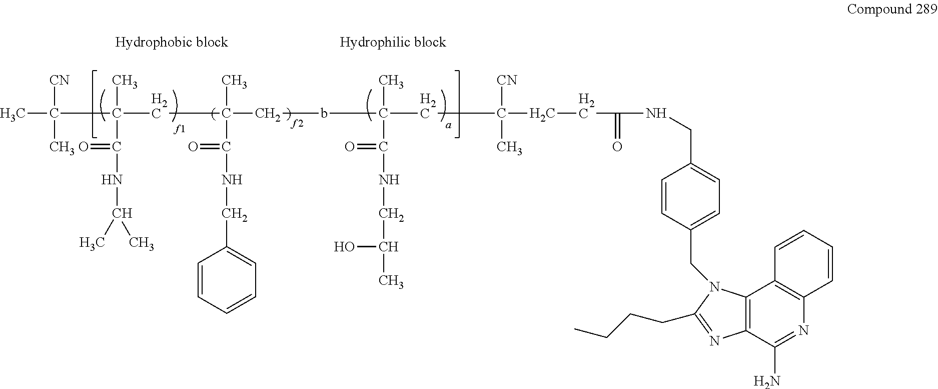

Compound 289. CN-p[(NIPMAM)_{f1}-co-(BnMAM)_{f2}]-b-p(HPMA)ₐ-2BXy. Hydrophilic terminus drug conjugated polymer was prepared by reacting CN-p[(NIPMAM)_{f1}-co-(BnMAM)_{f2}]-b-p(HPMA)ₐ-TT (Compound 232) with hydrophilic terminal functional group TT with small molecule drug 2BXy (Compound 32). This reaction was performed similarly to as described for Compound 237 with MA-b-Ala-TT side-chain co-monomer functionalization. Polymer was dissolved in DMSO to a concentration of 200 mg/mL and known mass of 2BXy pre-dissolved in DMSO was added at a 2:1 molar ratio to the terminal activated carboxylic acid concentration. Triethylamine was then added (10 equivalents to polymer activated carboxylic acid concentration) and the reaction was accelerated by heating to 40° C. for four hours. At four hours, a portion of the reaction mixture was sampled and analyzed by HPLC (Agilent, C-18 column with water/ACN gradient with 0.05% v/v TFA from 5-95% v/v ACN over 6 minutes with diode array detector monitoring absorbance). HPLC enabled reaction efficacy to be monitored by quantifying the amount of 2BXy remaining unreacted and the amount of 2-thiazoline-2-thione released (at 280 nm and specific elution time) relative to the theoretical amount of 2-thiazoline-2-thione that should be released. After confirmation of reaction efficacy, polymer was then purified using dialysis with a 10 kDa molecular weight cutoff (MWCO) regenerated cellulose dialysis tubing against 10% DMSO in MeOH followed by dialysis against MeOH and drying under vacuum. Polymer yield was then determined and the functionalized polymer was then dissolved into DMSO at 100 mg/mL. Thermo-responsive and hydrodynamic diameter properties are presented in Table 16.

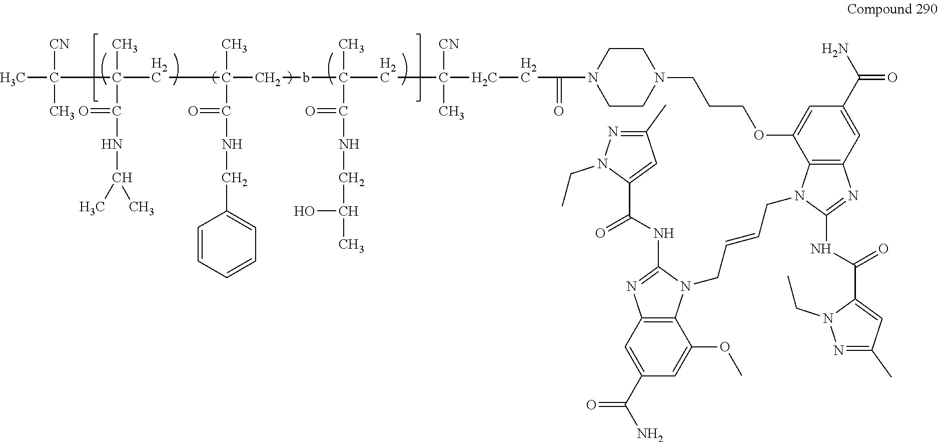

Compound 290. CN-p[(NIPMAM)_{f1}-co-(BnMAM)_{f2}]-b-p(HPMA)ₐ-pip-diABZI. Hydrophilic terminus drug conjugated polymer was prepared by reacting CN-p[(NIPMAM)_{f1}-co-(BnMAM)_{f2}]-b-p(HPMA)ₐ-TT (Compound 232) with hydrophilic terminal functional group TT with small molecule drug diABZI-pip (Compound 33). This reaction was performed similarly to as described for Compound 237 with MA-b-Ala-TT side-chain co-monomer functionalization. Polymer was dissolved in DMSO to a concentration of 200 mg/mL and known mass of diABZI-pip pre-dissolved in DMSO was added at a 2:1 molar ratio to the terminal activated carboxylic acid concentration. Triethylamine was then added (10 equivalents to polymer activated carboxylic acid concentration) and the reaction was accelerated by heating to 40° C. for four hours. At four hours, a portion of the reaction mixture was sampled and analyzed by HPLC (Agilent, C-18 column with water/ACN gradient with 0.05% v/v TFA from 5-95% v/v ACN over 6 minutes with diode array detector monitoring absorbance). HPLC enabled reaction efficacy to be monitored by quantifying the amount of 2BXy remaining unreacted and the amount of 2-thiazoline-2-thione released (at 280 nm and specific elution time) relative to the theoretical amount of 2-thiazoline-2-thione that should be released. After confirmation of reaction efficacy, polymer was then purified using dialysis with a 10 kDa molecular weight cutoff (MWCO) regenerated cellulose dialysis tubing against 10% v/v DMSO in MeOH followed by dialysis against MeOH and drying under vacuum. Polymer yield was then determined and the functionalized polymer was then dissolved into DMSO at 100 mg/mL. Thermo-responsive and hydrodynamic diameter properties are presented in Table 16.

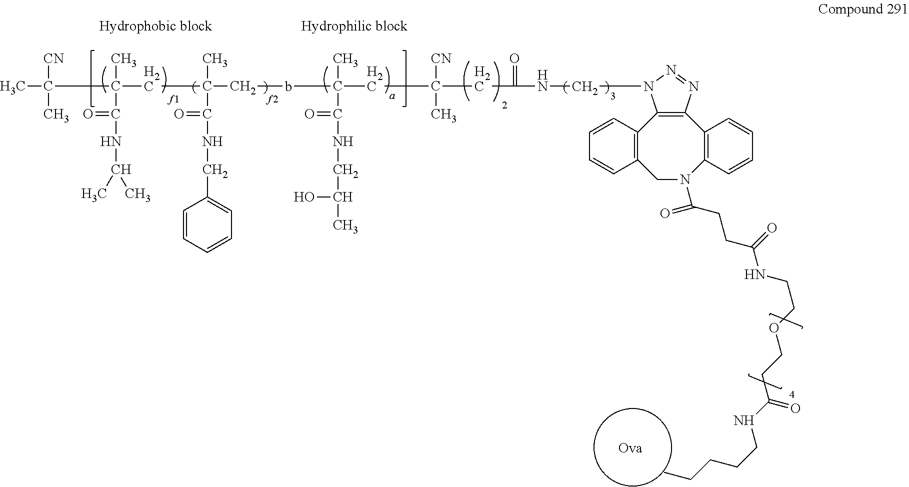

Compound 291. CN-p[(NIPMAM)_{f1}-co-(BnMAM)_{f2}]-b-p(HPMA)ₐ-PEG4-Ova. Ovalbumin was reacted to the hydrophilic terminus of a thermo-responsive polymer using Ova-PEG4-DBCO (Compound 29) and CN-p[(NIPMAM)_{f1}-co-(BnMAM)_{f2}]-b-p(HPMA)ₐ-N3 (Compound 229). For the reaction, CN-p[(NIPMAM)_{f1}-co-(BnMAM)_{f2}]-b-p(HPMA)ₐ-N3 (Compound 229) was dissolved into dimethacetamide at 200 mg/mL and sparged with argon gas for 20 minutes. Ova-PEG4-DBCO was dissolved in 1×PBS at a concentration of 40 mg/mL and similarly sparged with argon. The Ova-PEG4-DBCO was added to the polymer CN-p[(NIPMAM)_{f1}-co-(BnMAM)_{f2}]-b-p(HPMA)ₐ-N3 at a 2:1 molar ratio of DBCO to N3 and additional PBS was added to keep volume fraction of DMAc less than 15% total volume fraction. DMAc at this volume fraction did not impede solubility of ovalbumin protein and increased the solubility of the polymer by helping to prevent micelle formation and encourage reaction between the unimer form of the polymer and the protein. The solution was initially chilled on wet ice (approximately 4° C.) to prevent micelle formation and allowed to warm slowly to room temperature overnight. The reaction solution was then warmed to 40° C. for four hours. Separation of the protein conjugated polymer CN-p[(NIPMAM)_{f1}-co-(BnMAM)_{f2}]-b-p(HPMA)ₐ-PEG4-Ova from unconjugated polymer and unconjugated protein was achieved using size exclusion chromatography with 1×PBS as a mobile phase and a TSKgel G3000SW column (21.5 mm×30 cm) for separation. A preparatory HPLC with a flow rate of 6 mL/minute was used for fraction collection with a sample run-time of 18 minutes with the elution time and absorbance profile (protein absorbing at 280 nm) used to determine material identity in each fraction. Fractions containing CN-p[(NIPMAM)_{f1}-co-(BnMAM)_{f2}]-b-p(HPMA)ₐ-PEG4-Ova were then combined and concentrated three times using a spin column with 10 kDa MWCO, centrifuging for 30 minutes at 3000 rcf. To determine mass concentration, a portion of the crude product was initially separated washed with deionized water using a 10 kDa MWCO spin column to remove PBS salts. After purification, the CN-p[(NIPMAM)_{f1}-co-(BnMAM)_{f2}]-b-p(HPMA)ₐ-PEG4-Ova was concentrated using a 10 kDa MWCO spin column to a known volume and concentration and kept in PBS buffer with storage at −20° C. Thermo-responsive and hydrodynamic diameter properties are presented in Table 16.

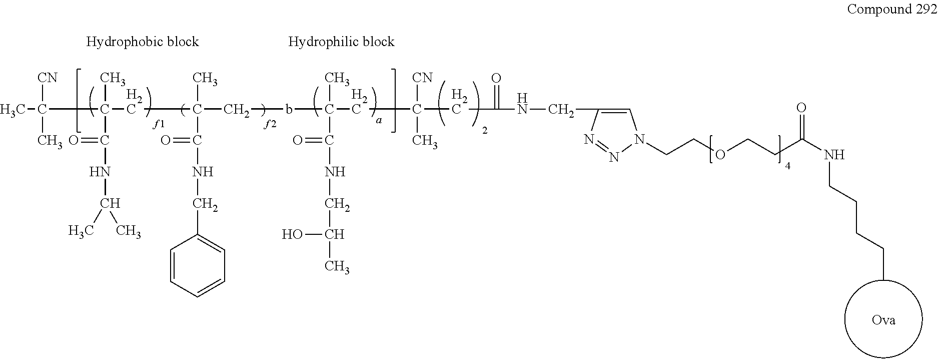

Compound 292. CN-p[(NIPMAM)_{f1}-co-(BnMAM)_{f2}]-b-p(HPMA)ₐ-PEG4-Ova. An alternative route to preparation of ovalbumin conjugated thermo-responsive polymer using Copper catalyzed alkyne azide cycloaddition. Ovalbumin was reacted to the hydrophilic terminus of a thermo-responsive polymer using Ova-PEG4-N3 (Compound 28) and CN-p[(NIPMAM)_{f1}-co-(BnMAM)_{f2}]-b-p(HPMA)ₐ-Pg (Compound 230). For the reaction, CN-p[(NIPMAM)_{f1}-co-(BnMAM)_{f2}]-b-p(HPMA)ₐ-Pg (Compound 230) was dissolved into dimethacetamide at 200 mg/mL and sparged with argon gas for 20 minutes. Ova-PEG4-N3 was dissolved in 1×PBS at a concentration of 40 mg/mL and similarly sparged with argon. Similarly to preparation of Compound 291, the Ova-PEG4-N3 was added to the polymer CN-p[(NIPMAM)_{f1}-co-(BnMAM)_{f2}]-b-p(HPMA)ₐ-Pg at a 2:1 molar ratio of Ova-PEG4-N3 to polymer-Pg and additional PBS was added to keep volume fraction of DMAc less than 15% total volume fraction. Copper ligand tris-hydroxypropyltriazolylmethylamine (THPTA; CAS: 760952-88-3) was dissolved in argon sparged DMF and used to dissolved Copper (I) Bromide (CAS: 7787-70-4) at a 2:1 molar ratio of THPA:CuBr. The CuBr solution was then added to the reaction mixture of Ova-PEG4-N3 and polymer-Pg and reacted as described for Compound 291. Separation of the protein conjugated polymer CN-p[(NIPMAM)_{f1}-co-(BnMAM)_{f2}]-b-p(HPMA)ₐ-PEG4-Ova from unconjugated polymer and unconjugated protein was achieved as described for Compound 291 with an added step of adding 5 equivalents (to CuBr) of chelating ligand disodium edetate (EDTA, CAS: 6381-92-6) to the reaction mixture prior to separation via preparatory HPLC. Thermo-responsive and hydrodynamic diameter properties are presented in Table 16.

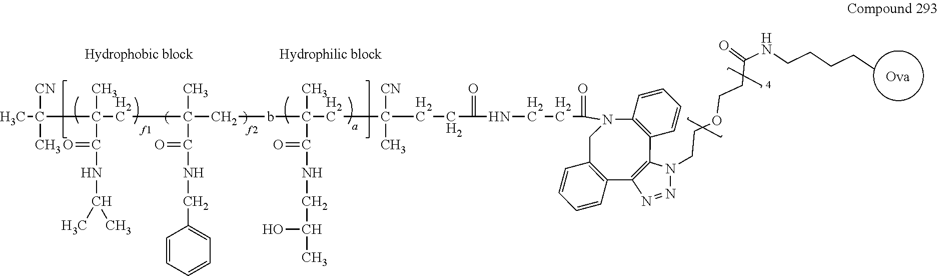

Compound 293. CN-p[(NIPMAM)_{f1}-co-(BnMAM)_{f2}]-b-p(HPMA)ₐ-PEG4-Ova. An alternative route to preparation of ovalbumin conjugated thermo-responsive polymer was performed using strain promoted alkyne azide cycloaddition between Ova-PEG4-N3 (Compound 28) and CN-p[(NIPMAM)_{f1}-co-(BnMAM)_{f2}]-b-p(HPMA)ₐ-DBCO (Compound 231). This reaction and purification was performed near identically as described for Compound 291 using a 2:1 molar ratio of the Ova-PEG4-N3 to CN-p[(NIPMAM)_{f1}-co-(BnMAM)_{f2}]-b-p(HPMA)ₐ-DBCO. This synthesis route has benefits over the route used for Compound 291 by avoiding exposing the DBCO moiety to aqueous conditions until the polymer-DBCO and protein-N3 are mixed together and over the route used for Compound 292 by avoiding the use of CuBr since Cu(II) has toxicity associated concerns. Thermo-responsive and hydrodynamic diameter properties are presented in Table 16.

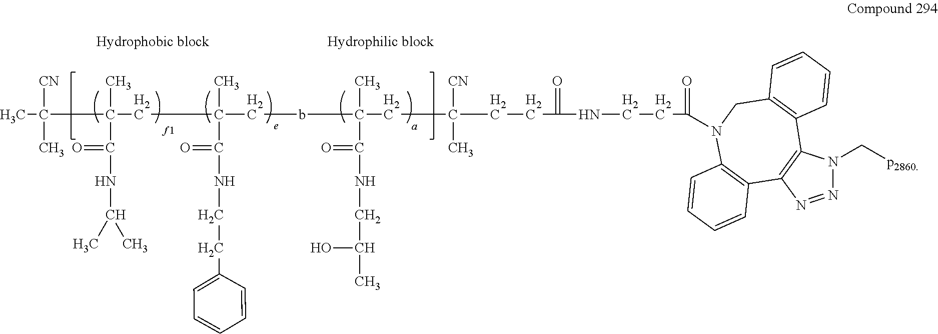

Compound 294. CN-p[(NIPMAM)_{f1}-co-(BnMAM)_{f2}]-b-p(HPMA)ₐ-p2860. Hydrophilic terminus peptide conjugated polymer with 20 mol % BnMAM co-monomer was prepared by reacting CN-p[(NIPMAM)_{f1}-co-(BnMAM)_{f1}]-b-p(HPMA)ₐ-DBCO (Compound 231) with peptide drug molecule p2860 (Compound 31) using strain promoted alkyne azide cycloaddition. The polymer terminal DBCO group containing a strained alkyne was reacted with the azide residue of p2860. This reaction was performed by dissolving polymer in DMSO to a concentration of 200 mg/mL and mixing with a known mass of peptide p2860 pre-dissolved in DMSO at a 2:1 molar ratio to the terminal polymer DBCO concentration. The reaction was then accelerated by heating to 40° C. for four hours. At four hours, a portion of the reaction mixture was sampled and analyzed by HPLC (Agilent, C-18 column with water/ACN gradient with 0.05% v/v TFA from 5-95% v/v ACN over 6 minutes with diode array detector monitoring absorbance). HPLC enabled reaction efficacy to be monitored by quantifying the amount of p2860 remaining unreacted. After confirmation of reaction efficacy, polymer was then purified from free peptide using dialysis with a 10 kDa molecular weight cutoff (MWCO) regenerated cellulose dialysis tubing against 10% DMSO in MeOH followed by dialysis against MeOH and drying under vacuum. Polymer yield was then determined and the functionalized polymer was then dissolved into DMSO at 100 mg/mL. Thermo-responsive and hydrodynamic diameter properties are presented in Table 16.

TABLE 16
DLS characterization results at low concentration for drug conjugate polymers Compounds 287-293.
D_{H} is the hydrodynamic diameter at either 20° C. (D_{H}^{20° C.}) or at 37° C. (D_{H}^{37° C.}) for diblock
copolymer dissolved in PBS at a concentration of 0.5 mg/mL. Polymers exhibiting a poorly defined
transition temperature are labelled as (Ind.) for indecisive, while polymers exhibiting no
temperature-responsive properties for the temperature range of 4° C.-37° C. are listed as (None).
Polymers not tested for temperature-responsive properties are listed as (N.D.) for no data.

| Cmpd | | Tₜᵣ | D_{H}^{20° C.} | D_{H}^{37° C.} |
|---|---|---|---|---|
| # | Structure | (° C.) | (nm) | (nm) |
| 287 | CN-p[(NIPMAM)_{f1}-co-(MA-b-Ala- | None | 33.9 | 35.0 |
| | 2BXy)ₑ]-b-p(HPMA)ₐ-p2860 | | | |
| 288 | CN-p[(NIPMAM)_{f1}-co-(BnMAM)_{f2}]-b- | 26 | 10.7 | 22.7 |
| | p(HPMA)ₐ-co-(MA-b-Ala-2BXy)ₐ]-p2860 | | | |
| 289 | CN-p[(NIPMAM)_{f1}-co-(BnMAM)_{f2}]-b- | 30 | 10.4 | 30.9 |
| | p(HPMA)ₐ-2BXy | | | |
| 290 | CN-p[(NIPMAM)_{f1}-co-(BnMAM)_{f2}]-b- | 31 | 15.3 | 25.3 |
| | p(HPMA)ₐ-pip-diABZI | | | |
| 291 | CN-p[(NIPMAM)_{f1}-co-(BnMAM)_{f2}]-b- | 28 | 7.4 | 40.4 |
| | p(HPMA)ₐ-PEG4-Ova | | | |
| 292 | CN-p[(NIPMAM)_{f1}-co-(BnMAM)_{f2}]-b- | 32 | 6.1 | 36.5 |
| | p(HPMA)ₐ-PEG4-Ova | | | |
| 293 | CN-p[(NIPMAM)_{f1}-co-(BnMAM)_{f2}]-b- | 29 | 5.7 | 41.1 |
| | p(HPMA)ₐ-PEG4-Ova | | | |
| 294 | CN-p[(NIPMAM)_{f1}-co-(BnMAM)_{f2}]-b- | 25 | 6.8 | 33.2 |
| | p(HPMA)ₐ-p2860 | | | |

### Example 6. Control Materials Used for Comparison

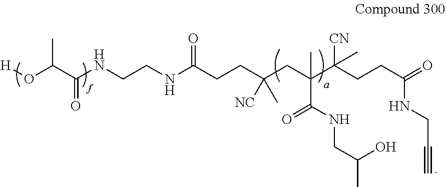

Compound 300. PLA-b-p(HPMA)ₐ-Pg. A non-thermo-responsive amphiphile using a poly(lactide) hydrophobic block was prepared. Poly(Lactide)-block-poly(hydroxypropylmethacrylamide) di-block copolymer (PLA-b-p(HPMA)ₐ-Pg) was synthesized via conjugation of 10 kDa poly(D,L-Lactide)-NH₂ (PLA) (PolySciTech, 10 kDa, Xₙ=140, AI041) with TT-p(HPMA)ₐ-Pg (Compound 124). For the reaction, PLA-NH₂ (6.24 mg, 0.62 μmol) was dissolved in 311 μL of tetrahydrofuran. TT-p(HPMA)-Pg (19.2 mg, 0.62 μmol) was dissolved in 192 μL of tetrahydrofuran with sonication. PLA and TT-p(HPMA)-Pg solutions were then combined and 10 equivalents of triethylamine (0.627 mg, 6.19 μmol) in tetrahydrofuran was added. The reaction mixture was heated to 40° C. for 6 hours. PLA-b-p(HPMA)-Pg was then isolated by precipitation in diethyl ether, enabling removal of any unreacted PLA-NH₂ blocks. The precipitated polymer was dried under vacuum to yield an off-white solid (19 mg, 75% yield). Reaction efficacy was confirmed by ¹H NMR.

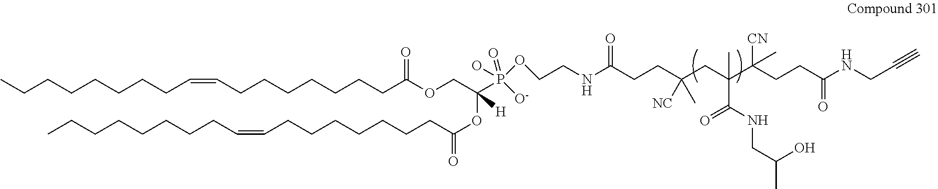

Compound 301. DOPE-b-p(HPMA)ₐ-Pg. A lipid tail hydrophobic block amphiphile with poly(hydroxypropylmethacrylamide) (DOPE-b-p(HPMA)ₐ-Pg) was synthesized by reacting TT-p(HPMA)-Pg (Compound 124) with DOPE. DOPE lipid (CAS 4004-05-1) was purchased from Avanti Polar Lipids. To synthesize, DOPE and TT-p(HPMA)-Pg were dissolved in anhydrous methanol at a 5:1 molar ratio with the addition of 10 equivalents of triethylamine and heated to 40° C. for 6 hours. DOPE-b-p(HPMA)-Pg was then isolated by precipitation in diethyl ether to remove excess DOPE lipid followed by drying in under vacuum overnight to yield an off-white solid (19.6 mg, 78.5% yield). Reaction efficacy was confirmed via ¹H NMR.

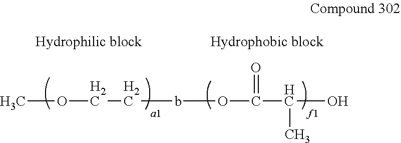

Compound 302. PEG(10k)-PLA(5k). Polyethylene glycol-block-poly(lactic acid) di-block copolymer was purchased from Polysciences (Product #: 25017) and used without modification. Block ratio was 10-b-5 kDa with degree of polymerization approximately 222-b-67 for PEG-b-PLA respectively.

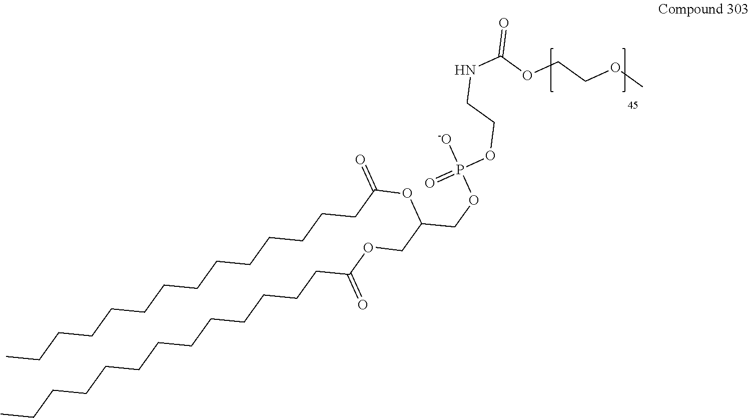

Compound 303. DMG-PEG2k. A poly(ethylene glycol) phospholipid, 1,2-dimyristoyl-rac-glycero-3-methoxypolyethylene glycol-2000 was purchased from Avanti Lipids (Product #: 880151, CAS 160743-62-4) and used without modification.

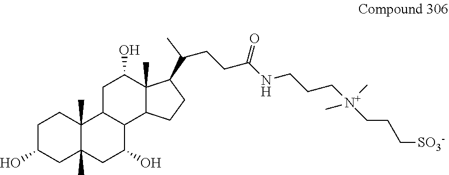

Compound 306. CHAPS. Cholestroyl lipid 3-[(3-Cholamidopropyl)dimethylammonio]-1-propanesulfonate was purchased from Avanti Lipids (Product #: 850500P, CAS 75621-03-3) and used without modification.

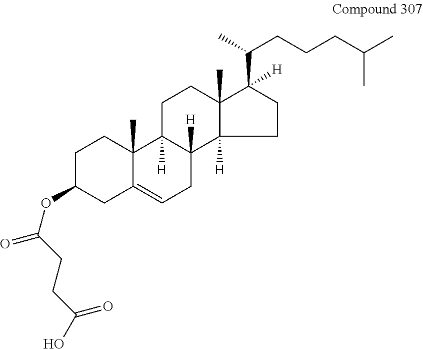

Compound 307. CHEMS Cholesteryl hemisuccinate was purchased from Avanti Lipids (Product #, CAS 75621-03-3) and used without modification.

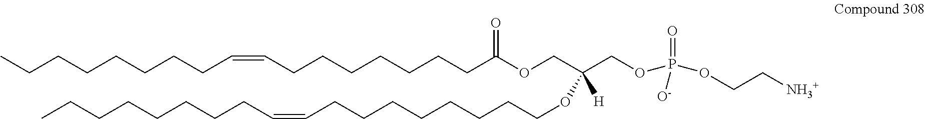 [SMILES: CCCCCCCC/C=C\CCCCCCCCO[C@H](COC(=O)CCCCCCC/C=C\CCCCCCCC)COP(=O)([O-])OCC[NH3+]]

Compound 308. DOPE. Phospholipid 1,2-dioleoyl-sn-glycero-3-phosphoethanolamine was purchased from Avanti Lipids (Product #: 850725C, CAS 4004-05-1) and used without modification.

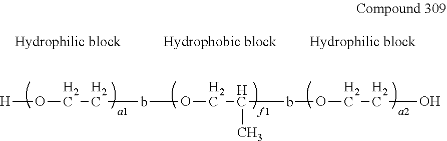

Compound 309. F127. Pluronic tri-block co-polymer poly(ethylene-glycol)-b-poly(propylene-glycol)-b-poly(ethylene-glycol) (F127) was purchased from Sigma Aldrich (Product #: P2443) and used without modification. Block lengths are approximately 101-56-101 for PEGₐ₁-PPO_{f1}-PEGₐ₂ respectively. F127 is also known as poloxamer 407.

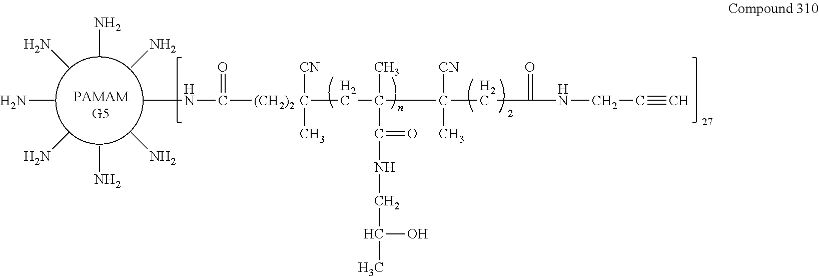

PAMAM(G5)-[p(HPMA30 kDa)]₂₇ PAMAM(G5)-[p(HPMA30 kDa)]₂₇ Compound 310. PAMAM(G5)-[p(HPMA30 kDa)]₂₇. Star-polymer was prepared by reacting PAMAM dendrimer generation 5 (CAS: 163442-68-0, 5 wt % in MeOH) with 31 kDa TT-p(HPMA)ₐ-Pg (Compound 124) as described in patent WO/2020/214858. Star-polymer was purified from unreacted polymer arms TT-p(HPMA)ₐ-Pg using a stirred cell with 100 kDa MWCO filtration. After separation, PAMAM(G5)-[p(HPMA30 kDa)]₂₇ had a Mn of 1061.90 kDa with PDI of 1.087, equating to a mean Star-polymer arm number of 26.9.

### Example 7

Hydrodynamic behavior, thermo-responsiveness, and solubility under aqueous conditions. Prepared polymers and control materials were characterized for nanoparticle hydrodynamic diameter by dynamic light scattering (DLS), thermo-responsivity and overall solubility in PBS.

TABLE 17
DLS characterization results at low concentration for diblock copolymers
(Compounds 200-236) and control materials (Compounds 124, 300-308).

| Cmpd | | Mol % | Block | Tₜᵣ | D_{H}^{20° C.} | D_{H}^{37°C.} |
|---|---|---|---|---|---|---|
| # | Structure | f2 or e1 | ratio | (° C.) | (nm) | (nm) |
| 200 | CN-p[(NIPMAM)_{f1}-co-(BnMAM)_{f2}]-b- | 20 | 1.73 | 27 | 8.1 | 48.6 |
| | p(HPMA)ₐ-DTB | | | | | |
| 201 | CN-p[(NIPMAM)_{f1}-co-(BnMAM)_{f2}]-b- | 10 | 0.61 | 34 | 7.5 | 83.0 |
| | p(HPMA)ₐ-DTB | | | | | |
| 202 | CN-p[(NIPMAM)_{f1}-co-(BnMAM)_{f2}]-b- | 10 | 1.51 | 38 | 9.1 | 71.4 |
| | p(HPMA)ₐ-DTB | | | | | |
| 203 | CN-p[(NIPMAM)_{f1}-co-(BnMAM)_{f2}]-b- | 10 | 2.45 | 38 | 11.5 | 107.7 |
| | p(HPMA)ₐ-DTB | | | | | |
| 204 | CN-p[(NIPMAM)_{f1}-co-(BnMAM)_{f2}]-b- | 15 | 0.62 | 28 | 8.1 | 98.6 |
| | p(HPMA)ₐ-DTB | | | | | |
| 205 | CN-p[(NIPMAM)_{f1}-co-(BnMAM)_{f2}]-b- | 15 | 1.49 | 33 | 10.9 | 69.7 |
| | p(HPMA)ₐ-DTB | | | | | |
| 206 | CN-p[(NIPMAM)_{f1}-co-(BnMAM)_{f2}]-b- | 20 | 0.74 | 25 | 7.9 | 53.0 |
| | p(HPMA)ₐ-DTB | | | | | |
| 207 | CN-p[(NIPMAM)_{f1}-co-(BnMAM)_{f2}]-b- | 20 | 1.42 | 28 | 10.3 | 53.3 |
| | p(HPMA)ₐ-DTB | | | | | |
| 208 | CN-p[(NIPMAM)_{f1}-co-(BnMAM)_{f2}]-b- | 20 | 1.78 | 28 | 11.7 | 66.9 |
| | p(HPMA)ₐ-DTB | | | | | |
| 209 | CN-p[(NIPMAM)_{f1}-co-(BnMAM)_{f2}]-b- | 25 | 1.69 | 21 | 10.8 | 38.8 |
| | p(HPMA)ₐ-DTB | | | | | |
| 210 | CN-p[(NIPMAM)_{f1}-co-(BnMAM)_{f2}]-b- | 30 | 2.79 | 24 | 5.3 | 38.9 |
| | p(HPMA)ₐ-DTB | | | | | |
| 211 | CN-p[(NIPMAM)_{f1}-co-(BnMAM)_{f2}]-b- | 30 | 0.90 | 18 | 18.2 | 30.9 |
| | p(HPMA)ₐ-DTB | | | | | |
| 212 | CN-p[(NIPMAM)_{f1}-co-(BnMAM)_{f2}]-b- | 50 | 2.44 | None | 33.3 | 36.7 |
| | p(HPMA)ₐ-DTB | | | | | |
| 213 | CN-p[(NIPMAM)_{f1}-co-(BnMAM)_{f2}]-b- | 50 | 0.65 | None | 22.3 | 20.4 |
| | p(HPMA)ₐ-DTB | | | | | |
| 214 | CN-p(BnMAM)_{f2}-b-p(HPMA) ₐ-DTB | 100 | 1.12 | None | 41.7 | 95.0 |
| 215 | CN-p(BnMAM)_{f2}-b-p(HPMA) ₐ-DTB | 100 | 1.84 | None | 48.3 | 40.5 |
| 216 | CN-p[(NIPMAM)_{f1}-co-(MA-b-Ala- | 20 | 0.79 | N.D. | N.D. | N.D. |
| | TT)ₑ]-b-p(HPMA)-DTB | | | | | |
| 217 | CN-p[(NIPMAM)_{f1}-co-(MA-EDA-Phe- | 5 | 2.12 | 35 | 12.0 | 36.8 |
| | Phe-Boc)_{f2}]-b-p(HPMA)ₐ-DTB | | | | | |
| 218 | CN-p[(NIPMAM)_{f1}-co-(MA- | 5 | 0.39 | 35 | 8.7 | 35.9 |
| | BTFMB)[1]-b-p(HPMA)ₐ-DTB | | | | | |
| 219 | CN-p[(NIPMAM)_{f1}-co-(MA- | 5 | 3.76 | Ind. | N.D. | N.D. |
| | BTFMB)_{f1}]-b-p(HPMA)ₐ-DTB | | | | | |
| 220 | CN-p[(HPMA)ₐ₁-co-(BnMAM)_{f1}]-b- | 20 | 2.24 | None | 7.9 | 8.7 |
| | p(HPMA)ₐ₂-DTB | | | | | |
| 221 | CN-p[(HPMA)ₐ₁-co-(BnMAM)_{f1}]-b- | 30 | 1.17 | None | 97.6 | 87.1 |
| | p(HPMA)ₐ₂-DTB | | | | | |
| 222 | CN-p[(HPMA)ₐ₁-co-(BnMAM)_{f1}]-b- | 50 | 0.14 | None | 87.1 | 76.1 |
| | p(HPMA)ₐ₂-DTB | | | | | |
| 223 | CN-p[(TEGMA)_{f1}-co-(BnMAM)_{f2}]-b- | 15 | 0.29 | 36 | 6.0 | 25.2 |
| | p(HPMA)ₐ]-DTB | | | | | |
| 224 | CN-p[(TEGMA)_{f1}-co-(BnMAM)_{f2}]-b- | 20 | 0.44 | 34 | 7.7 | 28.3 |
| | p(HPMA)ₐ]-DTB | | | | | |
| 225 | CN-p[(NIPMAM)_{f1}-co-(BnMAM)_{f2}]-b- | 20 | 2.16 | 21 | 11.9 | 39.0 |
| | p(HEMAM)ₐ-DTB | | | | | |
| 226 | CN-p[(NIPMAM)_{f1}-co-(BnMAM)_{f2}]-b- | 20 | 1.08 | 24 | 6.1 | 61.8 |
| | p(HEA)ₐ-DTB | | | | | |
| 227 | CN-p[(NIPMAM)_{f1}-co-(BnMAM)_{f2}]-b- | 20 | 3.87 | N.D. | N.D. | N.D. |
| | p[(HPMA)ₐ-co-(MA-b-Ala-TT)ₑ]-DTB | mol % | | | | |
| | | f2, 10 | | | | |
| | | mol % e | | | | |
| 228 | N3- p(HPMA)ₐ-b-p[(NIPMAM)_{f1}-co- | 20 | 1.32 | 23 | 9.5 | 33.1 |
| | (BnMAM)_{f2}]-DTB | | | | | |
| 229 | CN-p[(NIPMAM)_{f1}-co-(BnMAM)_{f2}]-b- | 20 | 1.72 | 27 | 8.4 | 41.8 |
| | p(HPMA)ₐ-N3 | | | | | |
| 230 | CN-p[(NIPMAM)_{f1}-co-(BnMAM)_{f2}]-b- | 20 | 1.72 | 22 | 8.7 | 36.4 |
| | p(HPMA)ₐ-Pg | | | | | |
| 231 | CN-p[(NIPMAM)_{f1}-co-(BnMAM)_{f2}]-b- | 20 | 1.72 | 23 | 7.1 | 33.8 |
| | p(HPMA)ₐ-DBCO | | | | | |
| 232 | CN-p[(NIPMAM)_{f1}-co-(BnMAM)_{f2}]-b- | 20 | 1.72 | N.D. | N.D. | N.D. |
| | p(HPMA)ₐ-TT | | | | | |
| 233 | CN-p[(NIPMAM)_{f1}-co-(MA-b-Ala- | 20 | 1.24 | N.D. | N.D. | N.D. |
| | TT)ₑ]-b-p(HPMA)ₐ-Pg | | | | | |
| 234 | CN-p[(NIPMAM)_{f1}-co-(MA-b-Ala- | 20 | 1.24 | N.D. | N.D. | N.D. |
| | TT)ₑ]-b-p(HPMA)ₐ-DBCO | | | | | |
| 235 | CN-p[(NIPMAM)_{f1}-co-(BnMAM)_{f2}]-b- | 20 | 1.97 | N.D. | N.D. | N.D. |
| | p[(HPMA)ₐ-co-(MA-b-Ala-TT)ₑ]-Pg | | | | | |
| 236 | CN-p[(NIPMAM)_{f1}-co-(BnMAM)_{f2}]-b- | 20 | 1.97 | N.D. | N.D. | N.D. |
| | p[(HPMA)ₐ-co-(MA-b-Ala-TT)ₑ]-Pg | | | | | |
| 124 | TT-p(HPMA)ₐ-Pg | 0 | N.A. | None | 4.9 | 5.4 |
| 300 | PLA-b-p(HPMA)ₐ-Pg | N.A. | N.A. | None | 7.3 | 7.9 |
| 301 | DOPE-p(HPMA)ₐ-Pg | N.A. | N.A. | None | 6.7 | 6.7 |
| 302 | PEG(10 k)-PLA(5 k) | N.A. | N.A. | None | 40.5 | 42.9 |
| 303 | DMG-PEG2k | N.A. | N.A. | None | 6.5 | 7.4 |
| 304 | CHAPS lipid | N.A. | N.A. | None | 5.9 | 5.0 |
| 305 | CHEMS lipid | N.A. | N.A. | None | 296.1 | 363.4 |
| 306 | DOPE lipid | N.A | N.A. | None | 143.5 | 146.9 |
| 307 | F127 (PEO-PPO-PEO) | N.A. | 3.6 | 33 | 3.4 | 21.1 |
| 308 | PAMAM(G5)-[p(HPMA30kDa)]₂₇ | N.A. | N.A. | None | 24.7 | 27.0 |

Block ratio used here refers to the degree of polymerization block ratio of hydrophilic block to hydrophobic block. D_{H} is the hydrodynamic diameter at either 20° C. (D_{H}^{20° C.}) or at 37ºC (D_{H}^{37° C.}) for diblock copolymer dissolved in PBS at a concentration of 0.5 mg/mL. Polymers exhibiting a poorly defined transition temperature are labelled as (Ind.) for indecisive, while polymers exhibiting no temperature-responsive properties for the temperature range of 4° C.-37° C. are listed as (None). Polymers not tested for temperature-responsive properties are listed as (N.D.) for no data.

TABLE 18
DLS characterization results demonstrating concentration dependent
responsive properties for di-block copolymers.

| | | | | | | 0.5 |
|---|---|---|---|---|---|---|
| | | | | Sol. | Sol. | mg/m |
| | | | Sol. | Limit | Limit | L |
| | | | Limit | D_{H}^{20° C.} | D_{H}^{37° C.} | D_{H}^{37° C.} |
| Cmpd # | Structure | Mol % f2 | (mg/mL) | (nm) | (nm) | (nm) |
| 200 | CN-p[(NIPMAM)_{f1}-co- | 20 | 200 | 6.9 | 7.2 | 48.6 |
| | (BnMAM)_{f2}]-b-p(HPMA)ₐ-DTB | | | | | |
| 210 | CN-p[(NIPMAM)_{f1}-co- | 30 | 150 | 6.0 | 7.6 | 38.9 |
| | (BnMAM)_{f2}]-b-p(HPMA)ₐ-DTB | | | | | |
| 211 | CN-p[(NIPMAM)_{f1}-co- | 30 | 150 | 7.7 | 5.8 | 30.9 |
| | (BnMAM)_{f2}]-b-p(HPMA)ₐ-DTB | | | | | |
| 212 | CN-p[(NIPMAM)_{f1}-co- | 50 | 100 | 7.0 | 7.3 | 36.7 |
| | (BnMAM)_{f2}]-b-p(HPMA)ₐ-DTB | | | | | |
| 213 | CN-p[(NIPMAM)_{f1}-co- | 50 | 100 | 29.2 | 37.3 | 20.4 |
| | (BnMAM)_{f2}]-b-p(HPMA)ₐ-DTB | | | | | |
| 214 | CN-p(BnMAM)f-b-p(HPMA) ₐ- | N.A. | 100 | Agg. | Agg. | 95.0 |
| | DTB | | | | | |
| 215 | CN-p(BnMAM)_{f1}-b-p(HPMA) ₐ- | N.A. | 100 | Agg. | Agg. | 40.5 |
| | DTB | | | | | |
| 220 | CN-p[(HPMA)ₐ₁-co- | 20 | 200 | Agg. | Agg. | 9.1 |
| | (BnMAM)_{f1}]-b-p(HPMA)ₐ₂- | | | | | |
| | DTB | | | | | |
| 221 | CN-p[(HPMA)ₐ₁-co- | 30 | 200 | Agg. | Agg. | 87.1 |
| | (BnMAM)_{f1}]-b-p(HPMA)ₐ₂- | | | | | |
| | DTB | | | | | |
| 222 | CN-p[(HPMA)ₐ₁-co- | 50 | 200 | Agg. | Agg. | 76.1 |
| | (BnMAM)_{f1}]-b-p(HPMA)ₐ₂- | | | | | |
| | DTB | | | | | |
| 124 | TT-p(HPMA)ₐ₁-Pg | N.A. | 200 | 4.9 | 5.8 | 5.4 |
| 300 | P(D,L-LA)-b-p(HPMA)-Pg | N.A. | 100 | 857.5 | N.D. | 7.9 |
| 301 | DOPE-p(HPMA)-Pg | N.A. | 100 | 968.5 | N.D. | 6.7 |
| 302 | PEG(10 k)-PLA(5 k) | N.A. | 100 | 184.2 | N.D. | 42.9 |
| 303 | DMG-PEG2k | N.A. | 100 | 7.0 | N.D. | 7.4 |
| 304 | CHAPS lipid | N.A. | 200 | 6.2 | N.D. | 5.0 |
| 305 | CHEMS lipid | N.A. | <10 | N.D. | N.D. | N.D. |
| 306 | DOPE lipid | N.A. | 12.5 | N.D. | N.D. | N.D. |
| 307 | F127 | N.A. | 50 | N.D. | N.D. | 11.2 |

Compound solubility in PBS (150 mM, pH 7.4) was determined by visual clarity and each compound was dissolved to maximum apparent solubility limit. At solubility limit, hydrodynamic diameter (D_{H}) was acquired at either 20º C. (D_{H}^{20° C.}) or at 37° C. (D_{H}^{37° C.}) for di-block copolymer dissolved in PBS. Compounds not tested for specific hydrodynamic diameter measurements are listed as (N.D.) for no data.

### Example 8—Impact of Hydrophobic Block Composition and Block Ratio on Transition Temperature and Particle Size of Temperature-Responsive Amphiphilic Block Copolymers

It has not been thoroughly investigated how various chemical parameters of amphiphilic block copolymers impact physical characteristics that determine their potential utility for use as drug carriers, particularly for applications requiring high drug concentrations and low viscosity. Therefore, a major objective of the studies described herein were to evaluate how various chemical parameters of amphiphilic block copolymers, including hydrophobic and hydrophilic block monomer composition, comonomer density, and block ratio, impact particle formation and particle characteristics, including size and polydispersity up to extremes of concentrations.

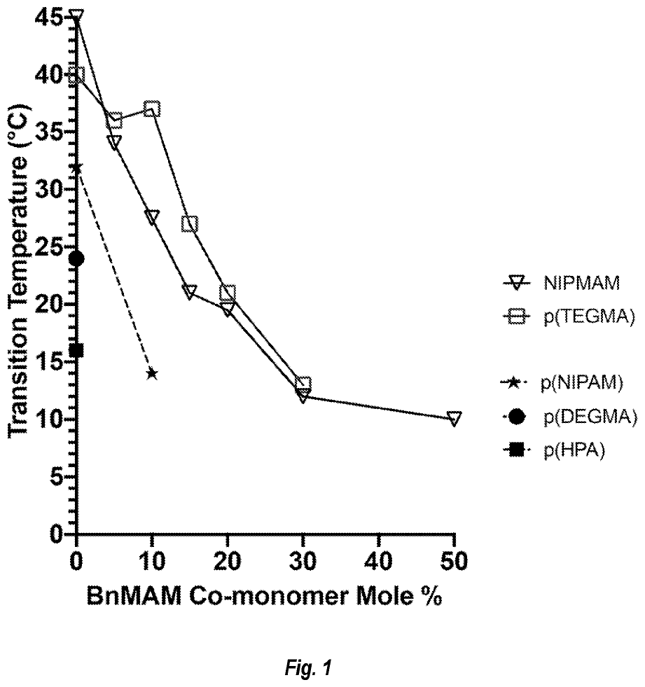

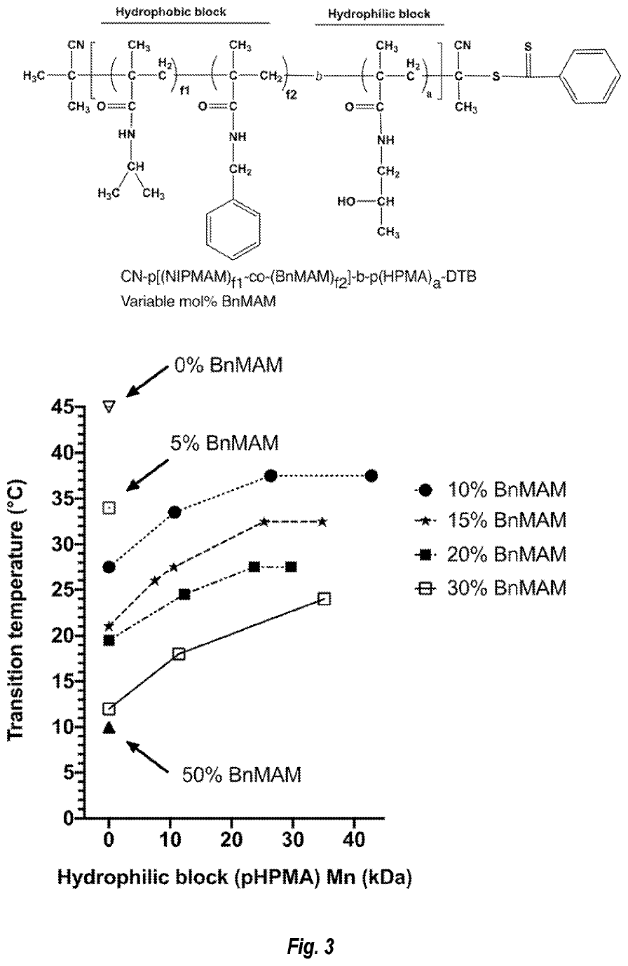

We first investigated the impact that monomer composition and density have on temperature-responsive copolymers. Four different class of hydrophobic methacrylamide-based monomers with either (i) lower alkyl, (ii) higher alkyl, (iii) aromatic or (iv) fluorinated substituent groups were copolymerized with different molar ratios of NIPMAM to generate NIPMAM co-polymers with varying densities of each of the different hydrophobic monomers. Notably, increasing density and molecular weight of the hydrophobic monomers were both inversely related to transition temperature of the resulting copolymers, with fluorinated hydrophobic monomers having the largest impact on transition temperature, followed by hydrophobic monomers comprising higher alkyl groups, aromatic groups and then lower alkyl groups. Accordingly, densities of about >15 mol % lower alkyl substituted hydrophobic monomers, 10-20 mol % aromatic substituted hydrophobic monomers, 1-10 mol % higher alkyl substituted hydrophobic monomers and 1-10 mol % fluorinated hydrophobic monomers were required to yield poly(NIPMAM)-based copolymers with transition temperature between about 20 to 34° C. (see: FIG. 1-5, 23-26 and Table 17), which enabled compositions that exist as monomers at room temperature but form particles at body temperature.

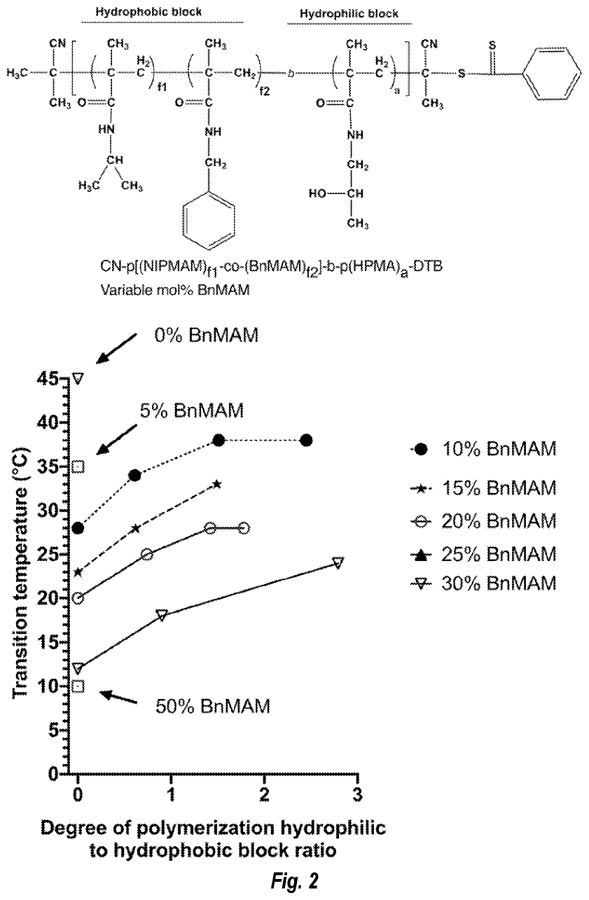

In addition to hydrophobic block monomer composition and density, the degree of polymerization block ratio was also found to impact the transition temperature as well as size of particles formed by amphiphilic block copolymers. For instance, while copolymers comprised of NIPMAM and BnMAM monomers without a hydrophilic block formed aggregates in aqueous solutions above their transition temperature, amphiphilic block copolymers with a block ratio greater than about 0.75:1 comprised of a hydrophilic block consisting of HPMA monomers and a hydrophobic block consisting of NIPMAM and BnMAM monomers formed stable nanoparticle micelles. However, the length of the hydrophilic block also impacted the transition temperature and size of particles formed by amphiphilic block copolymers (see: FIG. 2-5 and Table 17). Accordingly, for hydrophobic copolymers and amphiphilic block copolymers with a hydrophobic block consisting of NIPMAM and BnMAM, with BnMAM at densities between about 5 to 50 mol %, amphiphilic block copolymer with block ratio of about 1:1 had about a 5 to 7° C. higher transition temperature than the hydrophobic copolymer without a hydrophilic block (FIG. 2-5 and Table 17). Increasing the block ratio from about 1:1 to about 2.5:1 resulted in a further increase in transition temperature of about 2-5° C.; and, increasing the block ratio further, from about 2.5:1 to about 4:1 resulted in a further increase in transition temperature of about 1-3° C., suggesting that increasing hydrophilic block length approaches at asymptote for block ratios above 3:1 and that increasing the block ratio above 3:1 has only marginal further impact on the transition temperature.

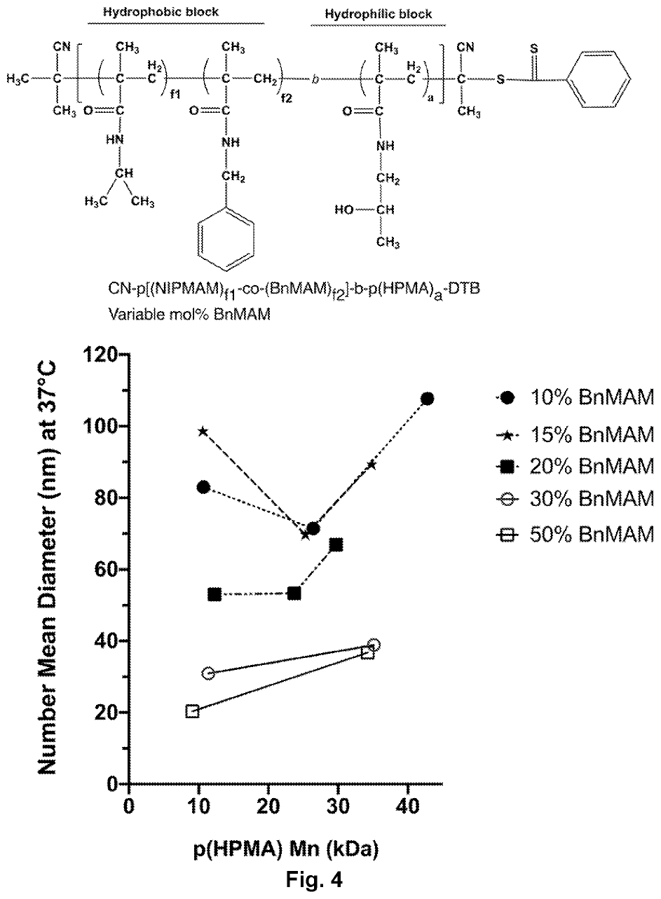

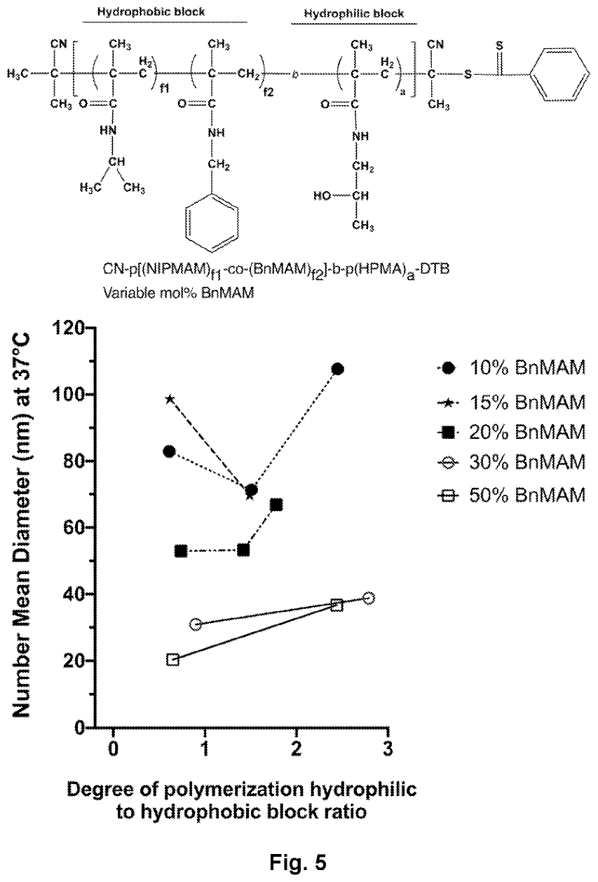

Block ratio and hydrophobic block composition were also found to impact the size of particles formed by amphiphilic block copolymers (FIG. 4,5 and Table 17). Unexpectedly, increasing block ratio and density of the second hydrophobic monomer (i.e., BnMAM) on p[(NIPMAM)_{f1}-co-(BnMAM)_{f2})]-b-p(HPMA)ₐ based amphiphilic block copolymers also had a tendency to drive formation of smaller particle (FIG. 4,5). Non-limiting explanations are that increasing hydrophilic block length stabilizes nanoparticle micelles and prevents formation of larger supramolecular associates, whereas increasing densities (mol %) of the second hydrophobic monomer drives formation of more densely packed particles with smaller hydrodynamic diameter.

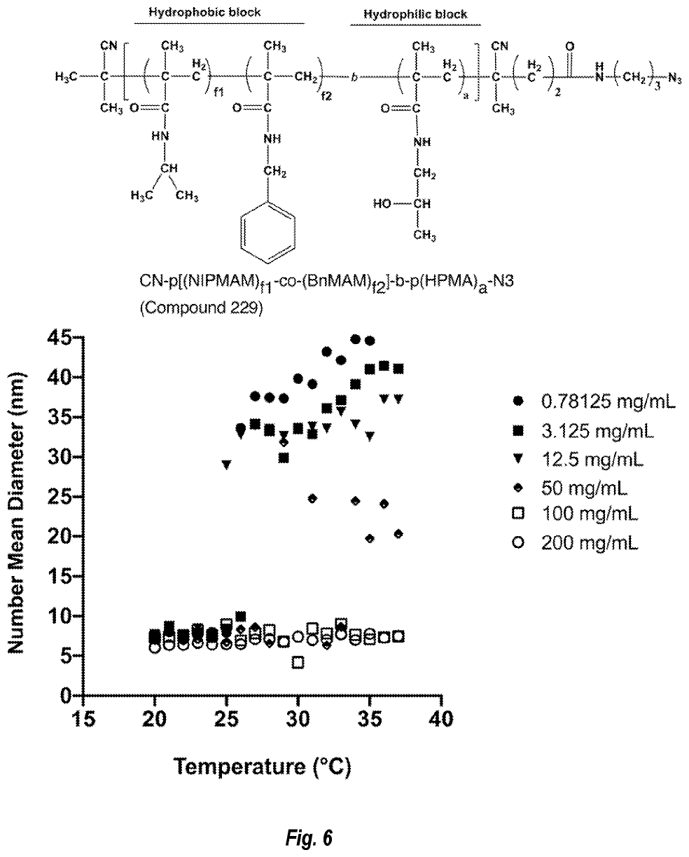

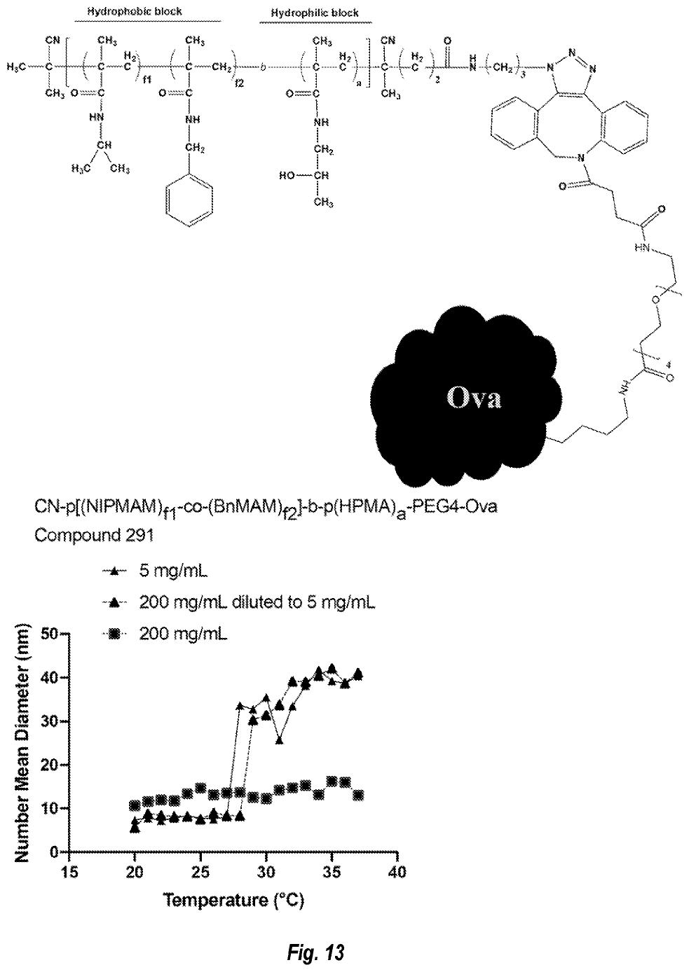

Based on the above results, Compound 200, which has a transition temperature of 28° C. and assembled into ˜53 nm diameters particles in solution above the transition temperature was selected as a representative preferred embodiment of temperature-responsive amphiphilic diblock copolymers. Compound 200 comprises p[(NIPMAM)_{f1}-co-(BnMAM)_{f2}]-b-p(HPMA)ₐ; has a number-average molecular weight (Mn) of about 40 kDa, hydrophilic to hydrophobic block ratio of 1.42 and density of second hydrophobic monomer (BnMAM) of 20 mol %. To evaluate feasibility of use of Compound 200 as an ocular drug delivery system, its hydrodynamic behavior was evaluated over the temperature range of 20 to 37° C. and at concentrations from 1 to 200 mg/mL (FIG. 6 and Table 18). These conditions are meant to model the scenario wherein at room temperature a 0.1 mL volume of an ocular drug delivery system at 200 mg/mL is injected through a narrow gauge (>27-ga) needle into the vitreous (˜4 mL), which is at 32° C. or higher. Notably: for concentrations below 50 mg/mL, the temperature-responsive amphiphilic diblock copolymer (Compound 200) formed stable micelles at temperatures above its transition temperature, whereas at concentrations at or above 50 mg/mL, particularly above 100 mg/mL, its existed as soluble single molecules (unimers) rather than micelles. These data show that temperature-responsive amphiphilic diblock copolymer comprising p[(NIPMAM)_{f1}-co-(BnMAM)_{f2}]-b-p(HPMA)ₐ are also concentration-responsive. These data suggest that Compound 200 and related amphiphilic diblock copolymers are concentration-responsive and provide the benefit that they can be highly concentrated to generate solutions of soluble single molecules that only form particles following dilution after injection into tissue. This property was similarly demonstrated for high (200 mg/mL) concentration thermo-responsive polymer with hydrophilic terminus conjugated protein as shown for p[(NIPMAM)_{f1}-co-(BnMAM)_{f2}]-b-p(HPMA)ₐ-PEG4-Ova (Compound 291) in FIG. 13.

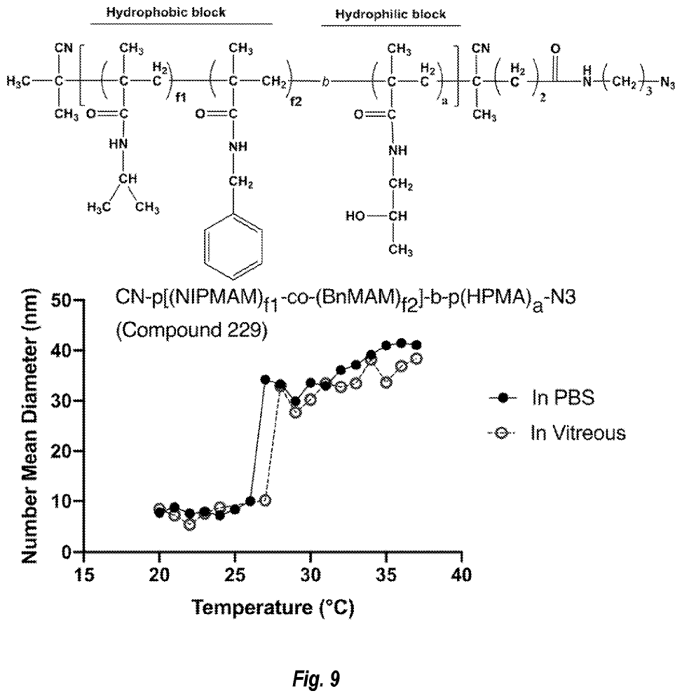

A potential concern for ocular drug delivery systems is that solutes in the vitreous could impact hydrodynamic behavior. Importantly, the hydrodynamic behavior of Compound 200 was found to be similar in both vitreous and PBS buffer (FIG. 9). Rabbit vitreous was liquefied (Bettelheim F. A. et al. Experimental Eye Research 2004, 79 (5), 713-718) to enable identification of nanoparticles in vitreous samples while retaining properties of high protein content vitreous. To mimic an intravitreal injection, Compound 229 at 200 mg/mL in PBS was injected into rabbit vitreous to yield a final polymer concentration of 5 mg/mL. Particle size of Compound 229 in vitreous and PBS over temperatures ranging from 20-37° C. was determined by DLS. The data show that preferred embodiments of temperature-responsive amphiphilic diblock copolymers forms micelles have similar transition temperature and hydrodynamic behavior in vitreous as compared with PBS.

Two potential concerns for any material being administered to patients are cytotoxicity to cells that are exposed and innate immunogenicity to the compounds being injected. The cellular viability of retinal pigmented epithelial like cells (ARPE19) and monocyte like cells (THP1) were assessed following exposure to thermo-responsive polymer at physiologically relevant concentrations.

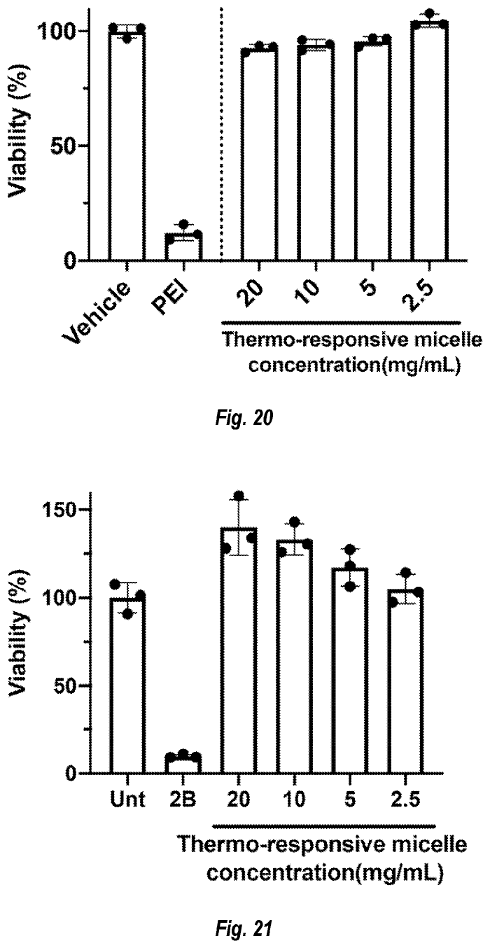

ARPE19 cell viability was not affected by concentration of thermo-responsive micelle ≤20 mg/mL concentration (FIG. 20). ARPE19 cells were grown in vitro in cell growth medium and exposed to thermo-responsive polymer micelle that was resuspended in cell growth medium at the concentrations specified up to 20 mg/mL. In contrast, the negative control, exposure to branched polyethyleneimine 25 kDa (CAS 9002-98-6) at a concentration of 0.04 mg/mL in growth medium, resulted in only 10-20% viability of ARPE19 cells. Cell viability was assessed using Promega CellTiter 96® Non-Radioactive Cell Proliferation Assay (MTT).

THP1 cell viability was likewise not affected by concentrations of thermo-responsive micelle ≤20 mg/mL in concentration (FIG. 21). THP1 cells were grown in vitro in cell growth medium and exposed to thermo-responsive polymer micelle that was resuspended in cell growth medium at the concentrations specified up to 20 mg/mL. In contrast, the negative control, branched immunostimulatory TLR agonist drug 2B at a concentration of 100 μM in growth medium, resulted in only 10-20% viability of THP1 cells. Cell viability was assessed using Promega CellTiter 96® Non-Radioactive Cell Proliferation Assay (MTT).

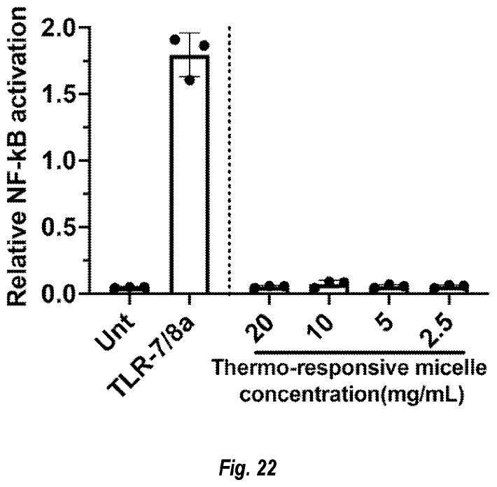

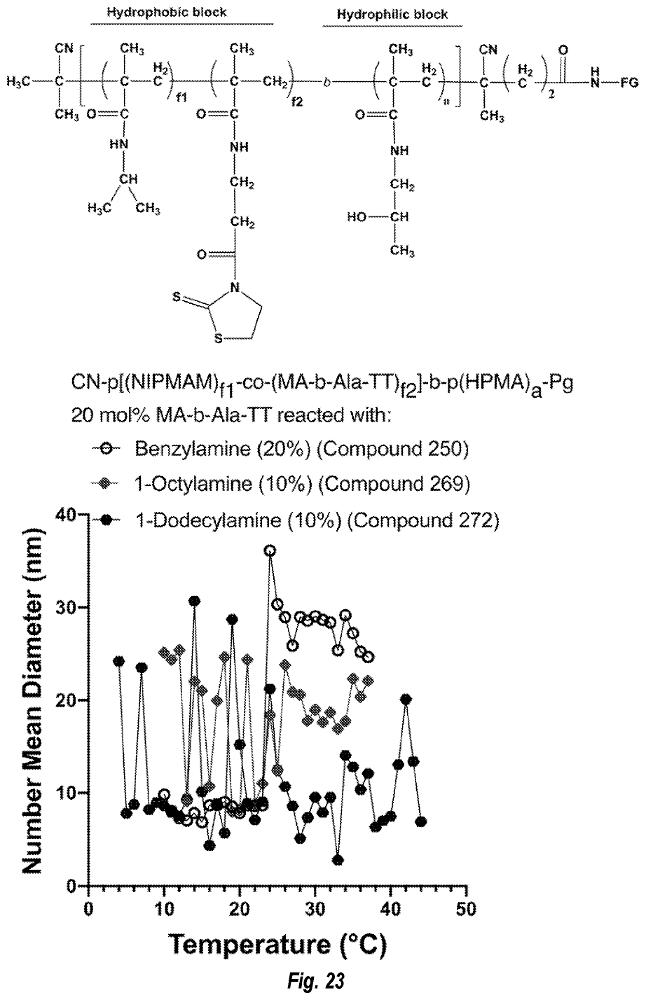

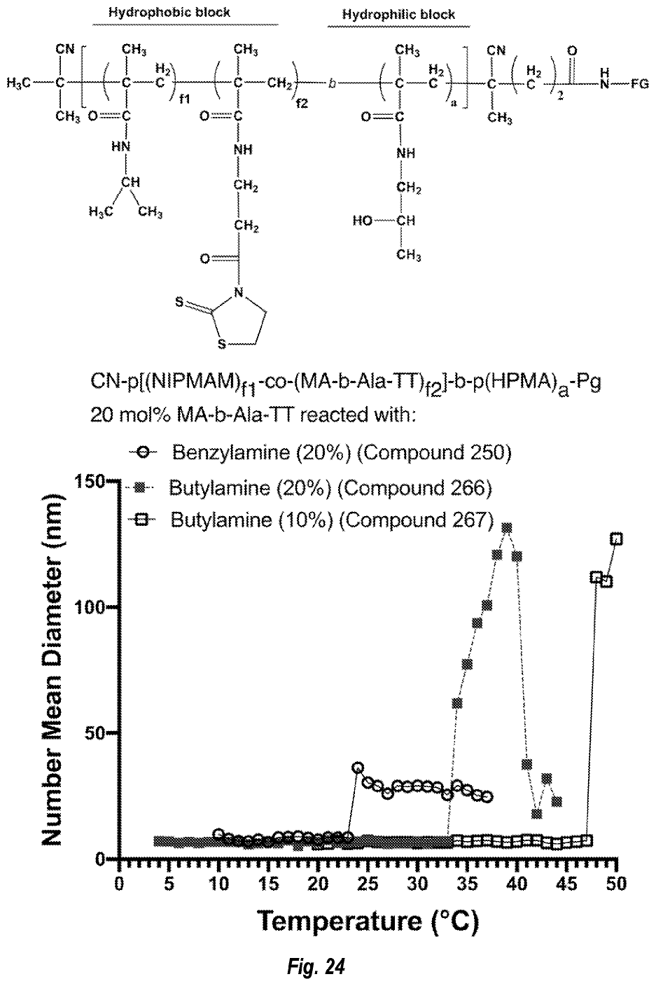

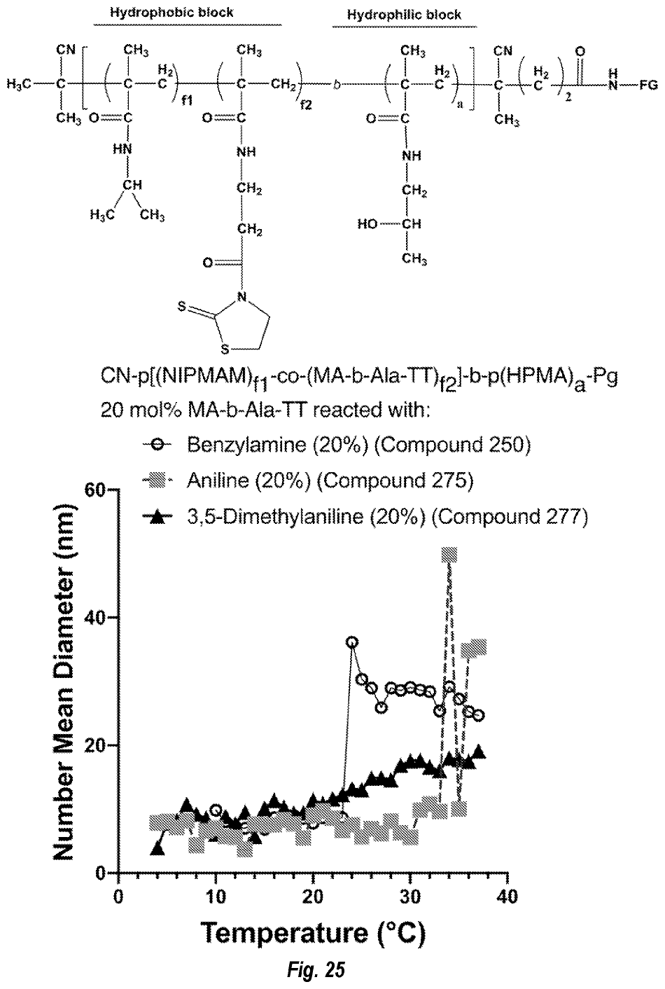

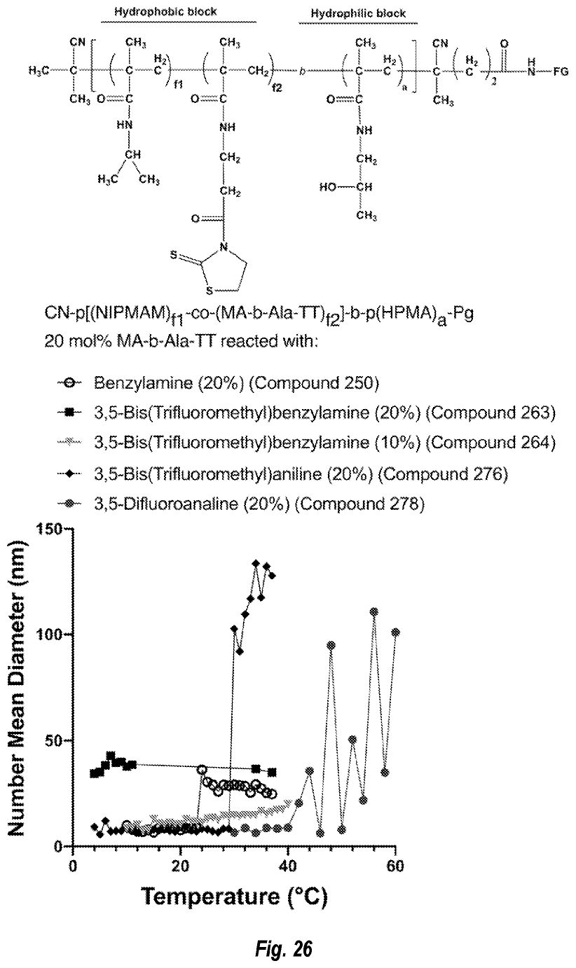

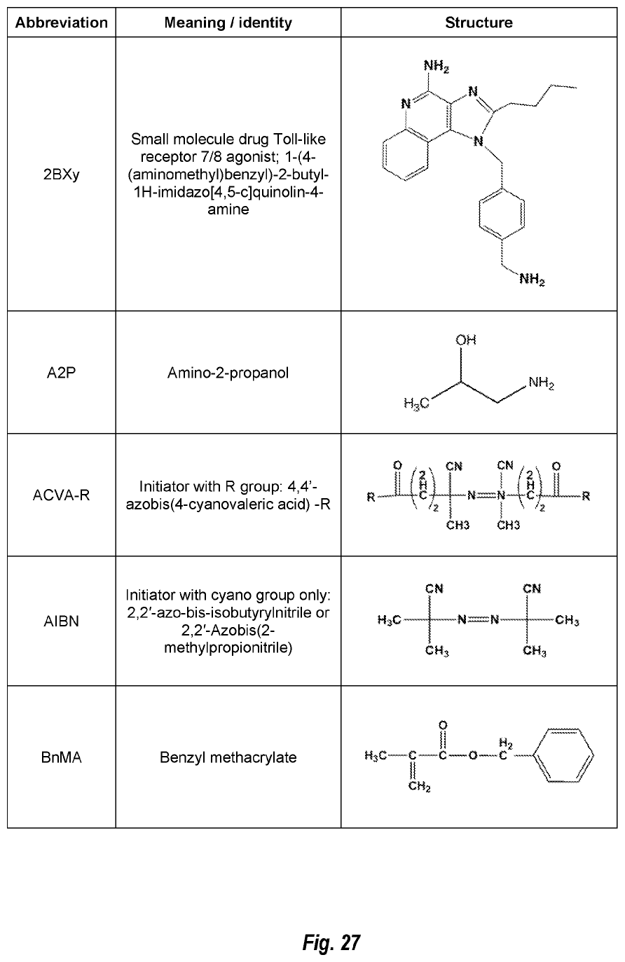

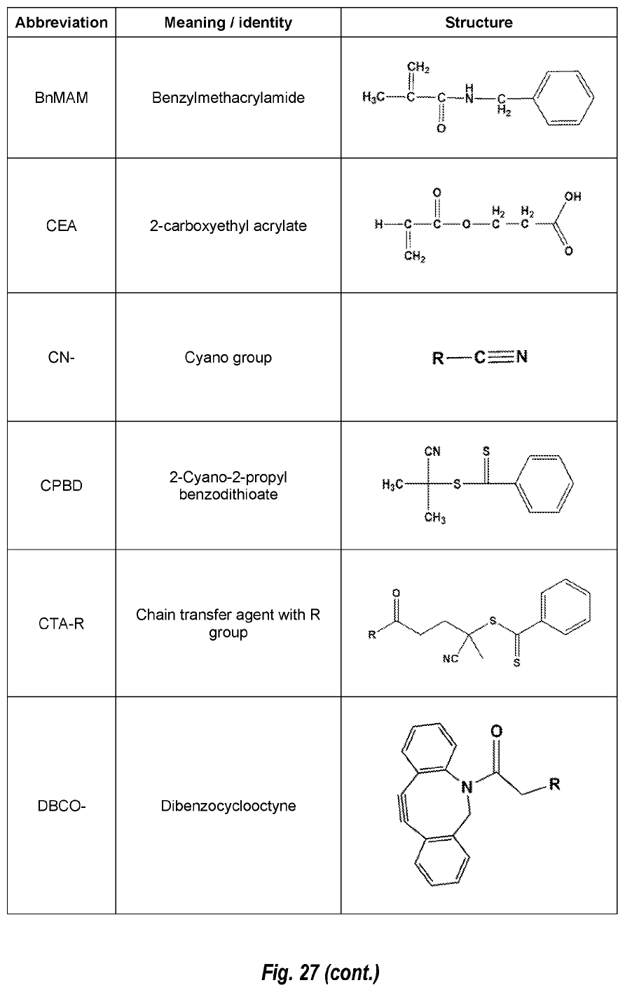

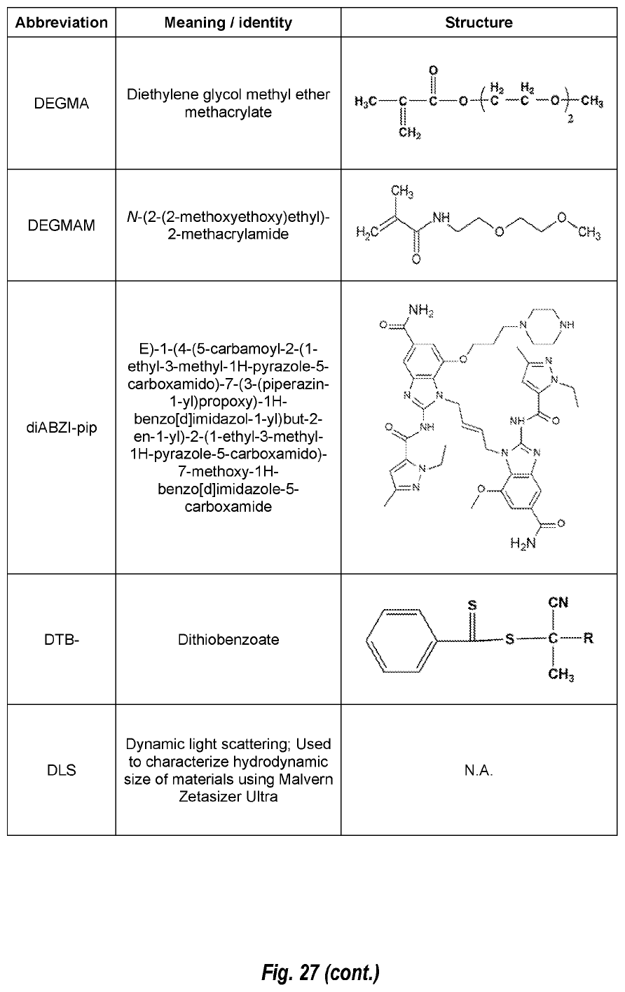

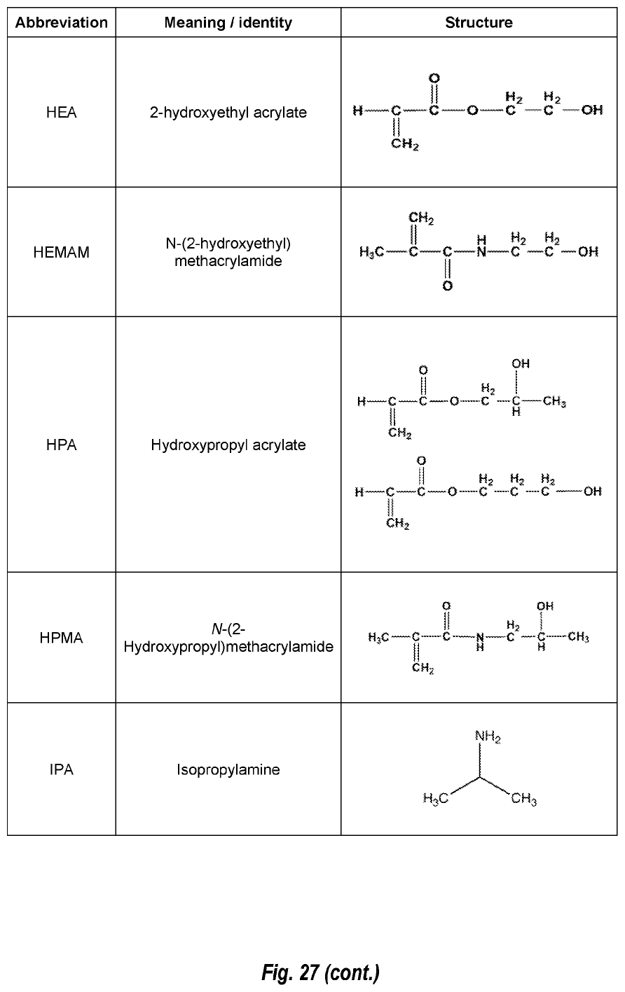

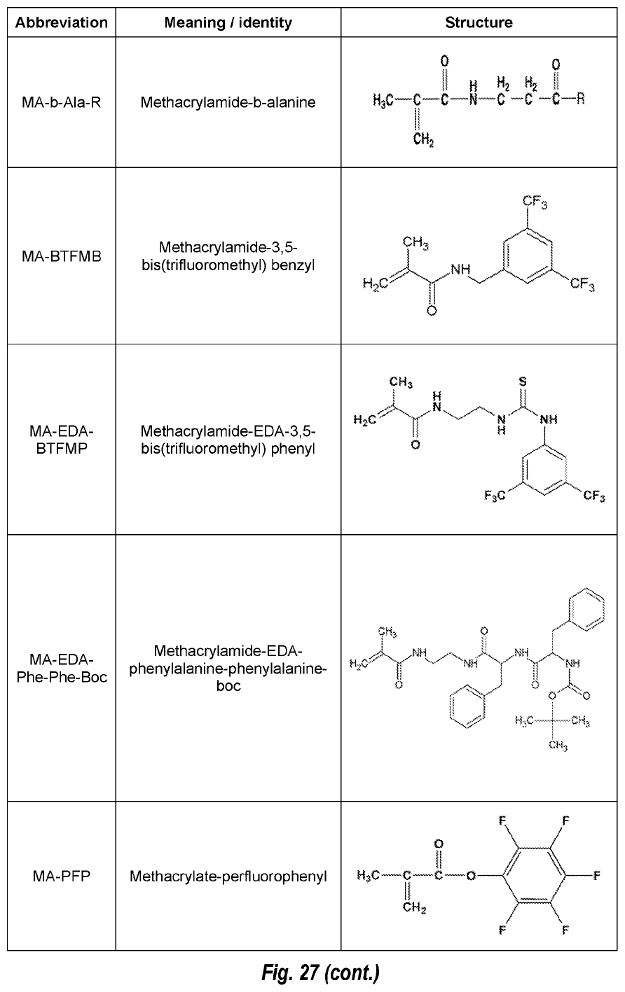

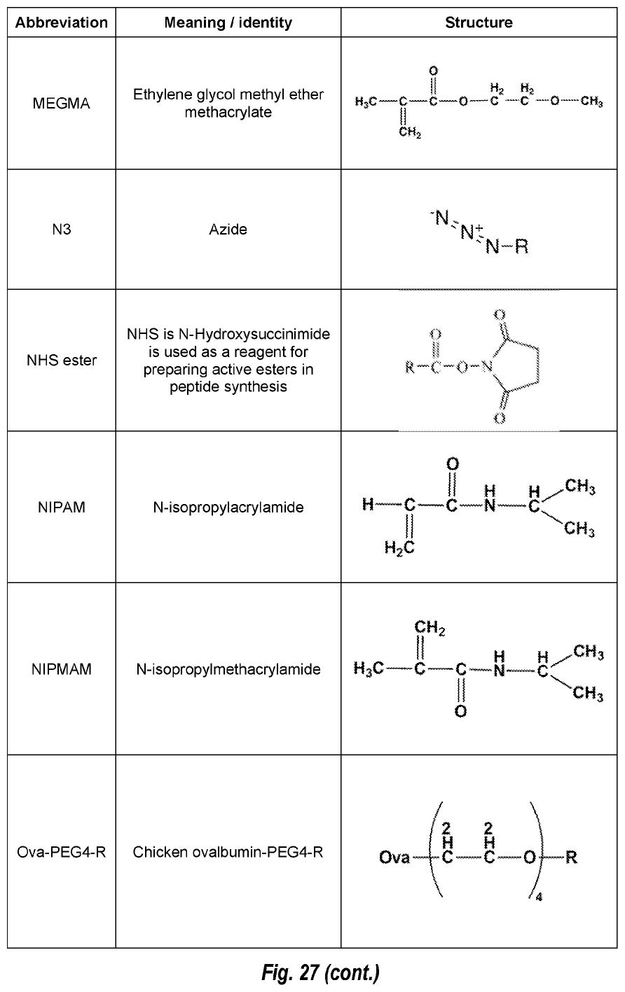

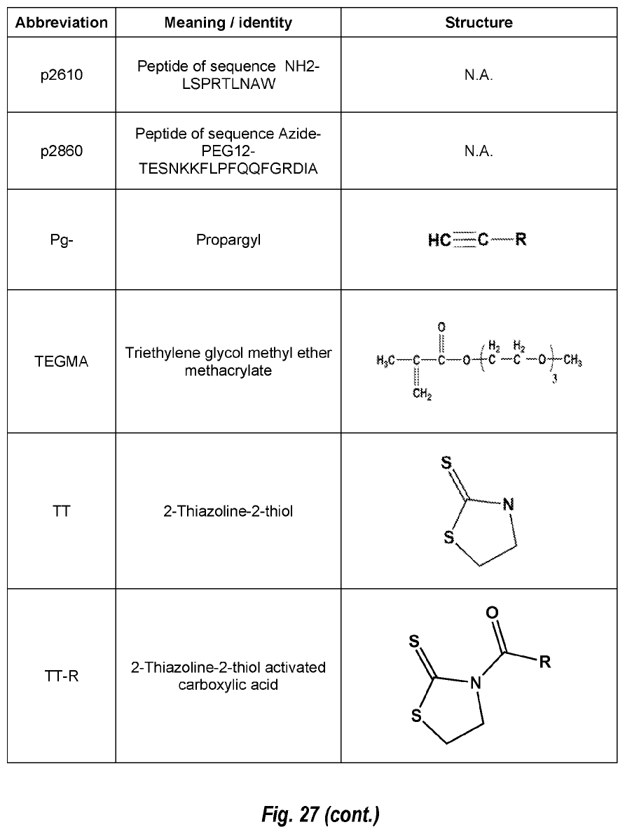

THP1 cells were also used for assessment of innate immune activation using thermo-responsive polymer micelle in the absence of any drug molecule CN-p[(NIPMAM)_{f1}-co-(BnMAM)_{f2}]-b-p(HPMA)ₐ-N3 (Compound 229), which was demonstrated to not stimulate innate immune recognition as measured by NF-κB activation in THP1 human monocyte cells (Invivogen NF-κB SEAP Reporter Monocytes) at concentrations up to 20 mg/mL in culture medium (FIG. 22). In contrast, the positive control for NF-κB activation, small molecule immunostimulant TLR7/8 agonist 2B, stimulated a strong NF-κB response.

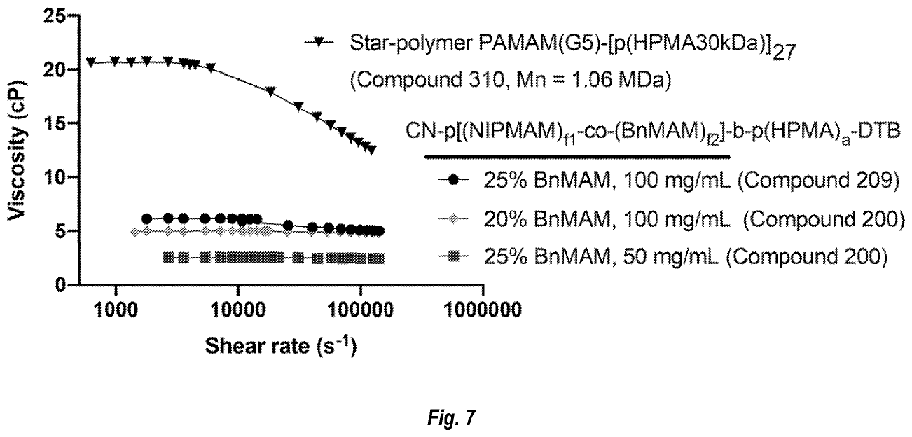

A potential concern for ocular drug delivery systems and other applications where injection of high concentration solution may be beneficial is the viscosity of the solution being injected, which must be sufficiently low (<100 cP and ideally <25 cP) to enable injection through narrow gauge needles. For comparison, high molecular weight Star-polymer PAMAM(G5)-[p(HPMA30 kDa)]₂₇ (Compound 310) was prepared as a negative example of a high molecular weight polymer with appreciable viscosity in aqueous solution. To assess viscosity of high concentrations of polymers, thermo-responsive polymer samples of structure CN-p[(NIPMAM)_{f1}-co-(BnMAM)_{f2}]-b-p(HPMA)ₐ-DTB with 20 mol % BnMAM (Compound 200) and 25 mol % BnMAM (Compound 209) were resuspended from powder into PBS, 150 mM, pH 7.4 at concentrations of 50 or 100 mg/mL using bath sonication. High molecular weight Star-polymer PAMAM(G5)-[p(HPMA30 kDa)]₂₇ (Compound 310) was similarly dissolved from powder into PBS, 150 mM, pH 7.4 at a concentration of 100 mg/mL. These solutions were analyzed using a VROC Initium by Rheosense (San Ramon, California) using E02 and B05 flow channels at shear rates between 630-144,000 sec⁻¹ to mimic shear rate observed while injecting through narrow gauge syringe needles at a temperature of 20° C. to mimic room temperature during injection (FIG. 7). Viscosity measurements demonstrated that the thermo-responsive polymers at 20° C. had a viscosity of approximately 5 cP at a concentration of 100 mg/mL and were effectively Newtonian fluids. In contrast, the high molecular Star-polymer had a viscosity of approximately 20 cP at 100 mg/mL and was shear thinning at shear rates above 10,000 s⁻¹. These results demonstrated the unimolecular behaviour of the thermo-responsive micelle at 20° C., as a macromolecular micelle assembly existing at 100 mg/mL concentration would be expected to have a dynamic viscosity closer to that of the high molecular weight Star-polymer and exhibit shear thinning viscosity.

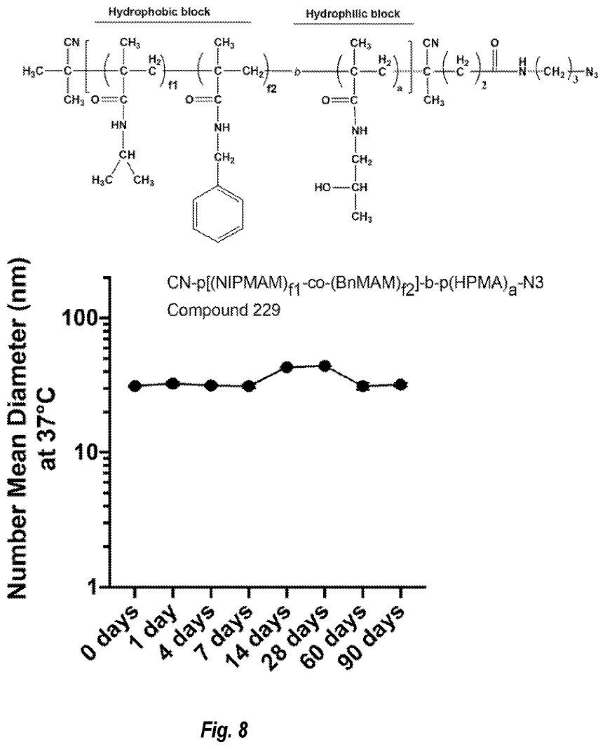

A potential concern for any nanoparticle solution being injected is stability over time in aqueous solution. For ocular application in the vitreous, stability over a period of months may be desirable. Stability of a temperature-responsive amphiphilic diblock copolymer, CN-p[(NIPMAM)_{f1}-co-(BnMAM)_{f2}]-b-p(HPMA)ₐ-N3 (i.e., Compound 229) in PBS, 150 mM, pH 7.4 at a concentration of 5 mg/mL was assessed over a time period of 90 days during which the polymer sample was incubated at 37° C. (FIG. 8). These results demonstrate the thermo-responsive polymer CN-p[(NIPMAM)_{f1}-co-(BnMAM)_{f2}]-b-p(HPMA)ₐ-N3 does not aggregate and does not undergo sufficient amide bond degradation to induce micelle destruction over a period of at least 90 days. These properties may be beneficial in a therapeutic injected intravitreously or by other routes.

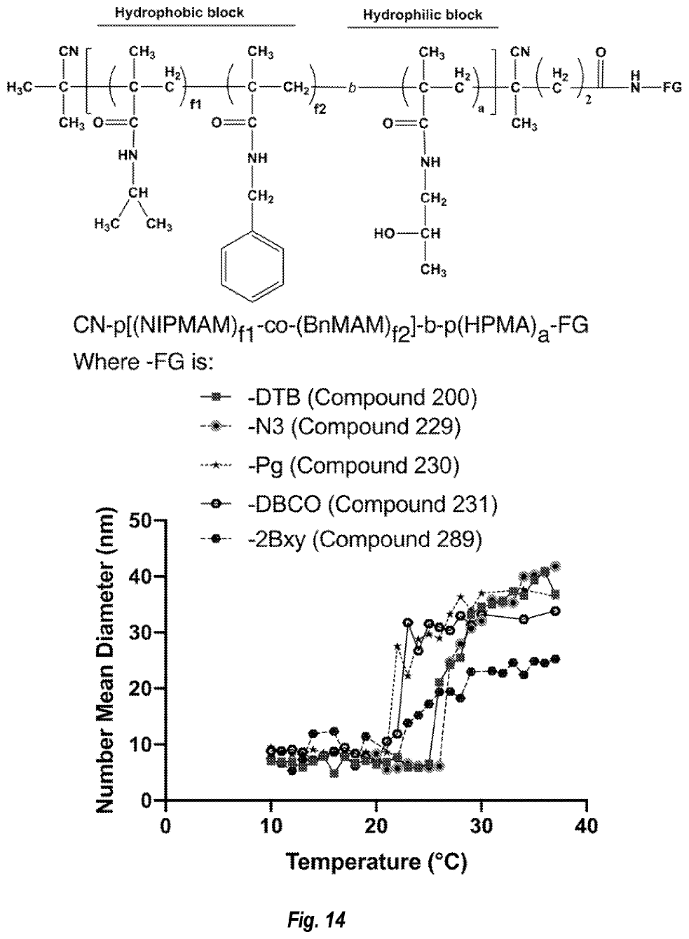

The ability to functionalize the hydrophilic terminus of a polymer with different functional groups (FG) or small molecules without influencing the overall hydrodynamic property of the resulting micelle may be beneficial for purposes of conjugation of drug molecules. Thermo-responsive polymer micelles with a hydrophilic terminus of DTB, N3, Pg, DBCO or 2BXy were shown to all be thermo-responsive with similar thermo-responsive transition temperatures and micelle hydrodynamic diameters (FIG. 14). Diblock thermo-responsive polymers with hydrophilic terminal groups (N3, Pg, DTB) typically yield a slightly larger micelle diameter compared to polymers with a hydrophobic terminal group (DBCO, 2Bxy), which also tended to have a slightly lower transition temperature.

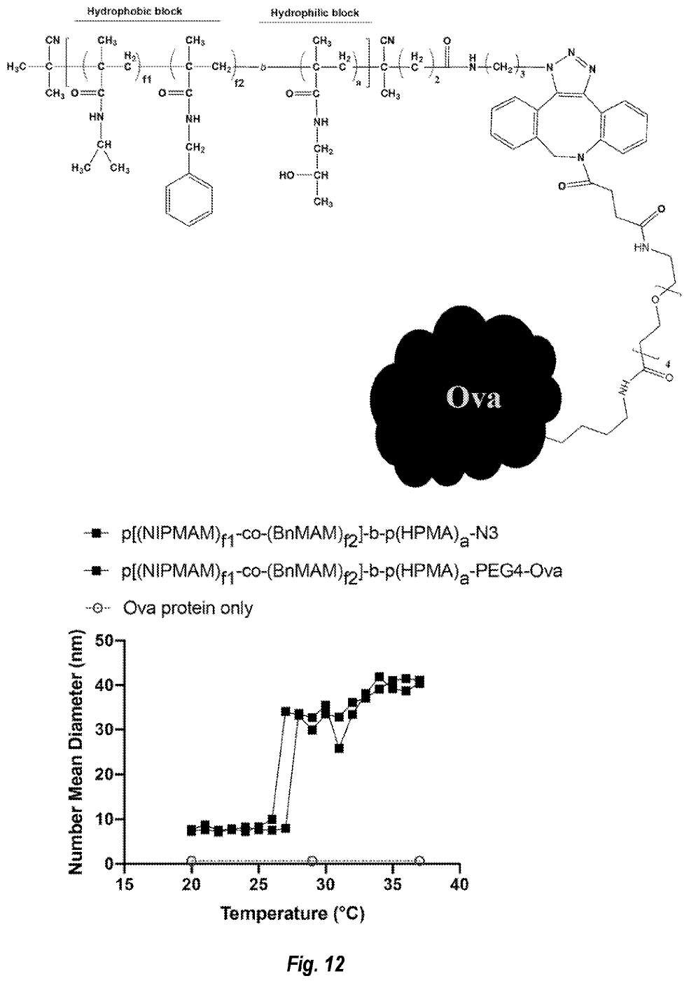

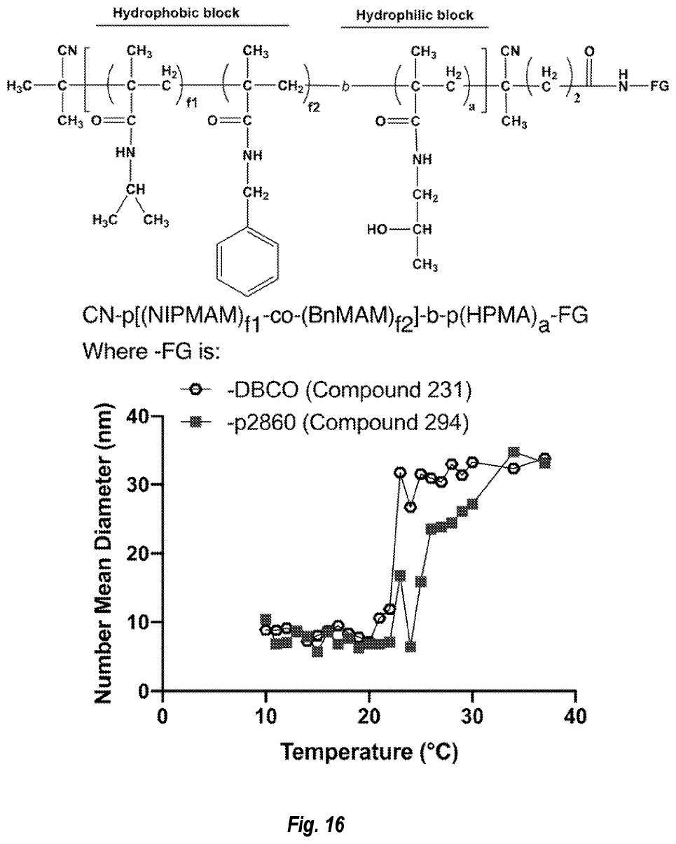

Attachment of hydrophilic biomolecules also impacted the transition temperature and size of particles formed by amphiphilic block copolymers. Accordingly, whereas attachment of hydrophilic small molecules or peptides to amphiphilic block copolymers had minimal impact on transition temperature and particle size, attachment of hydrophilic proteins and glycoproteins with greater than 10 kDa molecular weight appreciably impacted transition temperature, with proteins and glycoproteins greater than 40 kDa molecular weight resulting in an ˜5-10° C. increase in the transition temperature of amphiphilic block copolymers with between about 0.5:1 to 2.5:1 block ratios. A non-limiting explanation is that attachment of large biomolecules sterically hinders particle formation and/or has a solubilizing effect resulting in increased transition temperature. Attachment of small molecules was demonstrated to have minimal impact on the thermo-responsive transition temperature or micelle diameter (FIG. 14). Attachment of small peptides to co-monomers of the hydrophilic block or hydrophilic terminus was similarly demonstrated to have minimal influence on thermo-responsive transition temperature or micelle diameter (FIG. 16). Finally, conjugation of a globular protein (ovalbumin) to the hydrophilic terminus was demonstrated to increase the thermo-responsive transition temperature but only minimally influence micelle diameter (FIG. 12, Table 16).

Attachment of small molecule drugs to a thermo-responsive polymer may be beneficial for treating a variety of diseases. Attachment was performed using either MA-b-Ala-TT reactive co-monomer (Compound 11) with small molecule hydrophobic drugs 2BXy (Compound 32) and diABZI-pip (Compound 33) for polymer compounds described (Table 13, Table 14, Table 15). Small molecule hydrophobic drugs were attached to the hydrophobic block of a single block polymer Pg-p[(NIPMAM)_{f1}-co-(Ma-b-Ala-TT)ₑ]-DBCO (Compound 137), the hydrophobic block of a di-block thermo-responsive polymer CN-p[(NIPMAM)_{f}1-co-(MA-b-Ala-TT)ₑ]-b-p(HPMA)ₐ-Pg (Compound 233) and the hydrophilic block of a di-block thermo-responsive polymer CN-p[(NIPMAM)_{f1}-co-(BnMAM)_{f2}]-b-p[(HPMA)ₐ-co-(Ma-b-Ala-TT)ₑ]-Pg (Compound 236).

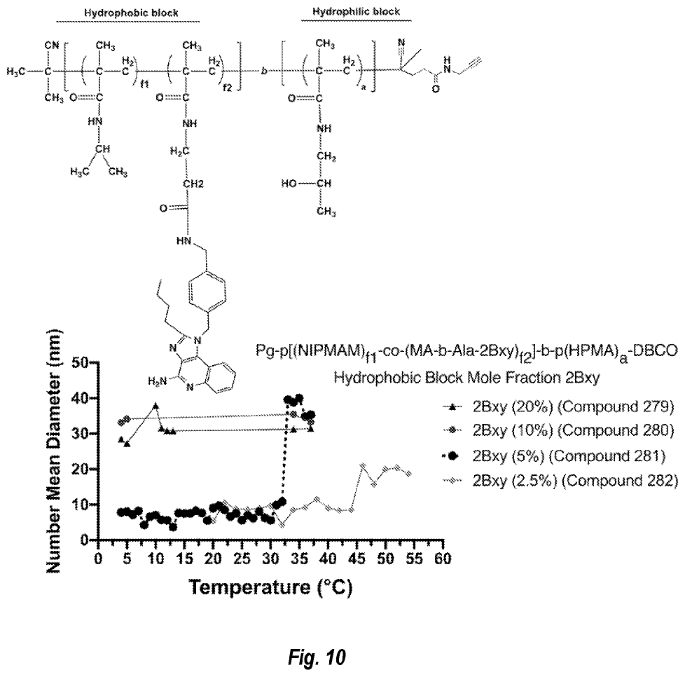

Reaction of CN-p[(NIPMAM)_{f1}-co-(MA-b-Ala-TT)ₑ]-b-p(HPMA)ₐ-Pg (Compound 233) with 2Bxy was demonstrated to yield either permanent micelles, thermo-responsive micelles or thermo-responsive unstable micelles depending on the mol % modification of the hydrophobic block with the small molecule drug (FIG. 10, Table 15). Reaction with 10 and 20 mol % 2Bxy co-monomer to yield CN-p[(NIPMAM)_{f1}-co-(MA-b-Ala-2Bxy)ₑ]-b-p(HPMA)ₐ-Pg (Compound 279 and Compound 280) yielded permanent micelles with no transition temperature between 4-37° C. Hydrophobic block modification with 5 mol % 2Bxy (Compound 281) yielded a thermo-responsive micelle with transition temperature of 33° C. Hydrophobic block modification with 2.5 mol % 2Bxy (Compound 282) yielded a thermo-responsive micelle with transition temperature of 46° C. These results demonstrate there is an optimal mole fraction of hydrophobic co-monomer drug that can be used to enable thermo-responsivity between 20-37° C.

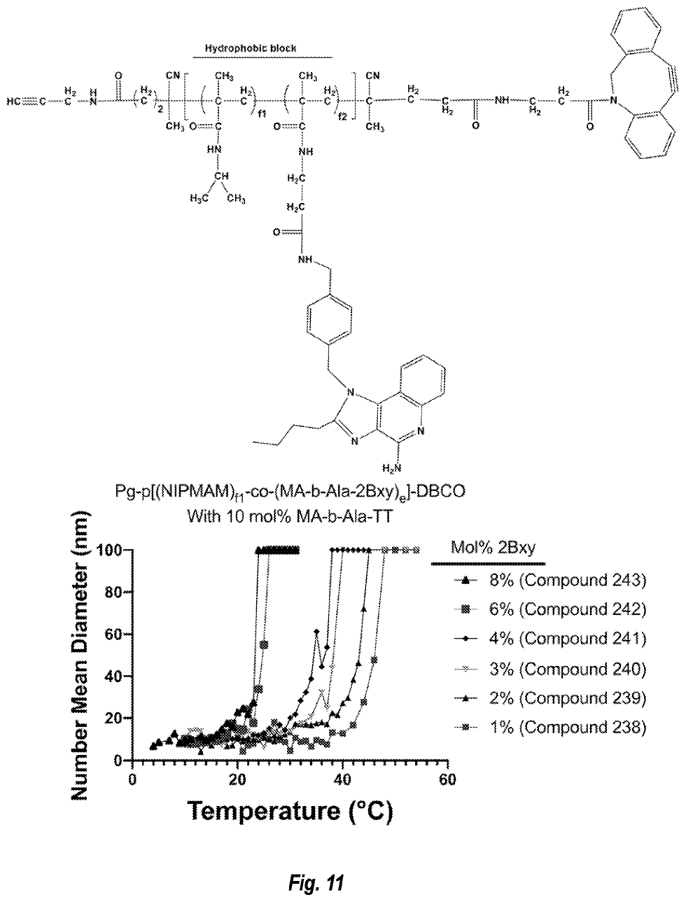

Reaction of Pg-p[(NIPMAM)_{f1}-co-(Ma-b-Ala-TT)ₑ]-DBCO (Compound 137) with 2Bxy was demonstrated to enable the thermo-responsive transition temperature where the single block polymer underwent aggregation dependent on the mol % of 2Bxy reacted to the polymer (FIG. 11, Table 15). Hydrophobic block modification with between 3-6 mol % 2Bxy co-monomer (Compounds 240-242) yielded a single block polymer with a transition temperature between 20-37° C. These results demonstrate there is an optimal mole fraction of hydrophobic co-monomer drug that can be used to enable thermo-responsivity between 20-37° C. Diameters detected >100 nm are shown as 100 nm for display purposes as the single block polymer forms an amorphous diameter aggregate.

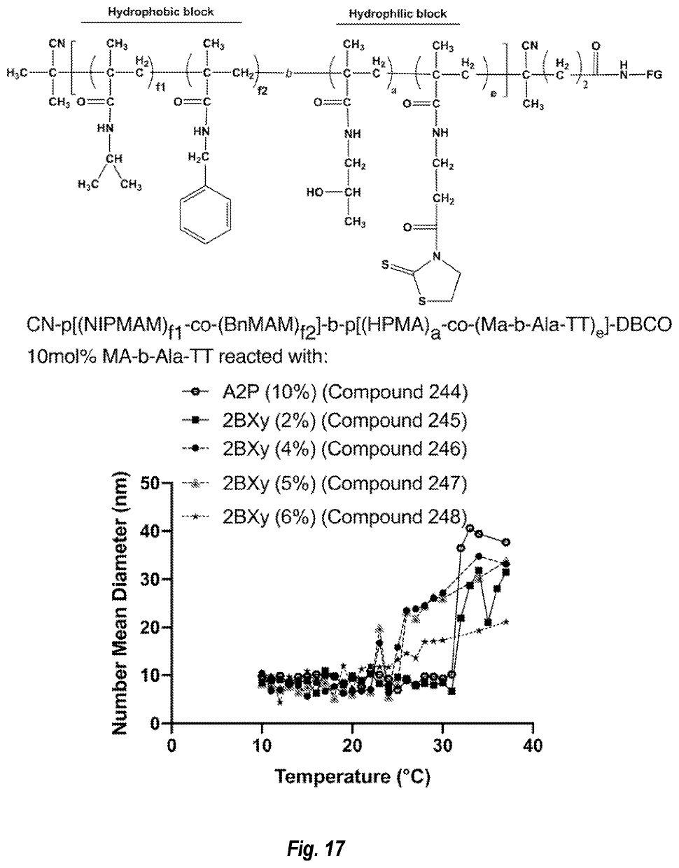

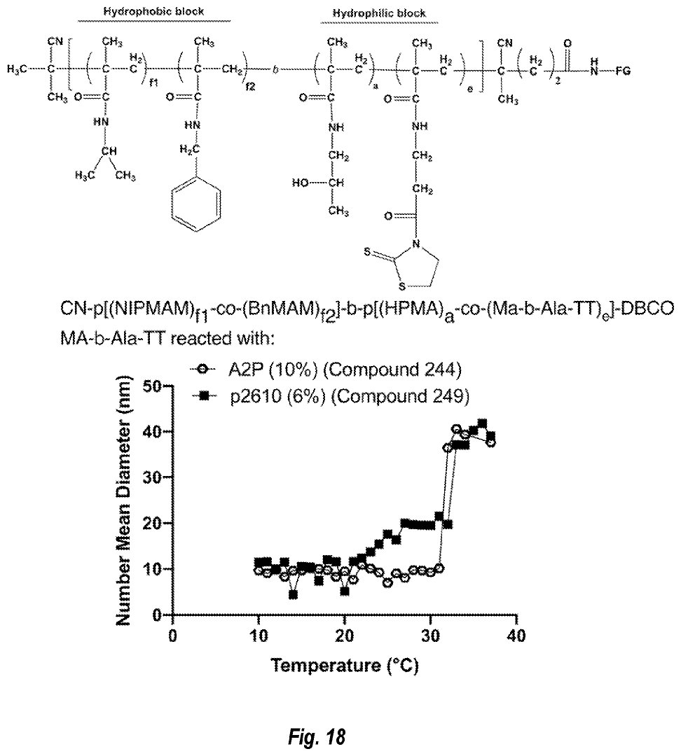

Reaction of di-block thermo-responsive polymer CN-p[(NIPMAM)_{f1}-co-(BnMAM)_{f2}]-b-p[(HPMA)ₐ-co-(Ma-b-Ala-TT)ₑ]-Pg (Compound 236) with 2BXy was demonstrated minimally influence micelle diameter and thermo-responsive transition temperature (FIG. 17, Table 15). The starting polymer had 10 mol % of the hydrophilic block as reactive co-monomer MA-b-Ala-TT, which was first reacted with a defined mole ratio of 2Bxy to yield fractionally modified hydrophilic block, followed by reaction with amino-2-propanol (CAS 78-96-6) to mimic HPMA co-monomers. Hydrophilic block modification with 2-5 mol % 2Bxy (Compounds 245, 246, 247) did not change the effective hydrodynamic diameter of the micelle at 37° C. and only yielded thermo-responsive polymers with defined transition temperatures between a unimer and micelle state. Hydrophilic block modification with 6 mol % 2Bxy yielded a polymer (Compound 248) that still appeared to form micelles but lacked a clear transition temperature and formed micelles of smaller diameter.

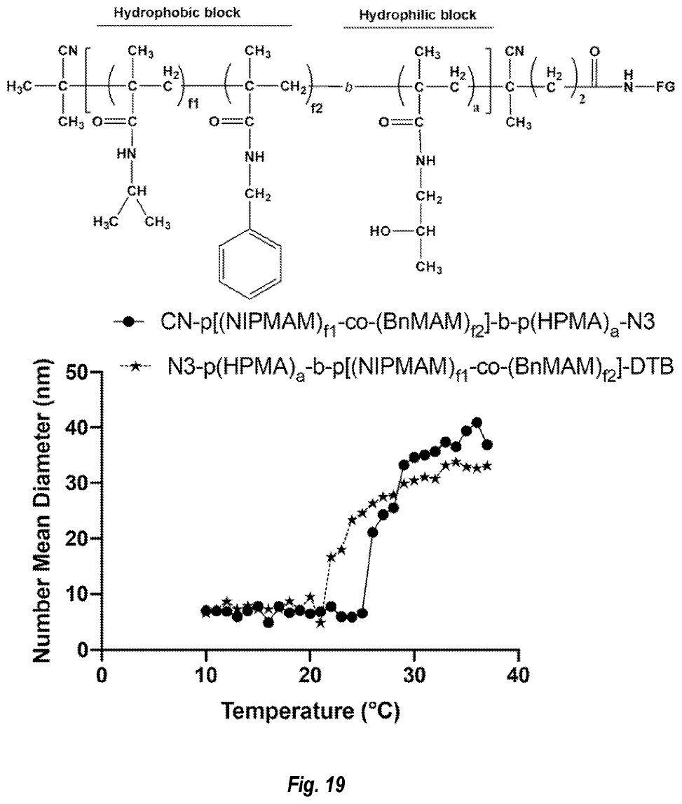

The direction of RAFT polymerization synthesis may provide benefits in production of polymers where the end terminal functionality is higher starting with either the hydrophilic or hydrophobic block of a di-block polymer. Altering the route of synthesis to synthesize the hydrophobic block of a thermo-responsive di-block polymer either first or second was demonstrated not by itself to influence micelle diameter or thermo-responsive transition temperature (FIG. 19, Table 16).

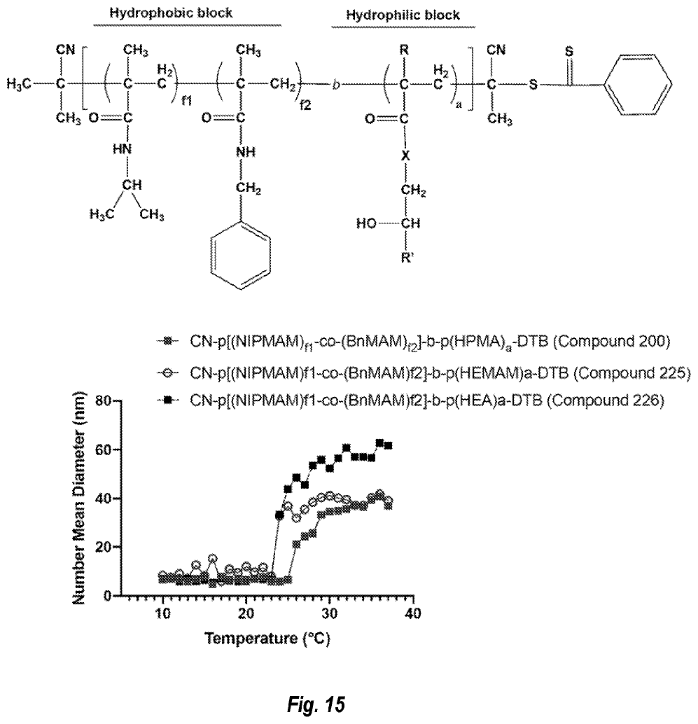

Alternative hydrophilic block monomers than HPMA may be preferred in some embodiments. Use of hydrophilic block monomers of HEA and HEMAM were demonstrated to yield functionally similar thermo-responsive di-block polymers that had similar thermo-responsive transition temperatures and micelle diameters to polymers using HPMA as a hydrophilic block (FIG. 15). These results demonstrate that the hydrophilic monomer used is not limited to hydrophilic methacrylamide monomers.

The foregoing disclosure has been described in some detail by way of illustration and example, for purposes of clarity and understanding. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

1. An amphiphilic block copolymer having any one of the formulas D-S—H, S(D)-H, S—H(D), D-S—H—S, D-S—H—S-D, S(D)-H—S, S(D)-H—S(D) or S—H(D)-S, wherein S is a hydrophilic block; H is a hydrophobic block; D is a drug molecule; ( ) denotes that D is bonded directly or indirectly as a side chain or as part of a side chain group to the adjacent S or H; and the hyphen, “-” (or sometimes “-”), denotes that each of the adjacent S, H or D are linked either directly to one another or indirectly to one another via a linker, additionally wherein the hydrophilic block comprises a first hydrophilic monomer and the hydrophobic block comprises a first hydrophobic monomer and a second hydrophobic monomer.

2. The amphiphilic block copolymer of claim 1, wherein the first hydrophobic monomer is selected from temperature-responsive monomers and the second hydrophobic monomer is selected from hydrophobic monomers comprising an aromatic group.

3. The amphiphilic block copolymer of claim 1 or claim 2, wherein the first hydrophobic monomer comprises (meth)acrylates or (meth)acrylamides of the formula CH₂=CR₁₂—C(O)—R₁₁ (“Formula IV”), or combinations thereof, wherein
each R₁₁ is independently —OR₁₃, —NHR₁₃ or —N(CH₃)R₁₃,
each R₁₂ is independently H or CH₃,
each R₁₃ is a hydrophobic substituent.

4. The amphiphilic block copolymer of claim 3, wherein R₁₃ is an aliphatic group having three or more carbon atoms, which may be linear or branched or saturated or unsaturated, including linear chains such as —(CH₂)ₗCH₃, wherein l is an integer from 3 to 19; branched chains such as CH(CH₃)₂, (CH₂)_{l*}CH(CH₃)₂, wherein l* is an integer from 1 to 11; and cyclic rings, such as (CH₂)ₗ₊(C₅H₉), (CH₂)ₗ₊(C₆H₁₁), (CH₂)ₗ₊(C₇H₁₃) or (CH₂)ₗ₊(C₈H₁₅), wherein l⁺ is an integer from 0 to 6.

5. The amphiphilic block copolymer of any one of claims 1 to 4, wherein the hydrophobic block comprises NIPMAM, NANPP, NVIBA, BEEP or TEGMA, or combinations thereof.

6. The amphiphilic block copolymer of any one of claims 1 to 5, wherein the first hydrophobic monomer is NIPMAM, NANPP, NVIBA, BEEP or TEGMA.

7. The amphiphilic block copolymer of any one of claims 1 to 6, wherein the second hydrophobic monomer comprises (meth)acrylates, or (meth)acrylamides of the formula CH₂=CR₁₂—C(O)—R₁₁ (“Formula IV”), or combinations thereof, wherein
each R₁₁ is independently —OR₁₃, —NHR₁₃ or —N(CH₃)R₁₃,
each R₁₂ is independently H or CH₃,
each R₁₃ is a hydrophobic substituent.

8. The amphiphilic block copolymer of claim 7, wherein R₁₃ is an aliphatic group having three or more carbon atoms, which may be linear or branched or saturated or unsaturated, including linear chains such as —(CH₂)ₗCH₃, wherein l is an integer from 3 to 19; branched chains such as CH(CH₃)₂, (CH₂)ₗ_{^{*}}CH(CH₃)₂, wherein l* is an integer from 1 to 11; and cyclic rings, such as (CH₂)ₗ₊(C₅H₉), (CH₂)ₗ₊(C₆H₁₁), (CH₂)ₗ₊(C₇H₁₃) or (CH₂)ₗ₊(C₈H₁₅), wherein l⁺ is an integer from 0 to 6.

9. The amphiphilic block copolymer of any one of claims 1 to 8, wherein the second hydrophobic monomer comprises aromatic groups.

10. The amphiphilic block copolymer of any one of claims 1 to 9, wherein the second hydrophobic monomer comprises phenyl, fused phenyl or heterocyclic aromatic groups, or combinations thereof.

11. The amphiphilic block copolymer of any one of claims 1 to 10, wherein the hydrophobic block comprises BnMAM.

12. The amphiphilic block copolymer of any one of claims 1 to 11, wherein the first hydrophobic monomer is NIPMAM and the second hydrophobic monomer is BnMAM.

13. The amphiphilic block copolymer of any one of claims 1 to 12, wherein the hydrophobic block is comprised of 50 to 95 mol % of the first hydrophobic monomer and of 5 to 50 mol % of the second hydrophobic monomer.

14. The amphiphilic block copolymer of any one of claims 1 to 13, wherein the hydrophobic block is comprised of 70 to 85 mol % of the first hydrophobic monomer and of 15 to 30 mol % of the second hydrophobic monomer.

15. The amphiphilic block copolymer of any one of claims 1 to 14, wherein the first hydrophilic monomer comprises (meth)acrylates or (meth)acrylamides, of the formula CH₂=CR₂—C(O)—R₁ (“Formula I”), or combinations thereof, wherein
each R₁ is independently —OR₃, —NHR₃ or —N(CH₃)R₃,
each R₂ is independently H or CH₃,
each R₃ is independently H (except for OR₃), CH₃, CH₂CH₃, CH₂CH₂OH, CH₂(CH₂)₂OH, CH₂CH(OH)CH₃, CHCH₃CH₂OH, or (CH₂CH₂O)ᵢH, where i is an integer from 1 to 10.

16. The amphiphilic block copolymer of any one of claims 1 to 15, wherein the hydrophilic block comprises HEA, HEMAM, HPMA, PEG, or combinations thereof.

17. The amphiphilic block copolymer of any one of claims 1 to 16, wherein the first hydrophilic monomer is HEA, HEMAM, HPMA or PEG.

18. An amphiphilic block copolymer having any one of the formulas D-S—H, S(D)-H, S—H(D), D-S—H—S, D-S—H—S-D, S(D)-H—S, S(D)-H—S(D) or S—H(D)-S, wherein S is a hydrophilic block; H is a hydrophobic block; D is a drug molecule; ( ) denotes that D is bonded directly or indirectly as a side chain or as part of a side chain group to the adjacent S or H; and the hyphen, “-” (or sometimes “-”), denotes that each of the adjacent S, H or D are linked either directly to one another or indirectly to one another via a linker, additionally wherein the hydrophilic block comprises a first hydrophilic monomer and the hydrophobic block comprises a first hydrophobic monomer comprising at least one aromatic group.

19. The amphiphilic block copolymer of claim 18, wherein the amphiphilic block copolymer comprises a second hydrophilic monomer present in the hydrophobic block, wherein the second hydrophilic monomer comprises (meth)acrylates or (meth)acrylamides, of the formula CH₂=CR₂—C(O)—R₁ (“Formula I”), or combinations thereof, wherein
each R₁ is independently —OR₃, —NHR₃ or —N(CH₃)R₃,
each R₂ is independently H or CH₃,
each R₃ is independently H (except for OR₃), CH₃, CH₂CH₃, CH₂CH₂OH, CH₂(CH₂)₂OH, CH₂CH(OH)CH₃, CHCH₃CH₂OH, or (CH₂CH₂O)ᵢH, where i is an integer from 1 to 5.

20. The amphiphilic block copolymer of claim 18 or 19, wherein the hydrophobic block comprises HEA, HEMAM, HPMA, PEG, or combinations thereof.

21. The amphiphilic block copolymer of claim 19 or 20, wherein the second hydrophilic monomer is HEA, HEMAM, HPMA or PEG.

22. The amphiphilic block copolymer of any one of claims 18 to 21, wherein the hydrophobic block further comprises a second hydrophobic monomer.

23. The amphiphilic block copolymer of claim 22, wherein the second hydrophobic monomer is a temperature-responsive monomer.

24. The amphiphilic block copolymer of claim 22 or 23, wherein the second hydrophobic monomer comprises (meth)acrylates or (meth)acrylamides of the formula CH₂=CR₁₂—C(O)—R₁₁ (“Formula IV”), or combinations thereof, wherein
each R₁₁ is independently —OR₁₃, —NHR₁₃ or —N(CH₃)R₁₃,
each R₁₂ is independently H or CH₃,
each R₁₃ is a hydrophobic substituent.

25. The amphiphilic block copolymer of claim 24, wherein R₁₃ is an aliphatic group having three or more carbon atoms, which may be linear or branched or saturated or unsaturated, including linear chains such as —(CH₂)ₗCH₃, wherein l is an integer from 3 to 19; branched chains such as CH(CH₃)₂, (CH₂)ₗ_{^{*}}CH(CH₃)₂, wherein l* is an integer from 1 to 11; and cyclic rings, such as (CH₂)₁₊(C₅H₉), (CH₂)ₗ₊(C₆H₁₁), (CH₂)₁₊(C₇H₁₃) or (CH₂)₁₊(CH₁₅), wherein l⁺ is an integer from 0 to 6.

26. The amphiphilic block copolymer of any one of claims 18 to 25, wherein the hydrophobic block comprises NIPMAM, NANPP, NVIBA, BEEP or TEGMA, or combinations thereof.

27. The amphiphilic block copolymer of any one of claims 22 to 26, wherein the second hydrophobic monomer is NIPMAM, NANPP, NVIBA, BEEP or TEGMA.

28. The amphiphilic block copolymer of any one of claims 18 to 27, wherein the first hydrophobic monomer comprises (meth)acrylates, or (meth)acrylamides of the formula CH₂=CR₁₂—C(O)—R₁₁ (“Formula IV”), or combinations thereof, wherein
each R₁₁ is independently —OR₁₃, —NHR₁₃ or —N(CH₃)R₁₃,
each R₁₂ is independently H or CH₃,
each R₁₃ is a hydrophobic substituent comprising at least one aromatic group.

29. The amphiphilic block copolymer of any one of claims 18 to 28, wherein the aromatic group of the first hydrophobic monomer comprises phenyl, fused phenyl or heterocyclic aromatic groups, or combinations thereof.

30. The amphiphilic block copolymer of any one of claims 18 to 29, wherein the hydrophobic block comprises BnMAM.

31. The amphiphilic block copolymer of any one of claims 18 to 30, wherein the first hydrophobic monomer is BnMAM.

32. The amphiphilic block copolymer of any one of claims 18 to 31, wherein the first hydrophobic monomer is BnMAM and the second hydrophobic monomer is NIPMAM, NANPP, NVIBA, BEEP or TEGMA.

33. The amphiphilic block copolymer of any one of claims 18 to 32, wherein the hydrophobic block is comprised of 10 to 100 mol % of the first hydrophobic monomer.

34. The amphiphilic block copolymer of any one of claims 18 to 33, wherein the hydrophobic block is comprised of 25 to 75 mol % of the first hydrophobic monomer.

35. The amphiphilic block copolymer of any one of claims 18 to 34, wherein the first hydrophilic monomer comprises (meth)acrylates or (meth)acrylamides, of the formula CH₂=CR₂—C(O)—R₁ (“Formula I”), or combinations thereof, wherein
each R₁ is independently —OR₃, —NHR₃ or —N(CH₃)R₃,
each R₂ is independently H or CH₃,
each R₃ is independently H (except for OR₃), CH₃, CH₂CH₃, CH₂CH₂OH, CH₂(CH₂)₂OH, CH₂CH(OH)CH₃, CHCH₃CH₂OH, or (CH₂CH₂O)ᵢH, where i is an integer from 1 to 5.

36. The amphiphilic block copolymer of any one of claims 18 to 35, wherein the hydrophilic block comprises HEA, HEMAM, HPMA, PEG, or combinations thereof.

37. The amphiphilic block copolymer of any one of claims 18 to 36, wherein the first hydrophilic monomer is HEA, HEMAM, HPMA or PEG.

38. An amphiphilic block copolymer having any one of the formulas D-S—H, S(D)-H, S—H(D), D-S—H—S, D-S—H—S-D, S(D)-H—S, S(D)-H—S(D) or S—H(D)-S, wherein S is a hydrophilic block; H is a hydrophobic block; D is a drug molecule; ( ) denotes that D is bonded directly or indirectly as a side chain or as part of a side chain group to the adjacent S or H; and the hyphen, “-” (or sometimes “-”), denotes that each of the adjacent S, H or D are linked either directly to one another or indirectly to one another via a linker, additionally wherein the hydrophilic block comprises a first hydrophilic monomer and the hydrophobic block comprises a first hydrophobic monomer and a second hydrophobic monomer, wherein the first hydrophobic monomer comprises temperature-responsive monomers and the second hydrophobic monomer comprises hydrophobic monomers comprising a fluorinated aromatic ring, or a fused aromatic ring, or combinations thereof.

39. The amphiphilic block copolymer of claim 38, wherein the hydrophobic block comprises NIPMAM, NANPP, NVIBA, BEEP or TEGMA, or combinations thereof.

40. The amphiphilic block copolymer of claim 38 or 39, wherein the first hydrophobic monomer is NIPMAM, NANPP, NVIBA, BEEP or TEGMA.

41. The amphiphilic block copolymer of any one of claims 38 to 40, wherein the hydrophobic block comprises N-3,4,5-trifluorobenzyl methacrylamide, N-2,3,4,5,6 pentafluorobenzyl methacrylamide, N-trifluoromethylbenzyl methacrylamide and N-bitrifluoromethylbenzyl methacrylamide.

42. The amphiphilic block copolymer of any one of claims 38 to 41, wherein the second hydrophobic monomer is chosen from N-3,4,5-trifluorobenzyl methacrylamide, N-2,3,4,5,6 pentafluorobenzyl methacrylamide, N-trifluoromethylbenzyl methacrylamide and N-bitrifluoromethylbenzyl methacrylamide.

43. The amphiphilic block copolymer of any one of claims 38 to 42, wherein the first hydrophobic monomer is NIPMAM and the second hydrophobic monomer is N-3,4,5-trifluorobenzyl methacrylamide, N-2,3,4,5,6 pentafluorobenzyl methacrylamide, N-trifluoromethylbenzyl methacrylamide or N-bitrifluoromethylbenzyl methacrylamide.

44. The amphiphilic block copolymer of any one of claims 38 to 43, wherein the hydrophobic block is comprised of 80 to 99 mol % of the first hydrophobic monomer and of 1 to 20 mol % of the second hydrophobic monomer.

45. The amphiphilic block copolymer of any one of claims 38 to 44, wherein the hydrophobic block is comprised of 90 to 99 mol % of the first hydrophobic monomer and of 1 to 10 mol % of the second hydrophobic monomer.

46. The amphiphilic block copolymer of any one of claims 38 to 45, wherein the hydrophilic block comprises HEA, HEMAM, HPMA, PEG, or combinations thereof.

47. The amphiphilic block copolymer of any one of claims 38 to 46, wherein the first hydrophilic monomer is HEA, HEMAM, HPMA or PEG.

48. The amphiphilic block copolymer of any one of claims 1 to 47, having a degree of polymerization block ratio of hydrophilic block to hydrophobic block of 0.5:1 to 4:1.

49. The amphiphilic block copolymer of any one of claims 1 to 48, having a degree of polymerization block ratio of hydrophilic block to hydrophobic block of 0.75:1 to 3:1.

50. The amphiphilic block copolymer of any one of claims 1 to 49, wherein the amphiphilic block copolymer has a molecular weight of about 5 kDa to about 60 kDa.

51. The amphiphilic block copolymer of any one of claims 1 to 50, wherein the amphiphilic block copolymer has a molecular weight of about 15 kDa to about 50 kDa.

52. The amphiphilic block copolymer of any one of claims 1 to 51, wherein the amphiphilic block copolymer has a molecular weight of about 25 kDa to about 45 kDa.

53. The amphiphilic block copolymer of any one of claims 1 to 52, wherein D is chosen from ocular drugs, steroidal and nonsteroidal anti-inflammatory drugs, senolytic drugs or immunomodulatory drugs.

54. The amphiphilic block copolymer of any one of claims 1 to 53, wherein D is linked directly or indirectly via a linker to an adjacent S or H.

55. The amphiphilic block copolymer of any one of claims 1 to 54, wherein the amphiphilic block copolymer has the formula D-S—H, D-S—H—S or D-S—H—S-D, and D is linked to an end of the hydrophilic block of the amphiphilic block copolymer.

56. The amphiphilic block copolymer of any one of claims 1 to 54, wherein the hydrophilic block further comprises a first reactive monomer that is distributed along the backbone of the hydrophilic block, and wherein the amphiphilic block copolymer has the formula S(D)-H, S(D)-H—S or S(D)-H—S(D) and D is linked to the amphiphilic block copolymer through the first reactive monomer.

57. The amphiphilic block copolymer of any one of claims 1 to 56, wherein the hydrophilic block further comprises a first charged monomer.

58. The amphiphilic block copolymer of any one of claims 1 to 57, wherein the hydrophilic block further comprises at least one negatively charged monomer.

59. The amphiphilic block copolymer of any one of claims 57 or 58, wherein the first charged monomer is a (meth)acrylate or a (meth)acrylamide of the formula CH₂=CR₅—C(O)—R₄ (“Formula II”), wherein
R₄ is —OR₆, —NHR₆ or —N(CH₃)R₆,
R₅ is H or CH₃,
R₆ is OH, (CH₂)ⱼNH₂, (CH₂)ⱼCH(NH₂)COOH, (CH₂)ⱼCOOH, (CH₂)ⱼPO₃H₂, (CH₂)ⱼOPO₃H₂, (CH₂)SO₃H, (CH₂)OSO₃H, (CH₂)ⱼB(OH)₂, CH₂CH₂N(CH₃)₂, CH[CH₂N(CH₃)₂]₂, CH(COOH)CH—CH₂COOH, [CH₂CH(CH₃)O]₅PO₃H₂, (CH₂)₃CH(OPO₃H₂)(CH₂)₂CH(OPO₃H₂)(CH₂)₃CH₃, C(CH₃)₂CH₂SO₃H, and C₆H₄B(OH)₂, wherein each j is an integer from 1 to 6.

60. The amphiphilic block copolymer of any one of claims 57 to 59, wherein the first charged monomer is wherein R₄=—OR₆, R₅=CH₃ and R₆=OH.

61. The amphiphilic block copolymer of any one of claims 1 to 60, wherein the hydrophilic block further comprises a reactive monomer.

62. The amphiphilic block copolymer of claim 61, wherein the reactive monomer comprises azide, alkyne, tetrazine, transcyclooctyne (TCO), protected hydrazine, ketone, aldehyde, hydroxyl, isocyanate, isothiocyanate, activated carboxylic acid, protected maleimide, thiol and/or amine groups.

63. The amphiphilic block copolymer of claim 61 or 62, wherein the reactive monomer comprises a (meth)acrylate or a (meth)acrylamide of the formula CH₂=CR₈—C(O)—R₇ (“Formula III”), wherein,
R₇ is —OR₉, —NHR₉ or —N(CH₃)R₉,
R₈ is H or CH₃, and
R₉ is (CH₂)ₖR₁₀, (CH₂)ₖC(O)NHR₁₀, (CH₂CH₂O)ₖCH₂CH₂C(O)NHR₁₀, where k is an integer from 0 to 6, and
each R₁₀ is independently chosen from (CH₂)ₕ-FG, (CH₂CH₂O)ₕCH₂CH₂—FG or (CH₂CH₂O)ₕCH₂CH₂—FG, where h is an integer from 0 to 10, and FG is any functional group

64. The amphiphilic block copolymer of claim 63, wherein FG is carboxylic acid, activated carboxylic acids, anhydride, amine, protected amines, OSi(CH₃), CCH, N₃, propargyl, halogen, an olefin, an endo cyclic olefins, CN, OH, or epoxy.

65. The amphiphilic block copolymer of any one of claims 63 to 64, wherein in the compound of Formula III R₇ is NHR₉, R₈ is CH₃, R₉ is (CH₂)ₖC(O)NHR₁₀, k is equal to 2 and R₁₀ is propargyl.

66. The amphiphilic block copolymer of any one of claims 1 to 65, wherein the hydrophilic block further comprises a reactive monomer linked to a CD22 agonist.

67. The amphiphilic block copolymer of any one of claims 1 to 66, wherein the amphiphilic block copolymer exists as unimers at concentrations greater than 50 mg/mL in aqueous solutions, and wherein the amphiphilic block copolymer exists as particles at concentrations of less than or equal to 50 mg/mL.

68. The amphiphilic block copolymer of any one of claims 1 to 67, wherein the amphiphilic block copolymer exists as unimers in aqueous solutions below a transition temperature, and wherein the amphiphilic block copolymer exists as particles in aqueous solutions above the transition temperature.

69. The amphiphilic block copolymer of claim 68, wherein the transition temperature is 1° C. or more to 37° C. or lower.

70. The amphiphilic block copolymer of claim 68 or 69, wherein the transition temperature is about 20° C. to about 34° C.

71. The amphiphilic block copolymer of any one of claims 68 to 70, wherein the particles are about 20 nm to 200 nm in diameter.

72. The amphiphilic block copolymer of any one of claims 68 to 71, wherein the particles are about 30 nm to 80 nm in diameter.

73. The amphiphilic block copolymer of any one of claims 68 to 72, wherein the particles are about 30 nm to 60 nm in diameter.

74. A solution comprising an aqueous solvent and unimers comprising the amphiphilic block copolymer of any one of claims 1 to 73.

75. The solution of claim 74, wherein the concentration of unimers is greater than 50 mg/mL.

76. The solution of claim 74, wherein the concentration of unimers is less than or equal to 50 mg/mL, and wherein the unimers form particles.

77. The solution of any one of claims 74 to 76, wherein the temperature of the solution is not higher than a transition temperature.

78. The solution of any one of claims 74 to 76, wherein the temperature of the solution is higher than a transition temperature, and wherein the unimers form particles.

79. The solution of claim 77 or 78, wherein the transition temperature is 1° C. or more to 37° C. or lower.

80. The solution of any one of claims 77 to 79, wherein the transition temperature is about 20° C. to about 34° C.

81. A solution comprising an aqueous solvent and particles comprising the amphiphilic block copolymer of any one of claims 1 to 73.

82. The solution of claim 81, having a concentration of unimers in the form of particles of less than or equal to 50 mg/mL.

83. The solution of claim 81 or 82, wherein the solution temperature is above a transition temperature.

84. The solution of claim 83, wherein the transition temperature is 1° C. or more to 37° C. or lower.

85. The solution of claim 83 or 84, wherein the transition temperature is about 20° C. to about 34° C.

86. The solution of any one of claims 76, and 78 to 85, wherein the particles have a diameter of about 20 nm to about 200 nm.

87. The solution of any one of claims 76, and 78 to 86, wherein the particles have a diameter of about 30 nm to about 80 nm.

88. The solution of any one of claims 76, and 78 to 87, wherein the particles have a diameter of about 30 nm to about 60 nm.

89. A method of delivering a drug comprising administering the amphiphilic block copolymer of any one of claims 1 to 73 or the solution of any one of claims 74 to 88 to the subject.

90. A method of delivering a drug to a subject in need of treatment comprising administering the amphiphilic block copolymer of any one of claims 1 to 73 or the solution of any one of claims 74 to 88 to the subject.

91. The method of claim 90, wherein administering is an ocular, intravitreal, suprachoroidal, intrabursal, intraarticular, periarticular, intraperitoneal, intrapericardial, intraperipleural, intrathecal, intraventricular, intravenous, subcutaneous or intradermal injection.

92. The method of claim 90 or 91, wherein the amphiphilic block copolymer or the solution is injected into a body cavity.

93. The method of any one of claims 90 to 92, wherein the amphiphilic block copolymer or the solution is injected into the eye or the knee.

94. The method of any one of claims 89 to 93, wherein the solution that is administered has a concentration of unimers greater than 50 mg/mL before administration.

95. The method of any one of claims 89 to 94, wherein the solution that is administered has a concentration of unimers of 50 mg/mL or less after administration.

96. The method of any one of claims 89 to 95, wherein the amphiphilic block copolymer or unimers exist in the form of particles after administration.
